(19) United States
(12) Patent Application Publication
(10) Pub. No.: US 2024/0287204 A1
(43) Pub. Date: Aug. 29, 2024

(54) ANTI-MERTK ANTIBODIES AND METHODS OF USE THEREOF

(71) Applicant: ALECTOR LLC, South San Francisco, CA (US)

(72) Inventors: Seung-Joo LEE, Benicia, CA (US); Spencer Liang, San Mateo, CA (US); Angie YEE, San Francisco, CA (US); Marina ROELL, Concord, CA (US); Arnon ROSENTHAL, Woodside, CA (US)

(21) Appl. No.: 18/392,569

(22) Filed: Dec. 21, 2023

Related U.S. Application Data
(62) Division of application No. 17/118,921, filed on Dec. 11, 2020, now Pat. No. 11,897,968.
(60) Provisional application No. 63/121,773, filed on Dec. 4, 2020.
(60) Provisional application No. 63/016,821, filed on Apr. 28, 2020.
(60) Provisional application No. 62/947,855, filed on Dec. 13, 2019.

(51) Int. Cl. C07K 16/40; A61K 39/00; A61P 35/00; C07K 16/28

(52) U.S. Cl. CPC C07K 16/40; A61P 35/00; C07K 16/2818; C07K 16/2827; A61K 2039/507; C07K 2317/56; C07K 2317/622; C07K 2317/76

(57) ABSTRACT

The present disclosure is generally directed to compositions that include antibodies, e.g., monoclonal, antibodies, antibody fragments, etc., that specifically bind a MerTK polypeptide, e.g., a mammalian MerTK or human MerTK, and use of such compositions in treating an individual in need thereof.

### CROSS REFERENCE TO RELATED APPLICATIONS

This application is a divisional application of U.S. application Ser. No. 17/118,921, filed Dec. 11, 2020, which claims the benefit of U.S. Provisional Application No. 62/947,855, filed Dec. 13, 2019, U.S. Provisional Application No. 63/016,821, filed Apr. 28, 2020, and U.S. Provisional Application No. 63/121,773, filed Dec. 4, 2020, each of which is herein incorporated by reference in its entirety.

### SUBMISSION OF SEQUENCE LISTING ON XML FILE

The content of the following submission on XML file is incorporated herein by reference in its entirety: a computer readable form (CRF) of the Sequence Listing (file name: 4503_0100004_SeqListing_ST26.xml, date created: Dec. 18, 2023, size: 636,944 bytes).

### FIELD OF THE PRESENT DISCLOSURE

The present disclosure relates to anti-MerTK antibodies and uses (e.g., therapeutic uses) of such antibodies.

### BACKGROUND OF THE PRESENT DISCLOSURE

Mer Tyrosine Kinase (MerTK) belongs to the TAM (Tyro3, Axl, and MerTK) family of receptor tyrosine kinases. MerTK is a single-pass type 1 transmembrane protein with an extracellular domain having two immunoglobulin (Ig)-like and two fibronectin (FN) type III motifs (Graham et al, 2014, Nat Rev Cancer, 14:769-785; Rothlin et al, 2015, Annu Rev Immunol, 33:355-391).

Several ligands of MerTK have been identified, including Protein S (ProS or ProS1), Growth arrest specific gene 6 (Gas6), Tubby, Tubby-like protein 1 (TULP-1), and Galectin-3. MerTK transduces signals from the extracellular space via activation following ligand binding, leading to MerTK tyrosine auto-phosphorylation (Cummings et al, 2013, Clin Cancer Res, 19:5275-5280; Verma et al, 2011, Mol Cancer Ther, 10:1763-1773) and subsequent ERK and AKT-associated signal transduction.

MerTK regulates various physiological processes including cell survival, migration, and differentiation. MerTK ligands ProS and Gas6 contribute to several oncogenic processes, such as cell survival, invasion, migration, chemo-resistance, and metastasis, in which their expression often correlates with poor clinical outcomes. Additionally, MerTK is implicated in numerous cancers, and MerTK or ProS deficiency is associated with anti-tumor effects (Cook et al, 2013, J Clin Invest, 123:3231-3242; Ubil et al, 2018, J Clin Invest, 128:2356-2369; Huey et al, 2016, Cancers, 8:101). However, MerTK is also expressed on retinal pigment epithelial cells and plays a critical role in clearing shed photoreceptor outer segment in the eye; loss of function mutations in MerTK result in retinitis pigmentosa and other retinal dystrophies (see, e.g. Al-khersan et al, Graefes Arch Clim Exp Ophthalmol, 2017, 255:1613-1619; Lorach et al, Nature Scientific Reports, 2018, 8:11312; Audo et al, Human Mutation, Wiley, 2018, 39:997-913; LaVail et al, Adv Exp Med Biol, 2016, 854:487-493).

MerTK plays an essential role in phagocytosis of apoptotic cells (efferocytosis) by phagocytic cells, leading to M2-like macrophage polarization, production of anti-inflammatory cytokines, and promoting an immunosuppressive tumor microenvironment. Reducing efferocytosis by phagocytic cells increases M1-like macrophage polarization, leading to the production of pro-inflammatory cytokines and an immune-active milieu. Modulating efferocytosis can provide an effective means for anti-tumor activity.

Anti-MerTK antibodies have been previously described in, e.g., International Patent Application Publication Nos: WO2020/214995, WO2020/076799, WO2020/106461, WO2020/176497, WO2019/084307, WO2019/005756, WO2016/106221, WO2016/001830, WO2009/062112, and WO2006/058202; and in, e.g., Dayoub and Brekken, 2020, Cell Communications and Signaling, 18:29; Zhou et al, 2020, Immunity, 52:1-17; Kedage et al, 2020, MABS, 12:e1685832; Cummings et al, 2014, Oncotarget, 5:10434-10445.

There is a need for novel therapeutic anti-MerTK antibodies that are effective at treating conditions such as cancer. The present disclosure meets this need by providing anti-MerTK antibodies effective at mediating anti-tumor immunity, reducing efferocytosis, and promoting M1-like macrophage polarization.

All references cited herein, including patent applications and publications, are hereby incorporated by reference in their entirety.

### SUMMARY OF THE PRESENT DISCLOSURE

The present disclosure is generally directed to anti-Mer Tyrosine Kinase (MerTK) antibodies and methods of using such antibodies. The methods provided herein find use in treating an individual having cancer. In some embodiments, the present disclosure provides a method for treating an individual having cancer, the method including administering to the individual in need thereof a therapeutically effective amount of an anti-MerTK antibody.

In one aspect, the present disclosure relates to an isolated anti-MerTK antibody that binds to a MerTK protein, wherein the antibody comprises a heavy chain variable region and a light chain variable region, wherein the heavy chain variable region comprises: a) an HVR-H1 comprising the amino acid sequence of SEQ ID NO:83; b) an HVR-H2 comprising an amino acid sequence selected from the group consisting of SEQ ID NOs: 329, 330, 331, 332, and 333; and c) an HVR-H3 comprising an amino acid sequence selected from the group consisting of SEQ ID NOs: 138, 334, 335, 336, 337, 338, and 339; and wherein the light chain variable region comprises: d) an HVR-L1 comprising an amino acid sequence selected from the group consisting of SEQ ID NOs: 158, 340, 341, 342, 343, and 344; e) an HVR-L2 comprising the amino acid sequence of SEQ ID NO:187; and f) an HVR-L3 comprising the amino acid sequence of SEQ ID NO:210.

In one aspect, the present disclosure relates to an isolated anti-MerTK antibody that binds to a MerTK protein, wherein the antibody comprises: (a) HVR-H1 comprising the amino acid sequence of SEQ ID NO:83; (b) HVR-H2 comprising the amino acid sequence of SEQ ID NO:99; (c) HVR-H3 comprising the amino acid sequence of SEQ ID NO: 138; (d) HVR-L1 comprising the amino acid sequence of SEQ ID NO:158; (e) HVR-L2 comprising the amino acid sequence of SEQ ID NO:187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:334; (d) HVR-L1 comprising the amino acid sequence of SEQ ID NO: 158; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:330; (c) HVR-H3 comprising the amino acid sequence of SEQ ID NO:138; (d) HVR-L1 comprising the amino acid sequence of SEQ ID NO:158; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:138; (d) HVR-L1 comprising the amino acid sequence of SEQ ID NO:340; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:138; (d) HVR-L1 comprising the amino acid sequence of SEQ ID NO:341; (e) HVR-L2 comprising the amino acid sequence of SEQ ID NO:187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:335; (d) HVR-L1 comprising the amino acid sequence of SEQ ID NO:341; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:331; (c) HVR-H3 comprising the amino acid sequence of SEQ ID NO:138; (d) HVR-L1 comprising the amino acid sequence of SEQ ID NO:341; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:138; (d) HVR-L1 comprising the amino acid sequence of SEQ ID NO:342; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:336; (d) HVR-L1 comprising the amino acid sequence of SEQ ID NO:341; (e) HVR-L2 comprising the amino acid sequence of SEQ ID NO:187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:337; (d) HVR-L1 comprising the amino acid sequence of SEQ ID NO:343; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:332; (c) HVR-H3 comprising the amino acid sequence of SEQ ID NO:338; (d) HVR-L1 comprising the amino acid sequence of SEQ ID NO:341; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:333; (c) HVR-H3 comprising the amino acid sequence of SEQ ID NO:334; (d) HVR-L1 comprising the amino acid sequence of SEQ ID NO:158; (e) HVR-L2 comprising the amino acid sequence of SEQ ID NO:187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:332; (c) HVR-H3 comprising the amino acid sequence of SEQ ID NO:336; (d) HVR-L1 comprising the amino acid sequence of SEQ ID NO:341; (e) HVR-L2 comprising the amino acid sequence of SEQ ID NO:187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:339; (d) HVR-L1 comprising the amino acid sequence of SEQ ID NO:344; (e) HVR-L2 comprising the amino acid sequence of SEQ ID NO: 187; and (f) HVR-L3 comprising the amino acid sequence of SEQ ID NO:210; or (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:138; (d) HVR-L1 comprising the amino acid sequence of SEQ ID NO:158; (e) HVR-L2 comprising the amino acid sequence of SEQ ID NO:187; and (f) HVR-L3 comprising the amino acid sequence of SEQ ID NO:210.

In one aspect, the present disclosure relates to an isolated anti-MerTK antibody that binds to a MerTK protein, wherein the antibody comprises a heavy chain variable domain (VH) and a light chain variable domain (VL), wherein the VH and VL are selected from the group consisting of: VH comprising the amino acid sequence of SEQ ID NO: 19 and VL comprising the amino acid sequence of SEQ ID NO:54; VH comprising the amino acid sequence of SEQ ID NO:234 and VL comprising the amino acid sequence of SEQ ID NO:247; VH comprising the amino acid sequence of SEQ ID NO:235 and VL comprising the amino acid sequence of SEQ ID NO:247; VH comprising the amino acid sequence of SEQ ID NO:236 and VL comprising the amino acid sequence of SEQ ID NO:248; VH comprising the amino acid sequence of SEQ ID NO:236 and VL comprising the amino acid sequence of SEQ ID NO:249; VH comprising the amino acid sequence of SEQ ID NO:237 and VL comprising the amino acid sequence of SEQ ID NO:249; VH comprising the amino acid sequence of SEQ ID NO:238 and VL comprising the amino acid sequence of SEQ ID NO:249; VH comprising the amino acid sequence of SEQ ID NO:239 and VL comprising the amino acid sequence of SEQ ID NO:250; VH comprising the amino acid sequence of SEQ ID NO:240 and VL comprising the amino acid sequence of SEQ ID NO:251; VH comprising the amino acid sequence of SEQ ID NO:241 and VL comprising the amino acid sequence of SEQ ID NO:252; VH comprising the amino acid sequence of SEQ ID NO:242 and VL comprising the amino acid sequence of SEQ ID NO: 249; VH comprising the amino acid sequence of SEQ ID NO:243 and VL comprising the amino acid sequence of SEQ ID NO:247; VH comprising the amino acid sequence of SEQ ID NO:244 and VL comprising the amino acid sequence of SEQ ID NO:251; VH comprising the amino acid sequence of SEQ ID NO:245 and VL comprising the amino acid sequence of SEQ ID NO:253; VH comprising the amino acid sequence of SEQ ID NO:246 and VL comprising the amino acid sequence of SEQ ID NO:247; and VH comprising the amino acid sequence of SEQ ID NO:246 and VL comprising the amino acid sequence of SEQ ID NO:254.

In one aspect, the present disclosure relates to an isolated anti-MerTK antibody that binds to a MerTK protein, wherein the antibody comprises a heavy chain variable region and a light chain variable region, wherein the heavy chain variable region comprises: a) an HVR-H1 comprising the amino acid sequence of SEQ ID NO:84; b) an HVR-H2 comprising an amino acid sequence selected from the group consisting of SEQ ID NOs: 99 and 329; and c) an HVR-H3 comprising the amino acid sequence of SEQ ID NO: 139; and wherein the light chain variable region comprises: d) an HVR-L1 comprising an amino acid sequence selected from the group consisting of SEQ ID NOs: 169 and 345; e) an HVR-L2 comprising the amino acid sequence of SEQ ID NO:195; and f) an HVR-L3 comprising the amino acid sequence of SEQ ID NO:219.

In one aspect, the present disclosure relates to an isolated anti-MerTK antibody that binds to a MerTK protein, wherein the antibody comprises (a) HVR-H1 comprising the amino acid sequence of SEQ ID NO:84; (b) HVR-H2 comprising the amino acid sequence of SEQ ID NO:329; (c) HVR-H3 comprising the amino acid sequence of SEQ ID NO: 139; (d) HVR-L1 comprising the amino acid sequence of SEQ ID NO: 169; (e) HVR-L2 comprising the amino acid sequence of SEQ ID NO:195; and (f) HVR-L3 comprising the amino acid sequence of SEQ ID NO:219; or (a) HVR-H1 comprising the amino acid sequence of SEQ ID NO:84; (b) HVR-H2 comprising the amino acid sequence of SEQ ID NO:99; (c) HVR-H3 comprising the amino acid sequence of SEQ ID NO:139; (d) HVR-L1 comprising the amino acid sequence of SEQ ID NO:345; (e) HVR-L2 comprising the amino acid sequence of SEQ ID NO: 195; and (f) HVR-L3 comprising the amino acid sequence of SEQ ID NO:219.

In one aspect, the present disclosure relates to an isolated anti-MerTK antibody that binds to a MerTK protein, wherein the antibody comprises a heavy chain variable domain (VH) and a light chain variable domain (VL), wherein the VH and VL are selected from the group consisting of: VH comprising the amino acid sequence of SEQ ID NO:255 and VL comprising the amino acid sequence of SEQ ID NO:257; and VH comprising the amino acid sequence of SEQ ID NO:256 and VL comprising the amino acid sequence of SEQ ID NO:258.

In one aspect, the present disclosure relates to an isolated anti-MerTK antibody that binds to a MerTK protein, wherein the antibody comprises a heavy chain variable region and a light chain variable region, wherein the heavy chain variable region comprises: a) an HVR-H1 comprising an amino acid sequence selected from the group consisting of SEQ ID NOs:95, 346, and 347; b) an HVR-H2 comprising an amino acid sequence selected from the group consisting of SEQ ID NOs: 119, 348, 349, 350, 351, 352, 353, and 354; and c) an HVR-H3 comprising an amino acid sequence selected from the group consisting of SEQ ID NOs: 151, 355, and 356; and wherein the light chain variable region comprises: d) an HVR-L1 comprising an amino acid sequence selected from the group consisting of SEQ ID NOs: 181, 341, 357, and 358; e) an HVR-L2 comprising an amino acid sequence selected from the group consisting of SEQ ID NOs:187 and 359; and f) an HVR-L3 comprising an amino acid sequence selected from the group consisting of SEQ ID NOs:208, 360, 361, and 362.

In one aspect, the present disclosure relates to an isolated anti-MerTK antibody that binds to a MerTK protein, wherein the antibody comprises (a) HVR-H1 comprising the amino acid sequence of SEQ ID NO:95; (b) HVR-H2 comprising the amino acid sequence of SEQ ID NO: 119; (c) HVR-H3 comprising the amino acid sequence of SEQ ID NO:151; (d) HVR-L1 comprising the amino acid sequence of SEQ ID NO:181; (e) HVR-L2 comprising the amino acid sequence of SEQ ID NO:187; and (f) HVR-L3 comprising the amino acid sequence of SEQ ID NO:208; (a) HVR-H1 comprising the amino acid sequence of SEQ ID NO:346; (b) HVR-H2 comprising the amino acid sequence of SEQ ID NO:348; (c) HVR-H3 comprising the amino acid sequence of SEQ ID NO:355; (d) HVR-L1 comprising the amino acid sequence of SEQ ID NO:341; (e) HVR-L2 comprising the amino acid sequence of SEQ ID NO: 187; and (f) HVR-L3 comprising the amino acid sequence of SEQ ID NO:360; (a) HVR-H1 comprising the amino acid sequence of SEQ ID NO:95; (b) HVR-H2 comprising the amino acid sequence of SEQ ID NO: 119; (c) HVR-H3 comprising the amino acid sequence of SEQ ID NO:355; (d) HVR-L1 comprising the amino acid sequence of SEQ ID NO:341; (e) HVR-L2 comprising the amino acid sequence of SEQ ID NO: 187; and (f) HVR-L3 comprising the amino acid sequence of SEQ ID NO:361; (a) HVR-H1 comprising the amino acid sequence of SEQ ID NO:95; (b) HVR-H2 comprising the amino acid sequence of SEQ ID NO:349; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO: 187; and (f) HVR-L3 comprising the amino acid sequence of SEQ ID NO:362; (a) HVR-H1 comprising the amino acid sequence of SEQ ID NO:95; (b) HVR-H2 comprising the amino acid sequence of SEQ ID NO: 119; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:359; and (f) HVR-L3 comprising the amino acid sequence of SEQ ID NO:362; (a) HVR-H1 comprising the amino acid sequence of SEQ ID NO:346; (b) HVR-H2 comprising the amino acid sequence of SEQ ID NO: 119; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:359; and (f) HVR-L3 comprising the amino acid sequence of SEQ ID NO:362; (a) HVR-H1 comprising the amino acid sequence of SEQ ID NO:95; (b) HVR-H2 comprising the amino acid sequence of SEQ ID NO:350 (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:359; and (f) HVR-L3 comprising the amino acid sequence of SEQ ID NO:362; (a) HVR-H1 comprising the amino acid sequence of SEQ ID NO:347; (b) HVR-H2 comprising the amino acid sequence of SEQ ID NO: 119; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:359; and (f) HVR-L3 comprising the amino acid sequence of SEQ ID NO:362; (a) HVR-H1 comprising the amino acid sequence of SEQ ID NO:95; (b) HVR-H2 comprising the amino acid sequence of SEQ ID NO: 119; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:187; and (f) HVR-L3 comprising the amino acid sequence of SEQ ID NO:362; (a) HVR-H1 comprising the amino acid sequence of SEQ ID NO:95; (b) HVR-H2 comprising the amino acid sequence of SEQ ID NO:352; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO: 187; and (f) HVR-L3 comprising the amino acid sequence of SEQ ID NO:362; (a) HVR-H1 comprising the amino acid sequence of SEQ ID NO:95; (b) HVR-H2 comprising the amino acid sequence of SEQ ID NO:353; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:359; and (f) HVR-L3 comprising the amino acid sequence of SEQ ID NO:362; (a) HVR-H1 comprising the amino acid sequence of SEQ ID NO:346; (b) HVR-H2 comprising the amino acid sequence of SEQ ID NO:354; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:359; and (f) HVR-L3 comprising the amino acid sequence of SEQ ID NO:363; (a) HVR-H1 comprising the amino acid sequence of SEQ ID NO:346; (b) HVR-H2 comprising the amino acid sequence of SEQ ID NO:348; (c) HVR-H3 comprising the amino acid sequence of SEQ ID NO:356; (d) HVR-L1 comprising the amino acid sequence of SEQ ID NO:357; (e) HVR-L2 comprising the amino acid sequence of SEQ ID NO:359; and (f) HVR-L3 comprising the amino acid sequence of SEQ ID NO:362; (a) HVR-H1 comprising the amino acid sequence of SEQ ID NO:346; (b) HVR-H2 comprising the amino acid sequence of SEQ ID NO:354; (c) HVR-H3 comprising the amino acid sequence of SEQ ID NO:356; (d) HVR-L1 comprising the amino acid sequence of SEQ ID NO:358; (e) HVR-L2 comprising the amino acid sequence of SEQ ID NO: 187; and (f) HVR-L3 comprising the amino acid sequence of SEQ ID NO:362; or (a) HVR-H1 comprising the amino acid sequence of SEQ ID NO:95; (b) HVR-H2 comprising the amino acid sequence of SEQ ID NO:348; (c) HVR-H3 comprising the amino acid sequence of SEQ ID NO:151; (d) HVR-L1 comprising the amino acid sequence of SEQ ID NO:181; (e) HVR-L2 comprising the amino acid sequence of SEQ ID NO:187; and (f) HVR-L3 comprising the amino acid sequence of SEQ ID NO:208.

In one aspect, the present disclosure relates to an isolated anti-MerTK antibody that binds to a MerTK protein, wherein the antibody comprises a heavy chain variable domain (VH) and a light chain variable domain (VL), wherein the VH and VL are selected from the group consisting of: VH comprising the amino acid sequence of SEQ ID NO:33 and VL comprising the amino acid sequence of SEQ ID NO:68; VH comprising the amino acid sequence of SEQ ID NO:259 and VL comprising the amino acid sequence of SEQ ID NO:274; VH comprising the amino acid sequence of SEQ ID NO:260 and VL comprising the amino acid sequence of SEQ ID NO: 275; VH comprising the amino acid sequence of SEQ ID NO:261 and VL comprising the amino acid sequence of SEQ ID NO:276; VH comprising the amino acid sequence of SEQ ID NO:262 and VL comprising the amino acid sequence of SEQ ID NO:277; VH comprising the amino acid sequence of SEQ ID NO:263 and VL comprising the amino acid sequence of SEQ ID NO:277; VH comprising the amino acid sequence of SEQ ID NO:264 and VL comprising the amino acid sequence of SEQ ID NO:277; VH comprising the amino acid sequence of SEQ ID NO:265 and VL comprising the amino acid sequence of SEQ ID NO:277; VH comprising the amino acid sequence of SEQ ID NO:266 and VL comprising the amino acid sequence of SEQ ID NO:277; VH comprising the amino acid sequence of SEQ ID NO:267 and VL comprising the amino acid sequence of SEQ ID NO:278; VH comprising the amino acid sequence of SEQ ID NO:268 and VL comprising the amino acid sequence of SEQ ID NO:279; VH comprising the amino acid sequence of SEQ ID NO:269 and VL comprising the amino acid sequence of SEQ ID NO:279; VH comprising the amino acid sequence of SEQ ID NO:264 and VL comprising the amino acid sequence of SEQ ID NO:280; VH comprising the amino acid sequence of SEQ ID NO:270 and VL comprising the amino acid sequence of SEQ ID NO:281; VH comprising the amino acid sequence of SEQ ID NO:265 and VL comprising the amino acid sequence of SEQ ID NO:282; VH comprising the amino acid sequence of SEQ ID NO:264 and VL comprising the amino acid sequence of SEQ ID NO:283; VH comprising the amino acid sequence of SEQ ID NO:271 and VL comprising the amino acid sequence of SEQ ID NO:284; VH comprising the amino acid sequence of SEQ ID NO:272 and VL comprising the amino acid sequence of SEQ ID NO:285; VH comprising the amino acid sequence of SEQ ID NO:271 and VL comprising the amino acid sequence of SEQ ID NO:286; VH comprising the amino acid sequence of SEQ ID NO:273 and VL comprising the amino acid sequence of SEQ ID NO:287; VH comprising the amino acid sequence of SEQ ID NO:273 and VL comprising the amino acid sequence of SEQ ID NO:288; and VH comprising the amino acid sequence of SEQ ID NO:273 and VL comprising the amino acid sequence of SEQ ID NO:289.

In one aspect, the present disclosure relates to an isolated anti-MerTK antibody that binds to a MerTK protein, wherein the antibody comprises a heavy chain variable region and a light chain variable region, wherein the heavy chain variable region comprises: a) an HVR-H1 comprising the amino acid sequence of SEQ ID NO:90; b) an HVR-H2 comprising an amino acid sequence selected from the group consisting of SEQ ID NOs: 122, 364, 365, 366, 367, and 368; and c) an HVR-H3 comprising an amino acid sequence selected from the group consisting of SEQ ID NOs: 155, 373, 374, and 375; and wherein the light chain variable region comprises: d) an HVR-L1 comprising an amino acid sequence selected from the group consisting of SEQ ID NOs: 184, 376, 377, 378, 379, 380, 381, 382, 383, 384, 385, 386, and 387; e) an HVR-L2 comprising an amino acid sequence selected from the group consisting of SEQ ID NOs:203, 388, 389, 390, and 391; and f) an HVR-L3 comprising an amino acid sequence selected from the group consisting of SEQ ID NOs:231, 392, 393, and 394.

In one aspect, the present disclosure relates to an isolated anti-MerTK antibody that binds to a MerTK protein, wherein the antibody comprises (a) HVR-H1 comprising the amino acid sequence of SEQ ID NO:90; (b) HVR-H2 comprising the amino acid sequence of SEQ ID NO: 122; (c) HVR-H3 comprising the amino acid sequence of SEQ ID NO: 155; (d) HVR-L1 comprising the amino acid sequence of SEQ ID NO: 184; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:373; (d) HVR-L1 comprising the amino acid sequence of SEQ ID NO:376; (e) HVR-L2 comprising the amino acid sequence of SEQ ID NO:388; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:155; (d) HVR-L1 comprising the amino acid sequence of SEQ ID NO:377; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:374; (d) HVR-L1 comprising the amino acid sequence of SEQ ID NO:378; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:95; (b) HVR-H2 comprising the amino acid sequence of SEQ ID NO: 119; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:359; and (f) HVR-L3 comprising the amino acid sequence of SEQ ID NO:362; (a) HVR-H1 comprising the amino acid sequence of SEQ ID NO:90; (b) HVR-H2 comprising the amino acid sequence of SEQ ID NO:365; (c) HVR-H3 comprising the amino acid sequence of SEQ ID NO:155; (d) HVR-L1 comprising the amino acid sequence of SEQ ID NO:276; (e) HVR-L2 comprising the amino acid sequence of SEQ ID NO:203; and (f) HVR-L3 comprising the amino acid sequence of SEQ ID NO:392; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:155; (d) HVR-L1 comprising the amino acid sequence of SEQ ID NO:376; (e) HVR-L2 comprising the amino acid sequence of SEQ ID NO:389; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:155; (d) HVR-L1 comprising the amino acid sequence of SEQ ID NO:379; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:366; (c) HVR-H3 comprising the amino acid sequence of SEQ ID NO:155; (d) HVR-L1 comprising the amino acid sequence of SEQ ID NO:380; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:373; (d) HVR-L1 comprising the amino acid sequence of SEQ ID NO:381; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:367; (c) HVR-H3 comprising the amino acid sequence of SEQ ID NO:155; (d) HVR-L1 comprising the amino acid sequence of SEQ ID NO:382; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO:155; (d) HVR-L1 comprising the amino acid sequence of SEQ ID NO:383; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:374; (d) HVR-L1 comprising the amino acid sequence of SEQ ID NO:384; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO:155; (d) HVR-L1 comprising the amino acid sequence of SEQ ID NO:376; (e) HVR-L2 comprising the amino acid sequence of SEQ ID NO:203; and (f) HVR-L3 comprising the amino acid sequence of SEQ ID NO:393; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:155; (d) HVR-L1 comprising the amino acid sequence of SEQ ID NO:378; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO:155; (d) HVR-L1 comprising the amino acid sequence of SEQ ID NO:376; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:369; (c) HVR-H3 comprising the amino acid sequence of SEQ ID NO:155; (d) HVR-L1 comprising the amino acid sequence of SEQ ID NO:378; (e) HVR-L2 comprising the amino acid sequence of SEQ ID NO:203; and (f) HVR-L3 comprising the amino acid sequence of SEQ ID NO:394; (a) HVR-H1 comprising the amino acid sequence of SEQ ID NO:90; (b) HVR-H2 comprising the amino acid sequence of SEQ ID NO:370; (c) HVR-H3 comprising the amino acid sequence of SEQ ID NO:155; (d) HVR-L1 comprising the amino acid sequence of SEQ ID NO:378; (e) HVR-L2 comprising the amino acid sequence of SEQ ID NO:203; and (f) HVR-L3 comprising the amino acid sequence of SEQ ID NO:394; (a) HVR-H1 comprising the amino acid sequence of SEQ ID NO:90; (b) HVR-H2 comprising the amino acid sequence of SEQ ID NO:371; (c) HVR-H3 comprising the amino acid sequence of SEQ ID NO:155; (d) HVR-L1 comprising the amino acid sequence of SEQ ID NO:178; (e) HVR-L2 comprising the amino acid sequence of SEQ ID NO:203; and (f) HVR-L3 comprising the amino acid sequence of SEQ ID NO:394; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO:155; (d) HVR-L1 comprising the amino acid sequence of SEQ ID NO:385; (e) HVR-L2 comprising the amino acid sequence of SEQ ID NO:203; and (f) HVR-L3 comprising the amino acid sequence of SEQ ID NO:394; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO:155; (d) HVR-L1 comprising the amino acid sequence of SEQ ID NO:378; (e) HVR-L2 comprising the amino acid sequence of SEQ ID NO:203; and (f) HVR-L3 comprising the amino acid sequence of SEQ ID NO:395; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO:155; (d) HVR-L1 comprising the amino acid sequence of SEQ ID NO:378; (e) HVR-L2 comprising the amino acid sequence of SEQ ID NO:390; and (f) HVR-L3 comprising the amino acid sequence of SEQ ID NO:394; (a) HVR-H1 comprising the amino acid sequence of SEQ ID NO:90; (b) HVR-H2 comprising the amino acid sequence of SEQ ID NO:372; (c) HVR-H3 comprising the amino acid sequence of SEQ ID NO:155; (d) HVR-L1 comprising the amino acid sequence of SEQ ID NO:378; (e) HVR-L2 comprising the amino acid sequence of SEQ ID NO:391; and (f) HVR-L3 comprising the amino acid sequence of SEQ ID NO:394; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO:375; (d) HVR-L1 comprising the amino acid sequence of SEQ ID NO:386; (e) HVR-L2 comprising the amino acid sequence of SEQ ID NO:203; and (f) HVR-L3 comprising the amino acid sequence of SEQ ID NO:394; or (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO:155; (d) HVR-L1 comprising the amino acid sequence of SEQ ID NO:387; (e) HVR-L2 comprising the amino acid sequence of SEQ ID NO:203; and (f) HVR-L3 comprising the amino acid sequence of SEQ ID NO:394.

In one aspect, the present disclosure relates to an isolated anti-MerTK antibody that binds to a MerTK protein, wherein the antibody comprises a heavy chain variable domain (VH) and a light chain variable domain (VL), wherein the VH and VL are selected from the group consisting of: VH comprising the amino acid sequence of SEQ ID NO:37 and VL comprising the amino acid sequence of SEQ ID NO:72; VH comprising the amino acid sequence of SEQ ID NO:290 and VL comprising the amino acid sequence of SEQ ID NO:309; VH comprising the amino acid sequence of SEQ ID NO:291 and VL comprising the amino acid sequence of SEQ ID NO:310; VH comprising the amino acid sequence of SEQ ID NO:292 and VL comprising the amino acid sequence of SEQ ID NO:311; VH comprising the amino acid sequence of SEQ ID NO:293 and VL comprising the amino acid sequence of SEQ ID NO:312; VH comprising the amino acid sequence of SEQ ID NO:294 and VL comprising the amino acid sequence of SEQ ID NO:313; VH comprising the amino acid sequence of SEQ ID NO:295 and VL comprising the amino acid sequence of SEQ ID NO:314; VH comprising the amino acid sequence of SEQ ID NO:296 and VL comprising the amino acid sequence of SEQ ID NO:315; VH comprising the amino acid sequence of SEQ ID NO:290 and VL comprising the amino acid sequence of SEQ ID NO:316; VH comprising the amino acid sequence of SEQ ID NO:297 and VL comprising the amino acid sequence of SEQ ID NO:317; VH comprising the amino acid sequence of SEQ ID NO:298 and VL comprising the amino acid sequence of SEQ ID NO:318; VH comprising the amino acid sequence of SEQ ID NO:292 and VL comprising the amino acid sequence of SEQ ID NO:319; VH comprising the amino acid sequence of SEQ ID NO:299 and VL comprising the amino acid sequence of SEQ ID NO:320; VH comprising the amino acid sequence of SEQ ID NO:300 and VL comprising the amino acid sequence of SEQ ID NO:311; VH comprising the amino acid sequence of SEQ ID NO:301 and VL comprising the amino acid sequence of SEQ ID NO:321; VH comprising the amino acid sequence of SEQ ID NO:302 and VL comprising the amino acid sequence of SEQ ID NO:322; VH comprising the amino acid sequence of SEQ ID NO:303 and VL comprising the amino acid sequence of SEQ ID NO:311; VH comprising the amino acid sequence of SEQ ID NO:304 and VL comprising the amino acid sequence of SEQ ID NO:322; VH comprising the amino acid sequence of SEQ ID NO:305 and VL comprising the amino acid sequence of SEQ ID NO:322; VH comprising the amino acid sequence of SEQ ID NO:301 and VL comprising the amino acid sequence of SEQ ID NO:323; VH comprising the amino acid sequence of SEQ ID NO:301 and VL comprising the amino acid sequence of SEQ ID NO:324; VH comprising the amino acid sequence of SEQ ID NO:301 and VL comprising the amino acid sequence of SEQ ID NO:325; VH comprising the amino acid sequence of SEQ ID NO:306 and VL comprising the amino acid sequence of SEQ ID NO:326; VH comprising the amino acid sequence of SEQ ID NO:307 and VL comprising the amino acid sequence of SEQ ID NO:327; and VH comprising the amino acid sequence of SEQ ID NO:308 and VL comprising the amino acid sequence of SEQ ID NO:328.

In one aspect, the present disclosure relates to an isolated anti-MerTK antibody that binds to a MerTK protein, wherein the antibody includes a heavy chain variable region and a light chain variable region, wherein the heavy chain variable region includes: an HVR-H1 comprising an amino acid sequence selected from the group consisting of SEQ ID NOs:75, 76, 77, 78, 79, 80, 81, 82, 83, 84, 85, 86, 87, 88, 89, 90, 91, 92, 93, 94, 95, 96, 97, and 98; an HVR-H2 comprising an amino acid sequence selected from the group consisting of SEQ ID NOs:99, 100, 101, 102, 103, 104, 105, 106, 107, 108, 109, 110, 111, 112, 113, 114, 115, 116, 117, 118, 119, 120, 121, 122, 123, and 124; and an HVR-H3 comprising an amino acid sequence selected from the group consisting of SEQ ID NOs:125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, and 157; and the light chain variable region includes: an HVR-L1 comprising an amino acid sequence selected from the group consisting of SEQ ID NOs:158, 159, 160, 161, 162, 163, 164, 165, 166, 167, 168, 169, 170, 171, 172, 173, 174, 175, 176, 177, 178, 179, 180, 181, 182, 183, 184, 185, and 186; an HVR-L2 comprising an amino acid sequence selected from the group consisting of SEQ ID NOs:187, 188, 189, 190, 191, 192, 193, 194, 195, 196, 197, 198, 199, 200, 201, 202, 203, 204, and 205; and an HVR-L3 comprising an amino acid sequence selected from the group consisting of SEQ ID NOs:207, 208, 209, 210, 211, 212, 213, 214, 215, 216, 217, 218, 219, 220, 221, 222, 223, 224, 225, 226, 227, 228, 229, 230, 231, 232, and 233.

In one aspect, the present disclosure relates to an isolated anti-MerTK antibody that binds to a MerTK protein, wherein the antibody comprises an HVR-H1, HVR-H2, HVR-H3, HVR-L1, HVR-L2, and HVR-L3 comprising the amino acid sequences of (i) SEQ ID NOs: 75, 99, 125, 158, 187, and 207, respectively; (ii) SEQ ID NOs: 76, 100, 126, 159, 187, and 208, respectively; (iii) SEQ ID NOs: 77, 101, 127, 160, 188, and 209, respectively; (iv) SEQ ID NOs: 75, 99, 128, 158, 187, and 210, respectively; (v) SEQ ID NOs: 78, 102, 129, 161, 189, and 211, respectively; (vi) SEQ ID NOs: 75, 103, 130, 158, 187, and 212, respectively; (vii) SEQ ID NOs: 77, 101, 127, 162, 188, and 209, respectively; (viii) SEQ ID NOs: 75, 99, 131, 163, 187, and 213, respectively; (ix) SEQ ID NOs: 79, 104, 132, 164, 190, and 214, respectively; (x) SEQ ID NOs: 80, 105, 133, 165, 191, and 215, respectively; (xi) SEQ ID NOs: 81, 106, 134, 166, 192, and 216, respectively; (xii) SEQ ID NOs: 82, 107, 135, 167, 193, and 217, respectively; (xiii) SEQ ID NOs: 77, 108, 136, 167, 194, and 209, respectively; (xiv) SEQ ID NOs: 75, 109, 137, 168, 187, and 218, respectively; (xv) SEQ ID NOs: 83, 99, 138, 158, 187, and 210, respectively; (xvi) SEQ ID NOs: 84, 99, 139, 169, 195, and 219, respectively; (xvii) SEQ ID NOs: 85, 99, 140, 170, 196, and 220, respectively; (xviii) SEQ ID NOs: 86, 99, 141, 171, 191, and 221, respectively; (xix) SEQ ID NOs:87, 110, 142, 172, 197, and 222, respectively; (xx) SEQ ID NOs: 88, 99, 143, 173, 191, and 223, respectively; (xxi) SEQ ID NOs: 89, 111, 143, 173, 198, and 221, respectively; (xxii) SEQ ID NOs: 90, 112, 144, 174, 199, and 224, respectively; (xxiii) SEQ ID NOs: 91, 113, 145, 175, 200, and 225, respectively; (xxiv) SEQ ID NOs: 90, 114, 146, 176, 201, and 226, respectively; (xxv) SEQ ID NOs: 92, 115, 147, 177, 195, and 227, respectively; (xxvi) SEQ ID NOs: 93, 116, 148, 178, 188, and 209, respectively; (xxvii) SEQ ID NOs: 94, 117, 149, 179, 195, and 228, respectively; (xxviii) SEQ ID NOs: 95, 118, 150, 180, 187, and 229, respectively; (xxix) SEQ ID NOs: 95, 119, 151, 181, 187, and 208, respectively; (xxx) SEQ ID NOs: 90, 120, 152, 182, 202, and 230, respectively; (xxxi) SEQ ID NOs: 96, 118, 153, 183, 202, and 230, respectively; (xxxii) SEQ ID NOs: 96, 121, 154, 181, 187, and 210, respectively; (xxxiii) SEQ ID NOs: 90, 122, 155, 184, 203, and 231, respectively; (xxxiv) SEQ ID NOs: 90, 122, 155, 184, 203, and 231, respectively; (xxxv) SEQ ID NOs:97, 123, 156, 185, 204, and 232, respectively; or (xxxvi) SEQ ID NOs: 98, 124, 157, 186, 205, and 233, respectively.

In some embodiments that may be combined with any of the embodiments provided herein, an anti-MerTK antibody of the present disclosure is an isolated antibody that binds to a MerTK protein, wherein the antibody comprises the HVR-H1, HVR-H2, HVR-H3, HVR-L1, HVR-L2 and HVR-L3 sequences of MTK-01, MTK-02, MTK-03, MTK-04, MTK-05, MTK-06, MTK-07, MTK-08, MTK-09, MTK-10, MTK-11, MTK-12, MTK-13, MTK-14, MTK-15, MTK-16, MTK-17, MTK-18, MTK-19, MTK-20, MTK-21, MTK-22, MTK-23, MTK-24, MTK-25, MTK-26, MTK-27, MTK-28, MTK-29, MTK-30, MTK-31, MTK-32, MTK-33, MTK-34, MTK-35, or MTK-36 antibody. In some embodiments, the HVRs are the Kabat-defined HVRs, the Chothia-defined HVRs, or the AbM-defined HVRs.

In some embodiments that may be combined with any of the embodiments provided herein, an anti-MerTK antibody of the present disclosure is an isolated antibody that binds to a MerTK protein, wherein the antibody comprises the HVR-H1, HVR-H2, HVR-H3, HVR-L1, HVR-L2 and HVR-L3 sequences of MTK-15.1, MTK-15.2, MTK-15.3, MTK-15.4, MTK-15.5, MTK-15.6, MTK-15.7, MTK-15.8, MTK-15.9, MTK-15.10, MTK-15.11, MTK-15.12, MTK-15.13, MTK-15.14, and MTK-15.15, MTK-16.1 and MTK-16.2, MTK-29.1, MTK-29.2, MTK-29.3, MTK-29.4, MTK-29.5, MTK-29.6, MTK-29.7, MTK-29.8, MTK-29.9, MTK-29.10, MTK-29.11, MTK-29.12, MTK-29.13, MTK-29.14, MTK-29.15, MTK-29.16, MTK-29.17, MTK-29.18, MTK-29.19, MTK-29.20, and MTK-29.21, MTK-33.1, MTK-33.2, MTK-33.3, MTK-33.4, MTK-33.5, MTK-33.6, MTK-33.7, MTK-33.8, MTK-33.9, MTK-33.10, MTK-33.11, MTK-33.12, MTK-33.13, MTK-33.14, MTK-33.15, MTK-33.16, MTK-33.17, MTK-33.18, MTK-33.19, MTK-33.20, MTK-33.21, MTK-33.22, MTK-33.23, or MTK-33.24 antibody. In some embodiments, the HVRs are the Kabat-defined HVRs, the Chothia-defined HVRs, or the AbM-defined HVRs.

In some embodiments that may be combined with any of the embodiments provided herein, an anti-MerTK antibody of the present disclosure is an isolated antibody that binds to a MerTK protein, wherein the antibody includes a heavy chain variable region, wherein the heavy chain variable region includes an amino acid sequence selected from the group consisting of SEQ ID NOs:5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, and 39.

In some embodiments that may be combined with any of the embodiments provided herein, an anti-MerTK antibody of the present disclosure is an isolated antibody that binds to a MerTK protein, wherein the antibody includes a heavy chain variable region, wherein the heavy chain variable region includes an amino acid sequence selected from the group consisting of SEQ ID NOs:234-246, 255, 256, 259-379, and 290-308.

In some embodiments that may be combined with any of the embodiments provided herein, an anti-MerTK antibody of the present disclosure is an isolated antibody that binds to a MerTK protein, wherein the antibody includes a light chain variable region, wherein the light chain variable region includes an amino acid sequence selected from the group consisting of SEQ ID NOs:40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, and 74.

In some embodiments that may be combined with any of the embodiments provided herein, an anti-MerTK antibody of the present disclosure is an isolated antibody that binds to a MerTK protein, wherein the antibody includes a light chain variable region, wherein the light chain variable region includes an amino acid sequence selected from the group consisting of SEQ ID NOs:247-254, 257, 258, 274-289, and 309-328.

In some embodiments that may be combined with any of the embodiments provided herein, an anti-MerTK antibody of the present disclosure is an isolated antibody that binds to a MerTK protein, wherein the antibody includes a heavy chain variable region and a light chain variable region, wherein the heavy chain variable region includes an amino acid sequence selected from SEQ ID NOs:5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, and 39, and the light chain variable region includes an amino acid sequence selected from SEQ ID NOs: 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, and 74.

In some embodiments that may be combined with any of the embodiments provided herein, an anti-MerTK antibody of the present disclosure is an isolated antibody that binds to a MerTK protein, wherein the antibody includes a heavy chain variable region and a light chain variable region, wherein the heavy chain variable region includes an amino acid sequence selected from 234-246, 255, 256, 259-379, and 290-308, and the light chain variable region includes an amino acid sequence selected from SEQ ID NOs: 247-254, 257, 258, 274-289, and 309-328.

In some embodiments that may be combined with any of the embodiments provided herein, an anti-MerTK antibody of the present disclosure is an isolated antibody that binds to a MerTK protein, wherein the antibody a heavy chain variable region and a light chain variable region comprising the amino acid sequences of SEQ ID NOs:5 and 40, respectively; SEQ ID NOs:6 and 41, respectively; SEQ ID NOs:7 and 42, respectively; respectively; SEQ ID NOs:8 and 43, respectively; SEQ ID NOs:9 and 44, respectively; SEQ ID NOs:10 and 45, respectively; SEQ ID NOs: 11 and 46, respectively; SEQ ID NOs:12 and 47, respectively; SEQ ID NOs:13 and 48, respectively; SEQ ID NOs:14 and 49, respectively; SEQ ID NOs:15 and 50, respectively; SEQ ID NOs:16 and 51, respectively; SEQ ID NOs:17 and 52, respectively; SEQ ID NOs:18 and 53, respectively; SEQ ID NOs:19 and 54, respectively; SEQ ID NOs:20 and 55, respectively; SEQ ID NOs:21 and 56, respectively; SEQ ID NOs:22 and 57, respectively; SEQ ID NOs:23 and 58, respectively; SEQ ID NOs:24 and 59, respectively; SEQ ID NOs:25 and 60, respectively; SEQ ID NOs:26 and 61, respectively; SEQ ID NOs:27 and 62, respectively; SEQ ID NOs:28 and 63, respectively; SEQ ID NOs:29 and 64, respectively; SEQ ID NOs:30 and 65, respectively; SEQ ID NOs:31 and 66, respectively; SEQ ID NOs:32 and 67, respectively; SEQ ID NOs:33 and 68, respectively; SEQ ID NOs:34 and 69, respectively; SEQ ID NOs:35 and 70, respectively; SEQ ID NOs:36 and 71, respectively; SEQ ID NOs:37 and 72, respectively; SEQ ID NOs:38 and 73, respectively; and SEQ ID NOs:39 and 74, respectively.

In one aspect, the present disclosure relates to an isolated antibody that binds to a MerTK protein, wherein the antibody competitively inhibits binding of one or more of the antibodies of any of the embodiments herein for binding to MerTK.

In another aspect, the present disclosure relates to an isolated antibody that binds to a MerTK protein, wherein the antibody binds essentially the same or an overlapping epitope on MerTK as an antibody of any of the embodiments herein.

In certain embodiments that may be combined with any of the embodiments herein, the MerTK protein is a mammalian protein or a human protein. In certain embodiments that may be combined with any of the embodiments herein, the MerTK protein is a wild-type protein. In certain embodiments that may be combined with any of the embodiments herein, the MerTK protein is a naturally occurring variant. In certain embodiments that may be combined with any of the embodiments herein, an anti-MerTK antibody binds to human MerTK and to cynomolgus monkey MerTK and/or murine MerTK. In certain embodiments that may be combined with any of the embodiments herein, an anti-MerTK antibody binds to human MerTK and murine MerTK, binds to human MerTK and cynomolgus MerTK, or binds to human MerTK, murine MerTK, and cynomolgus MerTK.

In some embodiments that may be combined with any of the embodiments herein, an anti-MerTK antibody of the present disclosure inhibits or reduces binding of one or more ligands to MerTK.

In some embodiments that may be combined with any of the embodiments herein, an anti-MerTK antibody of the present disclosure inhibits or reduces binding of ProS to MerTK. In some embodiments that may be combined with any of the embodiments herein, an anti-MerTK antibody of the present disclosure inhibits or reduces binding of ProS to MerTK by at least 20%, by at least 25%, by at least 30%, by at least 40%, by at least 50%, by at least 60%, by at least 70%, by at least 80%, by at least 90%, or by at least 95%. In some embodiments that may be combined with any of the embodiments herein, an anti-MerTK antibody of the present disclosure inhibits or reduces binding of ProS to MerTK with an IC50 value of 0.58 nM to 25.9 nM. %. In some embodiments that may be combined with any of the embodiments herein, an anti-MerTK antibody of the present disclosure inhibits or reduces binding of ProS to MerTK with an IC50 value of 0.58 nM to 1 nM, 1 nM to 2.5 nM, 2.5 nM to 5 nM, 5 nM to 10 nM, or 10 nM to 25 nM.

In some embodiments that may be combined with any of the embodiments herein, an anti-MerTK antibody of the present disclosure inhibits or reduces binding of Gas6 to MerTK. In some embodiments that may be combined with any of the embodiments herein, an anti-MerTK antibody of the present disclosure inhibits or reduces binding of Gas6 to MerTK by at least 20%, by at least 25%, by at least 30%, by at least 40%, by at least 50%, by at least 60%, by at least 70%, by at least 80%, by at least 90%, or by at least 95%. In some embodiments that may be combined with any of the embodiments herein, an anti-MerTK antibody of the present disclosure inhibits or reduces binding of Gas6 to MerTK with an IC50 value of 0.29 nM to 32 nM. In some embodiments that may be combined with any of the embodiments herein, an anti-MerTK antibody of the present disclosure inhibits or reduces binding of Gas6 to MerTK with an IC50 value of 0.29 nM to 1 nM, 1 nM to 5 nM, 5 nM to 10 nM, 10 nM to 25 nM, or 25 nM to 32 nM.

In some embodiments that may be combined with any of the embodiments herein, an anti-MerTK antibody of the present disclosure inhibits or reduces binding of Gas6 to MerTK and inhibits or reduces binding of ProS to MerTK. In some embodiments that may be combined with any of the embodiments herein, an anti-MerTK antibody of the present disclosure inhibits or reduces binding of Gas6 to MerTK and inhibits or reduces binding of ProS to MerTK by at least 20%, by at least 25%, by at least 30%, by at least 40%, by at least 50%, by at least 60%, by at least 70%, by at least 80%, by at least 90%, or by at least 95% and wherein the antibody reduces or inhibits binding of Gas6 to MerTK by at least 20%, by at least 25%, by at least 30%, by at least 40%, by at least 50%, by at least 60%, by at least 70%, by at least 80%, by at least 90%, or by at least 95%. In some embodiments that may be combined with any of the embodiments herein, an anti-MerTK antibody of the present disclosure inhibits or reduces binding of Gas6 to MerTK and inhibits or reduces binding of ProS to MerTK with an IC50 value of 0.58 nM to 25.9 nM and wherein the antibody reduces or inhibits binding of Gas6 to MetTK with an IC50 value of 0.29 nM to 32 nM.

In some embodiments that may be combined with any of the embodiments herein, an anti-MerTK antibody of the present disclosure inhibits or reduces binding of ProS to MerTK and does not inhibit or reduce binding of Gas6 to MerTK.

In some embodiments that may be combined with any of the embodiments herein, an anti-MerTK antibody of the present disclosure does not inhibit or reduce binding of ProS to MerTK and does not inhibit binding of Gas6 to MerTK.

In some embodiments that may be combined with any of the embodiments herein, an anti-MerTK antibody of the present disclosure reduces Gas6-mediated phosphorylation of AKT. In some embodiments that may be combined with any of the embodiments herein, an anti-MerTK antibody of the present disclosure reduces Gas6-mediated phosphorylation of AKT with an IC50 value of 0.019 nM to 7.74 nM. In some embodiments that may be combined with any of the embodiments herein, an anti-MerTK antibody of the present disclosure reduces Gas6-mediated phosphorylation of AKT with an IC50 value of 0.019 nM to 0.25 nM, 0.25 nM to 0.5 nM, 0.5 nM to 1 nM, 1 nM to 2.5 nM, 2.5 nM to 5 nM, or 5 nM to 7.74 nM.

In some embodiments that may be combined with any of the embodiments herein, an anti-MerTK antibody of the present disclosure binds to human MerTK with a binding affinity of 1.4 nM to 81 nM. In some embodiments that may be combined with any of the embodiments herein, an anti-MerTK antibody of the present disclosure binds to human MerTK with a binding affinity of 1.6 nM to 107 nM. In some embodiments that may be combined with any of the embodiments herein, an anti-MerTK antibody of the present disclosure binds to human MerTK with a binding affinity of 30 nM to 186 nM.

In some embodiments that may be combined with any of the embodiments herein, an anti-MerTK antibody of the present disclosure is at least 1.7 times, at least 2.3 times, at least 2.4 times, at least 5 times, at least 7.7 times, or at least 8.5 times more effective at reducing or inhibiting the binding of ProS to MerTK than at reducing or inhibiting the binding of Gas6 to MerTK.

In some embodiments that may be combined with any of the embodiments provided here, an anti-MerTK antibody of the present disclosure decreases or reduces efferocytosis by phagocytic cells (e.g., by at least 50%). In some embodiments that may be combined with any of the embodiments provided here, an anti-MerTK antibody of the present disclosure decreases or reduces efferocytosis by phagocytic cells by at least 60%, by at least 70%, by at least 80%, by at least 90%, or by at least 95%.%). In some embodiments that may be combined with any of the embodiments provided here, an anti-MerTK antibody of the present disclosure decreases or reduces efferocytosis by phagocytic cells with an IC50 value of 0.13 nM to 30 nM. In some embodiments that may be combined with any of the embodiments provided here, an anti-MerTK antibody of the present disclosure decreases or reduces efferocytosis by phagocytic cells with an IC50 value of of 0.13 nM to 1 nM, 1 nM to 5 nM, 5 nM to 10 nM, or 10 nM to 30 nM. In some embodiments, the phagocytic cells are macrophages. In some embodiments, the phagocytic cells are tumore-associated macrophages. In some embodiments, the phagocytic cells are dendritic cells.

In some embodiments that may be combined with any of the embodiments provided herein, an anti-MerTK antibody of the present disclosure increases or induces M1-like macrophage polarization. In some embodiments that may be combined with any of the embodiments provided herein, an anti-MerTK antibody of the present disclosure increases production, expression, and/or secretion of pro-inflammatory cytokines. In certain embodiments, the pro-inflammatory cytokines are tumor necrosis factor (TNF), interferon (IFN), or interleukin-12 (IL-12). In certain embodiments, the pro-inflammatory cytokines are chemokine (C—X—C motif) ligand 1 (CXCL-1, KC), monocyte chemoattractant protein-1 (MCP1, CCL2), macrophage inflammatory protein-1-alpha (MIP-Ilt, CCL3), macrophage inflammatory protein-1-beta (MIP-1β, CCL4), or interleukin-6 (IL-6). In some embodiments that may be combined with any of the embodiments provided herein, an anti-MerTK antibody of the present disclosure increases production, expression, and/or secretion of pro-inflammatory cytokines in vitro. In some embodiments that may be combined with any of the embodiments provided herein, an anti-MerTK antibody of the present disclosure increases production, expression, and/or secretion of pro-inflammatory cytokines in vivo.

In some embodiments that may be combined with any of the embodiments herein, the anti-MerTK antibody of the present disclosure is a monoclonal antibody. In some embodiments that may be combined with any of the embodiments herein, the antibody is a human antibody. In some embodiments that may be combined with any of the embodiments herein, the antibody is a humanized antibody. In some embodiments that may be combined with any of the embodiments herein, the antibody is a bispecific antibody. In some embodiments that may be combined with any of the embodiments herein, the antibody is a multivalent antibody. In some embodiments that may be combined with any of the embodiments herein, the antibody is a chimeric antibody.

In one aspect, the present disclosure relates to an isolated anti-MerTK antibody that binds to a MerTK protein, wherein the antibody is a humanized form of an anti-MerTK antibody provided herein.

In some embodiments that may be combined with any of the embodiments herein, the anti-MerTK antibody of the present disclosure is of the IgG class, the IgM class, or the IgA class. In some embodiments, the antibody is of the IgG class and has an IgG1, IgG2, or IgG4 isotype. In some embodiments, the antibody is of an IgG1 isotype with an Fc amino acid sequence selected from the group consisting of SEQ ID NOs: 396, 397, 398, 399, 400, and 401.

In certain embodiments that may be combined with any of the embodiments herein, the antibody is a full-length antibody. In certain embodiments that may be combined with any of the embodiments herein, the antibody is an antibody fragment. In certain embodiments that may be combined with any of the embodiments herein, the antibody is an antibody fragment that binds to an epitope on human MerTK or a mammalian MerTK protein. In certain embodiments that may be combined with any of the embodiments herein, the antibody fragment is a Fab, Fab′, Fab′-SH, F(ab′)2, Fv, or scFv fragment.

In another aspect, the present disclosure relates to an isolated nucleic acid including a nucleic acid sequence encoding an anti-MerTK antibody of any of the preceding embodiments. In some embodiments, the present disclosure relates to a vector including the nucleic acid of any of the preceding embodiments. In some embodiments, the present disclosure relates to an isolated host cell including the nucleic acid of any of the preceding embodiments or the vector of any of the preceding embodiments. In some embodiments, the present disclosure relates to an isolated host cell comprising (i) a nucleic acid comprising a nucleic acid sequence encoding the VH of an anti-MerTK antibody of any of the preceding embodiments and (ii) a nucleic acid comprising a nucleic acid sequence encoding the VL of the anti-MerTK antibody.

In another aspect, the present disclosure relates to a method of producing an antibody that binds to human MerTK antibody, including culturing the host cell of any of the preceding embodiments so that the anti-MerTK antibody is produced. In certain embodiments, the method further includes recovering the anti-MerTK antibody produced by the cell.

In another aspect, the present disclosure relates to a pharmaceutical composition including an anti-MerTK antibody of any one of the preceding embodiments and a pharmaceutically acceptable carrier.

In one aspect, the present disclosure relates to a method of treating an individual having cancer, the method comprising administering to an individual in need thereof a therapeutically effective amount of an anti-MerTK antibody of any of the preceding embodiments. In some embodiments, the cancer is colon cancer, ovarian cancer, liver cancer, or endometrial cancer. In some embodiments, the cancer is selected from sarcoma, bladder cancer, breast cancer, colon cancer, endometrial cancer, kidney cancer, renal cancer, leukemia, lung cancer, non-small cell lung cancer, melanoma, lymphoma, pancreatic cancer, prostate cancer, ovarian cancer, stomach cancer, thyroid cancer, cancer of the uterus, liver cancer, cervical cancer, testicular cancer, squamous cell carcinoma, glioma, glioblastoma, adenoma, and neuroblastoma. In some embodiments, the cancer is selected from glioblastoma multiforme, bladder carcinoma, and esophageal carcinoma. In some embodiments, the cancer is triple-negative breast carcinoma. In some embodiments, the cancer may be a primary tumor. In some embodiments, the cancer may be a metastatic tumor at a second site derived from any of the above types of cancer. In some embodiments, an anti-MerTK antibody of the present disclosure is useful for treating cancer in s subject in need thereof, wherein the cancer expresses MerTK. In some embodiments, the administration of an anti-MerTK antibody of the present disclosure which reduces efferocytosis does not lead to a retinal pathology in the individual. In some embodiments, the method further comprises administering an anti-PD-L1 antibody, an anti-PD-L2 antibody, or anti-PD antibody (e.g., simultaneously or sequentially) to the individual.

In one aspect, the present disclosure relates to a method of detecting MerTK in a sample comprising contacting said sample with an anti-MerTK antibody of any of the preceding embodiments, optionally wherein the method further comprises detecting the binding of the antibody to MerTK in the sample.

It is to be understood that one, some, or all of the properties of the various embodiments described herein may be combined to form other embodiments of the present disclosure. These and other aspects of the disclosure will become apparent to one of skill in the art. These and other aspects of the disclosure are further described by the detailed description that follows.

### BRIEF DESCRIPTION OF THE DRAWINGS

FIG. 1 sets forth data showing FACS analysis of human MerTK expression on dendritic cells (DCs) and macrophages from two human donors.

FIG. 2 sets forth data showing FACS analysis of human MerTK expression on various cell lines, including A375, THP-1, U937, SK-MEL-5, and CHO-huMerTK OE cells.

FIG. 3 sets forth data showing FACS analysis of human MerTK expression in monocytes, macrophages, and dendritic cells obtained from healthy human subjects or obtained from ovary, liver, or endometrial tumors obtained from human subjects.

FIG. 4 sets forth data showing the effect of anti-MerTK antibodies of the present disclosure on reducing efferocytosis in human macrophages.

FIG. 5 sets forth data showing the effect of anti-MerTK antibodies of the present disclosure on reducing efferocytosis in mouse bone marrow-derived macrophages.

FIG. 6 sets forth data showing anti-MerTK antibodies of the present disclosure bind SK-MEL-5 cells.

FIG. 7 sets forth data showing anti-MerTK antibodies of the present disclosure blocked Gas6 ligand binding to human MerTK in a dose-dependent manner.

FIG. 8 sets forth data showing anti-MerTK antibodies of the present disclosure blocked ProS ligand binding to human MerTK in a dose-dependent manner.

FIG. 9 sets forth data showing epitope binning results of anti-MerTK antibodies of the present disclosure.

FIGS. 10A and 10B set forth data showing anti-MerTK antibody treatment alone or in combination with anti-PD-L1 antibody treatment reduced tumor growth in vivo.

FIGS. 11A, 11B, 11C, and 11D set forth data showing changes in the level of MCP1, MIP-1α, MIP-1, and TNF in human macrophages following overnight treatment with anti-MerTK antibodies of the present disclosure.

FIGS. 12A and 12B set forth data showing reduced tumor growth in mice administered anti-PDL1 antibody in combination with anti-MerTK antibodies of the present disclosure.

FIG. 13 sets forth data showing reduced tumor growth in mice administered anti-PDL1 antibody in combination with anti-MerTK antibodies of the present disclosure.

FIGS. 14A, 14B, 14C, and 14D set forth data showing differences in tumor growth rate in mice administered anti-PDL1 antibody in combination with anti-MerTK antibodies of the present disclosure, plotted using a linear scale y-axis.

FIGS. 15A, 15B, 15C, and 15D set forth data showing differences in tumor growth rate in mice administered anti-PDL1 antibody in combination with anti-MerTK antibodies of the present disclosure, plotted using a log 2 scale y-axis.

FIG. 16 sets forth data showing survival curves of tumor-bearing mice administered anti-PDL1 antibody in combination with anti-MerTK antibodies of the present disclosure.

FIGS. 17A, 17B, 17C, 17D, and 17E set forth data showing changes in levels of chemokine (C—X—C motif) ligand 1 (CXCL-1, KC), monocyte chemoattractant protein-1 (MCP1, CCL2), macrophage inflammatory protein-1-alpha (MIP-1α, CCL3), macrophage inflammatory protein-1-beta (MIP-1β, CCL4), and interleukin-6 (IL-6) in plasma from mice following administration of anti-MerTK antibodies of the present disclosure.

FIGS. 18A, 18B, 18C, and 18D set forth data showing changes in levels of MCP1, MIP-1β, tumor necrosis factor (TNF), and interferon (IFN) in plasma from cynomolgus monkeys administered anti-MerTK antibodies of the present disclosure.

FIGS. 19A and 19B set forth data showing changes in the ratio of phosphor-AKT (pAKT) to total AKT (tAKT) in cells treated with various anti-MerTK antibodies of the present disclosure.

FIGS. 20A, 20B, 20C, 20D, and 20E set forth data showing changes in tumor volume in a MC38 tumor mouse model in vivo in animals administered anti-PDL-1 antibody in combination with various anti-MerTK antibodies of the present disclosure.

FIG. 21 sets forth data showing binding equilibrium dissociation constants (K_{D}) of anti-MerTK antibodies of the present disclosure to human (hu), murine (mu), and cynomolgus (cy) MerTK protein.

### DETAILED DESCRIPTION OF THE PRESENT DISCLOSURE

The present disclosure relates to anti-MerTK antibodies (e.g., monoclonal antibodies); methods of making and using such antibodies; pharmaceutical compositions comprising such antibodies; nucleic acids encoding such antibodies; and host cells comprising nucleic acids encoding such antibodies.

The techniques and procedures described or referenced herein are generally well understood and commonly employed using conventional methodology by those skilled in the art, such as, for example, the widely utilized methodologies such as those described in Sambrook et al. Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003); Monoclonal Antibodies: A Practical Approach (P. Shepherd and C. Dean, eds., Oxford University Press, 2000).

### I. Definitions

The terms “MerTK” or “MerTK polypeptide” or “MerTK protein” are used interchangeably herein refer herein to any native MerTK from any vertebrate source, including mammals such as primates (e.g., humans and cynomolgus monkeys (cynos)) and rodents (e.g., mice and rats), unless otherwise indicated. MerTK is also referred to as c-mer, MER, Proto-oncogene c-Mer, Receptor Tyrosine Kinase MerTK, Tyrosine-protein Kinase Mer, STK Kinase, RP38, and MGC133349. In some embodiments, the term encompasses both wild-type sequences and naturally occurring variant sequences, e.g., splice variants or allelic variants. In some embodiments, the term encompasses “full-length,” unprocessed MerTK as well as any form of MerTK that results from processing in the cell. In some embodiments, the MerTK is human MerTK. As used herein, the term “human MerTK” refers to a polypeptide with the amino acid sequence of SEQ ID NO: 1.

The terms “anti-MerTK antibody,” an “antibody that binds to MerTK,” and “antibody that specifically binds MerTK” refer to an antibody that is capable of binding MerTK with sufficient affinity such that the antibody is useful as a diagnostic and/or therapeutic agent in targeting MerTK. In one embodiment, the extent of binding of an anti-MerTK antibody to an unrelated, non-MerTK polypeptide is less than about 10% of the binding of the antibody to MerTK as measured, e.g., by a radioimmunoassay (RIA). In certain embodiments, an antibody that binds to MerTK has a dissociation constant (KD) of <1 μM, <100 nM, <10 nM, <1 nM, <0.1 nM, <0.01 nM, or <0.001 nM (e.g., 10⁻⁸ M or less, e.g. from 10⁻⁸ M to 10⁻¹³ M, e.g., from 10⁻⁹ M to 10⁻¹³ M). In certain embodiments, an anti-MerTK antibody binds to an epitope of MerTK that is conserved among MerTK from different species.

With regard to the binding of an antibody to a target molecule, the term “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a KD for the target of about any of 10⁻⁴ M or lower, 10⁻⁵ M or lower, 10⁻⁶ M or lower, 10⁻⁷ M or lower, 10⁻⁸ M or lower, 10⁻⁹ M or lower, 10⁻¹⁰ M or lower, 10⁻¹¹ M or lower, 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In one embodiment, the term “specific binding” refers to binding where a molecule binds to a particular polypeptide or epitope on a particular polypeptide without substantially binding to any other polypeptide or polypeptide epitope.

The term “immunoglobulin” (Ig) is used interchangeably with “antibody” herein. The term “antibody” herein is used in the broadest sense and specially covers monoclonal antibodies, polyclonal antibodies, multispecific antibodies (e.g., bispecific antibodies) including those formed from at least two intact antibodies, and antigen-binding antibody fragments so long as they exhibit the desired biological activity.

“Native antibodies” are usually heterotetrameric glycoproteins of about 150,000 Daltons, composed of two identical light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (VH) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, C T, 1994, page 71 and Chapter 6.

The light chain from any vertebrate species can be assigned to one of two clearly distinct types, called kappa (“K”) and lambda (“X”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”), and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three-dimensional configurations of different classes of immunoglobulins are well known and described generally in, for example, Abbas et al., Cellular and Molecular Immunology, 4^{th} ed. (W.B. Saunders Co., 2000).

The “variable region” or “variable domain” of an antibody, such as an anti-MerTK antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-MerTK antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody, such as a monoclonal anti-MerTK antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, but not limited to one or more of the following methods, immunization methods of animals including, but not limited to rats, mice, rabbits, guinea pigs, hamsters and/or chickens with one or more of DNA(s), virus-like particles, polypeptide(s), and/or cell(s), the hybridoma methods, B-cell cloning methods, recombinant DNA methods, and technologies for producing human or human-like antibodies in animals that have parts or all of the human immunoglobulin loci or genes encoding human immunoglobulin sequences.

The terms “full-length antibody,” “intact antibody” or “whole antibody” are used interchangeably to refer to an antibody, such as an anti-MerTK antibody of the present disclosure, in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibodyfragment” refers to a molecule other than an intact antibody that comprises a portion of an intact antibody that binds the antigen to which the intact antibody binds. Examples of antibody fragments include Fab, Fab′, F(ab′)₂ and Fv fragments; diabodies; linear antibodies (see U.S. Pat. 5641870, Example 2; Zapata et al., Protein Eng. 8(10):1057-1062 (1995)); single-chain antibody molecules and multispecific antibodies formed from antibody fragments.

Papain digestion of antibodies, such as anti-MerTK antibodies of the present disclosure, produces two identical antigen-binding fragments, called “Fab” fragments, and a residual “Fc” fragment, a designation reflecting the ability to crystallize readily. The Fab fragment consists of an entire light chain along with the variable region domain of the heavy chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both heavy chains held together by disulfides. The effector functions of antibodies are determined by sequences in the Fc region, the region which is also recognized by Fc receptors (FcR) found on certain types of cells.

“Functional fragments” of antibodies, such as anti-MerTK antibodies of the present disclosure, comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the variable domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains.

As used herein, a “chimeric antibody” refers to an antibody (immunoglobulin), such as a chimeric anti-MerTK antibody of the present disclosure, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies, such as humanized forms of anti-MerTK antibodies of the present disclosure, are chimeric antibodies comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one that possesses an amino-acid sequence corresponding to that of an antibody, such as an anti-MerTK antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries and yeast-display libraries. Human antibodies can be prepared by administering the antigen to a transgenic animal that has been modified to produce such antibodies in response to antigenic challenge, but whose endogenous loci have been disabled, e.g., immunized xenomice as well as generated via a human B-cell hybridoma technology.

The term “hypervariable region,” “HVR,” or “HV,” when used herein refers to the regions of an antibody-variable domain, such as that of an anti-MerTK antibody of the present disclosure, that are hypervariable in sequence and/or form structurally defined loops. Generally, antibodies comprise six HVRs; three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. Naturally occurring camelid antibodies consisting of a heavy chain only are functional and stable in the absence of light chain.

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the V_{L}, and 26-35 (H1), 50-65 or 49-65 (a preferred embodiment) (H2), and 93-102, 94-102, or 95-102 (H3) in the V_{H}. The variable-domain residues are numbered according to Kabat et al., supra, for each of these extended-HVR definitions.

“Framework” or “FR” residues are those variable-domain residues other than the HVR residues as herein defined.

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework may comprise the same amino acid sequence thereof, or it may comprise pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a V_{H}, preferable those changes occur at only three, two, or one of positions 71H, 73H and 78H; for instance, the amino acid residues at those positions may by 71A, 73T and/or 78A. In one embodiment, the V_{L} acceptor human framework is identical in sequence to the V_{L} human immunoglobulin framework sequence or human consensus framework sequence.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences ofProteins of Immunological Interest, 5th Ed. Public Health Service, National Institutes of Health, Bethesda, MD (1991). Examples include for the V_{L}, the subgroup may be subgroup kappa I, kappa II, kappa III or kappa IV as in Kabat et al., supra. Additionally, for the V_{H}, the subgroup may be subgroup I, subgroup II, or subgroup III as in Kabat et al., supra.

An “amino-acid modification” at a specified position, e.g., of an anti-MerTK antibody of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

“Fv” is the minimum antibody fragment which comprises a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains, which enables the sFv to form the desired structure for antigen binding.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least 80% homology with a native sequence Fc region and/or with an Fc region of a parent polypeptide, and most preferably at least 90% homology therewith, more preferably at least 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies.

As used herein, “percent (%) amino acid sequence identity” and “homology” with respect to a peptide, polypeptide or antibody sequence refers to the percentage of amino acid residues in a cantidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

The term “compete” when used in the context of antibodies that compete for the same epitope or overlapping epitopes means competition between antibody as determined by an assay in which the antibody being tested prevents or inhibits (e.g., reduces) specific binding of a reference molecule (e.g., a ligand, or a reference antibody) to a common antigen (e.g., MerTK or a fragment thereof). Numerous types of competitive binding assays can be used to determine if antibody competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antibody and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit (e.g., reduce) specific binding of a reference antibody to a common antigen by at least 20%, 30%, 40%, 50%, 60%, 70%, 80%, 85%, 90%, 95%, 97.5%, and/or near 100%.

As used herein, an “interaction” between a MerTK polypeptide and a second polypeptide encompasses, without limitation, protein-protein interaction, a physical interaction, a chemical interaction, binding, covalent binding, and ionic binding. As used herein, an antibody “inhibits interaction” between two polypeptides when the antibody disrupts, reduces, or completely eliminates an interaction between the two polypeptides. An antibody of the present disclosure, thereof, “inhibits interaction” between two polypeptides when the antibody thereof binds to one of the two polypeptides. In some embodiments, the interaction can be inhibited by at least any of 20%, 30%, 40%, 50%, 60%, 70%, 80%, 85%, 90%, 95%, 97.5%, and/or near 100%.

The term “epitope” includes any determinant capable of being bound by an antibody. An epitope is a region of an antigen that is bound by an antibody that targets that antigen, and when the antigen is a polypeptide, includes specific amino acids that directly contact the antibody. Most often, epitopes reside on polypeptides, but in some instances, can reside on other kinds of molecules, such as nucleic acids. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of polypeptides and/or macromolecules.

An “isolated” antibody, such as an isolated anti-MerTK antibody of the present disclosure, is one that has been identified, separated and/or recovered from a component of its production environment (e.g., naturally or recombinantly). Preferably, the isolated antibody is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the antibody will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T-cells since at least one component of the antibody's natural environment will not be present. Ordinarily, however, an isolated polypeptide or antibody will be prepared by at least one purification step.

An “isolated” nucleic acid molecule encoding an antibody, such as an anti-MerTK antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

“Carriers” as used herein include pharmaceutically acceptable carriers, excipients, or stabilizers that are nontoxic to the cell or mammal being exposed thereto at the dosages and concentrations employed.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic result. An effective amount can be provided in one or more administrations. An effective amount is also one in which any toxic or detrimental effects of the treatment are outweighed by the therapeutically beneficial effects. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “individual” for purposes of treatment refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the individual is human.

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration. In some embodiments, administration in conjunction is administration as a part of the same treatment regimen.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the present disclosure described herein include “comprising,” “consisting,” and “consisting essentially of” aspects and embodiments.

### I. Anti-MerTK Antibodies

Provided herein are anti-MerTK antibodies. Antibodies provided herein are useful, e.g., for the diagnosis or treatment of the MerTK associated disorders.

In one aspect, the present disclosure provides isolated (e.g., monoclonal) antibodies that bind to an epitope within a MerTK protein or polypeptide of the present disclosure. MerTK proteins or polypeptides of the present disclosure include, without limitation, a mammalian MerTK protein or polypeptide, human MerTK protein or polypeptide, mouse (murine) MerTK protein or polypeptide, and cynomogus MerTK protein or polypeptide. MerTK proteins and polypeptides of the present disclosure include naturally occurring variants of MerTK. In some embodiments, MerTK proteins and polypeptides of the present disclosure are membrane bound. In some embodiments, MerTK proteins and polypeptides of the present disclosure are a soluble extracellular domain of MerTK.

In some embodiments, MerTK is expressed in a cell. In some embodiments, MerTK is expressed in phagocytic cells, including without limitation, macrophages and dendritic cells. In some embodiments, MerTK is expressed in monocytes, natural killer cells, natural killer T cells, microglia, endothelial cells, megakaryocytes, and platelets. In some embodiments, high levels of MerTK expression are also found in ovary, prostate, testis, lung, retina, and kidney. Additionally, MerTK displays ectopic or overexpression in numerous cancers (Linger et al, 2008, Adv Cancer Res, 100:35-83).

### Antibody Activities

In some embodiments, an anti-MerTK antibody is provided that binds to human MerTK but does not bind to cynomolgus MerTK. In some embodiments, an anti-MerTK antibody is provided that binds to human MerTK but does not bind to murine MerTK. In some embodiments, an anti-MerTK antibody is provided that binds to human MerTK but does not bind to cynomolgus MerTK and does not bind to murine MerTK. In some embodiments, an anti-MerTK antibody is provided that binds to human MerTK and binds to cynomolgus MerTK. In some embodiments, an anti-MerTK antibody is provided that binds to human MerTK and binds to cynomolgus MerTK but does not bind to murine MerTK. In some embodiments, an anti-MerTK antibody is provide that binds to human MerTK and binds to murine MerTK. In some embodiments, an anti-MerTK antibody is provide that binds to human MerTK and binds to murine MerTK but does not bind cynomolgus MerTK. In some embodiments, an anti-MerTK antibody is provide that binds to human MerTK, cynomolgus MerTK, and murine MerTK. An anti-MerTk antibody that binds to human, cynomolgus, and murine MerTK is advantageous for performing in vivo studies in mammalian animal models of disease (e.g., cancer) as well as studies in non-human primates.

### MerTK Binding Partners

MerTK proteins of the present disclosure interact with (e.g., bind) one or more ligands or binding partners, including, without limitation, Protein S (ProS or ProS1), Growth arrest specific gene 6 (Gas6), Tubby, Tubby-like protein 1 (TULP-1), and Galectin-3. Anti-MerTK antibodies of the present disclosure can affect the interaction of MerTK with one or more of its various ligands and binding partners.

Anti-MerTK antibodies of the present disclosure that blocked both ProS binding and Gas6 binding to MerTK, that blocked only ProS binding to MerTK, or that did not block binding of either ProS or Gas6 binding to MerTK were identified. The ProS only blocking anti-MerTK antibodies did not bin with the ProS/Gas6 double blocking anti-MerTK antibodies (as shown in the Examples below), suggesting non-overlapping epitopes for these two classes of anti-MerTK antibodies identified herein.

Accordingly, in some embodiments, an anti-MerTK antibody of the present disclosure inhibits (i.e., blocks) or reduces binding between MerTK and one or more MerTK ligands. In some embodiments, an anti-MerTK antibody of the present disclosure inhibits or reduces binding of ProS to MerTK. In some embodiments, an anti-MerTK antibody of the present disclosure inhibits or reduces binding of ProS to MerTK but does not inhibit or reduce binding of Gas6 to MerTK. In some embodiments, an anti-MerTK antibody of the present disclosure inhibits or reduces binding of Gas6 to MerTK. In some embodiments, an anti-MerTK antibody of the present disclosure inhibits or reduces binding to Gas6 to MerTK but does not inhibit or reduce binding of ProS to MerTK. In yet other embodiments, an anti-MerTK antibody of the present disclosure inhibits or reduces binding of ProS to MerTK and inhibits or reduces binding of Gas6 to MerTK. In other embodiments, an anti-MerTK antibody of the present disclosure does not inhibit or reduce binding of ProS to MerTK and does not inhibit or reduce binding of Gas6 to MerTK.

An IC50 value for inhibition or blocking of Gas6 ligand binding to MerTK or for inhibition or blocking of ProS ligand binding to MerTK can be determined using methods known by one of skill in the art, such as that described herein in the Example below (e.g., Example 13). In some embodiments, an anti-MerTK antibody of the present disclosure inhibits or reduces Gas6 ligand binding or inhibits or reduces ProS ligand binding to MerTK in vitro. In some embodiments, an anti-MerTK antibody of the the present disclosure inhibits or reduces Gas6 ligand binding to human MerTK with an IC50 value in the range of 0.29 nM to 32 nM. In some embodiments, an anti-MerTK antibody of the present disclosure inhibits or reduces Gas6 ligand binding to human MerTK with an IC50 value in the range of 0.29 nM to 1.0 nM, in the range of 1.0 nM to 5 nM, in the range of 5 nM to 10 nM, in the range of 10 nM to 25 nM, or in the range of 25 nM to 32 nM. In some embodiments, an anti-MerTK antibody of the present disclosure inhibits or reduces ProS ligand binding to human MerTK with an IC50 value in the range of 0.58 nM to 25.9 nM. In some embodiments, an anti-MerTK antibody of the present disclosure inhibits or reduces ProS ligand binding to human MerTK with an IC50 value in the range of 0.58 nM to 1 nM, in the range of 1 nM to 2.5 nM, in the range of 2.5 nM to 5 nM, in the range of 5 nM to 10 nM, in the range of 10 nM to 25 nM.

The relative effectiveness of an antibody at reducing or inhibiting the binding of ProS and Gas6 to MerTK can be determined using such IC50 values. For example, an antibody that inhibits binding of Gas6 to MerTK at an IC50 value that is 1.7 times greater than its IC50 value for inhibiting binding of ProS to MerTK is 1.7 times more effective at reducing or inhibiting the binding of ProS to MerTK than at reducing or inhibiting the binding of Gas6 to MerTK. In some embodiments, an anti-MerTK antibody is at least 1.7 times, at least 2.3 times, at least 2.4 times, at least 5 times, at least 7.7 times, or at least 8.5 times more effective at reducing or inhibiting the binding of ProS to MerTK than at reducing or inhibiting the binding of Gas6 to MerTK.

In some embodiments, an anti-MerTK antibody of the present disclosure inhibits or reduces Gas6 ligand binding to MerTK by at least 20%, by at least 25, by at least 30%, by at least 40%, by at least 50%, by at least 60%, by at least 70%, by at least 80%, by at least 90%, or by at least 95% compared to Gas6 ligand binding to MerTK in the absence of an anti-MerTK antibody of the present disclosure. In some embodiments, an anti-MerTK antibody of the present disclosure inhibits or reduces ProS ligand binding to MerTK by at least 20%, by at least 25%, by at least 30%, by at least 40%, by at least 50%, by at least 60%, by at least 70%, by at least 80%, by at least 90%, or by at least 95% compared to ProS ligand binding to MerTK in the absence of an anti-MerTK antibody of the present disclosure. Percent inhibition or reduction of Gas6 ligand binding to MerTK or of ProS ligand binding to MerTK can be determined by measuring IC50 values using standard methods known by one of skill in the art, such as described herein in Example 13.

Further provided herein are methods of screening for anti-MerTK antibodies that bind MerTK and that block or reduce the interactions between MerTK and one or more MerTK ligands or binding partners.

pAKT

AKT activity is a downstream target of Gas6 binding to MerTK, Axl, or Tyro-3 receptors. For example, the binding of MerTK ligand Gas6 to MerTK induces AKT phosphorylation (pAKT) (see, e.g., Angelillo-Scherrer et al, 2008, J Clin Invest, 118:583-596; Moody et al, 2016, Int J Cancer, 139:1340-1349). Anti-MerTK antibodies of the present disclosure were effective at inhibiting Gas6-mediated phospho-AKT (pAKT) activity in human macrophages (e.g., M2c macrophages) in a dose-dependent manner. Accordingly, anti-MerTK antibodies of the present disclosure were effective at inhibiting Gas6-mediated MerTK signaling as evidenced by inhibition of pAKT levels. In some embodiments, an anti-MerTK antibody of the present disclosure inhibits or reduces Gas6-mediated pAKT activity in vitro.

The relative effectiveness of an anti-MerTK antibody at reducing or inhibiting pAKT activity in a cell can be determined by measuring the IC50 values. IC50 values for inhibition of Gas6-mediated pAKT activity can be determined using methods known by one of skill in the art, such as that described herein in Example 22 below. In some embodiments, an anti-MerTK antibody of the present disclosure inhibits Gas6-mediated pAKT activity in macrophages (e.g., M2c macrophages) with an IC50 value ranging from 0.019 nM to 7.74 nM. In some embodiments, an anti-MerTK antibody of the present disclosure inhibits Gas6-mediated pAKT activity in macrophages (e.g., M2c macrophages) with an IC50 value of 0.019 nM to 0.25 nM, 0.25 nM to 0.50 nM, 0.50 nM to 1.0 nM, 1.0 nM to 2.5 nM, 2.5 nM to 5.0 nM, or 5.0 nM to 10 nM.

### Efferocytosis

Efferocytosis refers to phagocytic clearance of dying or apoptotic cells. Efferocytosis can be accomplished by professional phagocytes (e.g., macrophages, dendritic cells, microglia), non-professional phagocytes (e.g., epithelial cells, fibroblasts, retinal pigment epithelial cells), or specialized phagocytes. (Elliott et al, 2017, J Immunol, 198:1387-1394). Efferocytosis leads to the removal of dead or dying cells before their membrane integrity is breached and their cellular contents leak into the surrounding tissue, thus preventing exposure of tissue to toxic enzymes, oxidants, and other intracellular components.

Apoptotic cells expose a variety of molecules on their cell surface (“eat-me” signals) that are recognized by receptors on phagocytic cells. One such “eat me” signaling molecules is phosphatidylserine (PtdSer), which is normally confined to the inner leaflet of the cell membrane. During apoptosis, PtdSer is exposed to the outer leaflet of the cell membrane. MerTK ligands ProS and Gas6 contain gamma-carboxylated glutamic acid residues near their N-terminal domains; gamma-carboxylation of the glutamic acid domain enables binding to phosphatidylserine. Gas6 or ProS bind to PtdSer on apoptotic cells and simultaneously bind MerTK on phagocytes. Such ligand engagement with MerTK activates efferocytosis.

As shown in the Examples below, anti-MerTK antibodies of the present disclosure were effective at reducing or decreasing efferocytosis in various phagocytic cells. Anti-MerTK antibodies that that block ProS only binding to MerTK showed robust inhibition of efferocytosis, having an IC50 comparable to anti-MerTK antibodies that block both ProS and Gas6 binding to MerTK. Considering that genetic deficiency of both ProS and Gas6 is required to develop vision loss by retinal degradation (ProS or Gas6 single genetic knockouts did not lead to retinal pathologies), anti-MerTK antibodies that are ProS-specific blockers may provide improved therapeutic index for use in anti-tumor responses without inducing eye toxicity issues.

Accordingly, in some embodiments, an anti-MerTK antibody of the present disclosure decreases or reduces efferocytosis by phagocytic cells. In some embodiments, an anti-MerTK antibody of the present disclosure decreases or reduces efferocytosis by macrophages. In some embodiments, an anti-MerTK antibody of the present disclosure decreases or reduces efferocytosis by dendritic cells. In some embodiments, an anti-MerTK antibody of the present disclosure decreases or reduces efferocytosis by bone marrow-derived macrophages. In some embodiments, an anti-MerTK antibody of the present disclosure decreases or reduces efferocytosis by phagocytic cells by at least 10%, by at least 20%, by at least 25%, by at least 30%, by at least 40%, by at least 50%, by at least 60%, by at least 70%, by at least 80%, by at least 90%, or by at least 95% compared to the level of efferocytosis in phagocytic cells in the absence of an anti-MerTK antibody. Percent reduction of efferocytosis can be determined using standard methods known to one of skill in the art, such as described herein in Example 11.

In some embodiments, an anti-MerTK antibody of the present disclosure decreases or reduces efferocytosis by a phagocytic cell (e.g., a human macrophage) with an IC50 in the range of about 0.13 nM to about 30 nM, as assessed by methods described herein in the Example below (e.g., Example 11). In some embodiments, an anti-MerTK antibody of the present disclosure decreases or reduces efferocytosis by a phagocytic cell with an IC50 value in the range of 0.13 nM to 1.0 nM, in the range of 1 nM to 5 nM, in the range of 5 nM to 10 nM, or in the range of 10 nM to 30 nM.

Blocking efferocytosis drives M1-like macrophage polarization, resulting in increased production of pro-inflammatory cytokines (e.g., TNF, IFN, IL-12) and recruitment of cytotoxic cells, such as CD8+ T cells and natural killer cells, that mediate anti-tumor immunity. By reducing efferocytosis by phagocytic cells, anti-MerTK antibodies of the present disclosure are effective at increasing M1-like macrophage polarization and at increasing production, expression, and/or secretion of pro-inflammatory cytokines/chemokines, including TNF, IFN, IL-6, IL-1, IL-12, chemokine (C—X—C motif) ligand 1 (CXCL-1, KC), monocyte chemoattractant protein-1 (MCP1, CCL2), macrophage inflammatory protein-1-alpha (MIP-1α, CCL3), and/or macrophage inflammatory protein-1-beta (MIP-1β, CCL4). Accordingly, in some embodiments, an anti-MerTK antibody of the present disclosure increases M1-like macrophage polarization. In some embodiments, an anti-MerTK antibody of the present disclosure increases macrophage production, expression, or secretion of one or more pro-inflammatory cytokines. In some embodiments, an anti-MerTK antibody of the present disclosure increases macrophage production, expression, or secretion of TNF, IFN, IL-6, IL-1, IL-12, CXCL-1, MCP1, MIP-1α, and/or MIP-1β.

Accordingly, in some embodiments, provided herein is a method for increasing pro-inflammatory cytokine production, expression, and/or secretion in a subject in need thereof, the method comprising administering to the subject an anti-MerTK antibody of the present disclosure such that the production, expression, and/or secretion of one or more pro-inflammatory cytokines in the subject is increased. In some embodiments, the production, expression, and/or secretion of TNF is increased. In some embodiments, the production, expression, and/or secretion of IFN is increased. In some embodiments, the production, expression, and/or secretion of IL-12 is increased. In some embodiments, the production, expression, and/or secretion of CXCL-1 is increased. In some embodiments, the production, expression, and/or secretion of MCP1 is increased. In some embodiments, the production, expression, and/or secretion of MIP-1α is increased. In some embodiments, the production, expression, and/or secretion of MIP-1β is increased.

In some embodiments, provided herein is a method for increasing M1-like macrophage polarization in a subject in need thereof, the method comprising administering to the subject an anti-MerTK antibody of the present disclosure such that M1-like macrophage polarization in the subject is increased. Increased M1-like macrophage polarization is measured by various methods known to one of skill in the art, such as increased production, expression, or secretion of one or more pro-inflammatory cytokines, including TNF, IFN, IL-12, CXCL1, MCP1, MIP-1α, and/or MIP-1.

A link between efferocytosis and cancer progression has been described. For example, blockade of efferocytosis using Annexin V, which blocks PtdSer from interacting with the efferocytosis machinery of phagocytes, sufficiently reduces tumor progression and metastasis (Stach et al, 2000, Cell Death Diff, 7:911; Bondanza et al, 2004, J Exp Med, 200:1157; Werfel and Cook, 2018, Sem Immunopathology, 40:545-554). Further, MerTK correlates with poor prognosis and survival in numerous human cancers, as does its PtdSer bridging ligand Gas6 (Graham et al, 2014, Nat Rev Cancer, 14:769; Linger et al, 2010, Expert Opin Ther Targets, 14:1073-1090; Wang et al, 2013, Oncogene, 32:872; Jansen et al, 2011, J Proteome Res, 11:728-735; Tworkoski et al, 2011, Mol Cancer Res, p. molcanres-0512; Graham et al, 2006, Clin Cancer Res, 12:2662-2669; Keating et al, 2006, Oncogene, 25:6092). Accordingly, anti-MerTK antibodies of the present disclosure, which reduce efferocytosis by phagocytic cells, are thus effective at reducing tumor progression and metastasis.

Cynomolgus studies indicated that in some instances, anti-MerTK antibodies that block binding to both Gas6 and ProS (e.g., anti-MerTK antibody MTK-16) showed less in vivo toxicity (e.g., weight loss) compared to that observed in cynomolgus monkeys administered an anti-MerTK antibody that blocks ProS binding to MerTK but does not block binding of Gas6 to MerTK (e.g., anti-MerTK antbody MTK-15 or MTK-29). Accordingly, in some embodiments, an anti-MertK antibody of the present disclosure that blocks binding of both Gas6 and ProS to MerTK may display less systemic in vivo toxitiy compared to an anti-MerTK antibody of the present disclosure that blocks binding of ProS to MerTK but does not block binding of Gas6 to MerTK. Anti-MerTk antibody MTK-16 (which blocks binding of both Gas6 and ProS to MerTK) binds to the Ig1 domain of MerTK protein, while anti-MerTK antibodies MTK-15 and MTK-29 (which block binding of ProS to MerTK but which do not block binding of Gas6 to MerTK) bind to both the Ig2 and the FN1 domain of MerTK protein. Accordingly, in some embodiments, an anti-MerTK antibody that binds to the Ig 1 domain of MerTK may display less systemic in vivo toxicity than an anti-MerTK antibody that binds to both the Ig2 and FN1 domains of MerTK.

### A. Exemplary Antibodies and Certain Other Antibody Embodiments

In some embodiments, provided herein are anti-MerTK antibodies comprising at least one, two, three, four, five, or six HVRs selected from: (a) HVR-H1 comprising an amino acid sequence selected from the group consisting of SEQ ID NOs:75, 76, 77, 78, 79, 80, 81, 82, 83, 84, 85, 86, 87, 88, 89, 90, 91, 92, 93, 94, 95, 96, 97, and 98; (b) HVR-H2 comprising an amino acid sequence selected from the group consisting of SEQ ID NOs:99, 100, 101, 102, 103, 104, 105, 106, 107, 108, 109, 110, 111, 112, 113, 114, 115, 116, 117, 118, 119, 120, 121, 122, 123, and 124; (c) HVR-H3 comprising an amino acid sequence selected from the group consisting of SEQ ID NOs:125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, and 157; (d) HVR-L1 comprising an amino acid sequence selected from the group consisting of SEQ ID NOs:158, 159, 160, 161, 162, 163, 164, 165, 166, 167, 168, 169, 170, 171, 172, 173, 174, 175, 176, 177, 178, 179, 180, 181, 182, 183, 184, 185, and 186; (e) HVR-L2 comprising an amino acid sequence selected from the group consisting of SEQ ID NOs:187, 188, 189, 190, 191, 192, 193, 194, 195, 196, 197, 198, 199, 200, 201, 202, 203, 204, and 205; and (f) HVR-L3 comprising an amino acid sequence selected from the group consisting of SEQ ID NOs:207, 208, 209, 210, 211, 212, 213, 214, 215, 216, 217, 218, 219, 220, 221, 222, 223, 224, 225, 226, 227, 228, 229, 230, 231, 232, and 233.

In some embodiments, provided herein are anti-MerTK antibodies comprising at least one, at least two, or all three V_{H} HVR sequences selected from (a) HVR-H1 comprising an amino acid sequence selected from the group consisting of SEQ ID NOs: 75, 76, 77, 78, 79, 80, 81, 82, 83, 84, 85, 86, 87, 88, 89, 90, 91, 92, 93, 94, 95, 96, 97, and 98; (b) HVR-H2 comprising an amino acid sequence selected from the group consisting of SEQ ID NOs: 99, 100, 101, 102, 103, 104, 105, 106, 107, 108, 109, 110, 111, 112, 113, 114, 115, 116, 117, 118, 119, 120, 121, 122, 123, and 124; and (c) HVR-H3 comprising an amino acid sequence selected from the group consisting of SEQ ID NOs: 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, and 157.

In some embodiments, provided herein are anti-MerTK antibodies comprising at least one, at least two, or all three V_{L} HVR sequences selected from (a) HVR-L1 comprising an amino acid sequence selected from the group consisting of SEQ ID NOs: 158, 159, 160, 161, 162, 163, 164, 165, 166, 167, 168, 169, 170, 171, 172, 173, 174, 175, 176, 177, 178, 179, 180, 181, 182, 183, 184, 185, and 186; (b) HVR-L2 comprising an amino acid sequence selected from the group consisting of SEQ ID NOs: 187, 188, 189, 190, 191, 192, 193, 194, 195, 196, 197, 198, 199, 200, 201, 202, 203, 204, and 205; and (c) HVR-L3 comprising an amino acid sequence selected from the group consisting of SEQ ID NOs: 207, 208, 209, 210, 211, 212, 213, 214, 215, 216, 217, 218, 219, 220, 221, 222, 223, 224, 225, 226, 227, 228, 229, 230, 231, 232, and 233.

In some embodiments, provided herein are anti-MerTK antibodies comprising (a) a V_{H} domain comprising at least one, at least two, or all three V_{H} HVR sequences selected from (i) HVR-H1 comprising an amino acid sequence selected from the group consisting of SEQ ID NOs: 75, 76, 77, 78, 79, 80, 81, 82, 83, 84, 85, 86, 87, 88, 89, 90, 91, 92, 93, 94, 95, 96, 97, and 98, (ii) HVR-H2 comprising an amino acid sequence selected from the group consisting of SEQ ID NOs: 99, 100, 101, 102, 103, 104, 105, 106, 107, 108, 109, 110, 111, 112, 113, 114, 115, 116, 117, 118, 119, 120, 121, 122, 123, and 124, and (iii) HVR-H3 comprising an amino acid sequence selected from the group consisting of SEQ ID NOs: 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, and 157, and (b) a V_{L} domain comprising at least one, at least two, or all three V_{L} HVR sequences selected from (i) HVR-L1 comprising an amino acid sequence selected from the group consisting of SEQ ID NOs: 158, 159, 160, 161, 162, 163, 164, 165, 166, 167, 168, 169, 170, 171, 172, 173, 174, 175, 176, 177, 178, 179, 180, 181, 182, 183, 184, 185, and 186, (ii) HVR-L2 comprising an amino acid sequence selected from the group consisting of SEQ ID NOs: 187, 188, 189, 190, 191, 192, 193, 194, 195, 196, 197, 198, 199, 200, 201, 202, 203, 204, and 205, and (iii) HVR-L3 comprising an amino acid sequence selected from the group consisting of SEQ ID NOs: 207, 208, 209, 210, 211, 212, 213, 214, 215, 216, 217, 218, 219, 220, 221, 222, 223, 224, 225, 226, 227, 228, 229, 230, 231, 232, and 233.

In some embodiments, provided herein are anti-MerTK antibodies comprising: (a) HVR-H1 comprising the amino acid sequence of SEQ ID NO:75; (b) HVR-H2 comprising the amino acid sequence of SEQ ID NO:99; (c) HVR-H3 comprising the amino acid sequence of SEQ ID NO:125; (d) HVR-L1 comprising the amino acid sequence of SEQ ID NO: 158; (e) HVR-L2 comprising the amino acid sequence of SEQ ID NO: 187; and (f) HVR-L3 comprising the amino acid sequence of SEQ ID NO:207; (a) HVR-H1 comprising the amino acid sequence of SEQ ID NO:76; (b) HVR-H2 comprising the amino acid sequence of SEQ ID NO: 100; (c) HVR-H3 comprising the amino acid sequence of SEQ ID NO: 126; (d) HVR-L1 comprising the amino acid sequence of SEQ ID NO: 159; (e) HVR-L2 comprising the amino acid sequence of SEQ ID NO: 187; and (f) HVR-L3 comprising the amino acid sequence of SEQ ID NO:208; (a) HVR-H1 comprising the amino acid sequence of SEQ ID NO:77; (b) HVR-H2 comprising the amino acid sequence of SEQ ID NO:101; (c) HVR-H3 comprising the amino acid sequence of SEQ ID NO: 127; (d) HVR-L1 comprising the amino acid sequence of SEQ ID NO: 160; (e) HVR-L2 comprising the amino acid sequence of SEQ ID NO:188; and (f) HVR-L3 comprising the amino acid sequence of SEQ ID NO:209; (a) HVR-H1 comprising the amino acid sequence of SEQ ID NO:75; (b) HVR-H2 comprising the amino acid sequence of SEQ ID NO:99; (c) HVR-H3 comprising the amino acid sequence of SEQ ID NO: 128; (d) HVR-L1 comprising the amino acid sequence of SEQ ID NO: 158; (e) HVR-L2 comprising the amino acid sequence of SEQ ID NO:187; and (f) HVR-L3 comprising the amino acid sequence of SEQ ID NO:210; (a) HVR-H1 comprising the amino acid sequence of SEQ ID NO:78; (b) HVR-H2 comprising the amino acid sequence of SEQ ID NO: 102; (c) HVR-H3 comprising the amino acid sequence of SEQ ID NO: 129; (d) HVR-L1 comprising the amino acid sequence of SEQ ID NO: 161; (e) HVR-L2 comprising the amino acid sequence of SEQ ID NO:189; and (f) HVR-L3 comprising the amino acid sequence of SEQ ID NO:211; (a) HVR-H1 comprising the amino acid sequence of SEQ ID NO:75; (b) HVR-H2 comprising the amino acid sequence of SEQ ID NO:103; (c) HVR-H3 comprising the amino acid sequence of SEQ ID NO:130; (d) HVR-L1 comprising the amino acid sequence of SEQ ID NO:158; (e) HVR-L2 comprising the amino acid sequence of SEQ ID NO:187; and (f) HVR-L3 comprising the amino acid sequence of SEQ ID NO:212; (a) HVR-H1 comprising the amino acid sequence of SEQ ID NO:77; (b) HVR-H2 comprising the amino acid sequence of SEQ ID NO:101; (c) HVR-H3 comprising the amino acid sequence of SEQ ID NO: 127; (d) HVR-L1 comprising the amino acid sequence of SEQ ID NO: 162; (e) HVR-L2 comprising the amino acid sequence of SEQ ID NO: 188; and (f) HVR-L3 comprising the amino acid sequence of SEQ ID NO:209; (a) HVR-H1 comprising the amino acid sequence of SEQ ID NO:75; (b) HVR-H2 comprising the amino acid sequence of SEQ ID NO:99; (c) HVR-H3 comprising the amino acid sequence of SEQ ID NO:131; (d) HVR-L1 comprising the amino acid sequence of SEQ ID NO: 163; (e) HVR-L2 comprising the amino acid sequence of SEQ ID NO:187; and (f) HVR-L3 comprising the amino acid sequence of SEQ ID NO:213; (a) HVR-H1 comprising the amino acid sequence of SEQ ID NO:79; (b) HVR-H2 comprising the amino acid sequence of SEQ ID NO:104; (c) HVR-H3 comprising the amino acid sequence of SEQ ID NO: 132; (d) HVR-L1 comprising the amino acid sequence of SEQ ID NO: 164; (e) HVR-L2 comprising the amino acid sequence of SEQ ID NO: 190; and (f) HVR-L3 comprising the amino acid sequence of SEQ ID NO:214; (a) HVR-H1 comprising the amino acid sequence of SEQ ID NO:80; (b) HVR-H2 comprising the amino acid sequence of SEQ ID NO:105; (c) HVR-H3 comprising the amino acid sequence of SEQ ID NO:133; (d) HVR-L1 comprising the amino acid sequence of SEQ ID NO: 165; (e) HVR-L2 comprising the amino acid sequence of SEQ ID NO:191; and (f) HVR-L3 comprising the amino acid sequence of SEQ ID NO:215; (a) HVR-H1 comprising the amino acid sequence of SEQ ID NO:81; (b) HVR-H2 comprising the amino acid sequence of SEQ ID NO: 106; (c) HVR-H3 comprising the amino acid sequence of SEQ ID NO:134; (d) HVR-L1 comprising the amino acid sequence of SEQ ID NO: 166; (e) HVR-L2 comprising the amino acid sequence of SEQ ID NO: 192; and (f) HVR-L3 comprising the amino acid sequence of SEQ ID NO:216; (a) HVR-H1 comprising the amino acid sequence of SEQ ID NO:82; (b) HVR-H2 comprising the amino acid sequence of SEQ ID NO: 107; (c) HVR-H3 comprising the amino acid sequence of SEQ ID NO:135; (d) HVR-L1 comprising the amino acid sequence of SEQ ID NO: 167; (e) HVR-L2 comprising the amino acid sequence of SEQ ID NO: 193; and (f) HVR-L3 comprising the amino acid sequence of SEQ ID NO:217; (a) HVR-H1 comprising the amino acid sequence of SEQ ID NO:77; (b) HVR-H2 comprising the amino acid sequence of SEQ ID NO:108; (c) HVR-H3 comprising the amino acid sequence of SEQ ID NO:136; (d) HVR-L1 comprising the amino acid sequence of SEQ ID NO: 167; (e) HVR-L2 comprising the amino acid sequence of SEQ ID NO: 194; and (f) HVR-L3 comprising the amino acid sequence of SEQ ID NO:209; (a) HVR-H1 comprising the amino acid sequence of SEQ ID NO:75; (b) HVR-H2 comprising the amino acid sequence of SEQ ID NO: 109; (c) HVR-H3 comprising the amino acid sequence of SEQ ID NO:137; (d) HVR-L1 comprising the amino acid sequence of SEQ ID NO: 168; (e) HVR-L2 comprising the amino acid sequence of SEQ ID NO: 187; and (f) HVR-L3 comprising the amino acid sequence of SEQ ID NO:218; (a) HVR-H1 comprising the amino acid sequence of SEQ ID NO:83; (b) HVR-H2 comprising the amino acid sequence of SEQ ID NO:999; (c) HVR-H3 comprising the amino acid sequence of SEQ ID NO:138; (d) HVR-L1 comprising the amino acid sequence of SEQ ID NO:158; (e) HVR-L2 comprising the amino acid sequence of SEQ ID NO:187; and (f) HVR-L3 comprising the amino acid sequence of SEQ ID NO:210; (a) HVR-H1 comprising the amino acid sequence of SEQ ID NO:84; (b) HVR-H2 comprising the amino acid sequence of SEQ ID NO:99; (c) HVR-H3 comprising the amino acid sequence of SEQ ID NO:139; (d) HVR-L1 comprising the amino acid sequence of SEQ ID NO: 169; (e) HVR-L2 comprising the amino acid sequence of SEQ ID NO: 195; and (f) HVR-L3 comprising the amino acid sequence of SEQ ID NO:219; (a) HVR-H1 comprising the amino acid sequence of SEQ ID NO:85; (b) HVR-H2 comprising the amino acid sequence of SEQ ID NO:99; (c) HVR-H3 comprising the amino acid sequence of SEQ ID NO: 140; (d) HVR-L1 comprising the amino acid sequence of SEQ ID NO: 170; (e) HVR-L2 comprising the amino acid sequence of SEQ ID NO: 196; and (f) HVR-L3 comprising the amino acid sequence of SEQ ID NO:222; (a) HVR-H1 comprising the amino acid sequence of SEQ ID NO:86; (b) HVR-H2 comprising the amino acid sequence of SEQ ID NO:99; (c) HVR-H3 comprising the amino acid sequence of SEQ ID NO:141; (d) HVR-L1 comprising the amino acid sequence of SEQ ID NO:171; (e) HVR-L2 comprising the amino acid sequence of SEQ ID NO:191; and (f) HVR-L3 comprising the amino acid sequence of SEQ ID NO:221; (a) HVR-H1 comprising the amino acid sequence of SEQ ID NO:87; (b) HVR-H2 comprising the amino acid sequence of SEQ ID NO:110; (c) HVR-H3 comprising the amino acid sequence of SEQ ID NO: 142; (d) HVR-L1 comprising the amino acid sequence of SEQ ID NO: 172; (e) HVR-L2 comprising the amino acid sequence of SEQ ID NO:197; and (f) HVR-L3 comprising the amino acid sequence of SEQ ID NO:222; (a) HVR-H1 comprising the amino acid sequence of SEQ ID NO:88; (b) HVR-H2 comprising the amino acid sequence of SEQ ID NO:99; (c) HVR-H3 comprising the amino acid sequence of SEQ ID NO: 143; (d) HVR-L1 comprising the amino acid sequence of SEQ ID NO: 173; (e) HVR-L2 comprising the amino acid sequence of SEQ ID NO:191; and (f) HVR-L3 comprising the amino acid sequence of SEQ ID NO:223; (a) HVR-H1 comprising the amino acid sequence of SEQ ID NO:89; (b) HVR-H2 comprising the amino acid sequence of SEQ ID NO:111; (c) HVR-H3 comprising the amino acid sequence of SEQ ID NO:143; (d) HVR-L1 comprising the amino acid sequence of SEQ ID NO: 173; (e) HVR-L2 comprising the amino acid sequence of SEQ ID NO: 198; and (f) HVR-L3 comprising the amino acid sequence of SEQ ID NO:221; (a) HVR-H1 comprising the amino acid sequence of SEQ ID NO:90; (b) HVR-H2 comprising the amino acid sequence of SEQ ID NO: 112; (c) HVR-H3 comprising the amino acid sequence of SEQ ID NO: 144; (d) HVR-L1 comprising the amino acid sequence of SEQ ID NO: 174; (e) HVR-L2 comprising the amino acid sequence of SEQ ID NO: 199; and (f) HVR-L3 comprising the amino acid sequence of SEQ ID NO:224; (a) HVR-H1 comprising the amino acid sequence of SEQ ID NO:91; (b) HVR-H2 comprising the amino acid sequence of SEQ ID NO: 113; (c) HVR-H3 comprising the amino acid sequence of SEQ ID NO: 145; (d) HVR-L1 comprising the amino acid sequence of SEQ ID NO:175; (e) HVR-L2 comprising the amino acid sequence of SEQ ID NO:200; and (f) HVR-L3 comprising the amino acid sequence of SEQ ID NO:225; (a) HVR-H1 comprising the amino acid sequence of SEQ ID NO:90; (b) HVR-H2 comprising the amino acid sequence of SEQ ID NO:114; (c) HVR-H3 comprising the amino acid sequence of SEQ ID NO: 146; (d) HVR-L1 comprising the amino acid sequence of SEQ ID NO: 176; (e) HVR-L2 comprising the amino acid sequence of SEQ ID NO:201; and (f) HVR-L3 comprising the amino acid sequence of SEQ ID NO:226; (a) HVR-H1 comprising the amino acid sequence of SEQ ID NO:92; (b) HVR-H2 comprising the amino acid sequence of SEQ ID NO: 115; (c) HVR-H3 comprising the amino acid sequence of SEQ ID NO: 147; (d) HVR-L1 comprising the amino acid sequence of SEQ ID NO: 177; (e) HVR-L2 comprising the amino acid sequence of SEQ ID NO: 195; and (f) HVR-L3 comprising the amino acid sequence of SEQ ID NO:227; (a) HVR-H1 comprising the amino acid sequence of SEQ ID NO:93; (b) HVR-H2 comprising the amino acid sequence of SEQ ID NO: 116; (c) HVR-H3 comprising the amino acid sequence of SEQ ID NO:148; (d) HVR-L1 comprising the amino acid sequence of SEQ ID NO:178; (e) HVR-L2 comprising the amino acid sequence of SEQ ID NO:188; and (f) HVR-L3 comprising the amino acid sequence of SEQ ID NO:209; (a) HVR-H1 comprising the amino acid sequence of SEQ ID NO:94; (b) HVR-H2 comprising the amino acid sequence of SEQ ID NO: 117; (c) HVR-H3 comprising the amino acid sequence of SEQ ID NO: 149; (d) HVR-L1 comprising the amino acid sequence of SEQ ID NO: 179; (e) HVR-L2 comprising the amino acid sequence of SEQ ID NO: 195; and (f) HVR-L3 comprising the amino acid sequence of SEQ ID NO:228; (a) HVR-H1 comprising the amino acid sequence of SEQ ID NO:95; (b) HVR-H2 comprising the amino acid sequence of SEQ ID NO:118; (c) HVR-H3 comprising the amino acid sequence of SEQ ID NO:150; (d) HVR-L1 comprising the amino acid sequence of SEQ ID NO: 180; (e) HVR-L2 comprising the amino acid sequence of SEQ ID NO: 187; and (f) HVR-L3 comprising the amino acid sequence of SEQ ID NO:229; (a) HVR-H1 comprising the amino acid sequence of SEQ ID NO:95; (b) HVR-H2 comprising the amino acid sequence of SEQ ID NO: 119; (c) HVR-H3 comprising the amino acid sequence of SEQ ID NO:151; (d) HVR-L1 comprising the amino acid sequence of SEQ ID NO:181; (e) HVR-L2 comprising the amino acid sequence of SEQ ID NO: 187; and (f) HVR-L3 comprising the amino acid sequence of SEQ ID NO:208; (a) HVR-H1 comprising the amino acid sequence of SEQ ID NO:90; (b) HVR-H2 comprising the amino acid sequence of SEQ ID NO: 120; (c) HVR-H3 comprising the amino acid sequence of SEQ ID NO: 152; (d) HVR-L1 comprising the amino acid sequence of SEQ ID NO:182; (e) HVR-L2 comprising the amino acid sequence of SEQ ID NO:202; and (f) HVR-L3 comprising the amino acid sequence of SEQ ID NO:230; (a) HVR-H1 comprising the amino acid sequence of SEQ ID NO:96; (b) HVR-H2 comprising the amino acid sequence of SEQ ID NO:118; (c) HVR-H3 comprising the amino acid sequence of SEQ ID NO:153; (d) HVR-L1 comprising the amino acid sequence of SEQ ID NO: 183; (e) HVR-L2 comprising the amino acid sequence of SEQ ID NO:202; and (f) HVR-L3 comprising the amino acid sequence of SEQ ID NO:230; (a) HVR-H1 comprising the amino acid sequence of SEQ ID NO:96; (b) HVR-H2 comprising the amino acid sequence of SEQ ID NO:121; (c) HVR-H3 comprising the amino acid sequence of SEQ ID NO:154; (d) HVR-L1 comprising the amino acid sequence of SEQ ID NO:181; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:90; (b) HVR-H2 comprising the amino acid sequence of SEQ ID NO: 122; (c) HVR-H3 comprising the amino acid sequence of SEQ ID NO: 155; (d) HVR-L1 comprising the amino acid sequence of SEQ ID NO: 184; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:97; (b) HVR-H2 comprising the amino acid sequence of SEQ ID NO: 123; (c) HVR-H3 comprising the amino acid sequence of SEQ ID NO: 156; (d) HVR-L1 comprising the amino acid sequence of SEQ ID NO: 185; (e) HVR-L2 comprising the amino acid sequence of SEQ ID NO:204; and (f) HVR-L3 comprising the amino acid sequence of SEQ ID NO:232; (a) HVR-H1 comprising the amino acid sequence of SEQ ID NO:98; (b) HVR-H2 comprising the amino acid sequence of SEQ ID NO:124; (c) HVR-H3 comprising the amino acid sequence of SEQ ID NO:157; (d) HVR-L1 comprising the amino acid sequence of SEQ ID NO: 186; (e) HVR-L2 comprising the amino acid sequence of SEQ ID NO:205; and (f) HVR-L3 comprising the amino acid sequence of SEQ ID NO:233.

In another aspect, an anti-MerTK antibody comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs:5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, and 39. In certain embodiments, a V_{H} sequence having at least 90%, 91%, 92%, 93%, 94%, 95%, 96%, 97%, 98%, or 99% identity to an amino acid sequence selected from the group consisting of SEQ ID NOs: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, and 39 contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted, and/or deleted in SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39. In certain embodiments, a total of 1 to 5 amino acids have been substituted, inserted and/or deleted in SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39. In certain embodiments, substitutions, insertions, or deletions occur in regions outside the HVRs (i.e., in the FRs). Optionally, the anti-MerTK antibody comprises the V_{H} sequence of SEQ ID NO: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39. including post-translational modifications of that sequence. In a particular embodiment, the V_{H} comprises one, two or three HVRs selected from: (a) HVR-H1 comprising an amino acid sequence selected from the group consisting of SEQ ID NOs:75-98, (b) HVR-H2 comprising an amino acid sequence selected from the group consisting of SEQ ID NOs:99-124, and (c) HVR-H3 comprising an amino acid sequence selected from the group consisting of SEQ ID NOs:125-157.

In another aspect, an anti-MerTK antibody is provided, wherein the antibody comprises a light chain variable domain (V_{L}) having at least 90%, 91%, 92%, 93%, 94%, 95%, 96%, 97%, 98%, 99%, or 100% sequence identity to an amino acid sequence selected from the group consisting of SEQ ID NOs:40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, and 74. In certain embodiments, a V_{L} sequence having at least 90%, 91%, 92%, 93%, 94%, 95%, 96%, 97%, 98%, or 99% identity to an amino acid sequence selected from the group consisting of SEQ ID NOs: 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, and 74, and contains substitutions (e.g., conservative substitutions), insertions, or deletions relative to the reference sequence, but an anti-MerTK antibody comprising that sequence retains the ability to bind to MerTK. In some embodiments, a total of 1 to 10 amino acids have been substituted, inserted and/or deleted in SEQ ID NO: 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74. In certain embodiments, a total of 1 to 5 amino acids have been substituted, inserted and/or deleted in SEQ ID NO: 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74. In certain embodiments, the substitutions, insertions, or deletions occur in regions outside the HVRs (i.e., in the FRs). Optionally, the anti-MerTK antibody comprises the V_{L} sequence of SEQ ID NO: 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74, including post-translational modifications of that sequence. In a particular embodiment, the V_{L} comprises one, two or three HVRs selected from (a) HVR-L1 comprising an amino acid sequence selected from the group consisting of SEQ ID NOs:158-186, (b) HVR-L2 comprising an amino acid sequence selected from the group consisting of SEQ ID NOs: 187-205, and (c) HVR-L3 comprising an amino acid sequence selected from the group consisting of SEQ ID NOs: 207-233.

In some embodiments, an anti-MerTK antibody is provided, wherein the antibody comprises a V_{H} as in any of the embodiments provided above, and a V_{L} as in any of the embodiments provided above. In some embodiments, provided herein are anti-MerTK antibodies, wherein the antibody comprises a V_{H} as in any of the embodiments provided above, and a V_{L} as in any of the embodiments provided above. In one embodiment, the antibody comprises the V_{H} and V_{L} sequences in SEQ ID NOs:5-39 and SEQ ID NOs:40-74, respectively, including post-translational modifications of those sequences.

In some embodiments, provided herein are anti-MerTK antibodies comprising a heavy chain variable domain (V_{H}) and a light chain variable domain (V_{L}), wherein the V_{H} and V_{L} are selected from the group consisting of: V_{H} comprising the amino acid sequence of SEQ ID NO:5 and V_{L} comprising the amino acid sequence of SEQ ID NO:40; V_{H} comprising the amino acid sequence of SEQ ID NO:6 and V_{L} comprising the amino acid sequence of SEQ ID NO:41; V_{H} comprising the amino acid sequence of SEQ ID NO:7 and V_{L} comprising the amino acid sequence of SEQ ID NO:42; V_{H} comprising the amino acid sequence of SEQ ID NO:8 and V_{L} comprising the amino acid sequence of SEQ ID NO:43; V_{H} comprising the amino acid sequence of SEQ ID NO:9 and V_{L} comprising the amino acid sequence of SEQ ID NO:44; V_{H} comprising the amino acid sequence of SEQ ID NO: 10 and V_{L} comprising the amino acid sequence of SEQ ID NO:45; V_{H} comprising the amino acid sequence of SEQ ID NO:11 and V_{L} comprising the amino acid sequence of SEQ ID NO:46; V_{H} comprising the amino acid sequence of SEQ ID NO: 12 and V_{L} comprising the amino acid sequence of SEQ ID NO:47; V_{H} comprising the amino acid sequence of SEQ ID NO: 13 and V_{L} comprising the amino acid sequence of SEQ ID NO:48; V_{H} comprising the amino acid sequence of SEQ ID NO: 14 and V_{L} comprising the amino acid sequence of SEQ ID NO:49; V_{H} comprising the amino acid sequence of SEQ ID NO: 15 and V_{L} comprising the amino acid sequence of SEQ ID NO:50; V_{H} comprising the amino acid sequence of SEQ ID NO:16 and V_{L} comprising the amino acid sequence of SEQ ID NO:51; V_{H} comprising the amino acid sequence of SEQ ID NO: 17 and V_{L} comprising the amino acid sequence of SEQ ID NO:52; V_{H} comprising the amino acid sequence of SEQ ID NO: 18 and V_{L} comprising the amino acid sequence of SEQ ID NO:53; V_{H} comprising the amino acid sequence of SEQ ID NO: 19 and V_{L} comprising the amino acid sequence of SEQ ID NO:54; V_{H} comprising the amino acid sequence of SEQ ID NO:20 and V_{L} comprising the amino acid sequence of SEQ ID NO:55; V_{H} comprising the amino acid sequence of SEQ ID NO:21 and V_{L} comprising the amino acid sequence of SEQ ID NO:56; V_{H} comprising the amino acid sequence of SEQ ID NO:22 and V_{L} comprising the amino acid sequence of SEQ ID NO:57; V_{H} comprising the amino acid sequence of SEQ ID NO:23 and V_{L} comprising the amino acid sequence of SEQ ID NO:58; V_{H} comprising the amino acid sequence of SEQ ID NO:24 and V_{L} comprising the amino acid sequence of SEQ ID NO:59; V_{H} comprising the amino acid sequence of SEQ ID NO:25 and V_{L} comprising the amino acid sequence of SEQ ID NO:60; V_{H} comprising the amino acid sequence of SEQ ID NO:26 and V_{L} comprising the amino acid sequence of SEQ ID NO:61; V_{H} comprising the amino acid sequence of SEQ ID NO:27 and V_{L} comprising the amino acid sequence of SEQ ID NO:62; V_{H} comprising the amino acid sequence of SEQ ID NO:28 and V_{L} comprising the amino acid sequence of SEQ ID NO:63; V_{H} comprising the amino acid sequence of SEQ ID NO:29 and V_{L} comprising the amino acid sequence of SEQ ID NO:64; V_{H} comprising the amino acid sequence of SEQ ID NO:30 and V_{L} comprising the amino acid sequence of SEQ ID NO:65; V_{H} comprising the amino acid sequence of SEQ ID NO:31 and V_{L} comprising the amino acid sequence of SEQ ID NO:66; V_{H} comprising the amino acid sequence of SEQ ID NO:32 and V_{L} comprising the amino acid sequence of SEQ ID NO:67; V_{H} comprising the amino acid sequence of SEQ ID NO:33 and V_{L} comprising the amino acid sequence of SEQ ID NO:68; V_{H} comprising the amino acid sequence of SEQ ID NO:34 and V_{L} comprising the amino acid sequence of SEQ ID NO:69; V_{H} comprising the amino acid sequence of SEQ ID NO:35 and V_{L} comprising the amino acid sequence of SEQ ID NO:70; V_{H} comprising the amino acid sequence of SEQ ID NO:36 and V_{L} comprising the amino acid sequence of SEQ ID NO:71; V_{H} comprising the amino acid sequence of SEQ ID NO:37 and V_{L} comprising the amino acid sequence of SEQ ID NO:72; V_{H} comprising the amino acid sequence of SEQ ID NO:38 and V_{L} comprising the amino acid sequence of SEQ ID NO:73; and V_{H} comprising the amino acid sequence of SEQ ID NO:39 and V_{L} comprising the amino acid sequence of SEQ ID NO:74.

In some embodiments, provided herein are anti-MerTK antibodies comprising at least one, two, three, four, five, or six HVRs selected from: (a) HVR-H1 comprising the amino acid sequence of SEQ ID NO:83; (b) HVR-H2 comprising an amino acid sequence selected from the group consisting of SEQ ID NOs:99, 329, 330, 331, 332, and 333; (c) HVR-H3 comprising an amino acid sequence selected from the group consisting of SEQ ID NOs: 138, 334, 335, 336, 337, 338, and 339; (d) HVR-L1 comprising an amino acid sequence selected from the group consisting of SEQ ID NOs: 158, 340, 341, 342, 343, and 344; (e) HVR-L2 comprising the amino acid sequence of SEQ ID NO:187; and (f) HVR-L3 comprising the amino acid sequence of SEQ ID NO:210.

In some embodiments, provided herein are anti-MerTK antibodies comprising at least one, at least two, or all three V_{H} HVR sequences selected from (a) HVR-H1 comprising the amino acid sequence of SEQ ID NO:83; (b) HVR-H2 comprising an amino acid sequence selected from the group consisting of SEQ ID NOs:99, 329, 330, 331, 332, and 333; and (c) HVR-H3 comprising an amino acid sequence selected from the group consisting of SEQ ID NOs: 138, 334, 335, 336, 337, 338, and 339.

In some embodiments, provided herein are anti-MerTK antibodies comprising at least one, at least two, or all three V_{L} HVR sequences selected from (a) HVR-L1 comprising an amino acid sequence selected from the group consisting of SEQ ID NOs: 158, 340, 341, 342, 343, and 344; (b) HVR-L2 comprising the amino acid sequence of SEQ ID NO: 187; and (c) HVR-L3 comprising the amino acid sequence of SEQ ID NO:210.

In some embodiments, provided herein are anti-MerTK antibodies comprising (a) a V_{H} domain comprising at least one, at least two, or all three V_{H} HVR sequences selected from (i) HVR-H1 comprising the amino acid sequence of SEQ ID NO: 83, (ii) HVR-H2 comprising an amino acid sequence selected from the group consisting of SEQ ID NOs: 99, 329, 330, 331, 332, and 333, and (iii) HVR-H3 comprising an amino acid sequence selected from the group consisting of SEQ ID NOs: 138, 334, 335, 336, 337, 338, and 339, and (b) a V_{L} domain comprising at least one, at least two, or all three V_{L} HVR sequences selected from (i) HVR-L1 comprising an amino acid sequence selected from the group consisting of SEQ ID NOs: 158, 340, 341, 342, 343, and 344, (ii) HVR-L2 comprising the amino acid sequence of SEQ ID NO: 187, and (iii) HVR-L3 comprising the amino acid sequence of SEQ ID NO: 210.

In some embodiments, provided herein are anti-MerTK antibodies comprising: (a) HVR-H1 comprising the amino acid sequence of SEQ ID NO:83; (b) HVR-H2 comprising the amino acid sequence of SEQ ID NO:99; (c) HVR-H3 comprising the amino acid sequence of SEQ ID NO:138; (d) HVR-L1 comprising the amino acid sequence of SEQ ID NO: 158; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:334; (d) HVR-L1 comprising the amino acid sequence of SEQ ID NO: 158; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:330; (c) HVR-H3 comprising the amino acid sequence of SEQ ID NO:138; (d) HVR-L1 comprising the amino acid sequence of SEQ ID NO:158; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:138; (d) HVR-L1 comprising the amino acid sequence of SEQ ID NO:340; (e) HVR-L2 comprising the amino acid sequence of SEQ ID NO:187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:138; (d) HVR-L1 comprising the amino acid sequence of SEQ ID NO:341; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:335; (d) HVR-L1 comprising the amino acid sequence of SEQ ID NO:341; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:331; (c) HVR-H3 comprising the amino acid sequence of SEQ ID NO:138; (d) HVR-L1 comprising the amino acid sequence of SEQ ID NO:341; (e) HVR-L2 comprising the amino acid sequence of SEQ ID NO:187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO: 138; (d) HVR-L1 comprising the amino acid sequence of SEQ ID NO:342; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:336; (d) HVR-L1 comprising the amino acid sequence of SEQ ID NO:341; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:337; (d) HVR-L1 comprising the amino acid sequence of SEQ ID NO:343; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:332; (c) HVR-H3 comprising the amino acid sequence of SEQ ID NO:338; (d) HVR-L1 comprising the amino acid sequence of SEQ ID NO:341; (e) HVR-L2 comprising the amino acid sequence of SEQ ID NO:187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:333; (c) HVR-H3 comprising the amino acid sequence of SEQ ID NO:334; (d) HVR-L1 comprising the amino acid sequence of SEQ ID NO:158; (e) HVR-L2 comprising the amino acid sequence of SEQ ID NO:187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:332; (c) HVR-H3 comprising the amino acid sequence of SEQ ID NO:336; (d) HVR-L1 comprising the amino acid sequence of SEQ ID NO:341; (e) HVR-L2 comprising the amino acid sequence of SEQ ID NO: 187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO:339; (d) HVR-L1 comprising the amino acid sequence of SEQ ID NO:344; (e) HVR-L2 comprising the amino acid sequence of SEQ ID NO:187; and (f) HVR-L3 comprising the amino acid sequence of SEQ ID NO:210; (a) HVR-H1 comprising the amino acid sequence of SEQ ID NO:83; (b) HVR-H2 comprising the amino acid sequence of SEQ ID NO:329; (c) HVR-H3 comprising the amino acid sequence of SEQ ID NO: 138; (d) HVR-L1 comprising the amino acid sequence of SEQ ID NO: 158; (e) HVR-L2 comprising the amino acid sequence of SEQ ID NO: 187; and (f) HVR-L3 comprising the amino acid sequence of SEQ ID NO:210.

In another aspect, an anti-MerTK antibody comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs:19, 234, 235, 236, 237, 238, 239, 240, 241, 242, 243, 244, 245, and 246. In certain embodiments, a V_{H} sequence having at least 90%, 91%, 92%, 93%, 94%, 95%, 96%, 97%, 98%, or 99% identity to an amino acid sequence selected from the group consisting of SEQ ID NOs: 19, 234, 235, 236, 237, 238, 239, 240, 241, 242, 243, 244, 245, and 246 contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted, and/or deleted in SEQ ID NO: 19, 234, 235, 236, 237, 238, 239, 240, 241, 242, 243, 244, 245, or 246. In certain embodiments, a total of 1 to 5 amino acids have been substituted, inserted and/or deleted in SEQ ID NO: 19, 234, 235, 236, 237, 238, 239, 240, 241, 242, 243, 244, 245, or 246. In certain embodiments, substitutions, insertions, or deletions occur in regions outside the HVRs (i.e., in the FRs). Optionally, the anti-MerTK antibody comprises the V_{H} sequence of SEQ ID NO: 19, 234, 235, 236, 237, 238, 239, 240, 241, 242, 243, 244, 245, or 246, including post-translational modifications of that sequence. In a particular embodiment, the V_{H} comprises one, two or three HVRs selected from: (a) HVR-H1 comprising the amino acid sequence of SEQ ID NO:83, (b) HVR-H2 comprising an amino acid sequence selected from the group consisting of SEQ ID NOs:99, 329, 330, 331, 332, and 333, and (c) HVR-H3 comprising an amino acid sequence selected from the group consisting of SEQ ID NOs:138, 334, 335, 336, 337, 338, and 339.

In another aspect, an anti-MerTK antibody is provided, wherein the antibody comprises a light chain variable domain (V_{L}) having at least 90%, 91%, 92%, 93%, 94%, 95%, 96%, 97%, 98%, 99%, or 100% sequence identity to an amino acid sequence selected from the group consisting of SEQ ID NOs:54, 247, 248, 249, 250, 251, 252, 253, and 254. In certain embodiments, a V_{L} sequence having at least 90%, 91%, 92%, 93%, 94%, 95%, 96%, 97%, 98%, or 99% identity to an amino acid sequence selected from the group consisting of SEQ ID NOs: 54, 247, 248, 249, 250, 251, 252, 253, and 254, and contains substitutions (e.g., conservative substitutions), insertions, or deletions relative to the reference sequence, but an anti-MerTK antibody comprising that sequence retains the ability to bind to MerTK. In some embodiments, a total of 1 to 10 amino acids have been substituted, inserted and/or deleted in SEQ ID NO: 54, 247, 248, 249, 250, 251, 252, 253, or 254. In certain embodiments, a total of 1 to 5 amino acids have been substituted, inserted and/or deleted in SEQ ID NO: 54, 247, 248, 249, 250, 251, 252, 253, or 254. In certain embodiments, the substitutions, insertions, or deletions occur in regions outside the HVRs (i.e., in the FRs). Optionally, the anti-MerTK antibody comprises the V_{L} sequence of SEQ ID NO: 54, 247, 248, 249, 250, 251, 252, 253, or 254, including post-translational modifications of that sequence. In a particular embodiment, the V_{L} comprises one, two or three HVRs selected from (a) HVR-L1 comprising an amino acid sequence selected from the group consisting of SEQ ID NOs:158, 340, 341, 342, 343, and 344, (b) HVR-L2 comprising the amino acid sequence of SEQ ID NO: 187, and (c) HVR-L3 comprising the amino acid sequence of SEQ ID NO: 210.

In some embodiments, an anti-MerTK antibody is provided, wherein the antibody comprises a V_{H} as in any of the embodiments provided above, and a V_{L} as in any of the embodiments provided above. In some embodiments, provided herein are anti-MerTK antibodies, wherein the antibody comprises a V_{H} as in any of the embodiments provided above, and a V_{L} as in any of the embodiments provided above. In one embodiment, the antibody comprises the V_{H} and V_{L} sequences in SEQ ID NOs:19, 234, 235, 236, 237, 238, 239, 240, 241, 242, 243, 244, 245, and 246 and SEQ ID NOs:54, 247, 248, 249, 250, 251, 252, 253, and 258, respectively, including post-translational modifications of those sequences.

In some embodiments, provided herein are anti-MerTK antibodies comprising a heavy chain variable domain (V_{H}) and a light chain variable domain (V_{L}), wherein the V_{H} and V_{L} are selected from the group consisting of: V_{H} comprising the amino acid sequence of SEQ ID NO:19 and V_{L} comprising the amino acid sequence of SEQ ID NO:54; V_{H} comprising the amino acid sequence of SEQ ID NO:234 and V_{L} comprising the amino acid sequence of SEQ ID NO:247; V_{H} comprising the amino acid sequence of SEQ ID NO:235 and V_{L} comprising the amino acid sequence of SEQ ID NO:247; V_{H} comprising the amino acid sequence of SEQ ID NO:236 and V_{L} comprising the amino acid sequence of SEQ ID NO:248; V_{H} comprising the amino acid sequence of SEQ ID NO:236 and V_{L} comprising the amino acid sequence of SEQ ID NO:249; V_{H} comprising the amino acid sequence of SEQ ID NO:237 and V_{L} comprising the amino acid sequence of SEQ ID NO:249; V_{H} comprising the amino acid sequence of SEQ ID NO:238 and V_{L} comprising the amino acid sequence of SEQ ID NO:249; V_{H} comprising the amino acid sequence of SEQ ID NO:239 and V_{L} comprising the amino acid sequence of SEQ ID NO:250; V_{H} comprising the amino acid sequence of SEQ ID NO:240 and V_{L} comprising the amino acid sequence of SEQ ID NO:251; V_{H} comprising the amino acid sequence of SEQ ID NO:241 and V_{L} comprising the amino acid sequence of SEQ ID NO:252; V_{H} comprising the amino acid sequence of SEQ ID NO:242 and V_{L} comprising the amino acid sequence of SEQ ID NO:249; V_{H} comprising the amino acid sequence of SEQ ID NO:243 and V_{L} comprising the amino acid sequence of SEQ ID NO:247; V_{H} comprising the amino acid sequence of SEQ ID NO:244 and V_{L} comprising the amino acid sequence of SEQ ID NO:251; V_{H} comprising the amino acid sequence of SEQ ID NO:245 and V_{L} comprising the amino acid sequence of SEQ ID NO:253; V_{H} comprising the amino acid sequence of SEQ ID NO:246 and V_{L} comprising the amino acid sequence of SEQ ID NO:247; and V_{H} comprising the amino acid sequence of SEQ ID NO:246 and V_{L} comprising the amino acid sequence of SEQ ID NO:254.

In some embodiments, provided herein are anti-MerTK antibodies comprising at least one, two, three, four, five, or six HVRs selected from: (a) HVR-H1 comprising the amino acid sequence of SEQ ID NO:84; (b) HVR-H2 comprising an amino acid sequence selected from the group consisting of SEQ ID NOs:99 and 329; (c) HVR-H3 comprising the amino acid sequence of SEQ ID NO:139; (d) HVR-L1 comprising an amino acid sequence selected from the group consisting of SEQ ID NOs:169 and 345; (e) HVR-L2 comprising the amino acid sequence of SEQ ID NO:195; and (f) HVR-L3 comprising the amino acid sequence of SEQ ID NO:219.

In some embodiments, provided herein are anti-MerTK antibodies comprising at least one, at least two, or all three V_{H} HVR sequences selected from (a) HVR-H1 comprising the amino acid sequence of SEQ ID NO:84; (b) HVR-H2 comprising an amino acid sequence selected from the group consisting of SEQ ID NOs:99 and 329; and (c) HVR-H3 comprising the amino acid sequence of SEQ ID NO: 139.

In some embodiments, provided herein are anti-MerTK antibodies comprising at least one, at least two, or all three V_{L} HVR sequences selected from (a) HVR-L1 comprising an amino acid sequence selected from the group consisting of SEQ ID NOs:169 and 345; (b) HVR-L2 comprising the amino acid sequence of SEQ ID NO: 195; and (c) HVR-L3 comprising the amino acid sequence of SEQ ID NO:219.

In some embodiments, provided herein are anti-MerTK antibodies comprising (a) a V_{H} domain comprising at least one, at least two, or all three V_{H} HVR sequences selected from (i) HVR-H1 comprising the amino acid sequence of SEQ ID NO: 84, (ii) HVR-H2 comprising an amino acid sequence selected from the group consisting of SEQ ID NOs: 99, 329, and (iii) HVR-H3 comprising the amino acid sequence of SEQ ID NO: 139, and (b) a V_{L} domain comprising at least one, at least two, or all three V_{L} HVR sequences selected from (i) HVR-L1 comprising an amino acid sequence selected from the group consisting of SEQ ID NOs: 169 and 345, (ii) HVR-L2 comprising the amino acid sequence of SEQ ID NO: 195, and (iii) HVR-L3 comprising the amino acid sequence of SEQ ID NO: 219.

In some embodiments, provided herein are anti-MerTK antibodies comprising: (a) HVR-H1 comprising the amino acid sequence of SEQ ID NO:84; (b) HVR-H2 comprising the amino acid sequence of SEQ ID NO:99; (c) HVR-H3 comprising the amino acid sequence of SEQ ID NO:139; (d) HVR-L1 comprising the amino acid sequence of SEQ ID NO: 169; (e) HVR-L2 comprising the amino acid sequence of SEQ ID NO: 195; and (f) HVR-L3 comprising the amino acid sequence of SEQ ID NO:219; (a) HVR-H1 comprising the amino acid sequence of SEQ ID NO:84; (b) HVR-H2 comprising the amino acid sequence of SEQ ID NO:329; (c) HVR-H3 comprising the amino acid sequence of SEQ ID NO: 139; (d) HVR-L1 comprising the amino acid sequence of SEQ ID NO: 169; (e) HVR-L2 comprising the amino acid sequence of SEQ ID NO: 195; and (f) HVR-L3 comprising the amino acid sequence of SEQ ID NO:219; and (a) HVR-H1 comprising the amino acid sequence of SEQ ID NO:84; (b) HVR-H2 comprising the amino acid sequence of SEQ ID NO:99; (c) HVR-H3 comprising the amino acid sequence of SEQ ID NO:139; (d) HVR-L1 comprising the amino acid sequence of SEQ ID NO:345; (e) HVR-L2 comprising the amino acid sequence of SEQ ID NO:195; and (f) HVR-L3 comprising the amino acid sequence of SEQ ID NO:219.

In another aspect, an anti-MerTK antibody comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs:20, 255, and 256. In certain embodiments, a V_{H} sequence having at least 90%, 91%, 92%, 93%, 94%, 95%, 96%, 97%, 98%, or 99% identity to an amino acid sequence selected from the group consisting of SEQ ID NOs: 20, 255, and 256 contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted, and/or deleted in SEQ ID NO: 20, 255, or 256. In certain embodiments, a total of 1 to 5 amino acids have been substituted, inserted and/or deleted in SEQ ID NO: 20, 255, or 256. In certain embodiments, substitutions, insertions, or deletions occur in regions outside the HVRs (i.e., in the FRs). Optionally, the anti-MerTK antibody comprises the V_{H} sequence of SEQ ID NO: 20, 255, or 256, including post-translational modifications of that sequence. In a particular embodiment, the V_{H} comprises one, two or three HVRs selected from: (a) HVR-H1 comprising the amino acid sequence of SEQ ID NO:84, (b) HVR-H2 comprising an amino acid sequence selected from the group consisting of SEQ ID NOs:99 and 329, and (c) HVR-H3 comprising the amino acid sequence of SEQ ID NO:139.

In another aspect, an anti-MerTK antibody is provided, wherein the antibody comprises a light chain variable domain (V_{L}) having at least 90%, 91%, 92%, 93%, 94%, 95%, 96%, 97%, 98%, 99%, or 100% sequence identity to an amino acid sequence selected from the group consisting of SEQ ID NOs:55, 257, and 258. In certain embodiments, a V_{L} sequence having at least 90%, 91%, 92%, 93%, 94%, 95%, 96%, 97%, 98%, or 99% identity to an amino acid sequence selected from the group consisting of SEQ ID NOs: 55, 257, and 258, and contains substitutions (e.g., conservative substitutions), insertions, or deletions relative to the reference sequence, but an anti-MerTK antibody comprising that sequence retains the ability to bind to MerTK. In some embodiments, a total of 1 to 10 amino acids have been substituted, inserted and/or deleted in SEQ ID NO: 55, 257, or 258. In certain embodiments, a total of 1 to 5 amino acids have been substituted, inserted and/or deleted in SEQ ID NO: 55, 257, or 258. In certain embodiments, the substitutions, insertions, or deletions occur in regions outside the HVRs (i.e., in the FRs). Optionally, the anti-MerTK antibody comprises the V_{L} sequence of SEQ ID NO: 55, 257, or 258, including post-translational modifications of that sequence. In a particular embodiment, the V_{L} comprises one, two or three HVRs selected from (a) HVR-L1 comprising an amino acid sequence selected from the group consisting of SEQ ID NOs:169 and 345, (b) HVR-L2 comprising the amino acid sequence of SEQ ID NO: 195, and (c) HVR-L3 comprising the amino acid sequence of SEQ ID NO: 219.

In some embodiments, an anti-MerTK antibody is provided, wherein the antibody comprises a V_{H} as in any of the embodiments provided above, and a V_{L} as in any of the embodiments provided above. In some embodiments, provided herein are anti-MerTK antibodies, wherein the antibody comprises a V_{H} as in any of the embodiments provided above, and a V_{L} as in any of the embodiments provided above. In one embodiment, the antibody comprises the V_{H} and V_{L} sequences in SEQ ID NOs:20, 255, and 256 and SEQ ID NOs:55, 257, and 258, respectively, including post-translational modifications of those sequences.

In some embodiments, provided herein are anti-MerTK antibodies comprising a heavy chain variable domain (V_{H}) and a light chain variable domain (V_{L}), wherein the V_{H} and V_{L} are selected from the group consisting of: V_{H} comprising the amino acid sequence of SEQ ID NO:20 and V_{L} comprising the amino acid sequence of SEQ ID NO:55; V_{H} comprising the amino acid sequence of SEQ ID NO:255 and V_{L} comprising the amino acid sequence of SEQ ID NO:257; and V_{H} comprising the amino acid sequence of SEQ ID NO:256 and V_{L} comprising the amino acid sequence of SEQ ID NO:258.

In some embodiments, provided herein are anti-MerTK antibodies comprising at least one, two, three, four, five, or six HVRs selected from: (a) HVR-H1 comprising an amino acid sequence selected from the group consisting of SEQ ID NOs:95, 346, and 347; (b) HVR-H2 comprising an amino acid sequence selected from the group consisting of SEQ ID NOs:119, 348, 349, 350, 351, 352, 353, and 354; (c) HVR-H3 comprising an amino acid sequence selected from the group consisting of SEQ ID NOs:151, 355, and 356; (d) HVR-L1 comprising an amino acid sequence selected from the group consisting of SEQ ID NOs:181, 341, 357, and 358; (e) HVR-L2 comprising an amino acid sequence selected from the group consisting of SEQ ID NOs:187 and 359; and (f) HVR-L3 comprising an amino acid sequence selected from the group consisting of SEQ ID NOs:208, 360, 361, and 362.

In some embodiments, provided herein are anti-MerTK antibodies comprising at least one, at least two, or all three V_{H} HVR sequences selected from (a) HVR-H1 comprising an amino acid sequence selected from the group consisting of SEQ ID NOs:95, 346, and 347; (b) HVR-H2 comprising an amino acid sequence selected from the group consisting of SEQ ID NOs:119, 348, 349, 350, 351, 352, 353, and 354; and (c) HVR-H3 comprising an amino acid sequence selected from the group consisting of SEQ ID NOs:151, 355, and 356.

In some embodiments, provided herein are anti-MerTK antibodies comprising at least one, at least two, or all three V_{L} HVR sequences selected from (a) HVR-L1 comprising an amino acid sequence selected from the group consisting of SEQ ID NOs:181, 341, 357, and 358; (b) HVR-L2 comprising an amino acid sequence selected from the group consisting of SEQ ID NOs:187 and 359; and (c) HVR-L3 comprising an amino acid sequence selected from the group consisting of SEQ ID NO:208. 360, 361, and 362.

In some embodiments, provided herein are anti-MerTK antibodies comprising (a) a V_{H} domain comprising at least one, at least two, or all three V_{H} HVR sequences selected from (i) HVR-H1 comprising an amino acid sequence selected from the group consisting of SEQ ID NOs: 95, 346, and 347, (ii) HVR-H2 comprising an amino acid sequence selected from the group consisting of SEQ ID NOs: 119, 348, 349, 350, 351, 352, 353, and 354, and (iii) HVR-H3 comprising an amino acid sequence selected from the group consisting of SEQ ID NOs: 151, 355, and 356, and (b) a V_{L} domain comprising at least one, at least two, or all three V_{L} HVR sequences selected from (i) HVR-L1 comprising an amino acid sequence selected from the group consisting of SEQ ID NOs: 181, 341, 357, and 358, (ii) HVR-L2 comprising an amino acid sequence selected from the group consisting of SEQ ID NOs: 187 and 359, and (iii) HVR-L3 comprising an amino acid sequence selected from the group consisting of SEQ ID NOs: 208, 360, 361, and 362.

In some embodiments, provided herein are anti-MerTK antibodies comprising: (a) HVR-H1 comprising the amino acid sequence of SEQ ID NO:95; (b) HVR-H2 comprising the amino acid sequence of SEQ ID NO: 119; (c) HVR-H3 comprising the amino acid sequence of SEQ ID NO:151; (d) HVR-L1 comprising the amino acid sequence of SEQ ID NO:181; (e) HVR-L2 comprising the amino acid sequence of SEQ ID NO: 187; and (f) HVR-L3 comprising the amino acid sequence of SEQ ID NO:208; (a) HVR-H1 comprising the amino acid sequence of SEQ ID NO:346; (b) HVR-H2 comprising the amino acid sequence of SEQ ID NO:348; (c) HVR-H3 comprising the amino acid sequence of SEQ ID NO:355; (d) HVR-L1 comprising the amino acid sequence of SEQ ID NO:341; (e) HVR-L2 comprising the amino acid sequence of SEQ ID NO: 187; and (f) HVR-L3 comprising the amino acid sequence of SEQ ID NO:360; (a) HVR-H1 comprising the amino acid sequence of SEQ ID NO:95; (b) HVR-H2 comprising the amino acid sequence of SEQ ID NO: 119; (c) HVR-H3 comprising the amino acid sequence of SEQ ID NO:355; (d) HVR-L1 comprising the amino acid sequence of SEQ ID NO:341; (e) HVR-L2 comprising the amino acid sequence of SEQ ID NO: 187; and (f) HVR-L3 comprising the amino acid sequence of SEQ ID NO:361; (a) HVR-H1 comprising the amino acid sequence of SEQ ID NO:95; (b) HVR-H2 comprising the amino acid sequence of SEQ ID NO:349; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:187; and (f) HVR-L3 comprising the amino acid sequence of SEQ ID NO:362; (a) HVR-H1 comprising the amino acid sequence of SEQ ID NO:95; (b) HVR-H2 comprising the amino acid sequence of SEQ ID NO:119; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:359; and (f) HVR-L3 comprising the amino acid sequence of SEQ ID NO:362; (a) HVR-H1 comprising the amino acid sequence of SEQ ID NO:346; (b) HVR-H2 comprising the amino acid sequence of SEQ ID NO: 119; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:359; and (f) HVR-L3 comprising the amino acid sequence of SEQ ID NO:362; (a) HVR-H1 comprising the amino acid sequence of SEQ ID NO:95; (b) HVR-H2 comprising the amino acid sequence of SEQ ID NO:350 (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:359; and (f) HVR-L3 comprising the amino acid sequence of SEQ ID NO:362; (a) HVR-H1 comprising the amino acid sequence of SEQ ID NO:347; (b) HVR-H2 comprising the amino acid sequence of SEQ ID NO: 119; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:359; and (f) HVR-L3 comprising the amino acid sequence of SEQ ID NO:362; (a) HVR-H1 comprising the amino acid sequence of SEQ ID NO:95; (b) HVR-H2 comprising the amino acid sequence of SEQ ID NO: 119; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO: 187; and (f) HVR-L3 comprising the amino acid sequence of SEQ ID NO:362; (a) HVR-H1 comprising the amino acid sequence of SEQ ID NO:95; (b) HVR-H2 comprising the amino acid sequence of SEQ ID NO:352; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:187; and (f) HVR-L3 comprising the amino acid sequence of SEQ ID NO:362; (a) HVR-H1 comprising the amino acid sequence of SEQ ID NO:95; (b) HVR-H2 comprising the amino acid sequence of SEQ ID NO:353; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:359; and (f) HVR-L3 comprising the amino acid sequence of SEQ ID NO:362; (a) HVR-H1 comprising the amino acid sequence of SEQ ID NO:346; (b) HVR-H2 comprising the amino acid sequence of SEQ ID NO:354; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:359; and (f) HVR-L3 comprising the amino acid sequence of SEQ ID NO:363; (a) HVR-H1 comprising the amino acid sequence of SEQ ID NO:346; (b) HVR-H2 comprising the amino acid sequence of SEQ ID NO:348; (c) HVR-H3 comprising the amino acid sequence of SEQ ID NO:356; (d) HVR-L1 comprising the amino acid sequence of SEQ ID NO:357; (e) HVR-L2 comprising the amino acid sequence of SEQ ID NO:359; and (f) HVR-L3 comprising the amino acid sequence of SEQ ID NO:362; (a) HVR-H1 comprising the amino acid sequence of SEQ ID NO:346; (b) HVR-H2 comprising the amino acid sequence of SEQ ID NO:354; (c) HVR-H3 comprising the amino acid sequence of SEQ ID NO:356; (d) HVR-L1 comprising the amino acid sequence of SEQ ID NO:358; (e) HVR-L2 comprising the amino acid sequence of SEQ ID NO: 187; and (f) HVR-L3 comprising the amino acid sequence of SEQ ID NO:362; (a) HVR-H1 comprising the amino acid sequence of SEQ ID NO:95; (b) HVR-H2 comprising the amino acid sequence of SEQ ID NO:348; (c) HVR-H3 comprising the amino acid sequence of SEQ ID NO:151; (d) HVR-L1 comprising the amino acid sequence of SEQ ID NO:181; (e) HVR-L2 comprising the amino acid sequence of SEQ ID NO: 187; and (f) HVR-L3 comprising the amino acid sequence of SEQ ID NO:208.

In another aspect, an anti-MerTK antibody comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs:33, 259, 260, 261, 262, 263, 264, 265, 266, 267, 268, 269, 270, 271, 272, and 273. In certain embodiments, a V_{H} sequence having at least 90%, 91%, 92%, 93%, 94%, 95%, 96%, 97%, 98%, or 99% identity to an amino acid sequence selected from the group consisting of SEQ ID NOs: 33, 259, 260, 261, 262, 263, 264, 265, 266, 267, 268, 269, 270, 271, 272, and 273 contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted, and/or deleted in SEQ ID NO: 33, 259, 260, 261, 262, 263, 264, 265, 266, 267, 268, 269, 270, 271, 272, or 273. In certain embodiments, a total of 1 to 5 amino acids have been substituted, inserted and/or deleted in SEQ ID NO: 33, 259, 260, 261, 262, 263, 264, 265, 266, 267, 268, 269, 270, 271, 272, or 273. In certain embodiments, substitutions, insertions, or deletions occur in regions outside the HVRs (i.e., in the FRs). Optionally, the anti-MerTK antibody comprises the V_{H} sequence of SEQ ID NO: 33, 259, 260, 261, 262, 263, 264, 265, 266, 267, 268, 269, 270, 271, 272, or 273, including post-translational modifications of that sequence. In a particular embodiment, the V_{H} comprises one, two or three HVRs selected from: (a) HVR-H1 comprising an amino acid sequence selected from the group consisting of SEQ ID NOs:95, 346, and 347, (b) HVR-H2 comprising an amino acid sequence selected from the group consisting of SEQ ID NOs:119, 348, 349, 350, 351, 352, 353, and 354, and (c) HVR-H3 comprising an amino acid sequence selected from the group consisting of SEQ ID NOs:151, 355, and 356.

In another aspect, an anti-MerTK antibody is provided, wherein the antibody comprises a light chain variable domain (V_{L}) having at least 90%, 91%, 92%, 93%, 94%, 95%, 96%, 97%, 98%, 99%, or 100% sequence identity to an amino acid sequence selected from the group consisting of SEQ ID NOs:68, 274, 275, 276, 277, 278, 279, 280, 281, 282, 283, 284, 285, 286, 287, 288, and 289. In certain embodiments, a V_{L} sequence having at least 90%, 91%, 92%, 93%, 94%, 95%, 96%, 97%, 98%, or 99% identity to an amino acid sequence selected from the group consisting of SEQ ID NOs: 68, 274, 275, 276, 277, 278, 279, 280, 281, 282, 283, 284, 285, 286, 287, 288, and 289, and contains substitutions (e.g., conservative substitutions), insertions, or deletions relative to the reference sequence, but an anti-MerTK antibody comprising that sequence retains the ability to bind to MerTK. In some embodiments, a total of 1 to 10 amino acids have been substituted, inserted and/or deleted in SEQ ID NO: 68, 274, 275, 276, 277, 278, 279, 280, 281, 282, 283, 284, 285, 286, 287, 288, or 289. In certain embodiments, a total of 1 to 5 amino acids have been substituted, inserted and/or deleted in SEQ ID NO: 68, 274, 275, 276, 277, 278, 279, 280, 281, 282, 283, 284, 285, 286, 287, 288, or 289. In certain embodiments, the substitutions, insertions, or deletions occur in regions outside the HVRs (i.e., in the FRs). Optionally, the anti-MerTK antibody comprises the V_{L} sequence of SEQ ID NO: 68, 274, 275, 276, 277, 278, 279, 280, 281, 282, 283, 284, 285, 286, 287, 288, or 289, including post-translational modifications of that sequence. In a particular embodiment, the V_{L} comprises one, two or three HVRs selected from (a) HVR-L1 comprising an amino acid sequence selected from the group consisting of SEQ ID NOs:181, 341, 357, and 358, (b) HVR-L2 comprising an amino acid sequence selected from the group consisting of SEQ ID NOs: 187 and 359, and (c) HVR-L3 comprising an amino acid sequence selected from the group consisting of SEQ ID NOs: 208, 360, 361, and 362.

In some embodiments, an anti-MerTK antibody is provided, wherein the antibody comprises a V_{H} as in any of the embodiments provided above, and a V_{L} as in any of the embodiments provided above. In some embodiments, provided herein are anti-MerTK antibodies, wherein the antibody comprises a V_{H} as in any of the embodiments provided above, and a V_{L} as in any of the embodiments provided above. In one embodiment, the antibody comprises the V_{H} and V_{L} sequences in SEQ ID NOs: 33, 259, 260, 261, 262, 263, 264, 265, 266, 267, 268, 269, 270, 271, 272, and 273 and SEQ ID NOs: 68, 274, 275, 276, 277, 278, 279, 280, 281, 282, 283, 284, 285, 286, 287, 288, and 289, respectively, including post-translational modifications of those sequences.

In some embodiments, provided herein are anti-MerTK antibodies comprising a heavy chain variable domain (V_{H}) and a light chain variable domain (V_{L}), wherein the V_{H} and V_{L} are selected from the group consisting of: V_{H} comprising the amino acid sequence of SEQ ID NO:33 and V_{L} comprising the amino acid sequence of SEQ ID NO:68; V_{H} comprising the amino acid sequence of SEQ ID NO:259 and V_{L} comprising the amino acid sequence of SEQ ID NO:274; V_{H} comprising the amino acid sequence of SEQ ID NO:260 and V_{L} comprising the amino acid sequence of SEQ ID NO:275; V_{H} comprising the amino acid sequence of SEQ ID NO:261 and V_{L} comprising the amino acid sequence of SEQ ID NO:276; V_{H} comprising the amino acid sequence of SEQ ID NO:262 and V_{L} comprising the amino acid sequence of SEQ ID NO:277; V_{H} comprising the amino acid sequence of SEQ ID NO:263 and V_{L} comprising the amino acid sequence of SEQ ID NO:277; V_{H} comprising the amino acid sequence of SEQ ID NO:264 and V_{L} comprising the amino acid sequence of SEQ ID NO:277; V_{H} comprising the amino acid sequence of SEQ ID NO:265 and V_{L} comprising the amino acid sequence of SEQ ID NO:277; V_{H} comprising the amino acid sequence of SEQ ID NO:266 and V_{L} comprising the amino acid sequence of SEQ ID NO:277; V_{H} comprising the amino acid sequence of SEQ ID NO:267 and V_{L} comprising the amino acid sequence of SEQ ID NO:278; V_{H} comprising the amino acid sequence of SEQ ID NO:268 and V_{L} comprising the amino acid sequence of SEQ ID NO:279; V_{H} comprising the amino acid sequence of SEQ ID NO:269 and V_{L} comprising the amino acid sequence of SEQ ID NO:279; V_{H} comprising the amino acid sequence of SEQ ID NO:264 and V_{L} comprising the amino acid sequence of SEQ ID NO:280; V_{H} comprising the amino acid sequence of SEQ ID NO:270 and V_{L} comprising the amino acid sequence of SEQ ID NO:281; V_{H} comprising the amino acid sequence of SEQ ID NO:265 and V_{L} comprising the amino acid sequence of SEQ ID NO:282; V_{H} comprising the amino acid sequence of SEQ ID NO:264 and V_{L} comprising the amino acid sequence of SEQ ID NO:283; V_{H} comprising the amino acid sequence of SEQ ID NO:271 and V_{L} comprising the amino acid sequence of SEQ ID NO:284; V_{H} comprising the amino acid sequence of SEQ ID NO:272 and V_{L} comprising the amino acid sequence of SEQ ID NO:285; V_{H} comprising the amino acid sequence of SEQ ID NO:271 and V_{L} comprising the amino acid sequence of SEQ ID NO:286; V_{H} comprising the amino acid sequence of SEQ ID NO:273 and V_{L} comprising the amino acid sequence of SEQ ID NO:287; V_{H} comprising the amino acid sequence of SEQ ID NO:273 and V_{L} comprising the amino acid sequence of SEQ ID NO:288; and V_{H} comprising the amino acid sequence of SEQ ID NO:273 and V_{L} comprising the amino acid sequence of SEQ ID NO:289.

In some embodiments, provided herein are anti-MerTK antibodies comprising at least one, two, three, four, five, or six HVRs selected from: (a) HVR-H1 comprising the amino acid sequence of SEQ ID NO:90; (b) HVR-H2 comprising an amino acid sequence selected from the group consisting of SEQ ID NOs:122, 364, 365, 366, 367, and 368; (c) HVR-H3 comprising an amino acid sequence selected from the group consisting of SEQ ID NOs:155, 373, 374, and 375; (d) HVR-L1 comprising an amino acid sequence selected from the group consisting of SEQ ID NOs:184, 376, 377, 378, 379, 380, 381, 382, 383, 384, 385, 386, and 387; (e) HVR-L2 comprising an amino acid sequence selected from the group consisting of SEQ ID NOs:203, 388, 389, 390, and 391; and (f) HVR-L3 comprising an amino acid sequence selected from the group consisting of SEQ ID NOs:231, 392, 393, and 394.

In some embodiments, provided herein are anti-MerTK antibodies comprising at least one, at least two, or all three V_{H} HVR sequences selected from (a) HVR-H1 comprising the amino acid sequence of SEQ ID NO:90; (b) HVR-H2 comprising an amino acid sequence selected from the group consisting of SEQ ID NOs: 122, 364, 365, 366, 367, and 368; and (c) HVR-H3 comprising an amino acid sequence selected from the group consisting of SEQ ID NOs: 155, 373, 374, and 375.

In some embodiments, provided herein are anti-MerTK antibodies comprising at least one, at least two, or all three V_{L} HVR sequences selected from (a) HVR-L1 comprising an amino acid sequence selected from the group consisting of SEQ ID NOs: 184, 376, 377, 378, 379, 380, 381, 382, 383, 384, 385, 386, and 387; (b) HVR-L2 comprising an amino acid sequence selected from the group consisting of SEQ ID NOs: 203, 388, 389, 390, and 391 and (c) HVR-L3 comprising an amino acid sequence selected from the group consisting of SEQ ID NOs: 203, 388, 389, 390, and 391

In some embodiments, provided herein are anti-MerTK antibodies comprising (a) a V_{H} domain comprising at least one, at least two, or all three V_{H} HVR sequences selected from (i) HVR-H1 comprising the amino acid sequence of SEQ ID NO: 90, (ii) HVR-H2 comprising an amino acid sequence selected from the group consisting of SEQ ID NOs: 122, 364, 365, 366, 367, and 368, and (iii) HVR-H3 comprising an amino acid sequence selected from the group consisting of SEQ ID NOs: 155, 373, 374, and 375, and (b) a V_{L} domain comprising at least one, at least two, or all three V_{L} HVR sequences selected from (i) HVR-L1 comprising an amino acid sequence selected from the group consisting of SEQ ID NOs: 184, 376, 377, 378, 379, 380, 381, 382, 383, 384, 385, 386, and 387, (ii) HVR-L2 comprising an amino acid sequence selected from the group consisting of SEQ ID NOs: 203, 388, 389, 390, and 391, and (iii) HVR-L3 comprising an amino acid sequence selected from the group consisting of SEQ ID NOs: 231, 392, 393, and 394.

In some embodiments, provided herein are anti-MerTK antibodies comprising: (a) HVR-H1 comprising the amino acid sequence of SEQ ID NO:90; (b) HVR-H2 comprising the amino acid sequence of SEQ ID NO:122; (c) HVR-H3 comprising the amino acid sequence of SEQ ID NO:155; (d) HVR-L1 comprising the amino acid sequence of SEQ ID NO: 184; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:373; (d) HVR-L1 comprising the amino acid sequence of SEQ ID NO:376; (e) HVR-L2 comprising the amino acid sequence of SEQ ID NO:388; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:155; (d) HVR-L1 comprising the amino acid sequence of SEQ ID NO:377; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:374; (d) HVR-L1 comprising the amino acid sequence of SEQ ID NO:378; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:95; (b) HVR-H2 comprising the amino acid sequence of SEQ ID NO:119; (c) HVR-H3 comprising the amino acid sequence of SEQ ID NO:356; (d) HVR-L1 comprising the amino acid sequence of SEQ ID NO:341; (e) HVR-L2 comprising the amino acid sequence of SEQ ID NO:359; and (f) HVR-L3 comprising the amino acid sequence of SEQ ID NO:362; (a) HVR-H1 comprising the amino acid sequence of SEQ ID NO:90; (b) HVR-H2 comprising the amino acid sequence of SEQ ID NO:365; (c) HVR-H3 comprising the amino acid sequence of SEQ ID NO:155; (d) HVR-L1 comprising the amino acid sequence of SEQ ID NO:276; (e) HVR-L2 comprising the amino acid sequence of SEQ ID NO:203; and (f) HVR-L3 comprising the amino acid sequence of SEQ ID NO:392; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:155; (d) HVR-L1 comprising the amino acid sequence of SEQ ID NO:376; (e) HVR-L2 comprising the amino acid sequence of SEQ ID NO:389; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:155; (d) HVR-L1 comprising the amino acid sequence of SEQ ID NO:379; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:366; (c) HVR-H3 comprising the amino acid sequence of SEQ ID NO: 155; (d) HVR-L1 comprising the amino acid sequence of SEQ ID NO:380; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:373; (d) HVR-L1 comprising the amino acid sequence of SEQ ID NO:381; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:367; (c) HVR-H3 comprising the amino acid sequence of SEQ ID NO:155; (d) HVR-L1 comprising the amino acid sequence of SEQ ID NO:382; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO:155; (d) HVR-L1 comprising the amino acid sequence of SEQ ID NO:383; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:374; (d) HVR-L1 comprising the amino acid sequence of SEQ ID NO:384; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO: 155; (d) HVR-L1 comprising the amino acid sequence of SEQ ID NO:376; (e) HVR-L2 comprising the amino acid sequence of SEQ ID NO:203; and (f) HVR-L3 comprising the amino acid sequence of SEQ ID NO:393; (a) HVR-H1 comprising the amino acid sequence of SEQ ID NO:90; (b) HVR-H2 comprising the amino acid sequence of SEQ ID NO:364; (c) HVR-H3 comprising the amino acid sequence of SEQ ID NO:155; (d) HVR-L1 comprising the amino acid sequence of SEQ ID NO:378; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO:155; (d) HVR-L1 comprising the amino acid sequence of SEQ ID NO:376; (e) HVR-L2 comprising the amino acid sequence of SEQ ID NO:203; and (f) HVR-L3 comprising the amino acid sequence of SEQ ID NO:231; (a) HVR-H1 comprising the amino acid sequence of SEQ ID NO:90; (b) HVR-H2 comprising the amino acid sequence of SEQ ID NO:369; (c) HVR-H3 comprising the amino acid sequence of SEQ ID NO:155; (d) HVR-L1 comprising the amino acid sequence of SEQ ID NO:378; (e) HVR-L2 comprising the amino acid sequence of SEQ ID NO:203; and (f) HVR-L3 comprising the amino acid sequence of SEQ ID NO:394; (a) HVR-H1 comprising the amino acid sequence of SEQ ID NO:90; (b) HVR-H2 comprising the amino acid sequence of SEQ ID NO:370; (c) HVR-H3 comprising the amino acid sequence of SEQ ID NO:155; (d) HVR-L1 comprising the amino acid sequence of SEQ ID NO:378; (e) HVR-L2 comprising the amino acid sequence of SEQ ID NO:203; and (f) HVR-L3 comprising the amino acid sequence of SEQ ID NO:394; (a) HVR-H1 comprising the amino acid sequence of SEQ ID NO:90; (b) HVR-H2 comprising the amino acid sequence of SEQ ID NO:371; (c) HVR-H3 comprising the amino acid sequence of SEQ ID NO:155; (d) HVR-L1 comprising the amino acid sequence of SEQ ID NO: 178; (e) HVR-L2 comprising the amino acid sequence of SEQ ID NO:203; and (f) HVR-L3 comprising the amino acid sequence of SEQ ID NO:394; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO:155; (d) HVR-L1 comprising the amino acid sequence of SEQ ID NO:385; (e) HVR-L2 comprising the amino acid sequence of SEQ ID NO:203; and (f) HVR-L3 comprising the amino acid sequence of SEQ ID NO:394; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO: 155; (d) HVR-L1 comprising the amino acid sequence of SEQ ID NO:378; (e) HVR-L2 comprising the amino acid sequence of SEQ ID NO:203; and (f) HVR-L3 comprising the amino acid sequence of SEQ ID NO:395; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO: 155; (d) HVR-L1 comprising the amino acid sequence of SEQ ID NO:378; (e) HVR-L2 comprising the amino acid sequence of SEQ ID NO:390; and (f) HVR-L3 comprising the amino acid sequence of SEQ ID NO:394; (a) HVR-H1 comprising the amino acid sequence of SEQ ID NO:90; (b) HVR-H2 comprising the amino acid sequence of SEQ ID NO:372; (c) HVR-H3 comprising the amino acid sequence of SEQ ID NO: 155; (d) HVR-L1 comprising the amino acid sequence of SEQ ID NO:378; (e) HVR-L2 comprising the amino acid sequence of SEQ ID NO:391; and (f) HVR-L3 comprising the amino acid sequence of SEQ ID NO:394; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO:375; (d) HVR-L1 comprising the amino acid sequence of SEQ ID NO:386; (e) HVR-L2 comprising the amino acid sequence of SEQ ID NO:203; and (f) HVR-L3 comprising the amino acid sequence of SEQ ID NO:394; (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO:155; (d) HVR-L1 comprising the amino acid sequence of SEQ ID NO:387; (e) HVR-L2 comprising the amino acid sequence of SEQ ID NO:203; and (f) HVR-L3 comprising the amino acid sequence of SEQ ID NO:394.

In another aspect, an anti-MerTK antibody comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs:37, 290, 291, 292, 293, 294, 295, 296, 297, 298, 299, 300, 301, 302, 303, 304, 305, 306, 307, and 308. In certain embodiments, a V_{H} sequence having at least 90%, 91%, 92%, 93%, 94%, 95%, 96%, 97%, 98%, or 99% identity to an amino acid sequence selected from the group consisting of SEQ ID NOs: 37, 290, 291, 292, 293, 294, 295, 296, 297, 298, 299, 300, 301, 302, 303, 304, 305, 306, 307, and 308 contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted, and/or deleted in SEQ ID NO: 37, 290, 291, 292, 293, 294, 295, 296, 297, 298, 299, 300, 301, 302, 303, 304, 305, 306, 307, or 308. In certain embodiments, a total of 1 to 5 amino acids have been substituted, inserted and/or deleted in SEQ ID NO: 37, 290, 291, 292, 293, 294, 295, 296, 297, 298, 299, 300, 301, 302, 303, 304, 305, 306, 307, or 308. In certain embodiments, substitutions, insertions, or deletions occur in regions outside the HVRs (i.e., in the FRs). Optionally, the anti-MerTK antibody comprises the V_{H} sequence of SEQ ID NO: 37, 290, 291, 292, 293, 294, 295, 296, 297, 298, 299, 300, 301, 302, 303, 304, 305, 306, 307, or 308, including post-translational modifications of that sequence. In a particular embodiment, the V_{H} comprises one, two or three HVRs selected from: (a) HVR-H1 comprising the amino acid sequence of SEQ ID NO:90, (b) HVR-H2 comprising an amino acid sequence selected from the group consisting of SEQ ID NOs:122, 364, 365, 366, 367, and 368, and (c) HVR-H3 comprising an amino acid sequence selected from the group consisting of SEQ ID NOs:155, 373, 374, and 375.

In another aspect, an anti-MerTK antibody is provided, wherein the antibody comprises a light chain variable domain (V_{L}) having at least 90%, 91%, 92%, 93%, 94%, 95%, 96%, 97%, 98%, 99%, or 100% sequence identity to an amino acid sequence selected from the group consisting of SEQ ID NOs:72, 309, 310, 311, 312, 313, 314, 315, 316, 316, 318, 319, 320, 321, 322, 323, 324, 325, 326, 327, and 328. In certain embodiments, a V_{L} sequence having at least 90%, 91%, 92%, 93%, 94%, 95%, 96%, 97%, 98%, or 99% identity to an amino acid sequence selected from the group consisting of SEQ ID NOs: 72, 309, 310, 311, 312, 313, 314, 315, 316, 316, 318, 319, 320, 321, 322, 323, 324, 325, 326, 327, and 328, and contains substitutions (e.g., conservative substitutions), insertions, or deletions relative to the reference sequence, but an anti-MerTK antibody comprising that sequence retains the ability to bind to MerTK. In some embodiments, a total of 1 to 10 amino acids have been substituted, inserted and/or deleted in SEQ ID NO: 72, 309, 310, 311, 312, 313, 314, 315, 316, 316, 318, 319, 320, 321, 322, 323, 324, 325, 326, 327, or 328. In certain embodiments, a total of 1 to 5 amino acids have been substituted, inserted and/or deleted in SEQ ID NO: 72, 309, 310, 311, 312, 313, 314, 315, 316, 316, 318, 319, 320, 321, 322, 323, 324, 325, 326, 327, or 328. In certain embodiments, the substitutions, insertions, or deletions occur in regions outside the HVRs (i.e., in the FRs). Optionally, the anti-MerTK antibody comprises the V_{L} sequence of SEQ ID NO: 72, 309, 310, 311, 312, 313, 314, 315, 316, 316, 318, 319, 320, 321, 322, 323, 324, 325, 326, 327, or 328, including post-translational modifications of that sequence. In a particular embodiment, the V_{L} comprises one, two or three HVRs selected from (a) HVR-L1 comprising an amino acid sequence selected from the group consisting of SEQ ID NOs:184, 376, 377, 378, 379, 380, 381, 382, 383, 384, 385, 386, and 387, (b) HVR-L2 comprising an amino acid sequence selected from the group consisting of SEQ ID NOs: 203, 388, 389, 390, and 391, and (c) HVR-L3 comprising an amino acid sequence selected from the group consisting of SEQ ID NOs: 231, 392, 393, and 394.

In some embodiments, an anti-MerTK antibody is provided, wherein the antibody comprises a V_{H} as in any of the embodiments provided above, and a V_{L} as in any of the embodiments provided above. In some embodiments, provided herein are anti-MerTK antibodies, wherein the antibody comprises a V_{H} as in any of the embodiments provided above, and a V_{L} as in any of the embodiments provided above. In one embodiment, the antibody comprises the V_{H} and V_{L} sequences in SEQ ID NOs: 37, 290, 291, 292, 293, 294, 295, 296, 297, 298, 299, 300, 301, 302, 303, 304, 305, 306, 307, and 308 and SEQ ID NOs: 72, 309, 310, 311, 312, 313, 314, 315, 316, 316, 318, 319, 320, 321, 322, 323, 324, 325, 326, 327, and 328, respectively, including post-translational modifications of those sequences.

In some embodiments, provided herein are anti-MerTK antibodies comprising a heavy chain variable domain (V_{H}) and a light chain variable domain (V_{L}), wherein the V_{H} and V_{L} are selected from the group consisting of: V_{H} comprising the amino acid sequence of SEQ ID NO:37 and V_{L} comprising the amino acid sequence of SEQ ID NO:72; V_{H} comprising the amino acid sequence of SEQ ID NO:290 and V_{L} comprising the amino acid sequence of SEQ ID NO:309; V_{H} comprising the amino acid sequence of SEQ ID NO:291 and V_{L} comprising the amino acid sequence of SEQ ID NO:310; V_{H} comprising the amino acid sequence of SEQ ID NO:292 and V_{L} comprising the amino acid sequence of SEQ ID NO:311; V_{H} comprising the amino acid sequence of SEQ ID NO:293 and V_{L} comprising the amino acid sequence of SEQ ID NO:312; V_{H} comprising the amino acid sequence of SEQ ID NO:294 and V_{L} comprising the amino acid sequence of SEQ ID NO:313; V_{H} comprising the amino acid sequence of SEQ ID NO:295 and V_{L} comprising the amino acid sequence of SEQ ID NO:314; V_{H} comprising the amino acid sequence of SEQ ID NO:296 and V_{L} comprising the amino acid sequence of SEQ ID NO:315; V_{H} comprising the amino acid sequence of SEQ ID NO:290 and V_{L} comprising the amino acid sequence of SEQ ID NO:316; V_{H} comprising the amino acid sequence of SEQ ID NO:297 and V_{L} comprising the amino acid sequence of SEQ ID NO:317; V_{H} comprising the amino acid sequence of SEQ ID NO:298 and V_{L} comprising the amino acid sequence of SEQ ID NO:318; V_{H} comprising the amino acid sequence of SEQ ID NO:292 and V_{L} comprising the amino acid sequence of SEQ ID NO:319; V_{H} comprising the amino acid sequence of SEQ ID NO:299 and V_{L} comprising the amino acid sequence of SEQ ID NO:320; V_{H} comprising the amino acid sequence of SEQ ID NO:300 and V_{L} comprising the amino acid sequence of SEQ ID NO:311; V_{H} comprising the amino acid sequence of SEQ ID NO:301 and V_{L} comprising the amino acid sequence of SEQ ID NO:321; V_{H} comprising the amino acid sequence of SEQ ID NO:302 and V_{L} comprising the amino acid sequence of SEQ ID NO:322; V_{H} comprising the amino acid sequence of SEQ ID NO:303 and V_{L} comprising the amino acid sequence of SEQ ID NO:311; V_{H} comprising the amino acid sequence of SEQ ID NO:304 and V_{L} comprising the amino acid sequence of SEQ ID NO:322; V_{H} comprising the amino acid sequence of SEQ ID NO:305 and V_{L} comprising the amino acid sequence of SEQ ID NO:322; V_{H} comprising the amino acid sequence of SEQ ID NO:301 and V_{L} comprising the amino acid sequence of SEQ ID NO:323; V_{H} comprising the amino acid sequence of SEQ ID NO:301 and V_{L} comprising the amino acid sequence of SEQ ID NO:324; V_{H} comprising the amino acid sequence of SEQ ID NO:301 and V_{L} comprising the amino acid sequence of SEQ ID NO:325; V_{H} comprising the amino acid sequence of SEQ ID NO:306 and V_{L} comprising the amino acid sequence of SEQ ID NO:326; V_{H} comprising the amino acid sequence of SEQ ID NO:307 and V_{L} comprising the amino acid sequence of SEQ ID NO:327; and V_{H} comprising the amino acid sequence of SEQ ID NO:308 and V_{L} comprising the amino acid sequence of SEQ ID NO:328.

In some embodiments, an anti-MerTK antibody of the present disclosure competitively inhibits binding of at least one reference antibody selected from MTK-01, MTK-02, MTK-03, MTK-04, MTK-05, MTK-06, MTK-07, MTK-08, MTK-09, MTK-10, MTK-11, MTK-12, MTK-13, MTK-14, MTK-15 (including MTK-15.1, MTK-15.2, MTK-15.3, MTK-15.4, MTK-15.5, MTK-15.6, MTK-15.7, MTK-15.8, MTK-15.9, MTK-15.10, MTK-15.11, MTK-15.12, MTK-15.13, MTK-15.14, and MTK-15.15), MTK-16 (including MTK-16.1 and MTK-16.2), MTK-17, MTK-18, MTK-19, MTK-20, MTK-21, MTK-22, MTK-23, MTK-24, MTK-25, MTK-26, MTK-27, MTK-28, MTK-29 (including MTK-29.1, MTK-29.2, MTK-29.3, MTK-29.4, MTK-29.5, MTK-29.6, MTK-29.7, MTK-29.8, MTK-29.9, MTK-29.10, MTK-29.11, MTK-29.12, MTK-29.13, MTK-29.14, MTK-29.15, MTK-29.16, MTK-29.17, MTK-29.18, MTK-29.19, MTK-29.20, and MTK-29.21), MTK-30, MTK-31, MTK-32, MTK-33 (including MTK-33.1, MTK-33.2, MTK-33.3, MTK-33.4, MTK-33.5, MTK-33.6, MTK-33.7, MTK-33.8, MTK-33.9, MTK-33.10, MTK-33.11, MTK-33.12, MTK-33.13, MTK-33.14, MTK-33.15, MTK-33.16, MTK-33.17, MTK-33.18, MTK-33.19, MTK-33.20, MTK-33.21, MTK-33.22, MTK-33.23, and MTK-33.24), MTK-34, MTK-35, and MTK-36, and any combination thereof, for binding to MerTK.

In some embodiments, an anti-MerTK antibody of the present disclosure binds to an epitope of human MerTK that is the same as or overlaps with the MerTK epitope bound by at least one reference antibody selected from MTK-01, MTK-02, MTK-03, MTK-04, MTK-05, MTK-06, MTK-07, MTK-08, MTK-09, MTK-10, MTK-11, MTK-12, MTK-13, MTK-14, MTK-15 (including MTK-15.1, MTK-15.2, MTK-15.3, MTK-15.4, MTK-15.5, MTK-15.6, MTK-15.7, MTK-15.8, MTK-15.9, MTK-15.10, MTK-15.11, MTK-15.12, MTK-15.13, MTK-15.14, and MTK-15.15), MTK-16 (including MTK-16.1 and MTK-16.2), MTK-17, MTK-18, MTK-19, MTK-20, MTK-21, MTK-22, MTK-23, MTK-24, MTK-25, MTK-26, MTK-27, MTK-28, MTK-29 (including MTK-29.1, MTK-29.2, MTK-29.3, MTK-29.4, MTK-29.5, MTK-29.6, MTK-29.7, MTK-29.8, MTK-29.9, MTK-29.10, MTK-29.11, MTK-29.12, MTK-29.13, MTK-29.14, MTK-29.15, MTK-29.16, MTK-29.17, MTK-29.18, MTK-29.19, MTK-29.20, and MTK-29.21), MTK-30, MTK-31, MTK-32, MTK-33 (including MTK-33.1, MTK-33.2, MTK-33.3, MTK-33.4, MTK-33.5, MTK-33.6, MTK-33.7, MTK-33.8, MTK-33.9, MTK-33.10, MTK-33.11, MTK-33.12, MTK-33.13, MTK-33.14, MTK-33.15, MTK-33.16, MTK-33.17, MTK-33.18, MTK-33.19, MTK-33.20, MTK-33.21, MTK-33.22, MTK-33.23, and MTK-33.24), MTK-34, MTK-35, and MTK-36. Detailed exemplary methods for mapping an epitope to which an antibody binds are provided in Morris (1996) “Epitope Mapping Protocols,” in Methods in Molecular Biology vol. 66 (Humana Press, Totowa, NJ).

In some embodiments, an anti-MerTK antibody of the present disclosure competitively inhibits binding of at least one reference antibody, or binds to an epitope of human MerTK that is the same as or overlaps with the MerTK epitope bound by at least one reference antibody, wherein the reference antibody is an anti-MerTK antibody comprising a heavy chain variable domain (V_{H}) and a light chain variable domain (V_{L}), wherein the V_{H} and V_{L} are selected from the group consisting of: V_{H} comprising the amino acid sequence of SEQ ID NO:5 and V_{L} comprising the amino acid sequence of SEQ ID NO:40; V_{H} comprising the amino acid sequence of SEQ ID NO:6 and V_{L} comprising the amino acid sequence of SEQ ID NO:41; V_{H} comprising the amino acid sequence of SEQ ID NO:7 and V_{L} comprising the amino acid sequence of SEQ ID NO:42; V_{H} comprising the amino acid sequence of SEQ ID NO:8 and V_{L} comprising the amino acid sequence of SEQ ID NO:43; V_{H} comprising the amino acid sequence of SEQ ID NO:9 and V_{L} comprising the amino acid sequence of SEQ ID NO:44; V_{H} comprising the amino acid sequence of SEQ ID NO: 10 and V_{L} comprising the amino acid sequence of SEQ ID NO:45; V_{H} comprising the amino acid sequence of SEQ ID NO: 11 and V_{L} comprising the amino acid sequence of SEQ ID NO:46; V_{H} comprising the amino acid sequence of SEQ ID NO:12 and V_{L} comprising the amino acid sequence of SEQ ID NO:47; V_{H} comprising the amino acid sequence of SEQ ID NO: 13 and V_{L} comprising the amino acid sequence of SEQ ID NO:48; V_{H} comprising the amino acid sequence of SEQ ID NO: 14 and V_{L} comprising the amino acid sequence of SEQ ID NO:49; V_{H} comprising the amino acid sequence of SEQ ID NO: 15 and V_{L} comprising the amino acid sequence of SEQ ID NO:50; V_{H} comprising the amino acid sequence of SEQ ID NO: 16 and V_{L} comprising the amino acid sequence of SEQ ID NO:51; V_{H} comprising the amino acid sequence of SEQ ID NO:17 and V_{L} comprising the amino acid sequence of SEQ ID NO:52; V_{H} comprising the amino acid sequence of SEQ ID NO: 18 and V_{L} comprising the amino acid sequence of SEQ ID NO:53; V_{H} comprising the amino acid sequence of SEQ ID NO: 19 and V_{L} comprising the amino acid sequence of SEQ ID NO:54; V_{H} comprising the amino acid sequence of SEQ ID NO:20 and V_{L} comprising the amino acid sequence of SEQ ID NO:55; V_{H} comprising the amino acid sequence of SEQ ID NO:21 and V_{L} comprising the amino acid sequence of SEQ ID NO:56; V_{H} comprising the amino acid sequence of SEQ ID NO:22 and V_{L} comprising the amino acid sequence of SEQ ID NO:57; V_{H} comprising the amino acid sequence of SEQ ID NO:23 and V_{L} comprising the amino acid sequence of SEQ ID NO:58; V_{H} comprising the amino acid sequence of SEQ ID NO:24 and V_{L} comprising the amino acid sequence of SEQ ID NO:59; V_{H} comprising the amino acid sequence of SEQ ID NO:25 and V_{L} comprising the amino acid sequence of SEQ ID NO:60; V_{H} comprising the amino acid sequence of SEQ ID NO:26 and V_{L} comprising the amino acid sequence of SEQ ID NO:61; V_{H} comprising the amino acid sequence of SEQ ID NO:27 and V_{L} comprising the amino acid sequence of SEQ ID NO:62; V_{H} comprising the amino acid sequence of SEQ ID NO:28 and V_{L} comprising the amino acid sequence of SEQ ID NO:63; V_{H} comprising the amino acid sequence of SEQ ID NO:29 and V_{L} comprising the amino acid sequence of SEQ ID NO:64; V_{H} comprising the amino acid sequence of SEQ ID NO:30 and V_{L} comprising the amino acid sequence of SEQ ID NO:65; V_{H} comprising the amino acid sequence of SEQ ID NO:31 and V_{L} comprising the amino acid sequence of SEQ ID NO:66; V_{H} comprising the amino acid sequence of SEQ ID NO:32 and V_{L} comprising the amino acid sequence of SEQ ID NO:67; V_{H} comprising the amino acid sequence of SEQ ID NO:33 and V_{L} comprising the amino acid sequence of SEQ ID NO:68; V_{H} comprising the amino acid sequence of SEQ ID NO:34 and V_{L} comprising the amino acid sequence of SEQ ID NO:69; V_{H} comprising the amino acid sequence of SEQ ID NO:35 and V_{L} comprising the amino acid sequence of SEQ ID NO:70; V_{H} comprising the amino acid sequence of SEQ ID NO:36 and V_{L} comprising the amino acid sequence of SEQ ID NO:71; V_{H} comprising the amino acid sequence of SEQ ID NO:37 and V_{L} comprising the amino acid sequence of SEQ ID NO:72; V_{H} comprising the amino acid sequence of SEQ ID NO:38 and V_{L} comprising the amino acid sequence of SEQ ID NO:73; and V_{H} comprising the amino acid sequence of SEQ ID NO:39 and V_{L} comprising the amino acid sequence of SEQ ID NO:74.

In some embodiments, an anti-MerTK antibody of the present disclosure comprises a full-length heavy chain amino acid sequence comprising a variable heavy chain amino acid sequence of SEQ ID NO: 234, 235, 236, 237, 238, 239, 240, 241, 242, 243, 244, 245, or 246, and a heavy chain Fc selected from the group consisting of SEQ ID NOs: 396-411; and comprises a full-length light chain amino acid sequence comprising a variable light chain amino acid sequence of SEQ ID NO:247, 248, 249, 250, 251, 252, 253, or 254, and a light chain Fc comprising the amino acid sequence of SEQ ID NO:412.

In some embodiments, an anti-MerTK antibody of the present disclosure comprises a full-length heavy chain amino acid sequence comprising a variable heavy chain amino acid sequence of SEQ ID NO:255 or 256 and a heavy chain Fc selected from the group consisting of SEQ ID NOs: 396-411; and comprises a full-length light chain amino acid sequence comprising a variable light chain amino acid sequence of SEQ ID NO:257 or 258 and a light chain Fc comprising the amino acid sequence of SEQ ID NO:412.

In some embodiments, an anti-MerTK antibody of the present disclosure comprises a full-length heavy chain amino acid sequence of SEQ ID NO:413, 414, 415, 416, 417, 418, 419, or 420 and a full-length light chain amino acid sequence of SEQ ID NO: 421. Accordingly, in some embodiments, an anti-MerTK antibody comprises the heavy chain amino acid sequence of SEQ ID NO:413 and the light chain amino acid sequence of SEQ ID NO:421; the heavy chain amino acid sequence of SEQ ID NO:414 and the light chain amino acid sequence of SEQ ID NO:421; the heavy chain amino acid sequence of SEQ ID NO:415 and the light chain amino acid sequence of SEQ ID NO:421; the heavy chain amino acid sequence of SEQ ID NO:416 and the light chain amino acid sequence of SEQ ID NO:421; the heavy chain amino acid sequence of SEQ ID NO:417 and the light chain amino acid sequence of SEQ ID NO:421; the heavy chain amino acid sequence of SEQ ID NO:418 and the light chain amino acid sequence of SEQ ID NO:421; the heavy chain amino acid sequence of SEQ ID NO:419 and the light chain amino acid sequence of SEQ ID NO:421; or the heavy chain amino acid sequence of SEQ ID NO:420 and the light chain amino acid sequence of SEQ ID NO:421.

In some embodiments, an anti-MerTK antibody of the present disclosure comprises a full-length heavy chain amino acid sequence of SEQ ID NO:422, 423, 424, 425, 426, 427, 428, or 429 and a full-length light chain amino acid sequence of SEQ ID NO: 430. Accordingly, in some embodiments, an anti-MerTK antibody comprises the heavy chain amino acid sequence of SEQ ID NO:422 and the light chain amino acid sequence of SEQ ID NO:430; the heavy chain amino acid sequence of SEQ ID NO:423 and the light chain amino acid sequence of SEQ ID NO:430; the heavy chain amino acid sequence of SEQ ID NO:424 and the light chain amino acid sequence of SEQ ID NO:430; the heavy chain amino acid sequence of SEQ ID NO:425 and the light chain amino acid sequence of SEQ ID NO:430; the heavy chain amino acid sequence of SEQ ID NO:426 and the light chain amino acid sequence of SEQ ID NO:430; the heavy chain amino acid sequence of SEQ ID NO:427 and the light chain amino acid sequence of SEQ ID NO:430; the heavy chain amino acid sequence of SEQ ID NO:428 and the light chain amino acid sequence of SEQ ID NO:430; or the heavy chain amino acid sequence of SEQ ID NO:429 and the light chain amino acid sequence of SEQ ID NO:430.

In some embodiments, an anti-MerTK antibody of the present disclosure comprises a full-length heavy chain amino acid sequence comprising a variable heavy chain amino acid sequence of SEQ ID NO:259, 260, 261, 262, 263, 264, 265, 266, 267, 268, 269, 270, 271, 272, or 273, and a heavy chain Fc selected from the group consisting of SEQ ID NOs: 396-411; and comprises a full-length light chain amino acid sequence comprising a variable light chain amino acid sequence of SEQ ID NO:274, 275, 276, 277, 278, 279, 280, 281, 282, 283, 284, 285, 286, 287, 288, or 289, and a light chain Fc comprising the amino acid sequence of SEQ ID NO:412.

In some embodiments, an anti-MerTK antibody of the present disclosure comprises a full-length heavy chain amino acid sequence comprising a variable heavy chain amino acid sequence of SEQ ID NO: 290, 291, 292, 293, 294, 295, 296, 297, 298, 299, 300, 301, 302, 303, 304, 305, 306, 307, or 308, and a heavy chain Fc selected from the group consisting of SEQ ID NOs: 396-411; and comprises a full-length light chain amino acid sequence comprising a variable light chain amino acid sequence of SEQ ID NO:309, 310, 311, 312, 313, 314, 315, 316, 317, 318, 319, 320, 321, 322, 323, 324, 325, 326, 327, or 328, and a light chain Fc comprising the amino acid sequence of SEQ ID NO:412.

In some embodiments, an anti-MerTK antibody of the present disclosure comprises a full-length heavy chain amino acid sequence of SEQ ID NO: 431, 432, 433, 434, 435, 436, 437, or 438 and a full-length light chain amino acid sequence of SEQ ID NO:439. Accordingly, in some embodiments, an anti-MerTK antibody comprises the heavy chain amino acid sequence of SEQ ID NO:431 and the light chain amino acid sequence of SEQ ID NO:439; the heavy chain amino acid sequence of SEQ ID NO:432 and the light chain amino acid sequence of SEQ ID NO:439; the heavy chain amino acid sequence of SEQ ID NO:433 and the light chain amino acid sequence of SEQ ID NO:439; the heavy chain amino acid sequence of SEQ ID NO:434 and the light chain amino acid sequence of SEQ ID NO:439; the heavy chain amino acid sequence of SEQ ID NO:435 and the light chain amino acid sequence of SEQ ID NO:439; the heavy chain amino acid sequence of SEQ ID NO:436 and the light chain amino acid sequence of SEQ ID NO:439; the heavy chain amino acid sequence of SEQ ID NO:437 and the light chain amino acid sequence of SEQ ID NO:439; or the heavy chain amino acid sequence of SEQ ID NO:438 and the light chain amino acid sequence of SEQ ID NO:439.

In some embodiments, an anti-MerTK antibody of the present disclosure comprises a full-length heavy chain amino acid sequence of SEQ ID NO: 464, 465, 466, 467, 468, 469, 470, and 471 and a full-length light chain amino acid sequence of SEQ ID NO:472. Accordingly, in some embodiments, an anti-MerTK antibody comprises the heavy chain amino acid sequence of SEQ ID NO:464 and the light chain amino acid sequence of SEQ ID NO:472; the heavy chain amino acid sequence of SEQ ID NO:465 and the light chain amino acid sequence of SEQ ID NO:472; the heavy chain amino acid sequence of SEQ ID NO:466 and the light chain amino acid sequence of SEQ ID NO:472; the heavy chain amino acid sequence of SEQ ID NO:467 and the light chain amino acid sequence of SEQ ID NO:472; the heavy chain amino acid sequence of SEQ ID NO:468 and the light chain amino acid sequence of SEQ ID NO:472; the heavy chain amino acid sequence of SEQ ID NO:469 and the light chain amino acid sequence of SEQ ID NO:472; the heavy chain amino acid sequence of SEQ ID NO:470 and the light chain amino acid sequence of SEQ ID NO:472; or the heavy chain amino acid sequence of SEQ ID NO:471 and the light chain amino acid sequence of SEQ ID NO:472.

In some embodiments, an anti-MerTK antibody of the present disclosure comprises a full-length heavy chain amino acid sequence of SEQ D NO: 440, 441, 442, 443, 444, 445, 446, or 447 and a full-length light chain amino acid sequence of SEQ ID NO:448. Accordingly, in some embodiments, an anti-MerTK antibody comprises the heavy chain amino acid sequence of SEQ ID NO:440 and the light chain amino acid sequence of SEQ ID NO:448; the heavy chain amino acid sequence of SEQ ID NO:441 and the light chain amino acid sequence of SEQ ID NO:448; the heavy chain amino acid sequence of SEQ ID NO:442 and the light chain amino acid sequence of SEQ ID NO:448; the heavy chain amino acid sequence of SEQ ID NO:443 and the light chain amino acid sequence of SEQ ID NO:448; the heavy chain amino acid sequence of SEQ ID NO:444 and the light chain amino acid sequence of SEQ ID NO:448; the heavy chain amino acid sequence of SEQ ID NO:445 and the light chain amino acid sequence of SEQ ID NO:448; the heavy chain amino acid sequence of SEQ ID NO:446 and the light chain amino acid sequence of SEQ ID NO:448; or the heavy chain amino acid sequence of SEQ ID NO:447 and the light chain amino acid sequence of SEQ ID NO:448.

In some embodiments, an anti-MerTK antibody of the present disclosure competitively inhibits binding of at least one reference antibody, or binds to an epitope of human MerTK that is the same as or overlaps with the MerTK epitope bound by at least one reference antibody, wherein the reference antibody is an anti-MerTK antibody comprising a heavy chain variable domain (V_{H}) and a light chain variable domain (V_{L}), wherein the V_{H} and V_{L} are selected from the group consisting of: V_{H} comprising the amino acid sequence of SEQ ID NO:19 and V_{L} comprising the amino acid sequence of SEQ ID NO:54; V_{H} comprising the amino acid sequence of SEQ ID NO:234 and V_{L} comprising the amino acid sequence of SEQ ID NO:247; V_{H} comprising the amino acid sequence of SEQ ID NO:235 and V_{L} comprising the amino acid sequence of SEQ ID NO:247; V_{H} comprising the amino acid sequence of SEQ ID NO:236 and V_{L} comprising the amino acid sequence of SEQ ID NO:248; V_{H} comprising the amino acid sequence of SEQ ID NO:236 and V_{L} comprising the amino acid sequence of SEQ ID NO:249; V_{H} comprising the amino acid sequence of SEQ ID NO:237 and V_{L} comprising the amino acid sequence of SEQ ID NO:249; V_{H} comprising the amino acid sequence of SEQ ID NO:238 and V_{L} comprising the amino acid sequence of SEQ ID NO:249; V_{H} comprising the amino acid sequence of SEQ ID NO:239 and V_{L} comprising the amino acid sequence of SEQ ID NO:250; V_{H} comprising the amino acid sequence of SEQ ID NO:240 and V_{L} comprising the amino acid sequence of SEQ ID NO:251; V_{H} comprising the amino acid sequence of SEQ ID NO:241 and V_{L} comprising the amino acid sequence of SEQ ID NO:252; V_{H} comprising the amino acid sequence of SEQ ID NO:242 and V_{L} comprising the amino acid sequence of SEQ ID NO:249; V_{H} comprising the amino acid sequence of SEQ ID NO:243 and V_{L} comprising the amino acid sequence of SEQ ID NO:247; V_{H} comprising the amino acid sequence of SEQ ID NO:244 and V_{L} comprising the amino acid sequence of SEQ ID NO:251; V_{H} comprising the amino acid sequence of SEQ ID NO:245 and V_{L} comprising the amino acid sequence of SEQ ID NO:253; V_{H} comprising the amino acid sequence of SEQ ID NO:246 and V_{L} comprising the amino acid sequence of SEQ ID NO:247; and V_{H} comprising the amino acid sequence of SEQ ID NO:246 and V_{L} comprising the amino acid sequence of SEQ ID NO:254.

In some embodiments, an anti-MerTK antibody of the present disclosure competitively inhibits binding of at least one reference antibody, or binds to an epitope of human MerTK that is the same as or overlaps with the MerTK epitope bound by at least one reference antibody, wherein the reference antibody is an anti-MerTK antibody comprising a heavy chain variable domain (V_{H}) and a light chain variable domain (V_{L}), wherein the V_{H} and V_{L} are selected from the group consisting of: V_{H} comprising the amino acid sequence of SEQ ID NO:20 and V_{L} comprising the amino acid sequence of SEQ ID NO:55; V_{H} comprising the amino acid sequence of SEQ ID NO:255 and V_{L} comprising the amino acid sequence of SEQ ID NO:257; and V_{H} comprising the amino acid sequence of SEQ ID NO:256 and V_{L} comprising the amino acid sequence of SEQ ID NO:258.

In some embodiments, an anti-MerTK antibody of the present disclosure competitively inhibits binding of at least one reference antibody, or binds to an epitope of human MerTK that is the same as or overlaps with the MerTK epitope bound by at least one reference antibody, wherein the reference antibody is an anti-MerTK antibody comprising a heavy chain variable domain (V_{H}) and a light chain variable domain (V_{L}), wherein the V_{H} and V_{L} are selected from the group consisting of: V_{H} comprising the amino acid sequence of SEQ ID NO:33 and V_{L} comprising the amino acid sequence of SEQ ID NO:68; V_{H} comprising the amino acid sequence of SEQ ID NO:259 and V_{L} comprising the amino acid sequence of SEQ ID NO:274; V_{H} comprising the amino acid sequence of SEQ ID NO:260 and V_{L} comprising the amino acid sequence of SEQ ID NO:275; V_{H} comprising the amino acid sequence of SEQ ID NO:261 and V_{L} comprising the amino acid sequence of SEQ ID NO:276; V_{H} comprising the amino acid sequence of SEQ ID NO:262 and V_{L} comprising the amino acid sequence of SEQ ID NO:277; V_{H} comprising the amino acid sequence of SEQ ID NO:263 and V_{L} comprising the amino acid sequence of SEQ ID NO:277; V_{H} comprising the amino acid sequence of SEQ ID NO:264 and V_{L} comprising the amino acid sequence of SEQ ID NO:277; V_{H} comprising the amino acid sequence of SEQ ID NO:265 and V_{L} comprising the amino acid sequence of SEQ ID NO:277; V_{H} comprising the amino acid sequence of SEQ ID NO:266 and V_{L} comprising the amino acid sequence of SEQ ID NO:277; V_{H} comprising the amino acid sequence of SEQ ID NO:267 and V_{L} comprising the amino acid sequence of SEQ ID NO:278; V_{H} comprising the amino acid sequence of SEQ ID NO:268 and V_{L} comprising the amino acid sequence of SEQ ID NO:279; V_{H} comprising the amino acid sequence of SEQ ID NO:269 and V_{L} comprising the amino acid sequence of SEQ ID NO:279; V_{H} comprising the amino acid sequence of SEQ ID NO:264 and V_{L} comprising the amino acid sequence of SEQ ID NO:280; V_{H} comprising the amino acid sequence of SEQ ID NO:270 and V_{L} comprising the amino acid sequence of SEQ ID NO:281; V_{H} comprising the amino acid sequence of SEQ ID NO:265 and V_{L} comprising the amino acid sequence of SEQ ID NO:282; V_{H} comprising the amino acid sequence of SEQ ID NO:264 and V_{L} comprising the amino acid sequence of SEQ ID NO:283; V_{H} comprising the amino acid sequence of SEQ ID NO:271 and V_{L} comprising the amino acid sequence of SEQ ID NO:284; V_{H} comprising the amino acid sequence of SEQ ID NO:272 and V_{L} comprising the amino acid sequence of SEQ ID NO:285; V_{H} comprising the amino acid sequence of SEQ ID NO:271 and V_{L} comprising the amino acid sequence of SEQ ID NO:286; V_{H} comprising the amino acid sequence of SEQ ID NO:273 and V_{L} comprising the amino acid sequence of SEQ ID NO:287; V_{H} comprising the amino acid sequence of SEQ ID NO:273 and V_{L} comprising the amino acid sequence of SEQ ID NO:288; and V_{H} comprising the amino acid sequence of SEQ ID NO:273 and V_{L} comprising the amino acid sequence of SEQ ID NO:289.

In some embodiments, an anti-MerTK antibody of the present disclosure competitively inhibits binding of at least one reference antibody, or binds to an epitope of human MerTK that is the same as or overlaps with the MerTK epitope bound by at least one reference antibody, wherein the reference antibody is an anti-MerTK antibody comprising a heavy chain variable domain (V_{H}) and a light chain variable domain (V_{L}), wherein the V_{H} and V_{L} are selected from the group consisting of: V_{H} comprising the amino acid sequence of SEQ ID NO:37 and V_{L} comprising the amino acid sequence of SEQ ID NO:72; V_{H} comprising the amino acid sequence of SEQ ID NO:290 and V_{L} comprising the amino acid sequence of SEQ ID NO:309; V_{H} comprising the amino acid sequence of SEQ ID NO:291 and V_{L} comprising the amino acid sequence of SEQ ID NO:310; V_{H} comprising the amino acid sequence of SEQ ID NO:292 and V_{L} comprising the amino acid sequence of SEQ ID NO:311; V_{H} comprising the amino acid sequence of SEQ ID NO:293 and V_{L} comprising the amino acid sequence of SEQ ID NO:312; V_{H} comprising the amino acid sequence of SEQ ID NO:294 and V_{L} comprising the amino acid sequence of SEQ ID NO:313; V_{H} comprising the amino acid sequence of SEQ ID NO:295 and V_{L} comprising the amino acid sequence of SEQ ID NO:314; V_{H} comprising the amino acid sequence of SEQ ID NO:296 and V_{L} comprising the amino acid sequence of SEQ ID NO:315; V_{H} comprising the amino acid sequence of SEQ ID NO:290 and V_{L} comprising the amino acid sequence of SEQ ID NO:316; V_{H} comprising the amino acid sequence of SEQ ID NO:297 and V_{L} comprising the amino acid sequence of SEQ ID NO:317; V_{H} comprising the amino acid sequence of SEQ ID NO:298 and V_{L} comprising the amino acid sequence of SEQ ID NO:318; V_{H} comprising the amino acid sequence of SEQ ID NO:292 and V_{L} comprising the amino acid sequence of SEQ ID NO:319; V_{H} comprising the amino acid sequence of SEQ ID NO:299 and V_{L} comprising the amino acid sequence of SEQ ID NO:320; V_{H} comprising the amino acid sequence of SEQ ID NO:300 and V_{L} comprising the amino acid sequence of SEQ ID NO:311; V_{H} comprising the amino acid sequence of SEQ ID NO:301 and V_{L} comprising the amino acid sequence of SEQ ID NO:321; V_{H} comprising the amino acid sequence of SEQ ID NO:302 and V_{L} comprising the amino acid sequence of SEQ ID NO:322; V_{H} comprising the amino acid sequence of SEQ ID NO:303 and V_{L} comprising the amino acid sequence of SEQ ID NO:311; V_{H} comprising the amino acid sequence of SEQ ID NO:304 and V_{L} comprising the amino acid sequence of SEQ ID NO:322; V_{H} comprising the amino acid sequence of SEQ ID NO:305 and V_{L} comprising the amino acid sequence of SEQ ID NO:322; V_{H} comprising the amino acid sequence of SEQ ID NO:301 and V_{L} comprising the amino acid sequence of SEQ ID NO:323; V_{H} comprising the amino acid sequence of SEQ ID NO:301 and V_{L} comprising the amino acid sequence of SEQ ID NO:324; V_{H} comprising the amino acid sequence of SEQ ID NO:301 and V_{L} comprising the amino acid sequence of SEQ ID NO:325; V_{H} comprising the amino acid sequence of SEQ ID NO:306 and V_{L} comprising the amino acid sequence of SEQ ID NO:326; V_{H} comprising the amino acid sequence of SEQ ID NO:307 and V_{L} comprising the amino acid sequence of SEQ ID NO:327; and V_{H} comprising the amino acid sequence of SEQ ID NO:308 and V_{L} comprising the amino acid sequence of SEQ ID NO:328.

In some embodiments, an anti-MerTK antibody of the present disclosure comprises: (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO: 155; (d) HVR-L1 comprising the amino acid sequence of SEQ ID NO:383; (e) HVR-L2 comprising the amino acid sequence of SEQ ID NO:203; and (f) HVR-L3 comprising the amino acid sequence of SEQ ID NO:232. In some embodiments, an anti-MerTK antibody of the present disclosure comprises a heavy chain variable domain (V_{H}) and a light chain variable domain (V_{L}), wherein the V_{H} comprises the amino acid sequence of SED ID NO: 298 and the V_{L} comprises the amino acid sequence of SEQ ID NO:318. In some embodiments, an anti-MerTK antibody of the present disclosure comprises a heavy chain and a light chain, wherein the heavy chain comprises the amino acid sequence of SEQ ID NO: 464 and the light chain comprises the amino acid sequence of SEQ ID NO:472. In some embodiments, an anti-MerTK antibody of the present disclosure comprises a heavy chain and a light chain, wherein the heavy chain comprises the amino acid sequence of SEQ ID NO: 465 and the light chain comprises the amino acid sequence of SEQ ID NO:472. In some embodiments, an anti-MerTK antibody of the present disclosure comprises a heavy chain and a light chain, wherein the heavy chain comprises the amino acid sequence of SEQ ID NO: 466 and the light chain comprises the amino acid sequence of SEQ ID NO:472. In some embodiments, an anti-MerTK antibody of the present disclosure comprises a heavy chain and a light chain, wherein the heavy chain comprises the amino acid sequence of SEQ ID NO: 467 and the light chain comprises the amino acid sequence of SEQ ID NO:472. In some embodiments, an anti-MerTK antibody of the present disclosure comprises a heavy chain and a light chain, wherein the heavy chain comprises the amino acid sequence of SEQ ID NO: 468 and the light chain comprises the amino acid sequence of SEQ ID NO:472. In some embodiments, an anti-MerTK antibody of the present disclosure comprises a heavy chain and a light chain, wherein the heavy chain comprises the amino acid sequence of SEQ ID NO: 469 and the light chain comprises the amino acid sequence of SEQ ID NO:472.

In some embodiments, an anti-MerTK antibody of the present disclosure comprises: (a) HVR-H1 comprising the amino acid sequence of SEQ ID NO:84; (b) HVR-H2 comprising the amino acid sequence of SEQ ID NO:99; (c) HVR-H3 comprising the amino acid sequence of SEQ ID NO:139; (d) HVR-L1 comprising the amino acid sequence of SEQ ID NO:345; (e) HVR-L2 comprising the amino acid sequence of SEQ ID NO: 195; and (f) HVR-L3 comprising the amino acid sequence of SEQ ID NO:219. In some embodiments, an anti-MerTK antibody of the present disclosure comprises a heavy chain variable domain (V_{H}) and a light chain variable domain (V_{L}), wherein the V_{H} comprises the amino acid sequence of SED ID NO: 256 and the V_{L} comprises the amino acid sequence of SEQ ID NO:258. In some embodiments, an anti-MerTK antibody of the present disclosure comprises a heavy chain and a light chain, wherein the heavy chain comprises the amino acid sequence of SEQ ID NO: 422 and the light chain comprises the amino acid sequence of SEQ ID NO:430. In some embodiments, an anti-MerTK antibody of the present disclosure comprises a heavy chain and a light chain, wherein the heavy chain comprises the amino acid sequence of SEQ ID NO: 423 and the light chain comprises the amino acid sequence of SEQ ID NO:430. In some embodiments, an anti-MerTK antibody of the present disclosure comprises a heavy chain and a light chain, wherein the heavy chain comprises the amino acid sequence of SEQ ID NO: 424 and the light chain comprises the amino acid sequence of SEQ ID NO:430. In some embodiments, an anti-MerTK antibody of the present disclosure comprises a heavy chain and a light chain, wherein the heavy chain comprises the amino acid sequence of SEQ ID NO: 425 and the light chain comprises the amino acid sequence of SEQ ID NO:430. In some embodiments, an anti-MerTK antibody of the present disclosure comprises a heavy chain and a light chain, wherein the heavy chain comprises the amino acid sequence of SEQ ID NO: 426 and the light chain comprises the amino acid sequence of SEQ ID NO:430. In some embodiments, an anti-MerTK antibody of the present disclosure comprises a heavy chain and a light chain, wherein the heavy chain comprises the amino acid sequence of SEQ ID NO: 427 and the light chain comprises the amino acid sequence of SEQ ID NO:430.

In some embodiments, an anti-MerTK antibody of the present disclosure comprises: (a) HVR-H1 comprising the amino acid sequence of SEQ ID NO:90; (b) HVR-H2 comprising the amino acid sequence of SEQ ID NO:368; (c) HVR-H3 comprising the amino acid sequence of SEQ ID NO: 155; (d) HVR-L1 comprising the amino acid sequence of SEQ ID NO:376; (e) HVR-L2 comprising the amino acid sequence of SEQ ID NO:203; and (f) HVR-L3 comprising the amino acid sequence of SEQ ID NO:393. In some embodiments, an anti-MerTK antibody of the present disclosure comprises a heavy chain variable domain (V_{H}) and a light chain variable domain (V_{L}), wherein the V_{H} comprises the amino acid sequence of SED ID NO: 299 and the V_{L} comprises the amino acid sequence of SEQ ID NO:320. In some embodiments, an anti-MerTK antibody of the present disclosure comprises a heavy chain and a light chain, wherein the heavy chain comprises the amino acid sequence of SEQ ID NO: 440 and the light chain comprises the amino acid sequence of SEQ ID NO:448. In some embodiments, an anti-MerTK antibody of the present disclosure comprises a heavy chain and a light chain, wherein the heavy chain comprises the amino acid sequence of SEQ ID NO: 441 and the light chain comprises the amino acid sequence of SEQ ID NO:448. In some embodiments, an anti-MerTK antibody of the present disclosure comprises a heavy chain and a light chain, wherein the heavy chain comprises the amino acid sequence of SEQ ID NO: 442 and the light chain comprises the amino acid sequence of SEQ ID NO:448. In some embodiments, an anti-MerTK antibody of the present disclosure comprises a heavy chain and a light chain, wherein the heavy chain comprises the amino acid sequence of SEQ ID NO: 443 and the light chain comprises the amino acid sequence of SEQ ID NO:448. In some embodiments, an anti-MerTK antibody of the present disclosure comprises a heavy chain and a light chain, wherein the heavy chain comprises the amino acid sequence of SEQ ID NO: 444 and the light chain comprises the amino acid sequence of SEQ ID NO:448. In some embodiments, an anti-MerTK antibody of the present disclosure comprises a heavy chain and a light chain, wherein the heavy chain comprises the amino acid sequence of SEQ ID NO: 445 and the light chain comprises the amino acid sequence of SEQ ID NO:448.

In some embodiments, an anti-MerTK antibody of the present disclosure comprises: (a) HVR-H1 comprising the amino acid sequence of SEQ ID NO:84; (b) HVR-H2 comprising the amino acid sequence of SEQ ID NO:329; (c) HVR-H3 comprising the amino acid sequence of SEQ ID NO: 139; (d) HVR-L1 comprising the amino acid sequence of SEQ ID NO: 169; (e) HVR-L2 comprising the amino acid sequence of SEQ ID NO: 195; and (f) HVR-L3 comprising the amino acid sequence of SEQ ID NO:219. In some embodiments, an anti-MerTK antibody of the present disclosure comprises a heavy chain variable domain (V_{H}) and a light chain variable domain (V_{L}), wherein the V_{H} comprises the amino acid sequence of SED ID NO: 225 and the V_{L} comprises the amino acid sequence of SEQ ID NO:257. In some embodiments, an anti-MerTK antibody of the present disclosure comprises a heavy chain and a light chain, wherein the heavy chain comprises the amino acid sequence of SEQ ID NO: 413 and the light chain comprises the amino acid sequence of SEQ ID NO:421. In some embodiments, an anti-MerTK antibody of the present disclosure comprises a heavy chain and a light chain, wherein the heavy chain comprises the amino acid sequence of SEQ ID NO: 414 and the light chain comprises the amino acid sequence of SEQ ID NO:421. In some embodiments, an anti-MerTK antibody of the present disclosure comprises a heavy chain and a light chain, wherein the heavy chain comprises the amino acid sequence of SEQ ID NO: 415 and the light chain comprises the amino acid sequence of SEQ ID NO:421. In some embodiments, an anti-MerTK antibody of the present disclosure comprises a heavy chain and a light chain, wherein the heavy chain comprises the amino acid sequence of SEQ ID NO: 416 and the light chain comprises the amino acid sequence of SEQ ID NO:421. In some embodiments, an anti-MerTK antibody of the present disclosure comprises a heavy chain and a light chain, wherein the heavy chain comprises the amino acid sequence of SEQ ID NO: 417 and the light chain comprises the amino acid sequence of SEQ ID NO:421. In some embodiments, an anti-MerTK antibody of the present disclosure comprises a heavy chain and a light chain, wherein the heavy chain comprises the amino acid sequence of SEQ ID NO: 418 and the light chain comprises the amino acid sequence of SEQ ID NO:421.

In some embodiments, an anti-MerTK antibody of the present disclosure binds to the extracellular domain (ECD) of human MerTK. In some embodiments, an anti-MerTK antibody of the present disclosure binds to the N-terminal domain of human MerTK. In some embodiments, an anti-MerTK antibody of the present disclosure binds to one or more amino acids within the amino acid sequence of SEQ ID NO:449. In some embodiments, an anti-MerTK antibody of the present disclosure binds to the immunoglobulin-like domain 1 (Ig1) of human MerTK. In some embodiments, an anti-MerTK antibody of the present disclosure binds to one or more amino acid residues within the amino acid sequence of SED ID NO:450. In some embodiments, an anti-MerTK antibody of the present disclosure binds to the immunoglobulin-like domain 2 (Ig2) of human MerTK. In some embodiments, an anti-MerTK antibody of the present disclosure binds to one or more amino acid residues within the amino acid sequence of SEQ ID NO:451. In some embodiments, an anti-MerTK antibody of the present disclosure binds to the fibronectin type III domain 1 (FN1) of human MerTK. In some embodiments, an anti-MerTK antibody of the present isclosure binds to one or more amino acid residues within the amino acid sequence of SEQ ID NO: 452. In some embodiments, an anti-MerTK antibody of the present disclosure binds to the juxta membrane domain (JM) of human MerTK. In some embodiments, an anti-MerTK antibody of the present disclosure binds to one or more amino acid residues within the amino acid sequence of SEQ ID NO:454. In some embodiments, an anti-MerTK antibody of the present disclosure binds to the extracellular domain of human MerTK protein but does not bind to the extracellular domain of human Axl protein.

In some embodiments, an anti-MerTK antibody of the present disclosure binds to one or more domains within the extracellular domain of human MerTK. In some embodiments, an anti-MerTK antibod of the present disclosure binds to the immunoglobulin-like 1 domain 1 (Ig1) and the fibronectin type III domain 1 (FN1) of human MerTK. In some embodiments, an anti-MerTK antibody of the present disclosure binds to the immunoglobulin-like domain 2 (Ig2) and the fibronectin type III domain 1 (FN1) of human MerTK.

In some embodiments, the anti-MerTK antibody according to any of the above embodiments is a monoclonal antibody, including a humanized and/or human antibody. In some embodiments, the anti-MerTK antibody is an antibody fragment, e.g., a Fv, Fab, Fab′, scFv, diabody, or F(ab′)2 fragment. In some embodiments, the anti-MerTK antibody is a substantially full-length antibody, e.g., an IgG1 antibody, IgG2a antibody or other antibody class or isotype as defined herein.

In some embodiments, an anti-MerTK antibody according to any of the above embodiments may incorporate any of the features, singly or in combination, as described in Sections 1-7 below:

### (1) Anti-MerTK Antibody Binding Affinity

In some embodiments of any of the antibodies provided herein, the antibody has a dissociation constant (K_{D}) of <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M). Dissociation constants may be determined through any analytical technique, including any biochemical or biophysical technique such as ELISA, surface plasmon resonance (SPR), bio-layer interferometry (see, e.g., Octet System by ForteBio), isothermal titration calorimetry (ITC), differential scanning calorimetry (DSC), circular dichroism (CD), stopped-flow analysis, and colorimetric or fluorescent protein melting analyses. In one embodiment, Kd is measured by a radiolabeled antigen binding assay (RIA). In some embodiment, an RIA is performed with the Fab version of an antibody of interest and its antigen, for example as described in Chen et al. J. Mol. Biol. 293:865-881(1999)). In some embodiments, K_{D} is measured using a BIACORE surface plasmon resonance assay, for example, an assay using a BIACORE-2000 or a BIACORE-3000 (BIAcore, Inc., Piscataway, NJ) is performed at 25° C. with immobilized antigen CM5 chips at ˜10 response units (RU). In some embodiments, the K_{D} is determined using a monovalent antibody (e.g., a Fab) or a full-length antibody. In some embodiments, the K_{D} is determined using a full-length antibody in a monovalent form.

In some embodiments, an anti-MerTK antibody of the present disclosure binds to human MerTK, wherein the K_{D} of binding to human MerTK is from about 1.4 nM to about 81 nM. In some embodiments, an anti-MerTK antibody binds to cyno MerTK, wherein the K_{D} of binding to cyno MerTK is from about 1.6 nM to about 107 nM. In some embodiments, an anti-MerTK antibody of the present disclosure binds to murine MerTK, wherein the K_{D} of binding to murine MerTK is from about 30 nM to about 186 nM.

### (2) Antibody Fragments

In some embodiments of any of the antibodies provided herein, the antibody is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP404097; WO 1993/01161; Hudson et al. Nat. Med. 9:129-134 (2003). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### (3) Chimeric and Humanized Antibodies

In some embodiments of any of the antibodies provided herein, the antibody is a chimeric antibody. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments of any of the antibodies provided herein, the antibody is a humanized antibody. Typically, a non-human antibody is humanized to reduce immunogenicity to humans, while retaining the specificity and affinity of the parental non-human antibody. In certain embodiments, a humanized antibody is substantially non-immunogenic in humans. In certain embodiments, a humanized antibody has substantially the same affinity for a target as an antibody from another species from which the humanized antibody is derived. See, e.g., U.S. Pat. Nos. 5,530,101, 5,693,761; 5,693,762; and 5,585,089. In certain embodiments, amino acids of an antibody variable domain that can be modified without diminishing the native affinity of the antigen binding domain while reducing its immunogenicity are identified. See, e.g., U.S. Pat. Nos. 5,766,886 and 5,869,619. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:161 9-1633 (2008), and are further described, e.g., in U.S. Pat. Nos. 5,821,337, 7,527,791, 6,982,321, and 7,087,409. Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA 89:4285 (1992); and Presta et al., J. Immunol. 151:2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al. J. Biol. Chem. 272:10678-10684 (1997) and Rosok et al. J Biol. Chem. 271:22611-22618 (1996)).

### (4) Human Antibodies

In some embodiments of any of the antibodies provided herein, the antibody is a human antibody. Human antibodies can be produced using various techniques known in the art. Human antibodies are described generally in van Dijk et al. Curr. Opin. Pharmacol. 5:368-74 (2001) and Lonberg Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human MAbs with the desired specificity can be produced and selected. Certain exemplary methods are described in U.S. Pat. No. 5,545,807, EP 546073, and EP 546073. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J Immunol. 133:3001 (1984) and Boerner et al. J Immunol. 147:86 (1991)). Human antibodies generated via human B-cell hybridoma technology are also described in Li et al. Proc. Natl. Acad. Sci. USA, 1 03:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al. Histology and Histopathology 20(3):927-937 (2005) and Vollmers et al. Methods and Findings in Experimental and Clinical Pharmacology 27(3):185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

In some embodiments of any of the antibodies provided herein, the antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed) ribosome display (CAT), yeast display (Adimab), and the like. In certain phage display methods, repertoires of V_{H} and V_{L} genes are separately cloned by polymerase chain reaction (PCR) and recombined randomly in phage libraries, which can then be screened for antigen-binding phage as described in Winter et al. Ann. Rev. Immunol. 12: 433-455 (1994). For example, a variety of methods are known in the art for generating phage display libraries and screening such libraries for antibodies possessing the desired binding characteristics. See also Sidhu et al. J. Mol. Biol. 338(2): 299-310, 2004; Lee et al. J. Mol. Biol. 340(5): 1073-1093, 2004; Fellouse Proc. Natl. Acad. Sci. USA 101(34):12467-12472 (2004); and Lee et al. J. Immunol. Methods 284(−2):1 19-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. EMIBO J. 12: 725-734 (1993). Finally, naive libraries can also be made synthetically by cloning unrearranged V-gene segments from stem cells, and using PCR primers comprising random sequence to encode the highly variable HVR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and US Patent Publication Nos. 2007/0292936 and 2009/0002360. Antibodies isolated from human antibody libraries are considered human antibodies or human antibody fragments herein.

### (5) Constant Regions Including Fc Regions

In some embodiments of any of the antibodies provided herein, the antibody comprises an Fc. In some embodiments, the Fc is a human IgG1, IgG2, IgG3, and/or IgG4 isotype. In some embodiments, the antibody is of the IgG class, the IgM class, or the IgA class.

In certain embodiments of any of the antibodies provided herein, the antibody has an IgG2 isotype. In some embodiments, the antibody contains a human IgG2 constant region. In some embodiments, the human IgG2 constant region includes an Fc region. In some embodiments, the antibody induces the one or more MerTK activities or independently of binding to an Fc receptor. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG4 isotype. In some embodiments, the antibody contains a human IgG4 constant region. In some embodiments, the human IgG4 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has a hybrid IgG2/4 isotype. In some embodiments, the antibody includes an amino acid sequence comprising amino acids 118 to 260 according to EU numbering of human IgG2 and amino acids 261-447 according to EU numbering of human IgG4 (WO 1997/11971; WO 2007/106585).

In some embodiments, the Fc region increases clustering without activating complement as compared to a corresponding antibody comprising an Fc region that does not comprise the amino acid substitutions. In some embodiments, the antibody induces one or more activities of a target specifically bound by the antibody. In some embodiments, the antibody binds to MerTK.

It may also be desirable to modify an anti-MerTK antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRI, and/or FcγRIII to reduce Antibody-dependent cell-mediated cytotoxicity. In some embodiments, the effector function is impaired by removing N-glycosylation of the Fc region (e.g., in the CH2 domain of IgG) of the antibody. In some embodiments, the effector function is impaired by modifying regions such as 233-236, 297, and/or 327-331 of human IgG as described in WO 99/58572 and Armour et al. Molecular Immunology 40: 585-593 (2003); Reddy et al. J. Immunology 164:1925-1933 (2000). In other embodiments, it may also be desirable to modify an anti-MerTK antibody of the present disclosure to modify effector function to increase finding selectivity toward the ITIM-containing FcgRIIb (CD32b) to increase clustering of MerTK antibodies on adjacent cells without activating humoral responses including Antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Other amino acid sequence modifications.

### (6) Antibody Variants

In some embodiments of any of the antibodies provided herein, amino acid sequence variants of the antibodies are contemplated. For example, it may be desirable to improve the binding affinity and/or other biological properties of the antibody.

### (i) Substitution, Insertion, and Deletion Variants

In some embodiments of any of the antibodies provided herein, antibody variants having one or more amino acid substitutions are provided. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody.

TABLE A
Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the polypeptide or antibody described herein, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al. J Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within +2 is included. In certain embodiments, those which are within ±1 are included, and in certain embodiments, those within +0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within +2 is included, in certain embodiments, those which are within ±1 are included, and in certain embodiments, those within +0.5 are included. One can also identify epitopes from primary amino acid sequences on the basis of hydrophilicity. These regions are also referred to as “epitopic core regions”.

In certain embodiments of the variant V_{H} and V_{L} sequences provided above, each HVR is unaltered.

Amino acid sequence insertions include amino- and/or carboxyl-terminal fusions ranging in length from one residue to polypeptides comprising a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Any cysteine residue outside the HVRs and not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

### (ii) Glycosylation Variants

In some embodiments of any of the antibodies provided herein, the antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered.

Native antibodies produced by mammalian cells typically comprise a branched, biantennary oligosaccharide that is generally attached by an N-linkage to Asn297 according to Kabat numbering of the CH2 domain of the Fc region. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the disclosure may be made in order to create antibody variants with certain improved properties.

In one embodiment, antibody variants are provided having a carbohydrate structure that lacks fucose attached (directly or indirectly) to an Fc region. See, e.g., US Patent Publication Nos. 2003/0157108 and 2004/0093621. Examples of publications related to “defucosylated” or “fucose-deficient” antibody variants include: US 2003/0157108; US 2003/0115614; US 2002/0164328; US 2004/0093621; US 2004/0132140; US 2004/0110704; US 2004/0110282; US 2004/0109865; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Led 3 CHO cells deficient in protein fucosylation (Ripka et al. Arch. Biochem. Biophys. 249:533-545 (1986); US 2003/0157108), and knockout cell lines, such as alpha-1,6-fucosyltransferase gene, FUT8, knockout CHO cells (see, e.g., Yamane-Ohnuki et al. Biotech. Bioeng. 87: 614 (2004) and Kanda et al. Biotechnol. Bioeng. 94(4):680-688 (2006)).

### (iii) Modified Constant Regions

In some embodiments of any of the antibodies provided herein, the antibody Fc is an antibody Fc isotypes and/or modifications. In some embodiments, the antibody Fc isotype and/or modification is capable of binding to Fc gamma receptor.

In some embodiments of any of the antibodies provided herein, the modified antibody Fc is an IgG1 modified Fc. In some embodiments, the IgG1 modified Fc comprises one or more modifications.

For example, in some embodiments, the IgG1 modified Fc comprises one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) CellImmunol, 200:16-26), G237A (Alegre et al. (1994) Transplantation 57:1537-1543. 31; Xu et al. (2000) CellImmunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, where the amino acid position is according to the EU numbering convention.

In some embodiments of any of the IgG1 modified Fc, the Fc comprises N297A mutation according to EU numbering. In some embodiments of any of the IgG1 modified Fc, the Fc comprises D265A and N297A mutations according to EU numbering. In some embodiments of any of the IgG1 modified Fc, the Fc comprises D270A mutations according to EU numbering. In some embodiments, the IgG1 modified Fc comprises L234A and L235A mutations according to EU numbering. In some embodiments of any of the IgG1 modified Fc, the Fc comprises L234A and G237A mutations according to EU numbering. In some embodiments of any of the IgG1 modified Fc, the Fc comprises L234A, L235A and G237A mutations according to EU numbering. In some embodiments of any of the IgG1 modified Fc, the Fc comprises one or more (including all) of P238D, L328E, E233, G237D, H268D, P271G and A330R mutations according to EU numbering. In some embodiments of any of the IgG1 modified Fc, the Fc comprises one or more of S267E/L328F mutations according to EU numbering. In some embodiments of any of the IgG1 modified Fc, the Fc comprises P238D, L328E, E233D, G237D, H268D, P271G and A330R mutations according to EU numbering. In some embodiments of any of the IgG1 modified Fc, the Fc comprises P238D, L328E, G237D, H268D, P271G and A330R mutations according to EU numbering. In some embodiments of any of the IgG1 modified Fc, the Fc comprises P238D, S267E, L328E, E233D, G237D, H268D, P271G and A330R mutations according to EU numbering. In some embodiments of any of the IgG1 modified Fc, the Fc comprises P238D, S267E, L328E, G237D, H268D, P271G and A330R mutations according to EU numbering. In some embodiments of any of the IgG1 modified Fc, the Fc comprises C226S, C229S, E233P, L234V, and L235A mutations according to EU numbering. In some embodiments of any of the IgG1 modified Fc, the Fc comprises L234F, L235E, and P331S mutations according to EU numbering. In some embodiments of any of the IgG1 modified Fc, the Fc comprises S267E and L328F mutations according to EU numbering. In some embodiments of any of the IgG1 modified Fc, the Fc comprises N325S and L328F mutations according to EU numbering. In some embodiments of any of the IgG1 modified Fc, the Fc comprises S267E mutations according to EU numbering. In some embodiments of any of the IgG1 modified Fc, the Fc comprises a substitute of the constant heavy 1 (CH1) and hinge region of IgG1 with CH1 and hinge region of IgG2 (amino acids 118-230 of IgG2 according to EU numbering) with a Kappa light chain.

In some embodiments of any of the IgG1 modified Fc, the Fc includes two or more amino acid substitutions that increase antibody clustering without activating complement as compared to a corresponding antibody having an Fc region that does not include the two or more amino acid substitutions. Accordingly, in some embodiments of any of the IgG1 modified Fc, the IgG1 modified Fc is an antibody comprising an Fc region, where the antibody comprises an amino acid substitution at position E430G and one or more amino acid substitutions in the Fc region at a residue position selected from: L234F, L235A, L235E, S267E, K322A, L328F, A330S, P331S, and any combination thereof according to EU numbering. In some embodiments, the IgG1 modified Fc comprises an amino acid substitution at positions E430G, L243A, L235A, and P331S according to EU numbering. In some embodiments, the IgG1 modified Fc comprises an amino acid substitution at positions E430G and P331S according to EU numbering. In some embodiments, the IgG1 modified Fc comprises an amino acid substitution at positions E430G and K322A according to EU numbering. In some embodiments, the IgG1 modified Fe comprises an amino acid substitution at positions E430G, A330S, and P331S according to EU numbering. In some embodiments, the IgG1 modified Fc comprises an amino acid substitution at positions E430G, K322A, A330S, and P331S according to EU numbering. In some embodiments, the IgG1 modified Fc comprises an amino acid substitution at positions E430G, K322A, and A330S according to EU numbering. In some embodiments, the IgG1 modified Fc comprises an amino acid substitution at positions E430G, K322A, and P331S according to EU numbering.

In some embodiments of any of the IgG1 modified Fc, the IgG1 modified Fc may further comprise herein may be combined with an A330L mutation (Lazar et al. Proc Natl Acad Sci USA, 103:4005-4010 (2006)), or one or more of L234F, L235E, and/or P331S mutations (Sazinsky et al. Proc Natl Acad Sci USA, 105:20167-20172 (2008)), according to the EU numbering convention, to eliminate complement activation. In some embodiments of any of the IgG1 modified Fc, the IgG1 modified Fc may further comprise one or more of A330L, A330S, L234F, L235E, and/or P331S according to EU numbering. In some embodiments of any of the IgG1 modified Fc, the IgG1 modified Fc may further comprise one or more mutations to enhance the antibody half-life in human serum (e.g., one or more (including all) of M252Y, S254T, and T256E mutations according to the EU numbering convention). In some embodiments of any of the IgG1 modified Fc, the IgG1 modified Fc may further comprise one or more of E430G, E430S, E430F, E430T, E345K, E345Q, E345R, E345Y, S440Y, and/or S440W according to EU numbering.

Other aspects of the present disclosure relate to antibodies having modified constant regions (i.e., Fc regions). An antibody dependent on binding to FcgR receptor to activate targeted receptors may lose its agonist activity if engineered to eliminate FcgR binding (see, e.g., Wilson et al. Cancer Cell 19:101-113 (2011); Armour at al. Immunology 40:585-593 (2003); and White et al. Cancer Cell 27:138-148 (2015)). As such, it is thought that an anti-MerTK antibody of the present disclosure with the correct epitope specificity can activate the target antigen, with minimal adverse effects, when the antibody has an Fc domain from a human IgG2 isotype (CH1 and hinge region) or another type of Fc domain that is capable of preferentially binding the inhibitory FcgRIIB r receptors, or a variation thereof.

In some embodiments of any of the antibodies provided herein, the modified antibody Fc is an IgG2 modified Fc. In some embodiments, the IgG2 modified Fc comprises one or more modifications. For example, in some embodiments, the IgG2 modified Fc comprises one or more amino acid substitutions (e.g., relative to a wild-type Fc region of the same isotype). In some embodiments of any of the IgG2 modified Fc, the one or more amino acid substitutions are selected from V234A (Alegre et al. Transplantation 57:1537-1543 (1994); Xu et al. Cell Immunol, 200:16-26 (2000)); G237A (Cole et al. Transplantation, 68:563-571 (1999)); H268Q, V309L, A330S, P331S (US 2007/0148167; Armour et al. Eur J Immunol 29: 2613-2624 (1999); Armour et al. The Haematology Journal 1(Suppl. 1):27 (2000); Armour et al. The Haematology Journal 1(Suppl. 1):27 (2000)), C219S, and/or C220S (White et al. Cancer Cell 27, 138-148 (2015)); S267E, L328F (Chu et al. Mol Immunol, 45:3926-3933 (2008)); and M252Y, S254T, and/or T256E according to the EU numbering convention. In some embodiments of any of the IgG2 modified Fe, the Fc comprises an amino acid substitution at positions V234A and G237A according to EU numbering. In some embodiments of any of the IgG2 modified Fc, the Fc comprises an amino acid substitution at positions C219S or C220S according to EU numbering. In some embodiments of any of the IgG2 modified Fc, the Fc comprises an amino acid substitution at positions A330S and P331S according to EU numbering. In some embodiments of any of the IgG2 modified Fc, the Fc comprises an amino acid substitution at positions S267E and L328F according to EU numbering.

In some embodiments of any of the IgG2 modified Fc, the Fc comprises a C127S amino acid substitution according to the EU numbering convention (White et al., (2015) Cancer Cell 27, 138-148; Lightle et al. Protein Sci. 19:753-762 (2010); and WO 2008/079246). In some embodiments of any of the IgG2 modified Fc, the antibody has an IgG2 isotype with a Kappa light chain constant domain that comprises a C214S amino acid substitution according to the EU numbering convention (White et al. Cancer Cell 27:138-148 (2015); Lightle et al. Protein Sci. 19:753-762 (2010); and WO 2008/079246).

In some embodiments of any of the IgG2 modified Fc, the Fc comprises a C220S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc comprises a C219S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc includes an IgG2 isotype heavy chain constant domain 1(CH1) and hinge region (White et al. Cancer Cell 27:138-148 (2015)). In certain embodiments of any of the IgG2 modified Fc, the IgG2 isotype CH1 and hinge region comprise the amino acid sequence of 118-230 according to EU numbering. In some embodiments of any of the IgG2 modified Fc, the antibody Fc region comprises a S267E amino acid substitution, a L328F amino acid substitution, or both, and/or a N297A or N297Q amino acid substitution according to the EU numbering convention.

In some embodiments of any of the IgG2 modified Fc, the Fc further comprises one or more amino acid substitution at positions E430G, E430S, E430F, E430T, E345K, E345Q, E345R, E345Y, S440Y, and S440W according to EU numbering. In some embodiments of any of the IgG2 modified Fc, the Fc may further comprise one or more mutations to enhance the antibody half-life in human serum (e.g., one or more (including all) of M252Y, S254T, and T256E mutations according to the EU numbering convention). In some embodiments of any of the IgG2 modified Fc, the Fc may further comprise A330S and P331S.

In some embodiments of any of the IgG2 modified Fc, the Fc is an IgG2/4 hybrid Fc. In some embodiments, the IgG2/4 hybrid Fc comprises IgG2 aa 118 to 260 and IgG4 aa 261 to 447. In some embodiments of any IgG2 modified Fe, the Fe comprises one or more amino acid substitutions at positions H268Q, V309L, A330S, and P331S according to EU numbering.

In some embodiments of any of the IgG1 and/or IgG2 modified Fc, the Fc comprises one or more additional amino acid substitutions selected from A330L, L234F; L235E, or P331S according to EU numbering; and any combination thereof.

In certain embodiments of any of the IgG1 and/or IgG2 modified Fc, the Fc comprises one or more amino acid substitutions at a residue position selected from C127S, L234A, L234F, L235A, L235E, S267E, K322A, L328F, A330S, P331S, E345R, E430G, S440Y, and any combination thereof according to EU numbering. In some embodiments of any of the IgG1 and/or IgG2 modified Fc, the Fc comprises an amino acid substitution at positions E430G, L243A, L235A, and P331S according to EU numbering. In some embodiments of any of the IgG1 and/or IgG2 modified Fc, the Fc comprises an amino acid substitution at positions E430G and P331S according to EU numbering. In some embodiments of any of the IgG1 and/or IgG2 modified Fc, the Fc comprises an amino acid substitution at positions E430G and K322A according to EU numbering. In some embodiments of any of the IgG1 and/or IgG2 modified Fc, the Fc comprises an amino acid substitution at positions E430G, A330S, and P331S according to EU numbering. In some embodiments of any of the IgG1 and/or IgG2 modified Fc, the Fc comprises an amino acid substitution at positions E430G, K322A, A330S, and P331S according to EU numbering. In some embodiments of any of the IgG1 and/or IgG2 modified Fc, the Fc comprises an amino acid substitution at positions E430G, K322A, and A330S according to EU numbering. In some embodiments of any of the IgG1 and/or IgG2 modified Fc, the Fc comprises an amino acid substitution at positions E430G, K322A, and P331S according to EU numbering. In some embodiments of any of the IgG1 and/or IgG2 modified Fc, the Fc comprises an amino acid substitution at positions S267E and L328F according to EU numbering. In some embodiments of any of the IgG1 and/or IgG2 modified Fc, the Fc comprises an amino acid substitution at position C127S according to EU numbering. In some embodiments of any of the IgG1 and/or IgG2 modified Fc, the Fc comprises an amino acid substitution at positions E345R, E430G and S440Y according to EU numbering.

In some embodiments of any of the antibodies provided herein, the modified antibody Fc is an IgG4 modified Fc. In some embodiments, the IgG4 modified Fc comprises one or more modifications. For example, in some embodiments, the IgG4 modified Fc comprises one or more amino acid substitutions (e.g., relative to a wild-type Fc region of the same isotype). In some embodiments of any of the IgG4 modified Fc, the one or more amino acid substitutions are selected from L235A, G237A, S229P, L236E (Reddy et al. J Immunol 164:1925-1933(2000)), S267E, E318A, L328F, M252Y, S254T, and/or T256E according to the EU numbering convention. In some embodiments of any of the IgG4 modified Fc, the Fc may further comprise L235A, G237A, and E318A according to the EU numbering convention. In some embodiments of any of the IgG4 modified Fc, the Fc may further comprise S228P and L235E according to the EU numbering convention. In some embodiments of any of the IgG4 modified Fc, the IgG4 modified Fc may further comprise S267E and L328F according to the EU numbering convention.

In some embodiments of any of the IgG4 modified Fc, the IgG4 modified Fc comprises may be combined with an S228P mutation according to the EU numbering convention (Angal et al. Mol Immunol. 30:105-108 (1993)) and/or with one or more mutations described in (Peters et al. J Biol Chem. 287(29):24525-33 (2012)) to enhance antibody stabilization.

In some embodiments of any of the IgG4 modified Fc, the IgG4 modified Fc may further comprise one or more mutations to enhance the antibody half-life in human serum (e.g., one or more (including all) of M252Y, S254T, and T256E mutations according to the EU numbering convention).

In some embodiments of any of the IgG4 modified Fc, the Fc comprises L235E according to EU numbering. In certain embodiments of any of the IgG4 modified Fc, the Fc comprises one or more amino acid substitutions at a residue position selected from C127S, F234A, L235A, L235E, S267E, K322A, L328F, E345R, E430G, S440Y, and any combination thereof, according to EU numbering. In some embodiments of any of the IgG4 modified Fc, the Fc comprises an amino acid substitution at positions E430G, L243A, L235A, and P331S according to EU numbering. In some embodiments of any of the IgG4 modified Fc, the Fc comprises an amino acid substitution at positions E430G and P331S according to EU numbering. In some embodiments of any of the IgG4 modified Fc, the Fc comprises an amino acid substitution at positions E430G and K322A according to EU numbering. In some embodiments of any of the IgG4 modified Fc, the Fc comprises an amino acid substitution at position E430 according to EU numbering. In some embodiments of any of the IgG4 modified Fc, the Fc region comprises an amino acid substitution at positions E430G and K322A according to EU numbering. In some embodiments of any of the IgG4 modified Fc, the Fc comprises an amino acid substitution at positions S267E and L328F according to EU numbering. In some embodiments of any of the IgG4 modified Fc, the Fc comprises an amino acid substitution at position C127S according to EU numbering. In some embodiments of any of the IgG4 modified Fc, the Fc comprises an amino acid substitution at positions E345R, E430G and S440Y according to EU numbering.

### (7) Other Antibody Modifications

In some embodiments of any of the antibodies, the antibody is a derivative. The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen binding protein can have a greater circulating half-life than an antigen binding protein that is not chemically modified. In certain embodiments, a chemically modified antigen binding protein can have improved targeting capacity for desired cells, tissues, and/or organs. In some embodiments, a derivative antigen binding protein is covalently modified to include one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative antigen binding protein comprises one or more polymer, including, but not limited to, monomethoxy-polyethylene glycol, dextran, cellulose, copolymers of ethylene glycol/propylene glycol, carboxymethylcellulose, polyvinyl pyrrolidone, poly-1, 3-dioxolane, poly-1,3,6-trioxane, ethylene/maleic anhydride copolymer, polyaminoacids (either homopolymers or random copolymers), poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers.

In certain embodiments, a derivative is covalently modified with polyethylene glycol (PEG) subunits. In certain embodiments, one or more water-soluble polymer is bonded at one or more specific position, for example at the amino terminus, of a derivative. In certain embodiments, one or more water-soluble polymer is randomly attached to one or more side chains of a derivative. In certain embodiments, PEG is used to improve the therapeutic capacity for an antigen binding protein. In certain embodiments, PEG is used to improve the therapeutic capacity for a humanized antibody. Certain such methods are discussed, for example, in U.S. Pat. No. 6,133,426, which is hereby incorporated by reference for any purpose.

Peptide analogs are commonly used in the pharmaceutical industry as non-peptide drugs with properties analogous to those of the template peptide. These types of non-peptide compound are termed “peptide mimetics” or “peptidomimetics.” Fauchere, J. Adv. Drug Res., 15:29 (1986); and Evans et al. J. Med. Chem., 30:1229 (1987), which are incorporated herein by reference for any purpose. Such compounds are often developed with the aid of computerized molecular modeling. Peptide mimetics that are structurally similar to therapeutically useful peptides can be used to produce a similar therapeutic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) can be used in certain embodiments to generate more stable peptides. In addition, constrained peptides comprising a consensus sequence or a substantially identical consensus sequence variation can be generated by methods known in the art (Rizo and Gierasch Ann. Rev. Biochem., 61:387 (1992), incorporated herein by reference for any purpose); for example, by adding internal cysteine residues capable of forming intramolecular disulfide bridges which cyclize the peptide.

Drug conjugation involves coupling of a biological active cytotoxic (anticancer) payload or drug to an antibody that specifically targets a certain tumor marker (e.g. a polypeptide that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue OncLive Jul. 5, 2012; ADC Review on antibody-drug conjugates; and Ducry et al. Bioconjugate Chemistry 21 (1):5-13 (2010).

### II. Nucleic Acids, Vectors, and Host Cells

Anti-MerTK antibodies of the present disclosure may be produced using recombinant methods and compositions, e.g., as described in U.S. Pat. No. 4,816,567. In some embodiments, isolated nucleic acids having a nucleotide sequence encoding any of the anti-MerTK antibodies of the present disclosure are provided. Such nucleic acids may encode an amino acid sequence comprising the V_{L} and/or an amino acid sequence comprising the V_{H} of the anti-MerTK antibody (e.g., the light and/or heavy chains of the antibody). In some embodiments, one or more vectors (e.g., expression vectors) comprising such nucleic acids are provided. In some embodiments, a host cell comprising such nucleic acid is also provided. In some embodiments, the host cell comprises (e.g., has been transduced with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody. In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-MerTK antibody of the present disclosure are provided. In some embodiments, the method includes culturing a host cell of the present disclosure comprising a nucleic acid encoding the anti-MerTK antibody, under conditions suitable for expression of the antibody. In some embodiments, the antibody is subsequently recovered from the host cell (or host cell culture medium).

For recombinant production of an anti-MerTK antibody of the present disclosure, a nucleic acid encoding the anti-MerTK antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors comprising a nucleic acid sequence encoding any of the anti-MerTK antibodies of the present disclosure, or cell-surface expressed fragments or polypeptides thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones comprising the vector. Suitable examples include plasmids and bacterial viruses, e.g., pUC18, pUC19, Bluescript (e.g., pBS SK+) and its derivatives, mpl8, mpl9, pBR322, pMB9, ColEl, pCR1, RP4, phage DNAs, and shuttle vectors such as pSA3 and pAT28. These and many other cloning vectors are available from commercial vendors such as BioRad, Strategene, and Invitrogen.

Suitable host cells for cloning or expression of antibody-encoding vectors include prokaryotic or eukaryotic cells. For example, anti-MerTK antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523. After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gemgross Nat. Biotech. 22:1409-1414 (2004); and Li et al. Nat. Biotech. 24:210⁻²¹⁵ (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodopterafrugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al. J Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al. Annals N. Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al. Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### II. Pharmaceutical Compositions/Formulations

Provided herein are pharmaceutical compositions and/or pharmaceutical formulations comprising the anti-MerTK antibodies of the present disclosure and a pharmaceutically acceptable carrier.

In some embodiments, pharmaceutically acceptable carrier preferably are nontoxic to recipients at the dosages and concentrations employed. The pharmaceutical compositions and/or pharmaceutical formulations to be used for in vivo administration can be sterile. This is readily accomplished by filtration through, e.g., sterile filtration membranes

Pharmaceutical compositions and/or pharmaceutical formulations provided herein are useful as a medicament, e.g., for treating cancer.

### IV. Therapeutic Uses

As disclosed herein, anti-MerTK antibodies of the present disclosure may be used for treating diseases and disorders. In some embodiments, the present disclosure provides methods for treating an individual having cancer comprising administering to the individual a therapeutically effective amount of an anti-MerTK antibody of the present disclosure.

Ectopic or expression of MerTK has been observed in various tumors; overexpression and activation of MerTK has been implicated in lymphoid leukemia, lymphoma, adenoma, melanoma, gastric, prostate, and breast cancers; and MerTK overexpression has been associated with metastasis. (Schlegel et al, 2013, J Clin Invest, 123:2257-2267; Tworkoski et al, 2013, Pigment Cell Melanoma, 26:527-541; Yi et al, 2017, Oncotarget, 8:96656-96667; Linger et al, 2013, Blood, 122:1599-1609; Lee-Sherick et al, 2013, Oncogene, 32:5359-5368; Brandao et al, 2013, Blood Cancer, 3:e101; Xie et al, 2015, Oncotarget, 6:9206-9219; Shi et al, 2018, J Hematology & Oncology, 11:43). Accordingly, modulating the activity of MerTK with an anti-MerTK antibody of the present disclosure is an effective means of treating cancer.

In certain aspects, provided herein are methods for treating cancer in a subject in need thereof, the method comprising administering to the subject an anti-MerTK antibody of the present disclosure, or a pharmaceutical composition comprising an anti-MerTK antibody of the present disclosure. In some embodiments, a method is provided for treating cancer in a subject in need thereof, the method comprising administering to the subject an anti-MerTK antibody of the present disclosure, wherein the anti-MerTK antibody reduces efferocytosis by phagocytic cells. In some embodiments, a method is provided for treating cancer in a subject in need thereof, the method comprising administering to the subject an anti-MerTK antibody of the present disclosure, wherein the anti-MerTK antibody induces M1-like macrophage polarization.

In some embodiments, the cancer is selected from sarcoma, bladder cancer, breast cancer, colon cancer, endometrial cancer, kidney cancer, renal cancer, leukemia, lung cancer, non-small cell lung cancer, melanoma, lymphoma, pancreatic cancer, prostate cancer, ovarian cancer, stomach cancer, thyroid cancer, cancer of the uterus, liver cancer, cervical cancer, testicular cancer, squamous cell carcinoma, glioma, glioblastoma, adenoma, and neuroblastoma. In some embodiments, the cancer is selected from glioblastoma multiforme, bladder carcinoma, and esophageal carcinoma. In some embodiments, the cancer is triple-negative breast carcinoma. In some embodiments, the cancer may be a primary tumor. In some embodiments, the cancer may be a metastatic tumor at a second site derived from any of the above types of cancer. In some embodiments, an anti-MerTK antibody of the present disclosure is useful for treating cancer in s subject in need thereof, wherein the cancer expresses MerTK.

In some embodiments, an anti-MerTK antibody of the present disclosure may be administered in conjunction with one or more therapeutic agents that act as a checkpoint inhibitor. In some embodiments, the method further includes administering to the individual at least one antibody that specifically binds to an inhibitory immune checkpoint molecule, and/or another standard or investigational anti-cancer therapy. In some embodiments, the inhibitory checkpoint molecule is selected from PD1, PD-L1, and PD-L2, In some embodiments, the at least one antibody that specifically binds to an inhibitory checkpoint molecule is administered in combination with the anti-MerTK antibody of the present disclosure

In some embodiments, the at least one antibody that specifically binds to an inhibitory checkpoint molecule is selected from an anti-PD-L1 antibody, an anti-PD-L2 antibody, and an anti-PD-1 antibody

In some embodiments, a subject or individual is a mammal. Mammals include, without limitation, domesticated animals (e.g., cows, sheep, cats, dogs, and horses), primates (e.g., humans and non-human primates such as monkeys), rabbits, and rodents (e.g., mice and rats). In some embodiments, the subject or individual is a human.

### V. Diagnostic Uses

In some embodiments of any of the antibodies, any of the anti-MerTK antibodies provided herein is useful for detecting the presence of MerTK in a sample or an individual. The term “detecting” as used herein encompasses quantitative or qualitative detection. Provided herein are methods of using the antibodies of this disclosure for diagnostic purposes, such as the detection of MerTK in an individual or in tissue samples derived from an individual. In some embodiments, the individual is a human. In some embodiments, the tissue sample is phagocytic cells (e.g., macrophages, dendritic cells), tumor tissue, cancer cells, etc.

The detection method may involve quantification of the antigen-bound antibody. Antibody detection in biological samples may occur with any method known in the art, including immunofluorescence microscopy, immunocytochemistry, immunohistochemistry, ELISA, FACS analysis, immunoprecipitation, or micro-positron emission tomography. In certain embodiments, the antibody is radiolabeled, for example with ¹⁸F and subsequently detected utilizing micro-positron emission tomography analysis. Antibody-binding may also be quantified in a patient by non-invasive techniques such as positron emission tomography (PET), X-ray computed tomography, single-photon emission computed tomography (SPECT), computed tomography (CT), and computed axial tomography (CAT).

### VI. Articles of Manufacture

Provided herein are articles of manufacture (e.g., kit) comprising an anti-MerTK antibody described herein. Article of manufacture may include one or more containers comprising an antibody described herein. Containers may be any suitable packaging including, but is not limited to, vials, bottles, jars, flexible packaging (e.g., sealed Mylar or plastic bags), and the like. The containers may be unit doses, bulk packages (e.g., multi-dose packages) or sub-unit doses.

In some embodiments, the kits may further include a second agent. In some embodiments, the second agent is a pharmaceutically-acceptable buffer or diluting agent including. In some embodiments, the second agent is a pharmaceutically active agent.

In some embodiments of any of the articles of manufacture, the article of manufactures further include instructions for use in accordance with the methods of this disclosure. The instructions generally include information as to dosage, dosing schedule, and route of administration for the intended treatment. In some embodiments, these instructions comprise a description of administration of the isolated antibody of the present disclosure (e.g., an anti-MerTK antibody described herein) to treat an individual having a disease, disorder, or injury, such as for example cancer, according to any methods of this disclosure. In some embodiments, the instructions include instructions for use of the anti-MerTK antibody and the second agent (e.g., second pharmaceutically active agent).

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Production of his-Conjugated and Murine Fc-Conjugated MerTK Polypeptides

Human, cyno, and murine MerTK polypeptides containing polyHis or TEVS/Thrombin/murine IgG2a-Fc tagged fusion proteins for use in the generation and characterization of anti-MerTK antibodies of the present disclosure were generated as follows. Nucleic acid encoding the extracellular domain (ECD) of human MerTK (SEQ ID NO: 2), cyno MerTK (SEQ ID NO: 3), and murine MerTK (SEQ ID NO: 4) were each cloned into a mammalian expression vector containing nucleic acid encoding a heterologous signal peptide as well as containing either a PolyHis Fc tag or TEVS/Thrombin/murine IgG2a Fc tag.

The amino acid sequences of human MerTK, human MerTK extracellular domain, cyno MerTK extracellular domain, and murine MerTK extracellular domain are set forth below.

Human MerTK amino acid sequence (SEQ ID NO: 1):
MGPAPLPLLLGLFLPALWRRAITEAREEAKPYPLFPGPFPGSLQTDHTP
LLSLPHASGYQPALMFSPTQPGRPHTGNVAIPQVTSVESKPLPPLAFKH
TVGHIILSEHKGVKFNCSISVPNIYQDTTISWWKDGKELLGAHHAITQF
YPDDEVTAIIASFSITSVQRSDNGSYICKMKINNEEIVSDPIYIEVQGL
PHFTKQPESMNVTRNTAFNLTCQAVGPPEPVNIFWVQNSSRVNEQPEKS
PSVLTVPGLTEMAVFSCEAHNDKGLTVSKGVQINIKAIPSPPTEVSIRN
STAHSILISWVPGFDGYSPFRNCSIQVKEADPLSNGSVMIFNTSALPHL
YQIKQLQALANYSIGVSCMNEIGWSAVSPWILASTTEGAPSVAPLNVTV
FLNESSDNVDIRWMKPPTKQQDGELVGYRISHVWQSAGISKELLEEVGQ
NGSRARISVQVHNATCTVRIAAVTRGGVGPFSDPVKIFIPAHGWVDYAP
SSTPAPGNADPVLIIFGCFCGFILIGLILYISLAIRKRVQETKFGNAFT
EEDSELVVNYIAKKSFCRRAIELTLHSLGVSEELQNKLEDVVIDRNLLI
LGKILGEGEFGSVMEGNLKQEDGTSLKVAVKTMKLDNSSQREIEEFLSE
AACMKDFSHPNVIRLLGVCIEMSSQGIPKPMVILPFMKYGDLHTYLLYS
RLETGPKHIPLQTLLKFMVDIALGMEYLSNRNFLHRDLAARNCMLRDDM
TVCVADFGLSKKIYSGDYYRQGRIAKMPVKWIAIESLADRVYTSKSDVW
AFGVTMWELATRGMTPYPGVQNHEMYDYLLHGHRLKQPEDCLDELYEIM
YSCWRTDPLDRPTFSVLRLQLEKLLESLPDVRNQADVIYVNTQLLESSE
GLAQGSTLAPLDLNIDPDSIIASCTPRAAISVVTAEVHDSKPHEGRYIL
NGGSEEWEDLTSAPSAAVTAEKNSVLPGERLVRNGVSWSHSSMLPLGSS
LPDELLFADDSSEGSEVLM

Human MerTK ECD amino acid sequence
(SEQ ID NO: 2):
MGPAPLPLLLGLFLPALWRRAITEAREEAKPYPLFPGPFPGSLQTDHTP
LLSLPHASGYQPALMFSPTQPGRPHTGNVAIPQVTSVESKPLPPLAFKH
TVGHIILSEHKGVKFNCSISVPNIYQDTTISWWKDGKELLGAHHAITQF
YPDDEVTAIIASFSITSVQRSDNGSYICKMKINNEEIVSDPIYIEVQGL
PHFTKQPESMNVTRNTAFNLTCQAVGPPEPVNIFWVQNSSRVNEQPEKS
PSVLTVPGLTEMAVFSCEAHNDKGLTVSKGVQINIKAIPSPPTEVSIRN
STAHSILISWVPGFDGYSPFRNCSIQVKEADPLSNGSVMIFNTSALPHL
YQIKQLQALANYSIGVSCMNEIGWSAVSPWILASTTEGAPSVAPLNVTV
FLNESSDNVDIRWMKPPTKQQDGELVGYRISHVWQSAGISKELLEEVGQ
NGSRARISVQVHNATCTVRIAAVTRGGVGPFSDPVKIFIPAHGWVDYAP
SSTPAPGNADPVLII

Cyno MerTK ECD amino acid sequence
(SEQ ID NO: 3):
MGLAPLPLPLLLGLFLPALWSRAITEAREEAKPYPLFPGPLPGSLQTDH
TSLLSLPHTSGYQPALMFSPTQPGRPYTGNVAIPRVTSAGSKLLPPLAF
KHTVGHIILSEHKDVKFNCSISVPNIYQDTTISWWKDGKELLGAHHAIT
QFYPDDEVTAIIASFSITSVQRSDNGSYICKMKINNEEIVSDPIYIEVQ
GLPHFTKQPESMNVTRNTAFNLTCQAVGPPEPVNIFWVQNSSRVNEQPE
KSPSVLTVPGLTEMAVFSCEAHNDKGLTVSKGVQINIKAIPSPPTEVSI
HNSTAHSILISWVPGFDGYSPFRNCSVQVKEVDPLSNGSVMIFNTSASP
HMYQIKQLQALANYSIGVSCMNEIGWSAVSPWILASTTEGAPSVAPLNV
TVFLNESRDNVDIRWMKPLTKRQAGELVGYRISHVWQSAGISKELLEEV
GQNNSRAQISVQVHNATCTVRIAAVTKGGVGPFSDPVKIFIPAHGWVDH
APSSTPAPGNADPVLII

Murine MerTK ECD amino acid sequence
(SEQ ID NO: 4):
MVLAPLLLGLLLLPALWSGGTAEKWEETELDQLFSGPLPGRLPVNHRPF
SAPHSSRDQLPPPQTGRSHPAHTAAPQVTSTASKLLPPVAFNHTIGHIV
LSEHKNVKFNCSINIPNTYQETAGISWWKDGKELLGAHHSITQFYPDEE
GVSIIALFSIASVQRSDNGSYFCKMKVNNREIVSDPIYVEVQGLPYFIK
QPESVNVTRNTAFNLTCQAVGPPEPVNIFWVQNSSRVNEKPERSPSVLT
VPGLTETAVFSCEAHNDKGLTVSKGVHINIKVIPSPPTEVHILNSTAHS
ILVSWVPGFDGYSPLQNCSIQVKEADRLSNGSVMVFNTSASPHLYEIQQ
LQALANYSIAVSCRNEIGWSAVSPWILASTTEGAPSVAPLNITVFLNES
NNILDIRWTKPPIKRQDGELVGYRISHVWESAGTYKELSEEVSQNGSWA
QIPVQIHNATCTVRIAAITKGGIGPFSEPVNIIIPEHSKVDYAPSSTPA
PGNTDSM

The human, cyno, and murine MerTK nucleic acid fusion constructs were transiently transfected into HEK293 cells. The recombinant fusion polypeptides were purified from the supernatants of the cells using Mabselect resin (GE Healthcare, Cat #17519902) following the manufacturer's instructions. Additionally, commercially available DDDDK-tagged human MerTK fusion polypeptide (Sino Biological, Wayne, PA, Cat #10298-HCCH) or human IgG1 Fc-tagged murine MerTK fusion proteins (R&D systems, Minneapolis, MA, Cat #591-NMR-100) were also used for anti-MerTK antibody characterization as described below.

### Example 2: Generation of Human and Murine MerTK Overexpressing CHO Cell Lines

Human MerTK and murine MerTK overexpressing CHO cell lines were prepared as follows. Human MerTK open reading frame (ORF) clone Lentivirus particle (Cat #RC215289L4V) and mouse MerTK ORF clone Lentivirus particle (Cat #MR225392L4V) (Origene, Rockville, MD) (both mGFP-tagged) were used for preparing human MerTK overexpressing CHO-K1 and murine MerTK overexpressing CHO-K1 stable cell line generation, respectively.

CHO cells were cultured in F12-K media (ATCC, Cat #ATCC 30-2004) containing 10% FBS (Gibco) until >80% confluent. The cells were then dissociated with Trypsin buffer (0.25% EDTA/Trypsin, Gibco, Cat #25200056) and plated at 70-80% confluency in 6-well plates 24 hours prior to transduction with either the human or murine MerTK lentivirus construct. The following day, cells were incubated with the lentiviral particle at 4° C. for 2 hours and then the plates were incubated at 37° C. in 5% CO₂. Two days later, puromycin (Invivogen, San Diego, CA, Cat #ant-pr-1) was added for selection; selected puromycin-resistant cells were frozen in Cell Recovery Freezing Medium (Gibco, Cat #12648010) for subsequent use.

For FACS analysis of these cell lines, human MerTK overexpressing CHO cells (CHO-huMerTK OE cells) and mouse MerTK overexpressing CHO cells (CHO-muMerTK OE cells) generated as described above were plated at 1-2×10′ cells per well in 96-well U-bottom plates and incubated with a commercially available mouse anti-human MerTK monoclonal antibody (BioLegend, Clone: 590H11G1E3, Cat #367608, San Diego, CA) or a commercially available rat anti-mouse MerTK monoclonal antibody (ThermoFisher, Clone: DS5MMER, Cat #12-5751-82) for 30 minutes on ice. Cells were rinsed twice with ice-cold FACS buffer (2% FBS+PBS) and then incubated with APC-conjugated goat anti-mouse antibody (Jackson ImmunoResearch, West Grove, PA, Cat #115-606-071) or goat-anti-rat antibody (Jackson ImmunoResearch, Cat #112-606-071) for 30 min on ice. Following the secondary antibody incubation, the cells were washed with ice-cold FACS buffer and then resuspended in a final volume of 50-200 μl of FACS buffer containing 0.25 μl/well propidium iodide (BD, Cat #556463). Analysis was performed using a FACS CantoII system (BD Biosciences).

The resulting human MerTK and murine MerTK overexpressing CHO cell lines were used for subsequent studies to characterize anti-MerTK antibodies as described below.

### Example 3: MerTK Expression Profile

MerTK expression was examined on various human cell types and tissues, including U937 cells (ATCC CRL-1593.2; human macrophage cell line), SK-MEL-5 cells (ATCC HTB-70; human melanoma cell line), A375 cells (ATCC CRL-1619; human melanoma cell line), THP-1 cells (ATCC TIB-202; human monocyte-like cell line), CHO-huMerTK OE cells, human monocytes, human monocyte-derived macrophages, and human monocyte-derived dendritic cells. Additionally, MerTK expression was examined on human monocytes, macrophages, and dendritic cells obtained from human tumor samples as outlined below.

Monocytes were isolated from peripheral blood mononuclear cells (PBMCs) from healthy human donors using RosetteSep monocyte isolation antibody cocktail (StemCell Technologies). The isolated human monocytes were either differentiated into human macrophages with 50 ng/mL M-CSF (Peprotech) or were differentiated into human dendritic cells (DCs) with 100 ng/mL GM-CSF and 100 ng/mL IL-4 (Peprotech). The human macrophages or human dendritic cells were incubated with a commercially available fluorochrome-conjugated anti-human MerTK antibody (BioLegend, clone 590H11G1E3) for 30 minutes on ice in the dark. Cells were washed twice in FACS buffer (PBS+2% FBS, 2 mM EDTA), and analysis of MerTK expression on the surface of the cells was performed using a BD FACS Cantoll system.

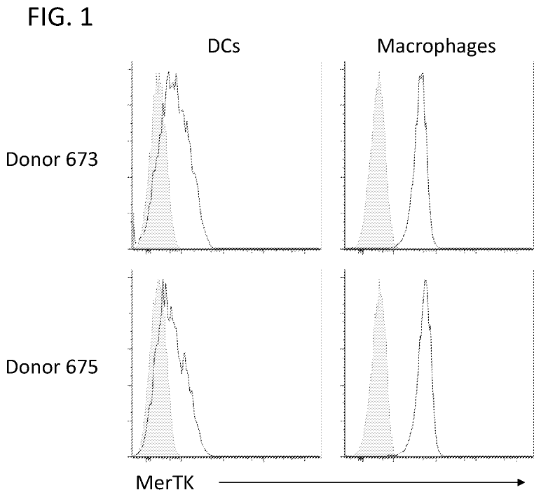

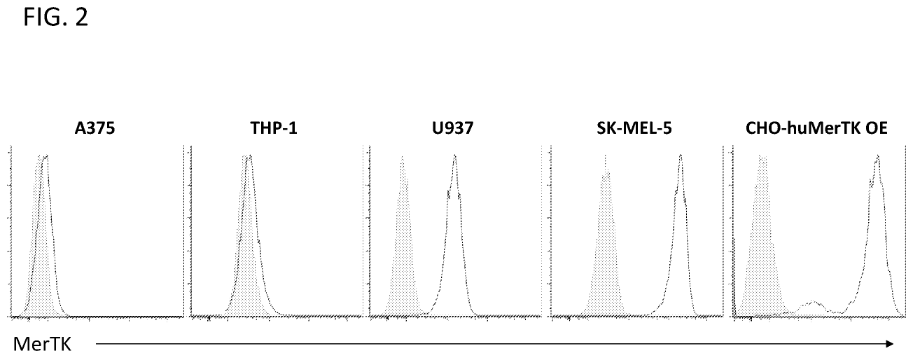

FIG. 1 shows results of FACS analysis of human MerTK expression on human myeloid cells (i.e., dendritic cells, macrophages). As shown in FIG. 1, dendritic cells (DCs) and macrophages from two different human donors displayed anti-MerTK antibody reactivity, indicating these cells express MerTK on their cell surface. FIG. 2 shows results of FACS analysis of human MerTK expression on various cell lines, including A375 cells, THP-1 cells, U937 cells, SK-MEL-5 cells, and CHO-huMerTK OE cells. As shown in FIG. 2, U937 cells, SK-MEL-5 cells, and CHO-huMerTK OE cells displayed anti-MerTK antibody reactivity, indicating that these cell lines express MerTK on their cell surface.

For analysis of MerTK expression on various myeloid cell types (e.g., monocytes, macrophages, dendritic cells) obtained from tumor samples, the following experiments were performed. Various tumor samples (from ovary, liver, and endometrial cancer) obtained from human subjects were cut into small pieces with a scalpel and transferred to GentleMACS C tubes (Miltenyi Biotec, Sunnyvale, CA) containing an enzyme mix. Samples were dissociated on GentleMACS (Miltenyi Biotec) as per the manufacturer's protocol. After dissociation, cells were filtered through a 100 m filter prior to FACS staining. Samples were then stained with Aqua Live Dead (Thermo Scientific, Cat #L34957) and a mixture of anti-human CD16 (Clone 3G8, BioLegend, Cat #302002), anti-human CD32 (Clone AT10, Thermo Scientific, Cat #MA1-81191), and anti-human CD64 (Clone 10.1, BioLegend, Cat #305002) antibodies at 4° C. for 30 min.

After washing with FACS buffer, the cells were then stained with the following antibodies for FACS sorting and analysis: anti-human CD45-Alexa Fluor 700 (Clone HI30, BioLegend, Cat #304024), anti-human CD3-APCCy7 (clone OKT3, BioLegend, Cat #317342), anti-human HLA-DR-BV711 (Clone L243, BioLegend, Cat #307644), anti-human CD15-BV605 (Clone W6D3, BioLegend, Cat #323032), anti-human CD14-BUV395 (Clone MoP9, BD bioscience, Cat #563561), anti-human CD19-BV650 (Clone HIB19, BioLegend, Cat #302238), anti-human CD56-PE Dazzle 594 (Clone HCD56, BioLegend, Cat #318348), anti-human CD1c-PerCPCy5.5 (Clone L161, BioLegend, Cat #331514), anti-human CD206-PECy5 (Clone 15-2, BioLegend, Cat #321108), and anti-human MerTK-BV421 (Clone 590H11G1E3, BioLegend, Cat #367604) at 4° C. for 30 min. Cells were washed with FACS buffer and acquired on a BD FACS Fortessa X20. All FACS data were analyzed using FlowJo software. Monocytes (defined and gated as CD45+CD3-CD14+), macrophages (defined and gated as CD45+CD3-CD14+CD206+), and dendritic cells (defined and gated as CD45+CD3-CD14-CD56-HLA-DR+CD1c+) were identified.

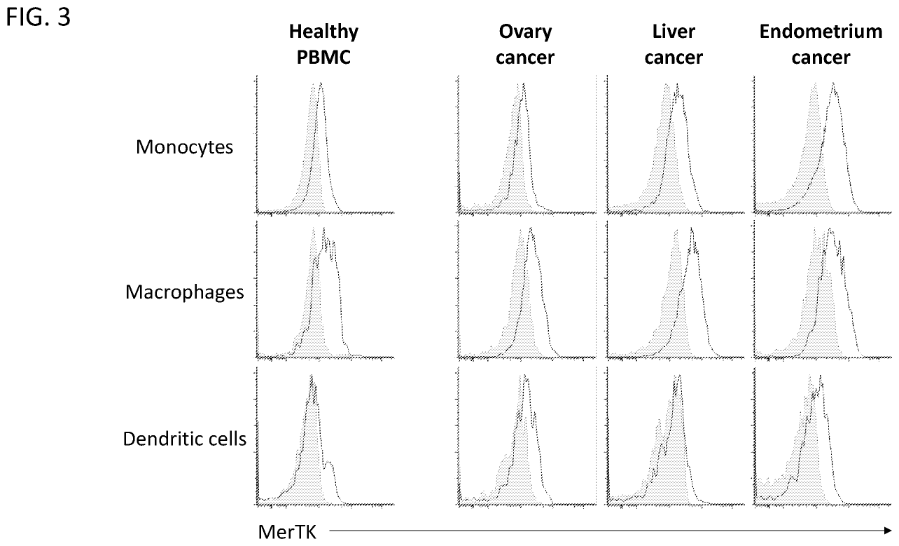

As shown in FIG. 3, MerTK expression was observed by FACS analysis in monocytes, macrophages, and dendritic cells obtained from peripheral blood mononuclear cells (PBMCs) from healthy human subjects. MerTK expression was also observed in tumor-derived monocytes, macrophages, and dendritic cells obtained from ovary, liver, and endometrial cancers obtained from human subjects.

Taken together, this data showed that MerTK expression was detected on various human cells and human cell lines, on monocytes, macrophages, and dendritic cells isolated from health human subjects, and on monocytes, macrophages, and dendritic cells obtained from human ovary, liver, and endometrial cancer tumor tissue.

### Example 4: Generation of Anti-MerTK Hybridoma Antibodies

In order to obtain antibodies against MerTK, the following experiments were performed in order to generate anti-MerTK hybridomas. BALB/c mice (Charles River Laboratories, Wilmington, MA) or MerTK knock-out (KO) mice (Jackson Laboratories, Bar Harbor, ME) were immunized twice a week by subcutaneous or intraperitoneal injections of purified extracellular domain polypeptides of human, cyno, and mouse MerTK (obtained as described above in Example 1) with or without adjuvant. A total of 8 injections were performed over 4 weeks. Three days following the final injection, spleens and lymph nodes were harvested from the mice for hybridoma cell line generation.

Lymphocytes from the spleens and lymph nodes of the immunized mice were isolated and then fused with P3X63Ag8.653 (CRL-1580, American Type Culture Collection, Rockville, MD) or SP2/mIL-6 (CRL-2016, American Type Culture Collection, Rockville, MD) mouse myeloma cells via electrofusion (Hybrimmune, BTX, Holliston, MA) and incubated at 37° C., 5% CO₂, overnight in Clonacell-HY Medium C (STEMCELL Technologies, Vancouver, BC, Canada, Cat #03803). The following day, the fused cells were centrifuged and resuspended in 10 ml of ClonaCell-HY Medium C with anti-mouse IgG Fc-FITC (Jackson ImmunoResearch, West Grove, PA) and then gently mixed with 90 ml of methylcellulose-based ClonaCell-HY Medium D (STEMCELL Technologies, Cat #03804) containing HAT components. The cells were plated into Nunc OmniTrays (Thermo Fisher Scientific, Rochester, NY) and allowed to grow at 37° C., 5% CO₂ for seven days. Fluorescent colonies were then selected and transferred into 96-well plates containing Clonacell-HY Medium E (STEMCELL Technologies, Cat #03805) using a Clonepix 2 (Molecular Devices, Sunnyvale, CA). In total, 1,728 IgG-secreting hybridoma clones were selected. After six days of culture, tissue culture supernatants from the hybridomas were screened by FACS analysis for specificity to bind human or mouse MerTK as described below.

### Example 5: Screening of Anti-MerTK Antibody Hybridoma Supernatants by FACS

Hybridoma culture supernatants from 1,728 hybridomas obtained as described above were screened for their ability to bind MerTK on various cell types, including CHO cells stably overexpressing human MerTK (CHO-huMerTK OE cells) or stably overexpressing mouse MerTK (CHO-muMerTK OE cells) (generated as described above), and CHO parental cells; U937 cells (ATCC CRL-1593.2), SK-MEL-5 cells (ATCC HTB-70) (which endogenously express human MerTK), J774A. 1 cells (ATCC TIB-67) (which endogenously express mouse MerTK), and A375 cells (ATCC CRL-1619). THP-1 cells (ATCC TIB-202), which have no or minimal expression of MerTK, served as non-MerTK expressing negative control cells in these experiments.

For screening of the hybridoma cell culture supernatants, a multiplexed FACS experimental design was utilized to determine binding of anti-MerTK antibodies to these multiple cell lines. Briefly, cells were stained with various concentrations and combinations of CellTrace cell proliferation dyes CFSE and Violet (ThermoFisher, Cat #C34554 and Cat #34557, respectively) to create uniquely barcoded cell populations. 70,000 cells of each barcoded cell type were aliquoted into 96-well U-bottom plates and incubated with 50d of hybridoma cell culture supernatant or 5 g/ml of commercially available purified mouse anti-human MerTK monoclonal antibody (BioLegend, Cat #367602; serving as a positive anti-MerTK antibody) on ice for 30 minutes. After this primary anti-MerTK hybridoma supernatant incubation with the various MerTK expressing cell types, the supernatants were removed via centrifugation, the cells were washed twice with 175 d of ice-cold FACS buffer (PBS+1% FBS+2 mM EDTA), and the cells were then further incubated on ice for 20 minutes with anti-mouse IgG Fc-allophycocyanin (APC) (Jackson Labs, Cat #115-136-071) (diluted 1:1000). Following this secondary antibody incubation, the cells were again washed twice with ice-cold FACS buffer and resuspended in a final volume of 30 μl of FACS buffer containing 0.25 μl/well propidium iodide (BD Biosciences, Cat #556463). Binding intensity on cells was analyzed using the FACS Canto system (BD Biosciences), with sorting gates drawn to exclude dead (i.e., propidium iodide-positive) cells. The ratio of APC Mean Fluorescence Intensity (MFI) on each barcoded cell population was determined for each anti-MerTK hybridoma supernatant tested.

From this specific hybridoma supernatant screen, a total of 308 anti-MerTK hybridoma clones were identified that displayed greater than 2-fold difference in binding (as determined by MFI) to cells stably overexpressing or endogenously expressing human or mouse MerTK compared to the binding observed on parental or negative control cell types. Anti-MerTK antibodies identified using this screen were further characterized as described below.

### Example 6: Screening of Anti-MerTK Antibody Hybridoma Supernatants by Recombinant MerTK Protein Binding Assay

Hybridoma culture supernatants from 1,728 hybridomas obtained as described above were screened for their ability to bind polyHis-tagged human, cyno, and mouse MerTK (prepared as described above in Example 1) as compared to binding to an irrelevant His-tagged control protein. Briefly, 96-well polystyrene plates were coated with 1p g/ml of human, cyno, or mouse poly-His-tagged MerTK polypeptide in coating buffer (0.05M carbonate buffer, pH 9.6, Sigma, Cat #C3041) overnight at 4° C. Coated plates were then blocked with ELISA diluent (PBS+0.5% BSA+0.05% Tween20) for one hour and washed three times with 300 μl of PBST (PBS+0.05% Tween20, Thermo 28352). The hybridoma cell culture supernatants or two commercially available purified mouse anti-human MerTK monoclonal antibodies (BioLegend Cat #367602; R&D Cat #MAB8912) were added (50 μl/well) to each well. After 30 mins incubation (room temperature, with shaking), the plates were washed three times with 300 μl of PBST. Anti-mouse IgG Fc-HRP (Jackson Immunoresearch, Cat #115-035-071) secondary antibody was diluted 1:5000 in ELISA diluent, added to each well at 50 μl/well, and incubated for 30 minutes at room temperature with shaking. After a final set of washes (3×300 μl in PBST), 50 μl/well of TMB substrate (BioFx, Cat #TMBW-1000-01) was added to the wells. The reaction was then quenched after 5-10 mins with 50 μl/well of stop solution (BioFx, Cat #BSTP-1000-01). The quenched reaction wells were detected for absorbance at 650 nm with a BioTek Synergy Microplate Reader using GEN5 2.04 software. From this hybridoma supernatant screen, a total of 326 anti-MerTK hybridoma clones were identified that displayed greater than 10-fold difference in binding to recombinant MerTK over background. Anti-MerTK antibodies identified using this screen were further characterized as described below.

### Example 7: MerTK ligand Gas6 and ligand ProS blocking assay using anti-MerTK hybridoma supernatants

Anti-MerTK antibody hybridoma supernatants identified as described above were evaluated for their ability to block binding of human Gas6 ligand and/or block binding of human ProS ligand to human MerTK by ELISA. Briefly, rabbit anti-human IgG antibody (Jackson ImmunoResearch, Cat #309-005-008) was coated at 2 g/ml onto high-protein binding plates at 4° C. overnight. After washing with 0.05% Tween20 in PBS three times, 5% BSA in PBS was added for 1 hour. Recombinant human MerTK-human Fc Chimera protein (R&D systems, Cat #391-MR-100) was added at 2 g/ml for 1 hour and plates were washed before the addition of 40 μl of anti-MerTK hybridoma supernatants and 40 μl of His tag-conjugated recombinant Gas6 (R&D systems, Cat #885-GSB-050) at 3 g/ml or His tag-conjugated recombinant ProS (R&D systems, Cat #9489-PS-100) at 20 g/ml. After a further incubation for 1 hour, plates were washed and incubated for 1 hour with HRP-conjugated anti-6× His tagged antibody (Abcam, Cambridge, MA, Cat #ab 1187). Plates were then washed and an HRP substrate, TMB, was added to develop the plates. The reaction was stopped by adding 501 2N H₂SO₄ and the OD was measured using a spectrophotometer (BioTek).

A total of 308 anti-MerTK hybridoma supernatant clones were screened for their ability to block Gas6 and/or block ProS ligand binding to MerTK. Twenty-nine (29) anti-MerTK hybridoma clones blocked the binding of both ProS ligand and Gas6 ligand to recombinant human MerTK protein. One hundred forty-five (145) anti-MerTK hybridoma clones blocked binding of ProS ligand to human MerTK protein only and did not block the binding of Gas6 ligand to recombinant human MerTK protein. The remainder of the 308 anti-MerTK hybridoma clones screened by this assay did not block either ProS ligand or Gas6 ligand binding to recombinant human MerTK protein in this assay. The hybridoma supernatants were characterized further regarding their ability to block efferocytosis by phagocytic cells as described below.

### Example 8: Efferocytosis Blocking Assay Using Anti-MerTK Hybridoma Supernatants

Anti-MerTK antibody hybridoma supernatants identified above as positive for MerTK binding reactivity were evaluated for their ability to block efferocytosis by human macrophages as follows. Human macrophages were differentiated from human monocytes for 7 days in the presence of human M-CSF. Macrophages were harvested (by scraping), resuspended in PBS, and plated on 96-well plates at 5×10⁴ cells/well. Cells were starved for 1 hour followed by the addition of anti-MerTK hybridoma supernatants to each well for 30 min at 37° C. Jurkat cells were treated with 1 M staurosporin (SigmaAldrich) for 3 hours at 37° C. (to induce apoptosis) and labeled with pHrodo (ThermoFisher) for 30 min at room temperature. After washing with PBS, pHrodo labeled Jurkat cells were added into each well at 1:4 ratio (1 macrophage cell to 4 Jurkat cells) for 1 hour. The plates were washed with PBS and then cells were stained with APC-conjugated anti-human CD14 for 30 minutes on ice in the dark. Cells were then washed twice in FACS buffer (PBS+2% FBS), and flow cytometry was performed on a BD FACS CantoII. Data were analyzed using FlowJo software. Macrophages that are capable of efferocytosis engulf the labeled apoptotic Jurkat cells, which are then be detected, whereas macrophages that are blocked from carrying out efferocytosis show decreased engulfment of the labeled apoptotic Jurkat cells.

In these experiments, efferocytosis-positive macrophages were identified by setting pHrodo CD14 double positive cells as an analysis gate and then applying this exact gate to all the samples. Baseline efferocytosis levels were established using macrophages cultured with media alone and this was set to 100% efferocytosis activity. Relative efferocytosis levels were calculated as a percent of efferocytosis observed in cells treated with media alone compared to that observed in cells treated with anti-MerTK hybridoma supernatants. In these experiments, the following additional anti-MerTK antibodies were used: mouse anti-human MerTK antibody H1 (BioLegend, Clone ID: 590H11G1E3, mouse IgG1) and human anti-human MerTK antibody M6 (disclosed in WO2016/106221).

Table 1 and Table 2 below show results from these efferocytosis experiments, as % efferocytosis (media alone was set to 100% efferocytosis). In Table 1 below, exemplary hybridoma supernatants tested are indicated on the left and labeled as hybridoma supernatant ID number; these supernatants were from hybridoma clones identified from immunization of wildtype BALB/c mice. In Table 2 below, exemplary hybridoma supernatants tested are indicated on the left and labeled as hybridoma supernatant ID number; these supernatants were from hybridoma clones identified from immunization of MerTK KO mice. In both Table 1 and Table 2, antibody ID refers to anti-MerTK antibodies of the present disclosure that were selected for additional characterization and thus given a specific anti-MerTK antibody name, as indicated. The third, fourth and fifth columns (“Donor” identification numbers) indicate the percent efferocytosis relative to media treatment alone. Note that in comparison to efferocytosis in macrophages in the absence of antibody (media treatment alone), no significant change in efferocytosis was observed in cells treated with isotype control mouse IgG1 antibody.

TABLE 1

| Hybridoma | | | | |
|---|---|---|---|---|
| supernatant | Antibody | Donor | Donor | Donor |
| ID | ID | 689 | 692 | 806 |
| 1 | | 89.4 | 80.8 | 94.4 |
| 2 | | 126.0 | 105.3 | 109.0 |
| 3 | MTK-01 | 34.1 | 34.2 | 70.1 |
| 4 | MTK-02 | 20.8 | 32.2 | 62.9 |
| 5 | | 68.9 | 101.6 | 73.1 |
| 6 | MTK-03 | 25.2 | 16.9 | 53.6 |
| 7 | MTK-04 | 23.8 | 25.6 | 58.3 |
| 8 | | 107.7 | 109.0 | 112.6 |
| 9 | | 86.3 | 109.0 | 88.3 |
| 10 | MTK-05 | 36.1 | 18.2 | 43.0 |
| 11 | MTK-06 | 16.1 | 16.0 | 67.5 |
| 12 | MTK-07 | 18.8 | 17.4 | 56.8 |
| 13 | | 68.9 | 101.6 | 73.1 |
| 14 | MTK-08 | 30.9 | 19.0 | 35.7 |
| 15 | | 65.4 | n.a. | 88.8 |
| 16 | MTK-09 | 19.9 | 17.6 | 50.5 |
| 17 | | 68.5 | 72.2 | 71.8 |
| 18 | MTK-10 | 18.8 | 12.6 | 50.0 |
| 19 | MTK-11 | 14.3 | 11.9 | 30.8 |
| 20 | MTK-12 | 14.6 | 12.4 | 38.6 |
| 21 | | 67.6 | 74.6 | 83.0 |
| 22 | MTK-13 | 21.8 | 12.2 | 45.6 |
| 23 | | 88.1 | 92.9 | 82.5 |
| 24 | MTK-14 | 25.2 | 17.5 | 55.3 |
| 25 | | 82.4 | n.a. | n.a. |
| 26 | MTK-15 | 27.2 | 23.7 | n.a. |
| 27 | | 86.8 | 61.8 | 78.9 |
| 28 | | 65.8 | 79.9 | n.a. |
| 29 | MTK-16 | 25.6 | n.a. | n.a. |
| 30 | MTK-17 | 22.7 | n.a. | n.a. |
| 31 | | 75.9 | 74.7 | 89.3 |
| 32 | MTK-18 | 24.2 | n.a. | n.a. |
| 33 | | 106.0 | 76.1 | 116.5 |
| 34 | MTK-19 | 48.0 | n.a. | n.a. |
| 35 | MTK-20 | 21.4 | n.a. | n.a. |
| 36 | | 98.1 | 86.1 | 85.7 |
| 37 | | 106.0 | 87.1 | 93.7 |
| 38 | | 74.1 | 62.8 | 90.5 |
| 39 | | 115.1 | 106.8 | 80.6 |
| 40 | | 108.1 | 62.0 | 100.2 |
| 41 | MTK-21 | 22.2 | n.a. | n.a. |
| 42 | MTK-22 | 25.7 | n.a. | n.a. |
| 43 | | 76.7 | 61.1 | 87.4 |
| 44 | | 97.2 | 71.8 | 69.4 |
| 45 | | 75.4 | 73.0 | 73.3 |
| 46 | MTK-23 | 17.0 | n.a. | n.a. |
| 47 | | 100.3 | n.a. | 95.9 |
| 48 | | 65.4 | n.a. | 71.6 |
| 49 | | 56.7 | 66.9 | 69.9 |
| 50 | | 63.7 | n.a. | 74.0 |
| | Media | 100.0 | 100.0 | 100.0 |
| | mIgG1 | 104.5 | 95.6 | 96.0 |
| | H1 | 31.2 | 16.5 | 57.9 |
| | M6 | 14.9 | n.a. | 35.1 |

n.a. means data not available

TABLE 2

| Hybridoma | | | | | |
|---|---|---|---|---|---|
| supernatant | Antibody | Donor | Donor | Donor | Donor |
| ID | ID | 914 | 915 | 916 | 783 |
| 1 | MTK-24 | 25.42 | 40.49 | 43.39 | 70.57 |
| 2 | | 86.61 | 66.80 | 72.73 | 106.69 |
| 3 | | 80.65 | 56.68 | 55.79 | 109.36 |
| 4 | MTK-25 | 44.05 | 31.01 | 25.50 | 21.37 |
| 5 | MTK-26 | 24.67 | 24.25 | 37.73 | 30.80 |
| 6 | | 83.04 | 73.28 | 68.18 | 103.01 |
| 7 | | 77.98 | 92.31 | 114.05 | 142.47 |
| 8 | | 100.30 | 92.71 | 102.07 | 123.08 |
| 9 | MTK-27 | 33.63 | 27.49 | 34.59 | 19.26 |
| 10 | | 78.87 | 63.56 | 73.14 | 92.31 |
| 11 | MTK-28 | 38.10 | 42.91 | 51.65 | 69.23 |
| 12 | | 96.73 | 87.85 | 102.48 | 123.08 |
| 13 | MTK-29 | 32.14 | 36.40 | 28.26 | 49.83 |
| 14 | | 97.92 | 73.68 | 80.99 | 122.07 |
| 15 | | 94.64 | 73.28 | 88.84 | 126.76 |
| 16 | | 106.55 | 86.23 | 97.93 | 130.43 |
| 17 | | 98.81 | 66.80 | 87.19 | 128.76 |
| 18 | | 101.79 | 95.55 | 100.00 | 115.72 |
| 19 | MTK-30 | 30.65 | 29.27 | 34.71 | 33.78 |
| 20 | MTK-31 | 41.37 | 37.85 | 32.60 | 32.78 |
| 31 | | 89.58 | 65.59 | 72.31 | 79.60 |
| 21 | | 79.17 | 47.37 | 68.60 | 93.98 |
| 22 | MTK-32 | 63.10 | 54.25 | 57.44 | 73.24 |
| 23 | MTK-33 | 25.54 | 22.35 | 36.61 | 13.55 |
| 24 | | 80.06 | 67.61 | 66.94 | 100.00 |
| 25 | | 68.15 | 51.42 | 55.37 | 62.21 |
| 26 | MTK-35 | 40.48 | 24.21 | 39.34 | 30.37 |
| 27 | | 86.61 | 64.37 | 88.02 | 119.40 |
| 28 | | 84.82 | 83.40 | 88.84 | 112.37 |
| 29 | | 78.57 | 61.94 | 73.97 | 122.41 |
| 30 | | 75.60 | 76.92 | 71.90 | 111.04 |
| 31 | | 75.30 | 78.95 | 76.86 | 117.39 |
| 32 | | 79.76 | 89.88 | 83.06 | 118.73 |
| 33 | | 72.02 | 75.30 | 68.60 | 98.33 |
| 34 | MTK-36 | 73.51 | 93.52 | 82.23 | 120.74 |
| 35 | | 71.73 | 82.59 | 74.79 | 128.76 |
| | Media alone | 100.00 | 100.00 | 100.00 | 100.00 |
| | M6 | 17.47 | 20.40 | 28.31 | 14.82 |
| | Isotype control | 88.99 | 104.05 | 104.55 | 93.65 |
| | H1 | 24.76 | 34.33 | 40.79 | 34.78 |

In Table 1 and Table 2, percent of efferocytosis by human macrophages from different donors is shown, with media alone (no antibody addition) set to 100% efferocytosis in both Tables. Although the degree of efferocytosis blocking activity by various anti-MerTK antibody hybridoma supernatants was different in macrophages obtained from each donor, the efferocytosis blocking trend for anti-MerTK antibodies was consistent among macrophages from different donors. As shown in Tables 1 and 2 above, anti-MerTK hybridoma supernatants of the present disclosure reduced efferocytosis by human macrophages to varying extents (e.g., reduced efferocytosis by about 50%-˜85%). These results indicated that anti-MerTK antibodies obtained as described herein were effective at reducing or blocking efferocytosis by phagocytic cells.

### Example 9: Molecular Cloning of Anti-MerTK Antibodies

Anti-MerTK antibodies from the hybridomas described above were subcloned as follows. 5×10⁵ hybridoma cells were harvested and washed with PBS and then the cell pellets were flash frozen in dry ice and stored at −20° C. Total RNA was extracted by using RNeasy Mini Kit (QIAGEN, Cat474104) following the manufacturer's protocol. cDNA was generated using Clontech's SMARTer RACE 5′/3′ Kit (Takara Bio USA, Cat4 634859) following the manufacturer's protocol. Variable heavy and light immunoglobulin regions were cloned separately by touchdown PCR using the 5′ UPM primer provided in the RACE kit and reverse primers recognizing the heavy chain and light chain constant regions. The resulting PCR products were purified and ligated into a pCR2.1-TOPO cloning vector (TOPO TA cloning Kit, Invitrogen Cat0450641) and transformed into Escherichia coli (E. coli) cells. Transformed colonies were isolated and the variable heavy chain (V_{H}) and variable light chain (V_{L}) nucleic acids were sequenced for each corresponding hybridoma cell line. Following the sequence determinations, variable heavy chain regions and variable light chain regions were amplified by PCR using primers containing endonuclease restriction sites and then subcloned into pLEV-123 (LakePharma, San Carlos, CA) mammalian expression vector encoding human IgG1I-Fc-LALAPS (human IgG1 Fc comprising amino acid substitutions L234A, L235A, and P331S by EU numbering) and IgG Kappa.

Amino acid sequences of the variable heavy chains and variable light chains of anti-MerTK antibodies of the present disclosure are provided below in Table 3. In Table 3, the CDRIHVR sequences (according to Kabat) are underlined.

TABLE 3

| | | SEQ ID | | SEQ ID |
|---|---|---|---|---|
| Antibody | Heavy Chain Variable | NO: | Light Chain Variable | NO: |
| MTK-01 | QIQLVQSGPELKKPGETVKISC | 5 | QIVLSQSPAILSASPGEKVTMTC | 40 |
| | KASGYTFTNYGMNWVKQAPGKG | | RASSSVSYMHWYQQKPGSSPKPW | |
| | LKCMGWINTYTGEPTYADDFKG | | IYATSNLASGVPARFSGSGSGTS | |
| | RFAFSLETSATTAYLQINNLKN | | YSLTISRVEAEDAATYYCQQWSS | |
| | EDTATYFCARRDRYTWFAYWGQ | | NPPTFGGGTKLEIK | |
| | GTLVTVSA | | | |
| MTK-02 | QIQLVQSGPELKOPGETVKISC | 6 | QIVLSQSPAILSASPGERVTMTC | 41 |
| | KASGYTFTDYGVNWVKQAPGKG | | RANSSVSYMHWYQQKPGSSPKPW | |
| | LKWMGWINTYSGEPTYAGDFKG | | IYATSNLASGVPARFSASGSGTS | |
| | RFAFSLESSASTAFLQINNLKN | | YSLTVSRVEAEDAATYYCQQWGS | |
| | EDMATYFCARRVRYWYFDVWGA | | NPFTFGSGTKLEIK | |
| | GTSVTVSS | | | |
| MTK-03 | DVQLQESGPGLVKPSQSLSLTC | 7 | QIVLTQSPAIMSAFPGEKVTITC | 42 |
| | SVTGYSITSGYYWNWIRQFPGN | | SASSSVNYMHWFQQKPGTSPKLW | |
| | TLEWMGYMSFDGDNKFNPSLKN | | IYSTSNLASGVPARFSGSGSGTS | |
| | RISITRDTSKNQFFLRLNSVTT | | YSLTISRMEAEDAATYYCQQRSS | |
| | EDTATYYCARGGYNYGSTEANW | | YPFTFGSGTKLEIK | |
| | GQGTLVTVSA | | | |
| MTK-04 | QIQLVQSGPELKKPGETVKISC | 8 | QIVLSQSPAFLSASPGEKVTMTC | 43 |
| | KASGYTFTNYGMNWVKQAPGKG | | RASSSVSYMHWYLQKPGSSPKPW | |
| | LKCMGWINTYTGEPTYADDFKG | | IYATSNLASGVPVRFSGSGSGTS | |
| | RFAFSLETSASTAYLQINNLKN | | YSLTISRVEAEDAATYYCQQWSS | |
| | EDMATYFCAKYDNYAWFAYWGQ | | NPRTFGGGTKLEIR | |
| | GTLVTVSA | | | |
| MTK-05 | QVOLQQSGAELVQPGTSVRLSC | 9 | DIQMTOSPASLSASVGETVTFTC | 44 |
| | KTSGYTFTSYWIQWVKQRPGQG | | RASENIFSYLAWYQQKQGKSPQL | |
| | LGWIGEIFPGTGTTYYNEKFKG | | LVYNAKTLAEGVPSRFSGSGSGT | |
| | KATLTIDTSSSTAYMQLSSLTS | | HFSLKINSLQPEDFGSYYCLHHY | |
| | EDSAVYFCARDGAYFDVWGAGT | | GTPLTFGAGTKLELK | |
| | TVTVSS | | | |
| MTK-06 | QIQLVQSGPELKKPGETVKISC | 10 | QIVLSQSPAILSASPGEKVTMTC | 45 |
| | KASGYTFTNYGMNWVKQAPGKG | | RASSSVSYMHWYQQKPGSSPKPW | |
| | LKWMGWINTNTGEPTYAEEFKG | | IYATSNLASGVPARFSGSGSGTS | |
| | RFAFSLETSASTAYLQINNLKN | | YSLTISRVEAEDAATYYCQQWSS | |
| | EDTATYFCARYYNYWYFDVWGA | | KPPTFGGGTKLEIK | |
| | GTTVTVSS | | | |
| MTK-07 | DVQLQESGPGLVKPSQSLSLTC | 11 | QIVLTQSPAIMSAFPGEKVTITC | 46 |
| | SVTGYSITSGYYWNWIRQFPGN | | SASSSVTYMHWFQQKPGTSPKLW | |
| | TLEWMGYMSFDGDNKFNPSLKN | | IFSTSNLASGVPARFSGSGSGTS | |
| | RISITRDTSKNQFFLRLNSVTT | | YSLTISRMEAEDAATYYCQQRSS | |
| | EDTATYYCARGGYNYGSTEANW | | YPFTFGSGTKLEIK | |
| | GQGTLVTVST | | | |
| MTK-08 | QIQLVQSGPELKKPGETVKISC | 12 | QIVLSQSPAILSASPGEKVTMTC | 47 |
| | KASGYTFTNYGMNWVKQAPGKG | | RATSSVSYMHWFQQKPGSSPKPW | |
| | LKWMGWINTYTGEPTYADDFKG | | IYATSNLASGVPARFSGSGSGTS | |
| | RFAFSLETSASTAYLQINNLKN | | YSLTISRVEAEDAAAYYCQHWSG | |
| | EDTATYFCAREVRYWYFDVWGA | | NPRTFGGGTKLEIK | |
| | GTTVTVSS | | | |
| MTK-09 | QVQLQQSGAELVRPGASVKISC | 13 | DIVMTQSHKFMSTSVGDRVSITC | 48 |
| | KAFGYTFTNHHIKWVKQRPGQG | | KASQDVTTSVAWYHQKPGQSPEL | |
| | LDWIGYIDPYNDYTTYNQNFKG | | LIYSASYRYTGVPDRFTGSGSGT | |
| | KATLTVDKSSSTAYMELSSLTS | | DFTFTISSVQAEDLAIYYCQQHY | |
| | EDSAVYYCARRAYDGYYVDWYF | | STPLTFGSGTKLQLK | |
| | DVWGAGTTVTVSS | | | |
| MTK-10 | QVTLKESGPGILQPSQTLSLTC | 14 | DIQMTOTTSSLSASLGDRVTISC | 49 |
| | SFSGFSLTTSGMGVGWIRQPSG | | SASQGISNYLNWFQQKPDGTVKL | |
| | KGLEWLAHIWSDDDKRSNPALK | | LIYYTSRLHSGVPSRFSGSGSGT | |
| | SRLTISQDSSTNQVFLKIASVD | | DFSLTISNLEPEDIATYYCQQYT | |
| | TADSATYYCSHLTPVREFAYWG | | KLPYTFGGGTKLEIK | |
| | PGTLVTVSE | | | |
| MTK-11 | QVQLKQSGPGLVQPSQSLSITC | 15 | QIVLTQSPAIMSASLGERVTMTC | 50 |
| | TVSGFSLTDYGVHWVRQSPGKG | | TASSSISSSYFHWYQQKPGSSPK | |
| | LEWLGVIWSSGTTDYNEAFISR | | LWIYSTSNLPSGVPARFSGSGSG | |
| | LSISKDNSKSHVFFKMNSLQAI | | TSYSLTISSMEAEDAATYYCHQY | |
| | DTAIYYCARKGHDPYAMDYWGQ | | YRSPLTFGAGTKLELK | |
| | GTSVTVSS | | | |
| MTK-12 | QVQLQQSGPQLVRPGASVKISC | 16 | QIVLTQSPAIMSASPGEKVTMTC | 51 |
| | KASGYSFTSHWMHWVKQRPGQG | | SASSSVSYMYWYQQKPRSSPRLL | |
| | LEWIGMIDPSDGESRLNQKFKD | | IYDTSNLASGVPVRFSGSGSGTS | |
| | KATLTVDKSSSTAYMQLSSPTF | | YSLTISRMEAEDAATYYCQQWSS | |
| | EDSAVYYCARGIYYYGITYAMD | | YPLTFGAGTKLELK | |
| | YWGQGTSVTVSS | | | |
| MTK-13 | DVQLQESGPGLVKPSQSLSLTC | 17 | QIVLTQSPPIMSASPGEKVTITC | 52 |
| | SVTGYSITSGYYWNWIRQFPGN | | SASSSVSYMYWFQQKPGTSPKLW | |
| | KLEWMGNIDYDGSNKYNPSLKN | | IYSTFNLASGVPARFSGSGSGTS | |
| | RISITRDTSKNHFFLKLNSVTP | | YSLTISRMEAEDVATYYCQQRSS | |
| | EDTATYFCARDGGNYRSFAYWG | | YPFTFGSGTKLEIK | |
| | QGTLVTVSA | | | |
| MTK-14 | QIQLVQSGPELKKPGETVKISC | 18 | QIVLSQSPAILSASPGEKVTMTC | 53 |
| | KASGYTFTNYGMNWVKQAPGKD | | RSSSSVSYMHWYQQKPGSSPKPW | |
| | LQWMGWINTYTGEPTYADDFTG | | IYATSNLASGVPGRFSGSGSGTS | |
| | RFAFSLETSASTAYLQINNLKN | | YSLTISRVEAEDAATYYCQQWGS | |
| | EDTATYFCAKGGHYAWFAYWGQ | | NPRTFGGGTKLEIK | |
| | GTLVTVSA | | | |
| MTK-15 | QIQLVQSGPELKKPGETVKISC | 19 | QIVLSQSPAILSASPGEKVTMTC | 54 |
| | KASGYTFTNYGVHWVKQAPGKG | | RASSSVSYMHWYQQKSGSSPKPW | |
| | LKWMGWINTYTGEPTYADDFKG | | IYATSNLASGVPARFSGSGSGTS | |
| | RFAFSLETSASTAYLQINNLKN | | YSLTISRVEAEDAATYYCQQWSS | |
| | EDMATYFCARGNRYAYMDYWGQ | | NPRTFGGGTKLEIK | |
| | GTSVTVSS | | | |
| MTK-16 | LIQLVQSGPELKKPGETVKISC | 20 | DIVMTQSPKFMSTSVGDRVSITC | 55 |
| | KASGYTFTNHGMNWVKQDPGKG | | KASQNVRTAVAWYKKKPGQSPKA | |
| | LKWMGWINTYTGEPTYADDFKG | | LINLASNRHTGVPDRFTGSGSGT | |
| | RFVFSMETSASAAFLQINNLKN | | DFTLTISNVQSEDLADYFCLQHW | |
| | EDTATYFCARKGVTAARYFDYW | | NYPLTFGGGTKLEIK | |
| | GQGTTLTVSS | | | |
| MTK-17 | QIQLVQSGPELKKPGETVKISC | 21 | DIQMTQSSSYLSVSLGGRVTITC | 56 |
| | KASGYTFTNFGMNWVKQAPGKG | | KASDHINNWLAWYQQKPGNAPRL | |
| | LKWMGWINTYTGEPTYADDFKG | | LISGATSLETGVPSRFSGSGSGK | |
| | RFAFSLETSASTAYLQINNLKN | | DYTLSITSLQTEDVATYYCQQYW | |
| | EDTATYFCSRGAVLRAGAMDCW | | STPRTFGGGTKLEIK | |
| | GQGTSVTVSS | | | |
| MTK-18 | QIQLVQSGPELKKPGETVKISC | 22 | DIQMTQTTSSLSASLGDRVTFSC | 57 |
| | KASGYTFTHYGMTWVKQAPGKG | | RASQAISNYLNWYQQKPDGTVKL | |
| | LKWMGWINTYTGEPTYADDFKG | | LIYYTSRLHSGVPSRFSGSGSGT | |
| | RFAFSLETSASTAYLQINNLKN | | DYSLTISNLEQEDIATYFCQQGN | |
| | EDTATYFCARGGGQLGLRPLDY | | TLPWTFGGGTKLEIK | |
| | WGQGTTLTVSS | | | |
| MTK-19 | EVQLQQSGPELVKPGASVKISC | 23 | DVVMTQSSLTLSVTIGQPASISC | 58 |
| | KTSGYTFTEYTMHWVKQSHGKS | | KSSQSLLDSDGKTYLNWLLQRPG | |
| | LEWLGGFNPNNVITSYNQRFQG | | QSPKRLIYLVSKLDSGVPDRFTG | |
| | RATLTVDKSSSTAYMELRSLTS | | SGSGTDFTLKISRVEAEDLGVYY | |
| | DDSAVYYCTRGDLLWSLLLPGN | | CWQGTHFPWTFGGGTKLEIK | |
| | YFDYWGQGTTLTVSS | | | |
| MTK-20 | QIQLVQSGPELKKPGETVKISC | 24 | DIQMTQTTSSLSASLGDRVTISC | 59 |
| | KASGYTFTNYGMTWVKQAPGKG | | RASQDINNYLNWYQQKPDGTVKL | |
| | LKWMGWINTYTGEPTYADDFKG | | LIYYTSRLHSGVPSRFSGSGSGT | |
| | RFALSLETSASTAYLQINNLKN | | DYSLTISNLEQEDIATYFCQQGN | |
| | EDTATYFCARGGGRLGLRPLDY | | TLPWTFGGGTKLEIK | |
| | WGQGTTLTVSS | | | |
| MTK-21 | QIQLVQSGPELKKPGETVKISC | 25 | DIQMTOTTSSLSASLGDRVTISC | 60 |
| | KASGYTFTNFGMTWVRQAPGMD | | RASQDINNYLNWYQQKPDGTVKL | |
| | LKWMGWINTYTGEPKYADDFKG | | LIYYTSSLHSGVPSRFSGSGSGT | |
| | RFALSLETSASTAYLQITNFKN | | DYSLTITNLEQEDIATYFCQQGN | |
| | EDTATYFCARGGGRLGLRPLDY | | TLPWTFGGGTRLEIK | |
| | WGQGTTLTVSS | | | |
| MTK-22 | QVQLQQPGADLVEPGASVKLSC | 26 | DVVMTQSHKFMSTSVGDRVSITC | 61 |
| | KASGYTFTSYWMHWVKQRPGQG | | KASQDVGTAIAWYQQKPGQSPKL | |
| | LEWIGEIDPSDSSSNYNQKFKG | | LIYWASTRHTGVPDRFTGSRSGT | |
| | KATLTVDKSSSTAYMHLNSLTS | | VFTLTISNVQSEDLADYFCQQYT | |
| | EDSAVYYCARRYYYGSLYFDNW | | SYPLTFGSGTKLEIK | |
| | GQGTTLTVSS | | | |
| MTK-23 | QVQLQQSGADLVRPGASVKISC | 27 | DIVMTQSHKFMSTPVGDRVSITC | 62 |
| | KAFGYTFTNHHINWVKQRPGQG | | KASRDVSTAVAWFHQKPGQSPKL | |
| | LDWIGNVDPYNDYSTYNQKFKG | | LIYSASYRSTGVPDRFTGSGSGT | |
| | KATLTVDKSSSTAYMELSSLTS | | DFTFTISSVQAEDLAVYYCQQHY | |
| | EDSAVYYCARRVYDGFYVDWYF | | SAPLTFGAGTKLELK | |
| | DVWGAGTTVTVSS | | | |
| MTK-24 | QVQLQQPGAELVKPGASVKLSC | 28 | DIQMTQSPASLSVSVGETVTITC | 63 |
| | KASGYTFTSYWMHWVKQRPGQG | | RASENIYSNLAWYQQKQGKSPQL | |
| | LEWIGMIHPNINTNYNEKFKSK | | LVYAATNLADGVPSRFSGSGSGT | |
| | ATLTVDISSSTAYMQLSSLTSE | | QYSLKINSLQSEDFGSYYCQHFW | |
| | DSAVYYCAKRSPYSNYDWYFDV | | GTPLTFGAGTKLELK | |
| | WGTGTTVTVSS | | | |
| MTK-25 | EVKLVESEGGLVQPGSSMKLSC | 29 | DIVMTQSQKFMSTSVGDRVSITC | 64 |
| | TASGFTFSDYYMAWVRQVPEKG | | KASQNVRTTVAWYQQKPGQSPKT | |
| | LEWVANINYEGSSTYYLGSLKS | | LIYLASNRHTGVPDRFTGSGSGT | |
| | RFIISRDNAENILYLQMSSLKS | | DFTLTISNVQSEDLADYFCLQLW | |
| | EDTATYYCARYYYGSVDYWGQG | | NYPWTFGGGTKLEIK | |
| | TTLTVSS | | | |
| MTK-26 | AVQFQESGPGLVKLSQSLSLTC | 30 | QIVLTQSPAIMSASPGEKVTIAC | 65 |
| | SVTGYSITSGYYWDWIRQFPGN | | SASSSVSFMHWFQQKPGTSPRLW | |
| | KLEWMGYISYDGSNNYNPSLKN | | IYSTSNLASGVPARFSGSGSGTS | |
| | RISITRDTSKNQFFLKLNSVTT | | YSLTISRMEAEDAATYYCQQRSS | |
| | EDTATYYCAREGGYYRSMDYWG | | YPFTFGSGTKLEIK | |
| | QGTSVTVSS | | | |
| MTK-27 | EVQLQQSGTELVRPGASVKLSC | 31 | DIVMTQSQKFMSTSVGDRVSITC | 66 |
| | TASGFNIKDDYMHWVQQRPEQG | | KASQNVRTSVAWYQQKPGQSPKA | |
| | LEWIGWIDPENGNTEYASKFQG | | LIYLASNRHTGVPDRFTGSGSGT | |
| | KATITADASSNTAYLQLSSLTS | | DFTLTISNVRSEDLADYFCLQHW | |
| | EDTAVYYCSTLFSNYFDYWGQG | | NYPYTFGGGTKLEIK | |
| | TTLTVSS | | | |
| MTK-28 | QIQLVQSGPELKKPGETVKISC | 32 | QIVLSQSPVILSASPGEKVTMTC | 67 |
| | KASGYTFTTYGMSWVKQAPGKG | | RASSSVTYMHWYQQQPGSSPKPW | |
| | LKWMGWINTYSGVPTYADDFKG | | IYATSNLASGVPARFSGSGSGTS | |
| | RFAFSLETSASTAYLQINTLKN | | YSLTISRVETEDAATYYCHQWSG | |
| | EDTTTYFCARFLRYYYFDYWGQ | | NPTFGGGTKLEIN | |
| | GTTLTVSS | | | |
| MTK-29 | QIQLVQSGPELKKPGETVKISC | 33 | QIVLSQSPAILSASPGEKVTMTC | 68 |
| | KSSGFTFTTYGMSWVKQAPGKG | | RATSSVGYMHWYQQKPGSSPKPW | |
| | LKWMGWINTYSGVPTYTDDFKG | | IYATSNLASGVPARFSGSGSGTS | |
| | RFAFSLETSASTASLQINNLKN | | YSLTISRVEAEHAATYYCQQWGS | |
| | EDTATYFCARYTNYGYFDYWGQ | | NPFTFGSGTKLEIK | |
| | GTTLTVSS | | | |
| MTK-30 | QVOLQQPGTELVRPGASVKLSC | 34 | DVLMTQTPLSLPVSLGDQASISC | 69 |
| | KASGYTFTSYWMHWVKQRPGQG | | RSSQSIVHSNGDTYLEWYLQKPG | |
| | LEWIGNLNPNNGGTYYNEKFKS | | QSPKLLIYKVSNRFSGVPDRFSG | |
| | KATLTVDKSSSTAYMQLSSLTS | | SGSGTDFTLKISRVEAEDLGVYY | |
| | EDSAVYYCARQILLYFDYWGQG | | CFQGSHVPWTFGGGTKLEIK | |
| | TTLTVSS | | | |
| MTK-31 | QIQLVQSGPELKKSGETVKISC | 35 | DVSMTQTPLSLPVSLGDQASISS | 70 |
| | KASGYTFTSYGMSWVKQAPGKG | | RSSQTIVHSNGNTYLEWYLQKPG | |
| | LKWMGWINTYSGVPTYADDFKG | | QSPKLLIYKVSNRFSGVPDRFSG | |
| | RFAFSLETSASTAYLQINNLKN | | SGSGTDFTLKISRVEAEDLGVYY | |
| | EDTATYFCASLSYDGSLYHAMD | | CFQGSHVPWTFGGGTKLEIK | |
| | YWGQGTSVTVSS | | | |
| MTK-32 | QIQLVQSGPELKKPGETVKISC | 36 | QIVLSQSPAILSASPGEKVTMTC | 71 |
| | KASGYTFTSYGMSWVHQAPGKG | | RATSSVGYMHWYQRKPGSSPKPW | |
| | LKWLGWINTYSGVPTYADDFRG | | IYATSNLASGVPARFSGSGSGTS | |
| | RFAFSLDTSVSTASLEINNLQN | | YSLTISRVEAEDAATYYCQQWSS | |
| | EDTATYFCAREDNWAWFAYWGQ | | NPRTFGGGTKLEIK | |
| | GTLVTVST | | | |
| MTK-33 | QVQLQQPGPELVRPGTSVKLSC | 37 | SIVMTQTPKFLLVSAGDRVIITC | 72 |
| | KASGYTFTSYWMHWIKQRPGQG | | KASQSVSNTVAWYQQKPGQSPKL | |
| | LEWIGVIDPSDNYINYNQKFKG | | LIYYASNRYTGVPDRFTGSGYGT | |
| | KATLTVDTSSSTAYLQLSSLTS | | DFTFTISTVQAEDLAVYFCQQDY | |
| | EDSAVYYCAREAGTRGYFDYWG | | RSPFTFGSGTQLEMK | |
| | QGTTLTVSS | | | |
| MTK-34 | QVQLQQPGPELVRPGTSVKLSC | 37 | SIVMTQTPKFLLVSAGDRVIITC | 72 |
| | KASGYTFTSYWMHWIKQRPGQG | | KASQSVSNTVAWYQQKPGQSPKL | |
| | LEWIGVIDPSDNYINYNQKFKG | | LIYYASNRYTGVPDRFTGSGYGT | |
| | KATLTVDTSSSTAYLQLSSLTS | | DFTFTISTVQAEDLAVYFCQQDY | |
| | EDSAVYYCAREAGTRGYFDYWG | | RSPFTFGSGTQLEMK | |
| | QGTTLTVSS | | | |
| MTK-35 | QVLLQQSGPELVKPGASVQLSC | 38 | EIVLTQSPTTMAASPGEKITITC | 73 |
| | KASDYTFTSYDIHWVKQRPGQG | | RASSSISSHYLHWYQQKPGFSPK | |
| | LEWIGWIYPRDGYTKYNEIFKG | | LLIYRTSNLASGVPARFSGSGSG | |
| | KATLTVDTSSTTAYMELHSLTS | | TSYSLTIGTMEAEDVATYYCQQG | |
| | EDSAVYFCARAYYTNWYYFDYW | | STIPLTFGAGTKLVLK | |
| | GQGTTLTVSS | | | |
| MTK-36 | QVTLKESGPGILQPSQTLSLTC | 39 | QIVLSQSPAILSAFPGEKVTMTC | 74 |
| | SFSGFSLSTFGMGVGWIRQPSG | | RATSSVRYMHWYQQKPGSSPKPW | |
| | KGLEWLAHIWWYDDKYYEPALK | | IYATYNLTSGVPARFSGSGSGTS | |
| | SRLTISKDSSKNQVFLKIANVD | | YSLTISRVEAEDAATYYCHQWSS | |
| | TADTATYYCARIYYGTSYRYFD | | NPYTFGGGTKLEIK | |
| | VWGTGTTVTVSS | | | |

The CDR sequences according to Kabat for the anti-MerTK antibodies of the present disclosure are provided below in Table 4 (heavy chain) and Table 5 (light chain).

TABLE 4

| | | SEQ | | SEQ | | SEQ |
|---|---|---|---|---|---|---|
| Antibody | HVR-H1 | ID NO: | HVR-H2 | ID NO: | HVR-H3 | ID NO: |
| MTK-01 | NYGMN | 75 | WINTYTGEPT | 99 | RDRYTWFAY | 125 |
| | | | YADDFKG | | | |
| MTK-02 | DYGVN | 76 | WINTYSGEPT | 100 | RVRYWYFDV | 126 |
| | | | YAGDFKG | | | |
| MTK-03 | SGYYWN | 77 | YMSFDGDNKF | 101 | GGYNYGSTE | 127 |
| | | | NPSLKN | | AN | |
| MTK-04 | NYGMN | 75 | WINTYTGEPT | 99 | YDNYAWFAY | 128 |
| | | | YADDFKG | | | |
| MTK-05 | SYWIQ | 78 | EIFPGTGTTY | 102 | DGAYFDV | 129 |
| | | | YNEKFKG | | | |
| MTK-06 | NYGMN | 75 | WINTNTGEPT | 103 | YYNYWYFDV | 130 |
| | | | YAEEFKG | | | |
| MTK-07 | SGYYWN | 77 | YMSFDGDNKF | 101 | GGYNYGSTE | 127 |
| | | | NPSLKN | | AN | |
| MTK-08 | NYGMN | 75 | WINTYTGEPT | 99 | EVRYWYFDV | 131 |
| | | | YADDFKG | | | |
| MTK-09 | NHHIK | 79 | YIDPYNDYTT | 104 | RAYDGYYVD | 132 |
| | | | YNQNFKG | | WYFDV | |
| MTK-10 | TSGMGVG | 80 | HIWSDDDKRS | 105 | LTPVREFAY | 133 |
| | | | NPALKS | | | |
| MTK-11 | DYGVH | 81 | VIWSSGTTDY | 106 | KGHDPYAMD | 134 |
| | | | NEAFIS | | Y | |
| MTK-12 | SHWMH | 82 | MIDPSDGESR | 107 | GIYYYGITY | 135 |
| | | | LNQKFKD | | AMDY | |
| MTK-13 | SGYYWN | 77 | NIDYDGSNKY | 108 | DGGNYRSFA | 136 |
| | | | NPSLKN | | Y | |
| MTK-14 | NYGMN | 75 | WINTYTGEPT | 109 | GGHYAWFAY | 137 |
| | | | YADDFTG | | | |
| MTK-15 | NYGVH | 83 | WINTYTGEPT | 99 | GNRYAYMDY | 138 |
| | | | YADDFKG | | | |
| MTK-16 | NHGMN | 84 | WINTYTGEPT | 99 | KGVTAARYF | 139 |
| | | | YADDFKG | | DY | |
| MTK-17 | NFGMN | 85 | WINTYTGEPT | 99 | GAVLRAGAM | 140 |
| | | | YADDFKG | | DC | |
| MTK-18 | HYGMT | 86 | WINTYTGEPT | 99 | GGGQLGLRP | 141 |
| | | | YADDFKG | | LDY | |
| MTK-19 | EYTMH | 87 | GFNPNNVITS | 110 | GDLLWSLLL | 142 |
| | | | YNQRFQG | | PGNYFDY | |
| MTK-20 | NYGMT | 88 | WINTYTGEPT | 99 | GGGRLGLRP | 143 |
| | | | YADDFKG | | LDY | |
| MTK-21 | NFGMT | 89 | WINTYTGEPK | 111 | GGGRLGLRP | 143 |
| | | | YADDFKG | | LDY | |
| MTK-22 | SYWMH | 90 | EIDPSDSSSN | 112 | RYYYGSLYF | 144 |
| | | | YNQKFKG | | DN | |
| MTK-23 | NHHIN | 91 | NVDPYNDYST | 113 | RVYDGFYVD | 145 |
| | | | YNQKFKG | | WYFDV | |
| MTK-24 | SYWMH | 90 | MIHPNINTNY | 114 | RSPYSNYDW | 146 |
| | | | NEKFKS | | YFDV | |
| MTK-25 | DYYMA | 92 | NINYEGSSTY | 115 | YYYGSVDY | 147 |
| | | | YLGSLKS | | | |
| MTK-26 | SGYYWD | 93 | YISYDGSNNY | 116 | EGGYYRSMD | 148 |
| | | | NPSLKN | | Y | |
| MTK-27 | DDYMH | 94 | WIDPENGNTE | 117 | LFSNYFDY | 149 |
| | | | YASKFQG | | | |
| MTK-28 | TYGMS | 95 | WINTYSGVPT | 118 | FLRYYYFDY | 150 |
| | | | YADDFKG | | | |
| MTK-29 | TYGMS | 95 | WINTYSGVPT | 119 | YTNYGYFDY | 151 |
| | | | YTDDFKG | | | |
| MTK-30 | SYWMH | 90 | NLNPNNGGTY | 120 | QILLYFDY | 152 |
| | | | YNEKFKS | | | |
| MTK-31 | SYGMS | 96 | WINTYSGVPT | 118 | LSYDGSLYH | 153 |
| | | | YADDFKG | | AMDY | |
| MTK-32 | SYGMS | 96 | WINTYSGVPT | 121 | EDNWAWFAY | 154 |
| | | | YADDFRG | | | |
| MTK-33 | SYWMH | 90 | VIDPSDNYIN | 122 | EAGTRGYFD | 155 |
| | | | YNQKFKG | | Y | |
| MTK-34 | SYWMH | 90 | VIDPSDNYIN | 122 | EAGTRGYFD | 155 |
| | | | YNQKFKG | | Y | |
| MTK-35 | SYDIH | 97 | WIYPRDGYTK | 123 | AYYTNWYYF | 156 |
| | | | YNEIFKG | | DY | |
| MTK-36 | TFGMGVG | 98 | HIWWYDDKYY | 124 | IYYGTSYRY | 157 |
| | | | EPALKS | | FDV | |

TABLE 5

| | | SEQ | | SEQ | | SEQ |
|---|---|---|---|---|---|---|
| Antibody | HVR-L1 | ID NO: | HVR-L2 | ID NO: | HVR-L3 | ID NO: |
| MTK-01 | RASSSVS | 158 | ATSNLAS | 187 | QQWSSNPPT | 207 |
| | YMH | | | | | |
| MTK-02 | RANSSVS | 159 | ATSNLAS | 187 | QQWGSNPFT | 208 |
| | YMH | | | | | |
| MTK-03 | SASSSVN | 160 | STSNLAS | 188 | QQRSSYPFT | 209 |
| | YMH | | | | | |
| MTK-04 | RASSSVS | 158 | ATSNLAS | 187 | QQWSSNPRT | 210 |
| | YMH | | | | | |
| MTK-05 | RASENIF | 161 | NAKTLAE | 189 | LHHYGTPLT | 211 |
| | SYLA | | | | | |
| MTK-06 | RASSSVS | 158 | ATSNLAS | 187 | QQWSSKPPT | 212 |
| | YMH | | | | | |
| MTK-07 | SASSSVT | 162 | STSNLAS | 188 | QQRSSYPFT | 209 |
| | YMH | | | | | |
| MTK-08 | RATSSVS | 163 | ATSNLAS | 187 | QHWSGNPRT | 213 |
| | YMH | | | | | |
| MTK-09 | KASQDVT | 164 | SASYRYT | 190 | QQHYSTPLT | 214 |
| | TSVA | | | | | |
| MTK-10 | SASQGIS | 165 | YTSRLHS | 191 | QQYTKLPYT | 215 |
| | NYLN | | | | | |
| MTK-11 | TASSSIS | 166 | STSNLPS | 192 | HQYYRSPLT | 216 |
| | SSYFH | | | | | |
| MTK-12 | SASSSVS | 167 | DTSNLAS | 193 | QQWSSYPLT | 217 |
| | YMY | | | | | |
| MTK-13 | SASSSVS | 167 | STFNLAS | 194 | QQRSSYPFT | 209 |
| | YMY | | | | | |
| MTK-14 | RSSSSVS | 168 | ATSNLAS | 187 | QQWGSNPRT | 218 |
| | YMH | | | | | |
| MTK-15 | RASSSVS | 158 | ATSNLAS | 187 | QQWSSNPRT | 210 |
| | YMH | | | | | |
| MTK-16 | KASQNVR | 169 | LASNRHT | 195 | LQHWNYPLT | 219 |
| | TAVA | | | | | |
| MTK-17 | KASDHIN | 170 | GATSLET | 196 | QQYWSTPRT | 220 |
| | NWLA | | | | | |
| MTK-18 | RASQAIS | 171 | YTSRLHS | 191 | QQGNTLPWT | 221 |
| | NYLN | | | | | |
| MTK-19 | KSSQSLL | 172 | LVSKLDS | 197 | WQGTHFPWT | 222 |
| | DSDGKTY | | | | | |
| | LN | | | | | |
| MTK-20 | RASQDIN | 173 | YTSRLHS | 191 | QQGNTLPW | 223 |
| | NYLN | | | | | |
| MTK-21 | RASQDIN | 173 | YTSSLHS | 198 | QQGNTLPWT | 221 |
| | NYLN | | | | | |
| MTK-22 | KASQDVG | 174 | WASTRHT | 199 | QQYTSYPLT | 224 |
| | TAIA | | | | | |
| MTK-23 | KASRDVS | 175 | SASYRST | 200 | QQHYSAPLT | 225 |
| | TAVA | | | | | |
| MTK-24 | RASENIY | 176 | AATNLAD | 201 | QHFWGTPLT | 226 |
| | SNLA | | | | | |
| MTK-25 | KASQNVR | 177 | LASNRHT | 195 | LQLWNYPWT | 227 |
| | TTVA | | | | | |
| MTK-26 | SASSSVS | 178 | STSNLAS | 188 | QQRSSYPFT | 209 |
| | FMH | | | | | |
| MTK-27 | KASQNVR | 179 | LASNRHT | 195 | LQHWNYPYT | 228 |
| | TSVA | | | | | |
| MTK-28 | RASSSVT | 180 | ATSNLAS | 187 | HQWSGNPT | 229 |
| | YMH | | | | | |
| MTK-29 | RATSSVG | 181 | ATSNLAS | 187 | QQWGSNPFT | 208 |
| | YMH | | | | | |
| MTK-30 | RSSQSIV | 182 | KVSNRFS | 202 | FQGSHVPWT | 230 |
| | HSNGDTY | | | | | |
| | LE | | | | | |
| MTK-31 | RSSQTIV | 183 | KVSNRFS | 202 | FQGSHVPWT | 230 |
| | HSNGNTY | | | | | |
| | LE | | | | | |
| MTK-32 | RATSSVG | 181 | ATSNLAS | 187 | QQWSSNPRT | 210 |
| | YMH | | | | | |
| MTK-33 | KASQSVS | 184 | YASNRYT | 203 | QQDYRSPFT | 231 |
| | NTVA | | | | | |
| MTK-34 | KASQSVS | 184 | YASNRYT | 203 | QQDYRSPFT | 231 |
| | NTVA | | | | | |
| MTK-35 | RASSSIS | 185 | RTSNLAS | 204 | QQGSTIPLT | 232 |
| | SHYLH | | | | | |
| MTK-36 | RATSSVR | 186 | ATYNLTS | 205 | HQWSSNPYT | 233 |
| | YMH | | | | | |

### Example 10: Production of Anti-MerTK Antibodies

Anti-MerTK hybridoma clones were cultured in serum free hybridoma media and the anti-MerTK antibodies in the supernatants purified on Hamilton STAR platform (Hamilton Company, Reno, NV) using Protein A tips (Phynexus Inc, San Jose, CA). Anti-MerTK antibodies were also produced via direct cloning of the variable gene regions obtained from the hybridomas into a recombinant expression plasmid for production of chimeric antibodies containing a human Fc domain (human IgG1 containing LALAPS amino acid substitutions described above). Using the Tuna293™ Process (LakePharma, San Carlos, CA), HEK293 cell were seeded into shake flasks and expanded using serum-free chemically defined media. The expression plasmids were transiently transfected into the cells and the culture supernatants were harvested 7 days later. After clarification by centrifugation and filtration, the anti-MerTK antibodies in the supernatants were purified via Protein A chromatography.

### Example 11: Blocking Efferocytosis with Recombinant Anti-MerTK Antibodies

The ability of anti-MerTK antibodies of the present disclosure to block efferocytosis by phagocytic cells (e.g., human macrophages or mouse bone-marrow derived macrophages) was evaluated as follows. Human macrophages were differentiated from human monocytes for 7 days in the presence of human M-CSF. Mouse bone marrow derived macrophages were differentiated from mouse bone marrow cells for 7 days in the presence of mouse M-CSF (Peprotech, Cat #315-02). After 7 days, human macrophages and mouse macrophages were harvested (by scraping), resuspended in PBS, and plated on 96-well plates at 5×10⁴ cells/well. For efferocytosis IC50 determinations, cells were starved for 1 hour followed by the addition of 10 μl of recombinant anti-MerTK antibody to each well for 30 min at 37° C. For other studies described herein, recombinant anti-MerTK antibodies were titrated to a final concentration range of between 66.6 nM to 4 pM and then each serially diluted antibody was added to each well for 30 min at 37° C.

Jurkat cells were treated with 1 M staurosporin (SigmaAldrich) for 3 hours at 37° C. (to induce apoptosis) and labeled with pHrodo (ThermoFisher) for 30 min at room temperature. After washing with PBS, pHrodo labeled Jurkat cells were added into each well at 1:4 ratio (1 macrophage: 4 Jurkat cells) for 1 hour. The plates were washed with PBS and then the cells were stained with APC-conjugated anti-human CD14 or anti-mouse CD11b for 30 minutes on ice in the dark. Cells were then washed twice in FACS buffer (PBS+2% FBS), and flow cytometry was performed on a BD FACS Canto II. Data were analyzed using FlowJo software.

In addition to anti-MerTK antibodies of the present disclosure, the following anti-MerTK antibodies were also used in these efferocytosis studies: rat anti-mouse MerTK antibody DS5MMER (eBioscience, Clone ID DS5MMER, rat IgG2a), mouse anti-human MerTK antibody H1 (BioLegend, Clone ID: 590H11G1E3, mouse IgG1), mouse anti-human MerTK antibody H2 (R&D systems, Clone ID: 125518, mouse IgG2b), mouse anti-human MerTK antibody H3 (R&D systems, Clone ID 125508, mouse IgG2b), mouse anti-human MerTK antibody H6 (eBioscience, Clone ID: A3KCAT, mouse IgG1), mouse anti-human MerTK antibody H7 (Sino Biological, Clone ID: 09, Mouse IgG2b), human anti-human MerTK antibody M6 (disclosed in WO2016/106221, huIgG1 LALAPS), and human anti-human MerTK antibody CDX AB2000-A7 (disclosed in WO2019/084307, huIgG1).

In these experiments, efferocytosis-positive macrophages were identified by setting pHrodo CD14 double positive cells as an analysis gate and then applying this exact gate to all the samples. Baseline efferocytosis levels were established using macrophages cultured with media alone and this was set to 1000 efferocytosis activity. Relative efferocytosis levels were calculated as a percent of efferocytosis observed in cells treated with media alone compared to that observed in cells treated with anti-MerTK antibodies.

In comparison to macrophages in the absence of antibody (media alone treatment), no significant change in efferocytosis with isotype control human IgG1 LALAPS, mouse IgG1, or mouse IgG2b was observed.

Efferocytosis blocking studies were performed by addition of 10 μg/ml of recombinant anti-MerTK antibody of the present disclosure to the macrophages. Table 6 and Table 7 show 00 efferocytosis of anti-MerTK antibodies of the present disclosure on efferocytosis activity by macrophages obtained from various human donors.

TABLE 6

| | % Efferocytosis at 10 μg/mL | | |
|---|---|---|---|
| Antibody | donor 922 | donor 923 | donor 924 |
| MTK-01 | 14.45 | 12.45 | 18.20 |
| MTK-02 | 4.27 | 8.11 | 6.59 |
| MTK-03 | 11.64 | 14.51 | 14.55 |
| MTK-04 | 21.24 | 15.71 | 20.20 |
| MTK-05 | 34.72 | 27.78 | 47.61 |
| MTK-06 | 14.41 | 10.71 | 13.79 |
| MTK-07 | 11.98 | 14.35 | 15.75 |
| MTK-08 | 9.51 | 11.25 | 10.86 |
| MTK-09 | 9.42 | 17.54 | 16.06 |
| MTK-10 | 2.55 | 5.00 | 3.84 |
| MTK-11 | 5.99 | 6.56 | 6.10 |
| MTK-12 | 5.78 | 0.57 | 3.85 |
| MTK-13 | 4.04 | 8.03 | 5.43 |
| MTK-14 | 8.67 | 12.53 | 11.03 |
| MTK-15 | 5.57 | 8.30 | 8.50 |
| MTK-16 | 1.79 | 2.68 | 1.40 |
| MTK-17 | 3.43 | 4.35 | 3.98 |
| MTK-18 | 3.33 | 2.91 | 2.92 |
| MTK-19 | 4.31 | 4.15 | 3.41 |
| MTK-20 | 4.08 | 4.42 | 4.13 |
| MTK-21 | 4.08 | 3.13 | 3.28 |
| MTK-22 | 4.94 | 1.65 | 4.29 |
| MTK-23 | 3.12 | 3.83 | 3.98 |
| huIgG1 LALAPS | 114.35 | 96.22 | 131.70 |
| muIgG1 | 119.37 | 101.26 | 97.44 |
| muIgG2b | 121.88 | 111.35 | 112.12 |
| M6 | 4.52 | 1.04 | 1.22 |
| H1 | 7.87 | 21.38 | 23.36 |
| H2 | 8.96 | 13.50 | 16.60 |
| H3 | 88.8 | 111.35 | 104.56 |
| H6 | 103.04 | 119.11 | 124.14 |
| H7 | 108.9 | 100.1 | 99.22 |

TABLE 7

| | % Efferocytosis at 10 μg/mL | |
|---|---|---|
| Antibody | donor 930 | donor 929 |
| MTK-24 | 26.16 | 11.29 |
| MTK-25 | 30.93 | 41.66 |
| MTK-26 | 23.85 | 13.07 |
| MTK-27 | 35.26 | 17.57 |
| MTK-28 | 33.40 | 19.38 |
| MTK-29 | 41.75 | 20.49 |
| MTK-30 | 38.66 | 20.36 |
| MTK-31 | 22.59 | 18.02 |
| MTK-32 | 48.56 | 28.05 |
| MTK-33 | 24.90 | 21.63 |
| MTK-35 | 30.03 | 19.52 |
| MTK-36 | 21.68 | 21.43 |
| Media | 96.49 | 93.73 |
| M6 | 21.49 | 13.17 |
| H1 | 24.09 | 14.31 |

As shown in Table 6 and Table 7 above, anti-MerTK antibodies of the present disclosure reduced or decreased efferocytosis by human macrophages (e.g., reduced efferocytosis by about 50% to about 98%). Although the efferocytosis blocking activity by anti-MerTK antibodies was different depending on donors, there was a general trend of each antibody.

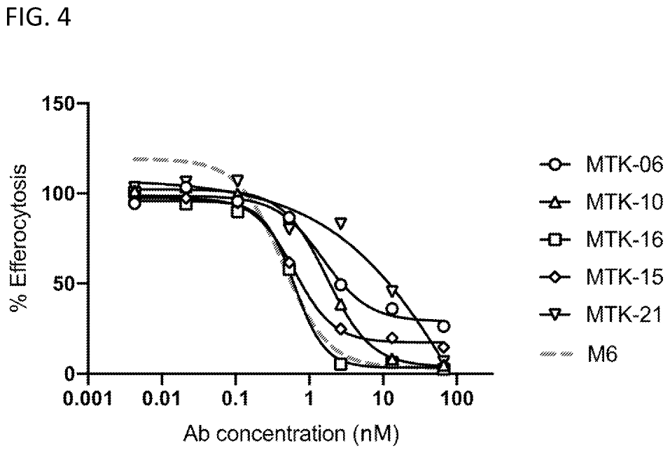

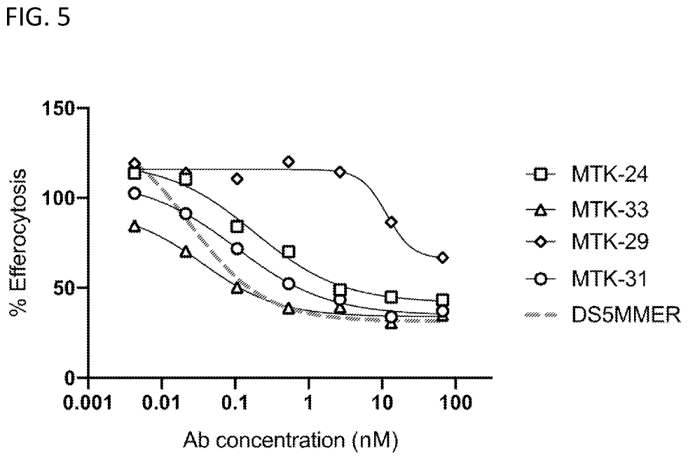

FIG. 4 and FIG. 5 shows efferocytosis blocking results of exemplary anti-MerTK antibodies of the present disclosure. As shown in FIG. 4, anti-MerTK antibodies MTK-06, MTK-10, MTK-15, MTK-16, and MTK-21 of the present disclosure inhibited efferocytosis of apoptotic cells by human macrophages in a dose dependent manner. These results indicated that anti-MerTK antibodies of the present disclosure are effective at reducing or decreasing efferocytosis activity by human macrophages.

As shown in FIG. 5, anti-MerTK antibodies MTK-24, MTK-29, MTK-31, and MTK-33 of the present disclosure inhibited efferocytosis of apoptotic cells by mouse bone marrow derived macrophages in a dose dependent manner. These results indicated that anti-MerTK antibodies of the present disclosure are effective at reducing or decreasing efferocytosis activity by mouse bone marrow derived macrophages.

Table 8 below shows IC50 values of anti-MerTK antibodies of the present disclosure for reducing or decreasing efferocytosis by human macrophages with a range of IC50 values. As shown in Table 8, anti-MerTK antibodies of the present disclosure reduced or decreased efferocytosis by human macrophages with an IC50 range of about 1.2 nM to about 28 nM.

TABLE 8

| | Efferocytosis |
|---|---|
| | IC50 (nM) |
| Antibody | donor 940 |
| MTK-01 | 6.91 |
| MTK-02 | 1.20 |
| MTK-03 | 14.16 |
| MTK-05 | 28.12 |
| MTK-06 | 0.77 |
| MTK-08 | 1.31 |
| MTK-10 | 1.45 |
| MTK-11 | 1.36 |
| MTK-13 | 4.90 |
| MTK-14 | 1.79 |
| MTK-15 | 0.86 |
| MTK-16 | 0.80 |
| MTK-17 | 7.76 |
| MTK-18 | 1.53 |
| MTK-19 | 10.50 |
| MTK-20 | 1.42 |
| MTK-21 | 14.11 |
| MTK-22 | 16.62 |
| M6 | 0.56 |

Table 9 and Table 10 show IC50 values of anti-MerTK antibodies of the present disclosure on efferocytosis activity by mouse macrophages and macrophages obtained from various human donors.

TABLE 9

| | Efferocytosis IC50 (nM) | | |
|---|---|---|---|
| Antibody | donor 673 | donor 960 | Mouse Mac |
| MTK-24 | 3.89 | 1.09 | 0.18 |
| MTK-26 | 0.54 | 0.43 | — |
| MTK-27 | 0.71 | 0.21 | — |
| MTK-28 | 0.15 | 0.08 | — |
| MTK-29 | 0.33 | 0.23 | 11.5 |
| MTK-30 | 0.45 | 0.13 | 0.10 |
| MTK-31 | 8.447 | 1.42E+24* | — |
| MTK-32 | 29.95 | 7.04 | — |
| MTK-33 | 0.25 | 0.33 | 0.03607 |
| MTK-35 | 19856 | 3.70 | — |
| MTK-36 | 0.41 | 0.44 | — |
| M6 | 0.11 | 0.12 | |
| CDX | n.a | 0.09402 | |
| AB2000-A7 | | | |
| DS5MMER | n.a | — | 0.02361 |

n.a. means data not available
*indicates aberrant value

TABLE 10
[nM]

| | donor | donor | donor | donor |
|---|---|---|---|---|
| Antibody | 993 | 996 | 896 | 897 |
| MTK-09 | 0.3418 | 0.7776 | 0.712 | 0.603 |
| MTK-23 | 0.1518 | 0.365 | 0.600 | 0.488 |
| MTK-25 | 0.1125 | 0.1584 | n.a | n.a |
| MTK-27 | 0.3386 | 0.5349 | n.a | n.a |
| MTK-31 | 0.9309 | 5.551 | n.a | n.a |
| MTK-36 | 0.128 | 0.2313 | n.a | n.a |
| CDX | 0.1099 | 0.2517 | n.a | n.a |
| AB2000-A7 | | | | |
| H1 | 0.4212 | 1.905 | 1.631 | 0.847 |
| H2 | 0.6921 | 1.474 | n.a | n.a |
| M6 | 0.04335 | 0.1086 | 0.222 | 0.213 |

n.a. means data not available

As shown in Table 8, Table 9, and Table 10 above, anti-MerTK antibodies of the present disclosure were effective at reducing efferocytosis by human macrophages and mouse macrophages. Additionally, these results showed that anti-MerTK antibodies of the present disclosure reduced efferocytosis by human macrophages at an IC50 of about 0.13 nM to about 30 nM.

### Example 12: Anti-MerTK Antibodies Bind to SK-MEL-5 Cells and CHO-muMerTK OE Cells

To determine whether recombinant anti-human MerTK antibodies of the present disclosure bind to human MerTK endogenously expressed on SK-MEL-5 cells and to CHO cells overexpressing mouse MerTK (CHO-muMerTK OE cells), the following experiments were performed. SK-MEL-5 cells and CHO-muMerTK OE cells were plated at 40,000 cells/well. Anti-MerTK antibodies were titrated to a final concentration range of between 66.6 nM to 4 pM and then added to the cells. After 30 min on ice, cells were washed and then stained with APC-conjugated mouse anti-human Kappa antibody (Southern Biotech Cat #9230-11, Birmingham, AL) and a viability die (Life Technologies) in the presence of Fc block solution on ice for 30 minutes, and then washed twice with cold FACS buffer (2% FBS in PBS). Cells were fixed with 4% paraformaldehyde in PBS. Stained cells were acquired on a BD FACS Canto II cytometer and the mean fluorescence intensity (MFI) was calculated with Flowlo software and K value was calculated with Prism software. Kd values in these results are similar to EC50 values but are calculated by Prism software from data generated by FACS analysis. A lower Kd value reflects higher binding of antibody to cells.

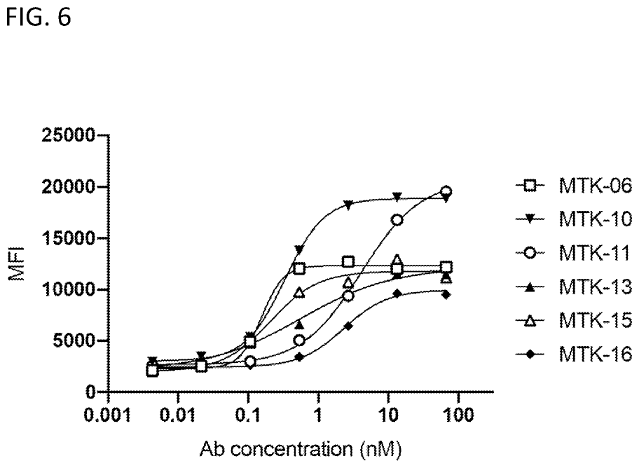

FIG. 6 shows anti-human MerTK antibodies MTK-06, MTK-10, MTK-11, MTK-13, MTK-15, and MTK-16 bind to MerTK endogenously expressed on SK-MEL-5 in a dose dependent manner.

Table 11, Table 12, and Table 13 below show Kd values from FACS analysis of anti-MerTK antibodies binding to SK-MEL-5 cells (Table 11 and Table 12) and to CHO-muMerTK-OE cells (Table 13).

TABLE 11

| Antibody | FACS Kd value (nM) |
|---|---|
| MTK-01 | 0.049 |
| MTK-02 | 0.024 |
| MTK-03 | 2.64 |
| MTK-04 | 0.037 |
| MTK-06 | 0.005 |
| MTK-07 | 3.21 |
| MTK-08 | 0.079 |
| MTK-10 | 0.076 |
| MTK-11 | 3.54 |
| MTK-12 | 0.009 |
| MTK-13 | 0.68 |
| MTK-14 | 0.021 |
| MTK-15 | 0.19 |
| MTK-16 | 1.66 |
| MTK-17 | 12.68 |
| MTK-18 | 0.38 |
| MTK-19 | 3.07 |
| MTK-20 | 0.11 |
| MTK-21 | 23.36 |
| MTK-22 | 2.78E−06* |
| M6 | 0.27 |
| H1 | 0.22 |
| H2 | 0.03 |

*indicates aberrant value

TABLE 12

| Antibody | FACS Kd value (nM) |
|---|---|
| MTK-24 | 0.59 |
| MTK-26 | 1.38 |
| MTK-27 | 2.55 |
| MTK-28 | 0.52 |
| MTK-29 | 0.14 |
| MTK-30 | 0.47 |
| MTK-31 | 2.311E−11* |
| MTK-32 | 0.40 |
| MTK-33 | 0.93 |
| MTK-35 | 1.29 |
| MTK-36 | 1.42 |
| M6 | 5.302E−13* |
| CDX | 0.19 |
| AB2000-A7 | |
| H1 | 0.06436 |
| H2 | 2.455E−08* |

*indicates aberrant value

TABLE 13

| Antibody | FACS Kd value (nM) |
|---|---|
| MTK-24 | 0.22 |
| MTK-29 | 5.22 |
| MTK-30 | 0.24 |
| MTK-31 | 2.913 |
| MTK-32 | 2.455E−08* |
| MTK-33 | 0.53 |

*indicates aberrant value

Anti-MerTK antibodies binds to SK-MEL-5 or CHO-muMerTK overexpressing cells in a dose dependent manner with Kd value in the picomolar to nanomolar range. Anti-MerTK antibody such as MTK-06 or MTK-12 showed better binding to SK-MEL-5 compared to M6 antibody. Data in Tables 11, 12, and 13 above also show that anti-MerTK antibodies MTK-24, MTK-29, MTK-30, MTK-31, and MTK-33 bind to human MerTK endogenously expressed on human SK-MEL-5 cells and to murine MerTK recombinantly expressed in CHO cells.

### Example 13: Anti-MerTK Antibody Blocking of Ligand Gas6 and Ligand ProS Binding to MerTK

To determine the ligand blocking activity of anti-MerTK antibodies of the present disclosure, ligand blocking assays was performed to test whether a given anti-MerTK antibody blocks binding of ligand Gas6 or ligand ProS to human MerTK.

Rabbit anti-human IgG polyclonal antibody (Jackson ImmunoResearch, Cat #309-005-008) was captured to a high-protein binding plate at 2 g/ml 4° C. overnight. After washing with 0.05% Tween20 in PBS three times, 5% BSA in PBS was added for 1 hour. Recombinant human MerTK human Fc Chimera protein (R&D systems, Cat #391-MR-100) was added at 2 g/ml for 1 hour and the plates were then washed. Anti-MerTK antibodies were titrated to final concentration range of between 66.6 nM to 4 pM and then 40 μl of serially diluted antibody and 40 μl of His tag-conjugated recombinant Gas6 protein (R&D systems, Cat #885-GSB-050) at 3 g/ml or His tag-conjugated recombinant ProS protein (R&D systems, Cat #9489-PS-100) at 20 g/ml. After a further incubation for 1 hour, plates were washed and incubated for 1 hour with HRP-conjugated anti-6× His tag antibody (Abcam, Cat #ab1187). Plates were then washed and an HRP substrate (TMB) was used to develop the plates. After sufficient color change was observed, the reaction was stopped by adding 50.1 2N H₂SO₄ and the OD was measured using a spectrophotometer (BioTek).

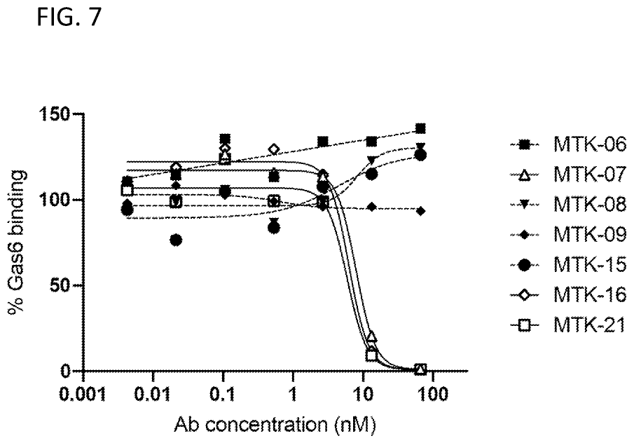

FIG. 7 shows that exemplary anti-MerTK antibodies of the present disclosure (MTK-07, MTK-16, and MTK-21) inhibited (e.g., blocked) Gas6 ligand binding to human MerTK in a dose dependent manner. As shown in FIG. 7, anti-MerTK antibodies MTK-06, MTK-08, MTK-09, and MTK-15 did not block Gas6 ligand binding to human MerTK.

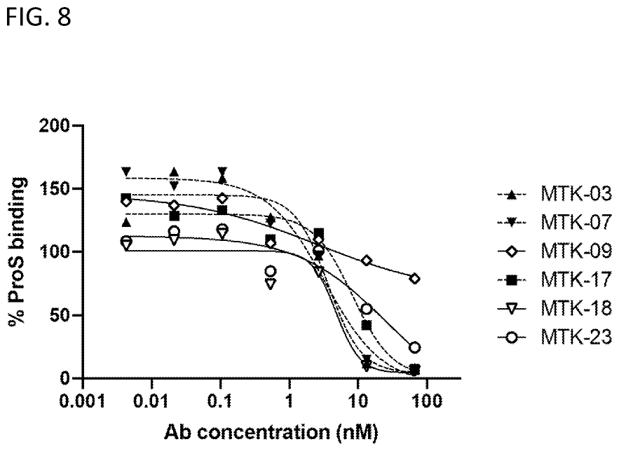

FIG. 8 shows that exemplary anti-MerTK antibodies of the present disclosure (MTK-03, MTK-07, MTK-09, MTK-17, MTK-18, and MTK-23) inhibited (e.g., blocked) ProS ligand binding to human MerTK in a dose dependent manner.

From these experiments, IC50 values [nM] were determined using OD450 values obtained from the dose-response curves analyzed with Prism software. Table 14 and Table 15 show IC50 values determined for anti-MerTK antibody blocking of Gas6 ligand binding and ProS ligand binding to MerTK. Anti-MerTK antibody MTK-22 did not block Gas6 ligand binding or ProS ligand biding to MerTK; however, anti-MerTK antibody MTK-22 was effective at reducing efferocytosis as shown in Examples above. Dashes indicate lack of significant blocking; “n.a.” indicates data not available.

TABLE 14

| | Gas6 ligand | ProS ligand |
|---|---|---|
| Antibody | blocking IC50 | blocking IC50 |
| MTK-01 | — | 2.85 |
| MTK-02 | — | 3.94 |
| MTK-03 | 8.53 | 3.65 |
| MTK-04 | — | 12.81 |
| MTK-05 | — | 6.51 |
| MTK-06 | — | 2.17 |
| MTK-07 | 7.97 | 3.27 |
| MTK-08 | — | 3.80 |
| MTK-09 | — | 7.84 |
| MTK-10 | 32.38 | 3.80 |
| MTK-11 | — | 24.24 |
| MTK-12 | 25.97 | 3.36 |
| MTK-13 | 18.93 | 3.76 |
| MTK-14 | n.a. | 7.61 |
| MTK-15 | — | 4.20 |
| MTK-16 | 6.32 | 8.59 |
| MTK-17 | 14.47 | 8.35 |
| MTK-18 | 4.22 | 4.76 |
| MTK-19 | n.a. | 11.27 |
| MTK-20 | 4.96 | 5.16 |
| MTK-21 | 5.98 | 13.50 |
| MTK-23 | — | 25.92 |

TABLE 15

| | Gas6 blocking | ProS blocking |
|---|---|---|
| Antibody | IC50 | IC50 |
| MTK-24 | 0.74 | 0.5792 |
| MTK-26 | 2.93 | 1.203 |
| MTK-27 | 1.42 | 2.543 |
| MTK-28 | — | 2.781 |
| MTK-29 | — | 2.664 |
| MTK-30 | 1.26 | 1.572 |
| MTK-31 | 1.29 | 1.51E+15* |
| MTK-32 | — | 2.781 |
| MTK-33 | n.a. | 0.6005 |
| MTK-35 | 0.31 | 0.924 |
| MTK-36 | 0.29 | 1.402 |
| M6 | 0.28 | 1.339 |
| CDX AB2000-A7 | 0.49 | 2.514 |
| H1 | — | 2.513 |

*indicates aberrant value

In these studies, thirty-five (35) anti-MerTK antibodies of the present disclosure were tested for their ability to block Gas6 ligand binding and/or ProS ligand binding to human MerTK. Nineteen (19) anti-MerTK antibodies of the present disclosure (MTK-03, MTK-07, MTK-10, MTK-12, MTK-13, MTK-16, MTK-17, MTK-18, MTK-20, MTK-21, MTK-24, MTK-25, MTK-26, MTK-27, MTK-30, MTK-31, MTK-33, MTK-35, and MTK-36) blocked the binding of both ProS ligand and Gas6 ligand to recombinant human MerTK protein (herein referred to as ProS/Gas6 double blockers); fifteen (15) anti-MerTK antibodies of the present disclosure (MTK-01, MTK-02, MTK-04, MTK-05, MTK-06, MTK-08, MTK-09, MTK-11, MTK-14, MTK-15, MTK-19, MTK-23, MTK-28, MTK-29, MTK-32) blocked ProS ligand binding (but did not block Gas6 ligand binding) to recombinant human MerTK (herein referred to as ProS specific blockers). Anti-MerTK antibody MTK-22 did not block either ProS ligand or Gas6 ligand binding to recombinant human MerTK protein as measured in this assay; however, anti-MerTK antibody MTK-22 was effective at reducing efferocytosis as shown in Examples above. Anti-MerTK antibody M6 and anti-MerTK antibody CDX AB2000-A7 displayed ProS ligand and Gas6 ligand blocking. Anti-MerTK antibody H1 and anti-MerTK antibody H2 displayed ProS ligand blocking only; neither of these antibodies displayed Gas6 ligand blocking.

These results suggested that anti-MerTK antibodies of the present disclosure can selectively modulate Gas6 and ProS binding to MerTK and can selectively modulate Gas6 and ProS activity.

### Example 14: Binding Kinetics of Anti-MerTK Antibodies

Binding kinetics of human anti-MerTK IgG1 LALAPS antibodies of the present disclosure to human, cyno, and murine MerTK were evaluated using a Carterra LSA instrument (Carterra, Salt Lake City, UT). Briefly, anti-MerTK antibodies were prepared by diluting to 10 μg/ml in 10 mM Acetate, pH 4.25 (Carterra), at 300 μl/well. A HC200M sensor chip (Carterra) was activated using the single channel flow cell with a 7-minute injection of a 1:1:1 mixture of 100 mM MES pH 5.5, 100 mM sulfo-NHS, 400 mM EDC (all reconstituted in MES pH 5.5; 100 μl of each mixed in vial immediately before running assay). After switching to the multi-channel array flow cell, the antibodies were injected over the activated chip in a 96-spot array for 15 minutes. The remaining unconjugated active groups on the chip were then blocked by injecting 1M Ethanolamine pH 8.5 (Carterra) for 7 minutes using the single channel flow cell.

After priming with running buffer (HBS-TE, Carterra) with 0.5 mg/ml BSA (Sigma), the immobilized anti-MerTK antibodies were tested for their ability to bind to several forms of recombinant MerTK extracellular domain, including human, cynomolgus, and mouse orthologs as described above. Estimates of affinity were generated by injecting each analyte over the entire antibody array using the single channel flow cell. MerTK analytes were diluted to 33.3, 100, and 300 nM in running buffer, and injected in serial from lowest to highest concentration without regeneration. Two buffer blanks were run between each series (one species per series). Data were processed and analyzed using NextGenKIT high-throughput kinetics analysis software (Carterra).

The equilibrium dissociation constants (K_{D}) were then calculated from the fitted association and dissociation rate constants (k-on and k-off) for anti-MerTK antibodies of the present disclosure. Binding kinetic analysis was also performed on anti-MerTK antibodies H1 (BioLegend, Clone ID: 590H11G1E3), H2 (R&D systems, Clone ID: 125518), M6 (WO2016/106221), and CDX AB2000-A7 hIgG1 (WO2019/084307). The K_{D} values are summarized in Table 16 below.

TABLE 16

| | K_{D} (nM) | K_{D} (nM) | K_{D} (nM) |
|---|---|---|---|
| Antibody | human | cyno | mouse |
| MTK-01 | NF | 99 | NB |
| MTK-02 | 22 | 24 | NB |
| MTK-03 | 54 | 55 | NB |
| MTK-04 | 33 | 44 | NB |
| MTK-05 | 6.7 | NB | NB |
| MTK-06 | 18 | 17 | NB |
| MTK-07 | 43 | 53 | NB |
| MTK-08 | 37 | 42 | NB |
| MTK-10 | 2.0 | 1.9 | NB |
| MTK-11 | 3.0 | 2.9 | NB |
| MTK-12 | 5.3 | NB | NB |
| MTK-13 | 37 | 39 | NB |
| MTK-14 | 45 | 53 | NB |
| MTK-15 | 2.7 | 3.0 | NB |
| MTK-16 | 1.4 | 1.6 | NB |
| MTK-17 | 25 | 24 | NB |
| MTK-18 | 18 | 18 | NB |
| MTK-19 | 2.4 | 12 | 130 |
| MTK-20 | 9.3 | 9.2 | NB |
| MTK-21 | 40 | 43 | NB |
| MTK-22 | 54 | LB | NB |
| MTK-24 | 41 | 44 | 55 |
| MTK-26 | 30 | 30 | NB |
| MTK-27 | 71 | 71 | NB |
| MTK-28 | 4.1 | 4.5 | NB |
| MTK-30 | 23 | 24 | 30 |
| MTK-31 | 81 | 91 | 165 |
| MTK-32 | 59 | 107 | 186 |
| MTK-33 | 6.5 | 6.6 | 75 |
| MTK-35 | 14 | 13 | NB |
| MTK-36 | 6.4 | 5.9 | NB |
| CDX AB2000-A7 | 38 | 52 | NB |
| H1 | 7.2 | 6.8 | NB |
| H2 | 18 | 18 | NB |
| H3 | 151 | LB | NB |
| H6 | 26 | 116 | NB |
| H7 | 6.1 | 10 | NB |
| M6 | 5.6 | 5.4 | NB |

NB = No binding; LB = Low binding; NF = No fit, meaning the data obtained did not fit a 1:1 binding equilibrium model.

These results showed that anti-MerTK antibodies of the present disclosure showed a range of affinities to MerTK and species binding specificity. In particular, affinity of anti-MerTK antibodies of the present disclosure for binding to human MerTK ranged from 1.4 nm to 81 nM; affinity of anti-MerTK antibodies of the present disclosure for binding to cyno MerTK ranged from 1.6 nm to 107 nM; and affinity of anti-MerTK antibodies of the present disclosure for binding to murine MerTK ranged from 30 nM to 186 nM.

### Example 15: Cross-Reactivity of Anti-MerTK Antibodies to Human, Cyno, and Mouse MerTK

Species cross-reactivity of anti-MerTK antibodies of the present disclosure was determined from the binding kinetic analysis data described above. The results of species binding cross-reactivity analysis are shown below in Table 17.

TABLE 17

| Human | | Human-Mouse |
|---|---|---|
| MerTK | Human-Cyno MerTK | MerTK |
| Reactive | Cross Reactive | Cross Reactive |
| MTK-05 | MTK-01, MTK-02, MTK-03, | MTK-19, MTK-24, |
| MTK-12 | MTK-04, MTK-06, MTK-07, | MTK-30, MTK-31, |
| | MTK-08, MTK-09, MTK-10, | MTK-32, MTK-33 |
| | MTK-11, MTK-13, MTK-14, | |
| | MTK-15, MTK-16, MTK-17, | |
| | MTK-18, MTK-19, MTK-20, | |
| | MTK-21, MTK-22, MTK-24, | |
| | MTK-26, MTK-27, MTK-28, | |
| | MTK-30, MTK-31, MTK-32, | |
| | MTK-33, MTK-34, MTK-35, | |
| | MTK-36 | |
| | CDX AB2000-A7, H1, H2, | |
| | H7, M6 | |
| | Weak cross reactivity: | |
| | H3, H6 | |

These binding experiments showed that the majority of the anti-MerTK antibodies of the present disclosure tested showed binding cross-reactivity to both human and cyno MerTK (MTK-01, MTK-02, MTK-03, MTK-04, MTK-06, MTK-07, MTK-08, MTK-09, MTK-10, MTK-11, MTK-13, MTK-14, MTK-15, MTK-16, MTK-17, MTK-18, MTK-19, MTK-20, MTK-21, MTK-22, MTK-24, MTK-26, MTK-27, MTK-28, MTK-30, MTK-31, MTK-32, MTK-33, MTK-34, MTK-35, and MTK-36). Two anti-MerTK antibodies of the present disclosure showed specific binding only to human MerTK and did not show binding to cyno or mouse MerTK (antibody MTK-05 and antibody MTK-12). Six anti-MerTK antibodies of the present disclosure tested displayed cross-reactivity to both human and mouse MerTK (MTK-19, MTK-24, MTK-30, MTK-31, MTK-32, and MTK-33). Six anti-MerTK antibodies of the present disclosure tested displayed cross-reactivity to human, cyno, and mouse MerTK (MTK-19, MTK-24, MTK-30, MTK-31, MTK-32, and MTK-33). Additionally, Example 12 above showed that anti-MerTK antibodies MTK-24 and MTK-29 also bind to human and murine MerTK.

### Example 16: Epitope Binning Analysis of Anti-MerTK Antibodies

Epitope binning analysis was performed on the anti-MerTK antibodies of the present disclosure by performing a tandem injection approach using a Carterra LSA instrument (Carterra, Salt Lake City, UT). Briefly, hybridoma purified mouse anti-MerTK antibodies of the present disclosure, anti-MerTK antibodies H1, H2, H3, H6, and M6, and anti-his IgG were diluted to 15 μg/ml in 10 mM Acetate, pH 4.75 (Carterra), at 300 μl/well. A second set of samples was prepared by 5-fold dilution of the antibodies to 3 μg/ml in the same buffer. A HC200M sensor chip (Carterra) was activated using the single channel flow cell with a 7-minute injection of a 1:1:1 mixture of 100 mM MES pH 5.5, 100 mM sulfo-NHS, 400 mM EDC (as described above). After switching to the multi-channel array flow cell, the 15 μg/ml dilutions of antibodies were injected over the activated chip in a 96-spot array for 15 minutes. A second array was printed by injection of the 3 μg/ml dilutions of the antibodies in a second 96-spot array. The remaining unconjugated active groups on the chip were then blocked by injecting 1M Ethanolamine pH 8.5 (Carterra) over the entire chip surface for 7 minutes using the single channel flow cell.

After priming with running buffer (HBS-TE, Carterra) containing 0.5 mg/ml BSA (Sigma), the immobilized antibodies were tested for their ability to bind to recombinant human MerTK extracellular domain. Bound MerTK was removed by two 30-second injections of 0.425% phosphoric acid (Carterra). For each cycle, MerTK was injected over the chip, followed by a test antibody diluted to 30 μg/ml in running buffer. Data were processed and analyzed using Epitope high-throughput binning analysis software (Carterra). Antibodies which were able to bind antigen captured by an immobilized antibody were designated as “sandwich” or “pairing” antibodies, and these antibodies were assigned into a different epitope bin from that of the immobilized antibody. A matrix of pairing and non-pairing antibodies was constructed from the binding results of these experiments, which allowed for an epitope bin landscape of the anti-MerTK antibodies to be generated.

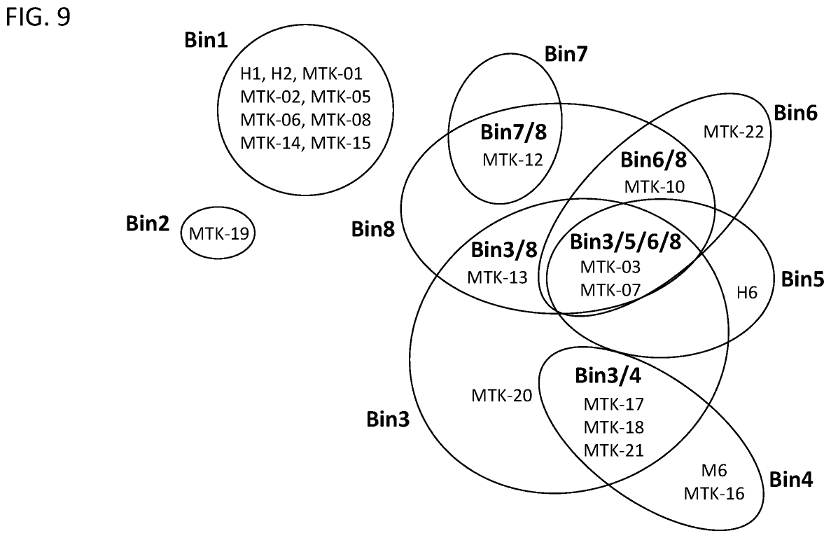

The epitope bins identified from these studies for the anti-MerTK antibodies are summarized in FIG. 9. The results showed that most of the ProS ligand-specific blockers belong to Bin 1 group except MTK-19, which is included in Bin 2. Anti-MerTK control antibody H1, a ProS-specific blocker belongs to Bin 1 group. All the ProS/Gas6 double blockers belong to Bin 3 through Bin 8 groups. Some of antibodies recognize partially adjacent epitopes, so have overlapping binning. For example, MTK-03 and MTK-07 have most broad overlapping epitopes of Bin 3, Bin 5, Bin 6, and Bin 8. Anti-MerTK control antibody M6, a ProS/Gas6 double blocker belongs to Bin 4 and H6 belongs to Bin 5 group. These results suggested that ProS and Gas6 may bind to different epitopes or regions on MerTK.

### Example 17: Anti-MerTK Antibody Inhibition of Tumor Growth In Vivo

An in vivo efficacy study was performed using MC38 mouse syngeneic colon cancer model to determine the effect of anti-MerTK antibodies on tumor growth. MC38 cells were resuspended at 5×10⁶ cells/ml in PBS. 100 μl of cells was injected subcutaneously on the shaved right flank of C57BL/6 mice. When tumor size reached approximately 60-100 mm³ in volume, mice were randomized based on tumor volumes into treatment (N=10) or control groups. Treatment groups received 10 mg/kg of control antibodies, 3 mg/kg of anti-PDL1 (clone: BM1) alone, 10 mg/kg of anti-MerTK antibody DS5MMER alone, or 3 mg/kg of anti-PDL1 μlus anti-MerTK antibody DS5MMER together. Following the initiation of antibody dosing, mice were weighed, and tumor volume was measured using a caliber twice per week. The study was terminated when tumors in the control group reached approximately 2000 mm³.

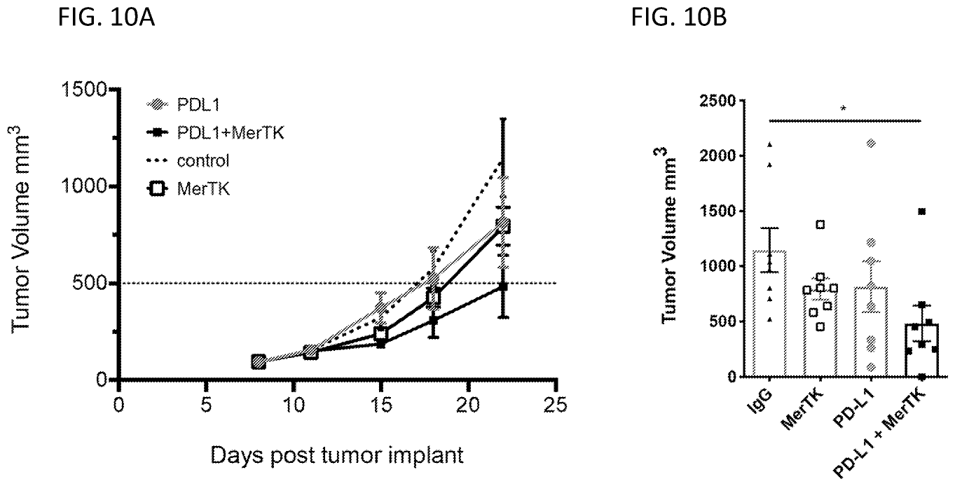

FIG. 10A and 10B show that either anti-MerTK antibody DS5MMER treatment alone or combination treatment with anti-PDL1 antibody reduced tumor growth in vivo. These results indicated that anti-MerTK antibodies are effective at reducing tumor volume and delaying tumor growth in vivo. These results further showed that anti-MerTK antibodies, when used in combination with an anti-PD-L1 antibody, displayed greater reduction of tumor volume and growth compared to either antibody alone.

### Example 18: Modulating Pro-Inflammatory Cytokine Production

The effect of anti-MerTK antibodies on pro-inflammatory cytokine production in myeloid cells was examined as follows. To generate monocyte-derived macrophages and dendritic cells, human primary monocytes were isolated from heparinized human blood (Blood Centers of the Pacific) using RosetteSep Human Monocyte Enrichment Cocktail (STEMCELL Technologies), according to the manufacturer's protocol. Monocytes were seeded in RPMI (Invitrogen) containing 10% Fetal Bovine Serum (Hyclone) and 50 ng/mL IL-4 and GM-CSF (Peprotech) to induce differentiation of dendritic cells or 50 ng/mL M-CSF (Biolegend) to induce differentiation of macrophages. After 6-7 days, dendritic cells or macrophages were harvested by scraping cells. Macrophages or dendritic cells were plated on 96-well plates at 5×10⁴cells/well and cultured with 10 g/mL of antibody at 37° C. for overnight. The following day, supernatants were collected and a 13-plex human proinflammatory chemokine and human macrophages/microglia LEGENDplex bead array (BioLegend) was used according to the manufacturer's protocol. All samples were run on BD FACS Canto II and analysis was performed using LEGENDplex analysis software (BioLegend).

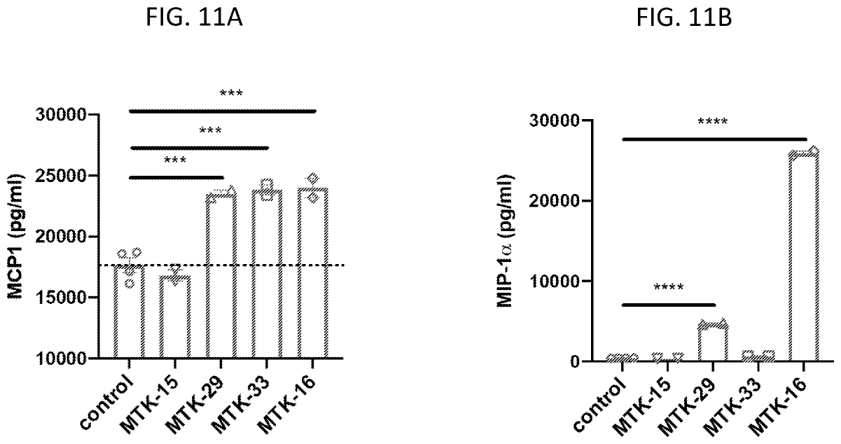

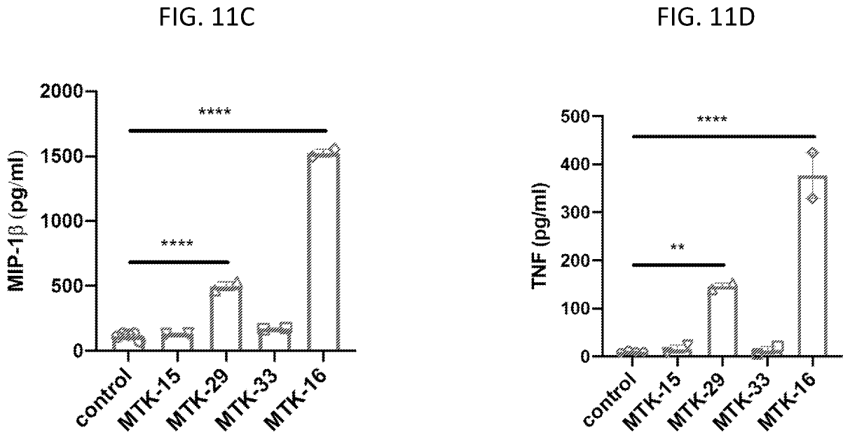

FIGS. 11A, 11B, 11C, and 11D show changes in the level of MCP1 (CCL2), MIP-1α(CCL3), MIP-1β (CCL4), and TNF, respectively, from human macrophages following overnight treatment with anti-MerTK antibody MTK-15, MTK-16, MTK-29, or MTK-33. Anti-MerTK antibodies MTK-16 and MTK-29 markedly increased the level of MCP1, MIP-1α, MIP-1P, and TNF in human macrophages. The level of MCP1 significantly increased in response to anti-MerTK antibody MTK-33 compared to that observed upon treatment with control antibody. These results demonstrated that anti-MerTK antibodies MTK-15, MTK-16, MTK-29, and MTK-33 induce pro-inflammatory chemokine or cytokine production in human macrophages in a MerTK-dependent way. These results further showed that anti-MerTK antibodies of the present disclosure are effective at increasing pro-inflammatory cytokines and chemokines in myeloid cells, indicative that anti-MerTK antibodies of the present disclosure are effective at activating myeloid cells (e.g, macrophages). These results also showed that anti-MerTK antibodies of the present disclosure are effective at increasing M1-like macrophage polarization and thus mediating anti-tumor immunity.

### Example 19: Effect of Anti-MerTK Antibody Treatment in Colon Cancer In Vivo

The following studies were performed to test the ability of anti-MerTK antibodies of the present disclosure to reduce tumor burden in vivo. In these experiments, 400,000 MC38 mouse colon cancer cells were implanted subcutaneously on the shaved right flank of C57BL/6 mice. When tumor size reached approximately 80-120 mm³ in volume, mice were grouped based on tumor volume to ensure the distribution of starting tumor volume was similar for analyses purposes. Anti-MerTK antibody MTK-29 and MTK33, or control antibody, were administrated via intraperitoneal injection at a dose of 10 mg/kg, twice a week (N=9 per group). At the same time, anti-PDL1 antibody (clone BM1) was administered intraperitoneally at 3 mg/kg to all the treatment groups twice a week. Tumor volume was measured three times per week using a caliber. Mice were humanely euthanized when either tumor volume reached approximately 2000 mm³ or ulceration occurred. Two independent in vivo experiments were performed in these studies.

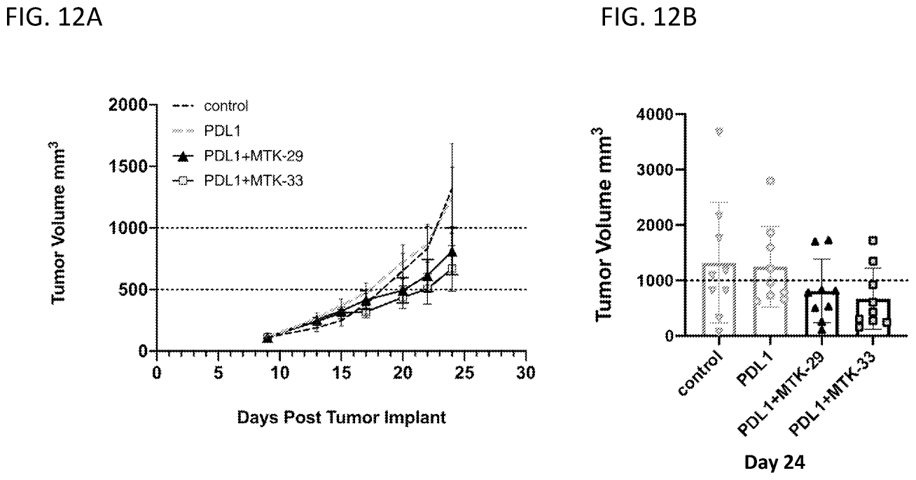

FIG. 12A and FIG. 12B show that treatment of mice with anti-PDL1 antibody and either anti-MerTK antibody MTK-29 or MTK-33 reduced tumor growth rate (shown as tumor volume over time) compared to that observed in mice treated with either anti-PDL1 antibody alone or with control antibody. These results showed that anti-MerTK antibody MTK-29 and MTK-33 are synergistic at reducing tumor volume (reduced tumor growth rate) when administered in combination with anti-PDL1 antibody compared to that observed upon treatment with anti-PDL1 antibody alone.

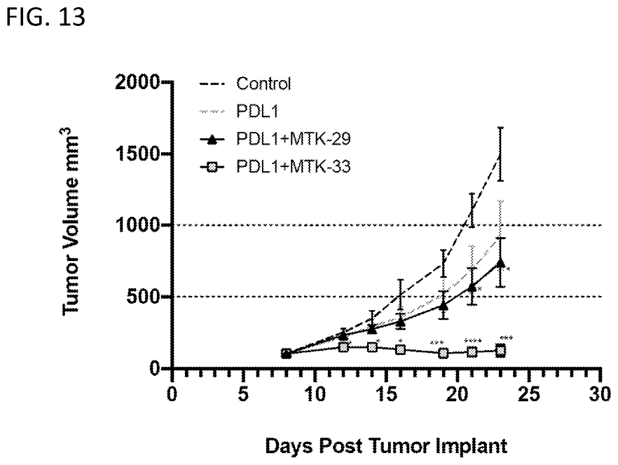

A separate set of in vivo experiments was also performed, using a combination of anti-PDL1 antibody and anti-MerTK antibody MTK-29 or anti-MerTK antibody MTK-33. As shown in FIG. 13, administration of anti-PDL1 antibody lead to reduced tumor volume compared to that observed with control antibody treatment. Administration of a combination of anti-PDL1 antibody and either anti-MerTK antibody MTK-29 or MTK-33 resulted in a greater reduction in tumor volume compared to mice administered anti-PDL1 antibody alone. Specifically, anti-MerTK antibody MTK-29 administered in combination with anti-PDL1 antibody resulted in a significantly greater (p≤0.05) reduction in tumor growth rate compared to that observed in mice treated with anti-PDL1 antibody alone. Treatment with anti-PDL1 antibody in combination with anti-MerTK antibody MTK-33, in particular, significantly reduced the growth rate of MC38 colon cancer cells in these studies compared to that observed in mice treated with anti-PDL1 antibody alone. Animals treated with 20 mg/kg of anti-MerTK antibody MTK-33 as mono-therapy also showed a reduction in tumor volume compared to that observed in animals treated with control antibody (data not shown).

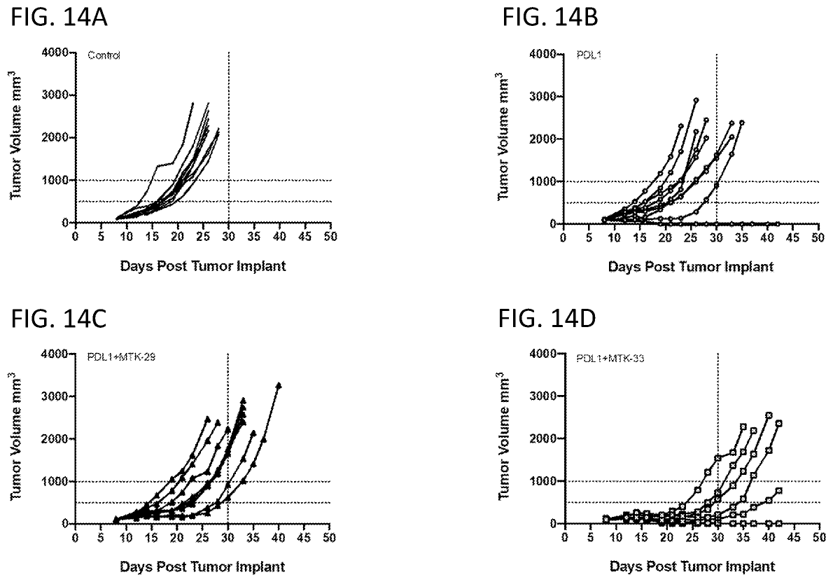

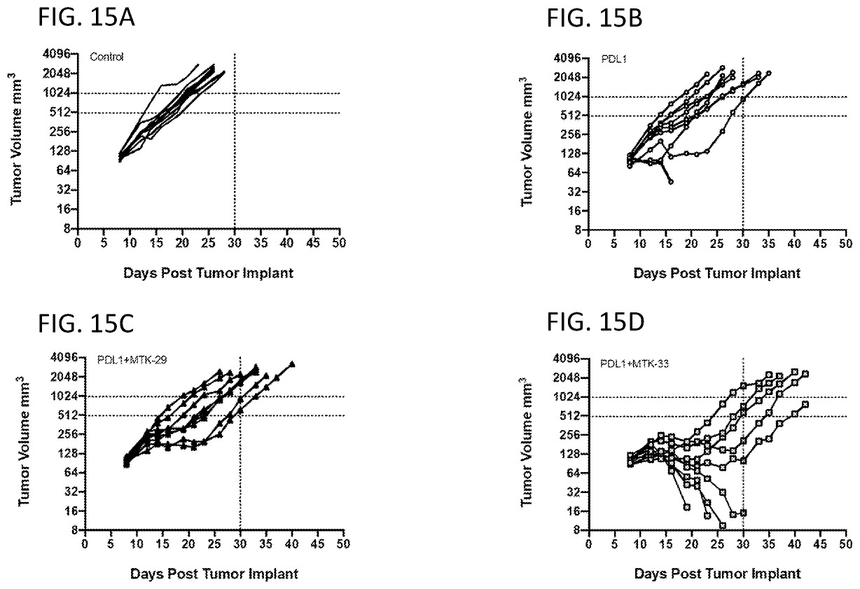

FIGS. 14A, 14B, 14C, and 14D (as well as FIGS. 15A, 15B, 15C, and 15D) show the differences in tumor growth rate (tumor volume overtime) of individual mice in control, anti-PDL1 antibody alone, anti-PDL1 antibody in combination with anti-MerTK antibody MTK-29, and anti-PDL1 antibody in combination with anti-MerTK antibody MTK-33, respectively. FIGS. 14A, 14B, 14C, and 14D are plotted using a linear scale on the y-axis. FIGS. 15A, 15B, 15C, and 15D are these same results plotted using a Log2 scale on the y-axis. As shown in these Figures, combination treatment with anti-PDL1 antibody and either anti-MerTK antibody MTK-29 or MTK-33 resulted in near complete regression of tumors in 4 out of 9 mice examined in these studies.

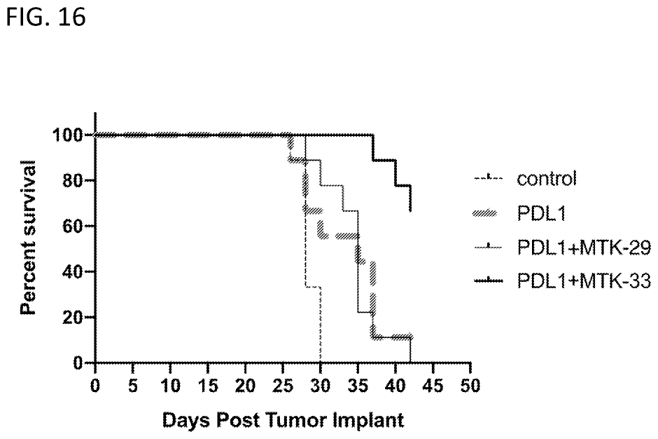

FIG. 16 shows a survival curve of the tumor-bearing mice from these studies. Median survival time of mice administered control antibody was 28 days; median survival time of mice administered anti-PDL1 antibody alone was 35 days; median survival time of mice administered anti-PDL1 antibody in combination with anti-MerTK antibody MTK-29 was 35 days; and median survival time of mice administered anti-PDL1 antibody in combination with anti-MerTK antibody MTK-33 extends beyond the final 42-day post tumor implant time-point: 6 out of 9 mice were still alive on day 42 following this combination treatment. Taken together, the results presented in FIGS. 12A, 12B, 13, 14A, 14B, 14C, 14D, 15A, 15B, 15C, 15D, and 16 demonstrated that anti-MerTK antibodies MTK-29 and MTK-33 in combination with anti-PDL1 antibody have enhanced efficacy in inhibiting tumor growth and improving cancer immunotherapy compared to treatment with anti-PDL1 antibody alone.

### Example 20: Modulation of Pro-Inflammatory Cytokine or Chemokine Production in Mice

The effect of anti-MerTK antibodies of the present disclosure on pro-inflammatory cytokine or chemokine production in mice was examined as follows. Control antibody or anti-MerTK antibody MTK-24, MTK-29, MTK-30, or MTK-33 was administered to C57BL/6 mice via intraperitoneal injection at a dose of 20 mg/kg (N=5 per group). One hour later, blood was withdrawn from each animal for use in measuring changes in pro-inflammatory cytokine or chemokine levels. LEGENDplex mouse proinflammatory chemokine panel (Biolegend, Catalog #740451) and LEGENDplex mouse macrophage/microglia panel (Biolegend, Catalog #740846) were used according to the manufacturer's instructions as follows. Briefly, serum samples from the mice were diluted 4-fold with Assay Buffer. Standard Cocktail was serially diluted with Assay Buffer. For setting up the standard, 25 μl of Matrix B, 25 μl of standard solution, and 25 μl of Mixed Beads were added. For setting up sample wells, 25 μl of Assay Buffer, 25 μl of diluted serum samples, and 25 μl of Mixed Beads were added. Each plate was placed on a plate shaker at 500 rpm for 2 hours. After washing the plates, 25 μl of Detection Antibodies was added to each well, and then plate was placed on a plate shaker for 1 hour. 25 μl of SA-PE was added, and then plate was shaken for 30 minutes. After washing, samples were resuspended with 1× Wash Buffer and acquired on a BD FACS CantoII (Becton Dickinson, San Jose, CA) and data were analyzed with LEGENDplex software (Biolegend).

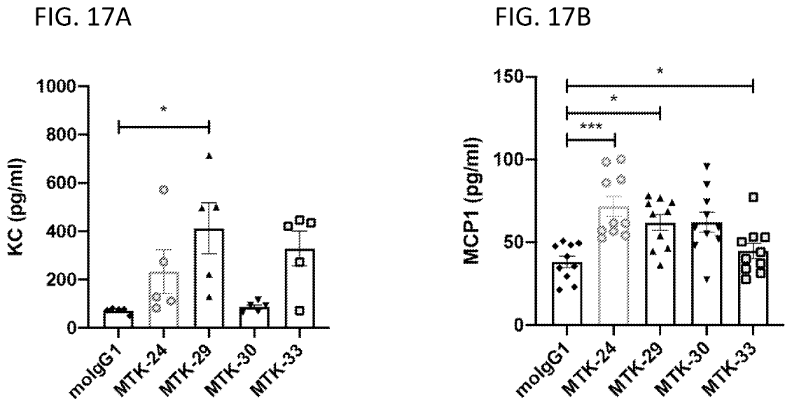

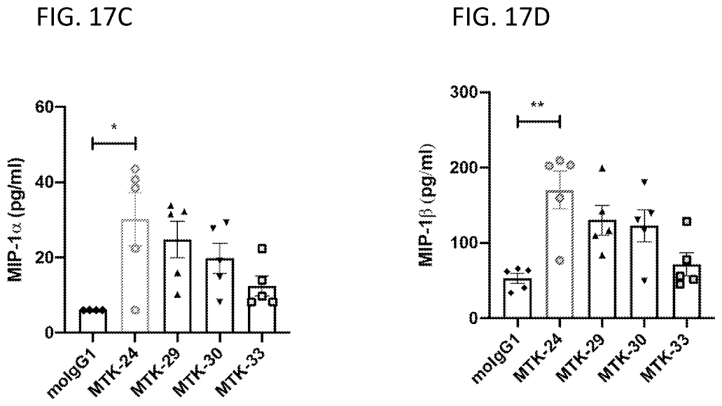

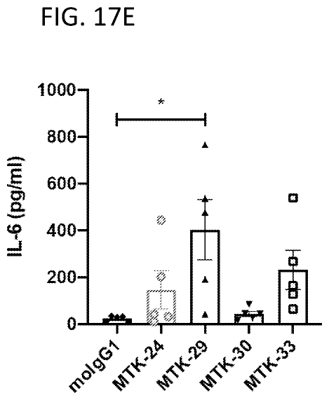

FIGS. 17A, 17B, 17C, 17D, and 17E show the levels of KC (CXCL1), MCP1 (CCL2), MIP-lt (CCL3), MIP-1β (CCL4), and IL-6, respectively, in mice following administration of anti-MerTK antibodies of the present disclosure. As shown in these Figures, the level of each of these chemokines or cytokines increased in response to anti-MerTK antibody MTK-24, MTK-29, MTK-30, and MTK-33 administration in mice compared to that observed in mice administered control antibody. These results showed that anti-MerTK antibody MTK-24, MTK-29, MTK-30, or MTK-33 induced pro-inflammatory chemokine or cytokine production in mouse peripheral cells in a MerTK-dependent way. These results also demonstrated that anti-MerTK antibodies of the present disclosure are effective at increasing pro-inflammatory chemokine/cytokine levels in vivo, thus providing further evidence that anti-MerTK antibodies of the present disclosure are effective at activating an anti-tumor immune response.

### Example 21: Modulation of Pro-Inflammatory Cytokine or Chemokine Production in Non-Human Primates

The effect of anti-MerTK antibodies of the present disclosure on pro-inflammatory cytokine or chemokine production in vivo was tested in cynomolgus monkeys as follows. Anti-MerTK antibody MTK-15, MTK-16, or MTK-21 was given to cynomolgus monkeys via intravenous injection at a dose of 10 mg/kg (N=3 per group). Blood was withdrawn before dosing (pre-dose) and at 1 hour, 6 hours, 12 hours, 24 hours, 72 hours, 168 hours, and 216 hours post-dose. Levels of pro-inflammatory cytokines and chemokines were determined using LEGENDplex NHP Inflammation panel (Biolegend, Catalog #740389) and LEGENDplex NHP Chemokine/cytokine panel (Biolegend, Catalog #740317) according to the manufacturer's instructions. Briefly, plasma samples were diluted 4-fold with Assay Buffer. The Standard Cocktail was serially diluted with Assay Buffer. For setting up standard wells, 25 μl of Matrix B, 25 μl of standard solution, and 25 μl of Mixed Beads was added. For setting up sample wells, 25 μl of Assay Buffer, 25 μl of diluted plasma samples, and 25 μl of Mixed Beads was added. Plates were placed on a plate shaker at 500 rpm for 2 hours. After washing, 25 μl of Detection Antibodies was added, and then plate was shaken for 1 hour. 25 μl of SA-PE was added and then plate was shaken for 30 minutes. After washing, samples were resuspended with 1× Wash Buffer and acquired on a BD FACS CantoII (Becton Dickinson, San Jose, CA) and data were analyzed with LEGENDplex software (Biolegend). Graphs were plotted as fold increase in cytokine/chemokine levels at each specific time point over cytokine/chemokine levels at pre-dose.

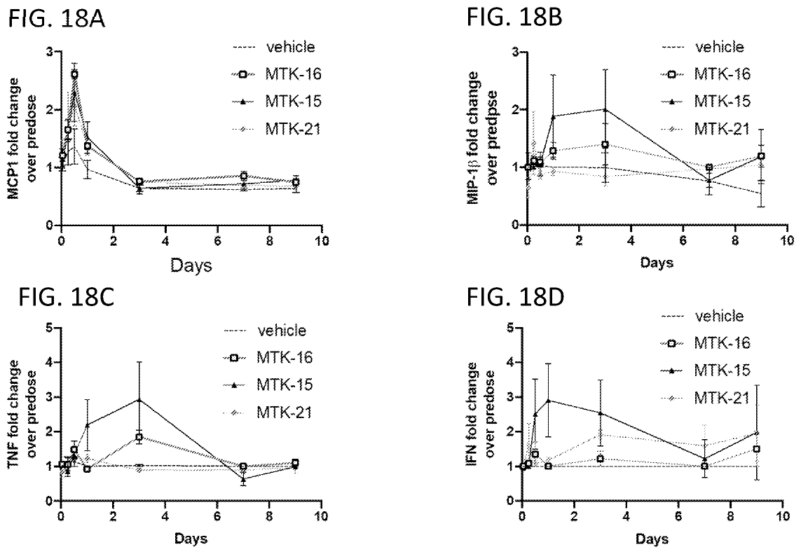

FIGS. 18A, 18B, 18C, and 18D show the time course of changes in levels of MCP1 (CCL2), MIP-1β (CCL4), TNF, and IFN, respectively, in plasma obtained from cynomolgus monkeys administered anti-MerTK antibody MTK-15, MTK-16, or MTK-21, or vehicle control. The peak levels of MCP1 occurred around 12 hours post-dose. Administration of anti-MerTK antibody MTK-15 resulted in an increase in the levels of MIP-1β, TNF, and IFN up to 72 hours post-dose. Taken together, these results demonstrated that anti-MerTK antibody MTK15, MTK-16, or MTK-21 increased pro-inflammatory chemokine or cytokine production in cynomolgus monkey peripheral cells in a MerTK-dependent way. These results further indicated that anti-MerTK antibodies of the present disclosure are effective at increasing MCP1, MIP-1b, TNF, and IFN (which are pro-inflammatory cytokines/chemokines) in vivo in non-human primates.

Two additional studies were performed in cynomolgus monkeys administered anti-MerTK antibody MTK-15 or anti-MerTK antibody MTK-29 (both of which block binding of ProS to MerTK but do not block binding of Gas6 to MerTK, and both of which bind to the Ig2 and FN1 domains of MerTK) or administered anti-MerTK antibody MTK-16 (which blocks binding of both ProS and Gas6 to MerTK and which binds to the Ig1 domain of MerTK). In these preliminary studies, animals administered anti-MerTK antibody MTK-16 displayed decreased systemic toxicity compared to animals administered anti-MerTK antibody MTK-15 or anti-MerTK antibody MTK-29. These preliminary results suggested that anti-MerTK antibodies that block binding of both ProS and Gas6 to MerTK may provide a better in vivo safety profile than anti-MerTK antibodies that block binding of ProS to MerTK but that do not block binding of Gas6 to MerTK. These preliminary results also suggested that anti-MerTK antibodies that bind to the Ig 1 domain of MerTK may provide a better in vivo safety profile than anti-MerTK antibodies that bind to both the Ig2 and FN1 domains of MerTK.

### Example 22: Phospho-AKT Assay in Human Macrophages

The ability of anti-MerTK antibodies of the present disclosure to block phospho-AKT signaling in the presence of human Gas6 (huGas6) protein was evaluated as follows. Differentiated human macrophages were treated with 100 nM Dexamethasone (Sigma-Aldrich, Cat #D4902) and 10 ng/ml huM-CSF (R&D systems, Cat #216MC25CF) for 2 days to polarize into M2c macrophages. 100,000 cells were seeded in 96-well plate. Next day, cells were serum starved for 4 hours. Cells were treated with anti-MerTK antibodies for 30 min at 37° C. and then 200 nM of huGas6 (R&D systems, Cat #885-GSB-050) was added to the cells. After 30 min incubation, cells were lysed to determine phospho-AKT (pAKT) (CisBio, Cat #63ADK082PEG) or total AKT (CisBio, Cat #64NKTPEG) Homogeneous Time Resolved Fluorescence (HTRF) assays following the manufacturer's instructions. The pAKT signal was normalized to total AKT signal to quantitiate the final pAKT activity.

TABLE 18

| | Average IC50 (nM) From |
|---|---|
| Antibody | two different donors |
| MTK-01 | 1.28 |
| MTK-03 | 0.019 |
| MTK-05 | 0.2464 |
| MTK-07 | 0.371 |
| MTK-09 | 1.665 |
| MTK-11 | 2.627 |
| MTK-12 | 1.283 |
| MTK-15 | 2.8 |
| MTK-16 | 7.74 |
| MTK-17 | 3.422 |
| MTK-18 | 1.30 |
| MTK-19 | 0.902 |
| MTK-20 | 1.483 |
| MTK-22 | 0.531 |
| MTK-29 | 5.37 |
| MTK-33 | 4.28 |

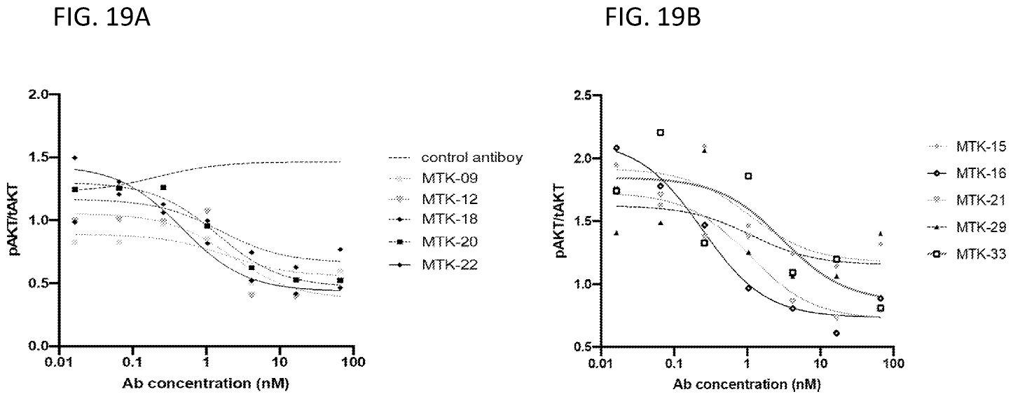

FIGS. 19A and 19B show exemplary anti-MerTK antibodies of the present disclosure (MTK-9, MTK-12, MTK-15, MTK-16, MTK-18, MTK-20, MTK-21, MTK-22, MTK-29, and MTK-33) inhibited Gas6-mediated pAKT activity in a dose-dependent manner. Table 18 above shows IC50 values determined for anti-MerTK antibody blocking of Gas6-mediated pAKT activity. As shown in Table 18. anti-MerTK antibodies of the present disclosure inhibited ligand (e.g., Gas6) mediated MerTK signaling. These results further showed that anti-MerTK antibodies of the present disclosure inhibited Gas6-mediated MerTK signaling, as measured by pAKT levels, with IC50 values ranging from 0.019 nM to 7.74 nM.

### Example 23: Domain Binding Analysis of Anti-MerTK Antibodies

The following studies were performed to analyze the binding sites of various anti-MerTK antibodies of the present disclosure on human MerTK.

MerTK is a member of the TAM family, whose members share a unique domain structure containing an N-terminal region (N), two Immunoglobulin-like domains (Ig 1 and Ig2), two Fibronectin type III domains (FN1 and FN2), a juxta-membrane region (JM), and an intracellular tyrosine kinase domain. MerTK also contains less-structured regions both at the N-terminus of the protein and in the juxta-membrane (JM) region of the MerTK protein; cleavage at the juxta-membrane region leads to release of soluble MerTK extracellular domain (ECD). Human MerTK ECD can be divided into the following domains, the amino acid sequences of which are shown below in Table 19:

TABLE 19

| | | SEQ ID |
|---|---|---|
| Human MerTK domain | Sequence | NO: |
| N-terminal domain (N) | AITEAREEAKPYPLFPGPFPGSLQTDHTPLLSLPHASGY | 449 |
| of human MerTK ECD | QPALMFSPTQPGRPHTGNVAIPQVTSVE | |
| Immunoglobulin-like | SKPLPPLAFKHTVGHIILSEHKGVKFNCSISVPNIYQDT | 450 |
| domain (Ig1) of human | TISWWKDGKELLGAHHAITQFYPDDEVTAIIASFSITSV | |
| MerTK ECD | QRSDNGSYICKMKINNEEIVSDPIYIEVQ | |
| Immunoglobulin-like | GLPHFTKQPESMNVTRNTAFNLTCQAVGPPEPVNIFWVQ | 451 |
| domain (Ig2) of human | NSSRVNEQPEKSPSVLTVPGLTEMAVFSCEAHNDKGLTV | |
| MerTK ECD | SKGVQIN | |
| Fibronectin type III | IKAIPSPPTEVSIRNSTAHSILISWVPGFDGYSPFRNCS | 452 |
| domain (FN1) of human | IQVKEADPLSNGSVMIFNTSALPHLYQIKQLQALANYSI | |
| MerTK ECD | GVSCMNEIGWSAVSPWILAST | |
| Fibronectin type III | TEGAPSVAPLNVTVFLNESSDNVDIRWMKPPTKQQDGEL | 453 |
| domain (FN2) of human | VGYRISHVWQSAGISKELLEEVGQNGSRARISVQVHNAT | |
| MerTK ECD | CTVRIAAVTRGGVGPFSDPV | |
| Juxta membrane domain | KIFIPAHGWVDYAPSSTPAPGNADPVLII | 454 |
| region (JM) of human | | |
| MerTK ECD | | |

Axl protein, another member of the TAM family, shares a common domain structure to that of MerTK, having the following domains and associated amino acid sequences in its ECD:

TABLE 20

| | | SEQ ID |
|---|---|---|
| Human Axl domain | Sequence | NO: |
| N-terminal domain (N) | APRGTQAEESPFVGNPGNITGARGLTG | 455 |
| of human Axl ECD | | |
| Immunoglobulin-like | TLRCQLQVQGEPPEVHWLRDGQILELADSTQTQVPLGED | 456 |
| domain (Ig1) of human | EQDDWIVVSQLRITSLQLSDTGQYQCLVFLGHQTFVSQP | |
| Axl ECD | GYVGLE | |
| Immunoglobulin-like | GLPYFLEEPEDRTVAANTPFNLSCQAQGPPEPVDLLWLQ | 457 |
| domain (Ig2) of human | DAVPLATAPGHGPQRSLHVPGLNKTSSFSCEAHNAKGVT | |
| Axl ECD | TSRTATITVLP | |
| Fibronectin type III | QQPRNLHLVSRQPTELEVAWTPGLSGIYPLTHCTLQAVL | 458 |
| domain (FN1) of human | SDDGMGIQAGEPDPPEEPLTSQASVPPHQLRLGSLHPHT | |
| Axl ECD | PYHIRVACTSSQGPSSWTHWLPVETPEG | |
| Fibronectin type III | VPLGPPENISATRNGSQAFVHWQEPRAPLQGTLLGYRLA | 459 |
| domain (FN2) of human | YQGQDTPEVLMDIGLRQEVTLELQGDGSVSNLTVCVAAY | |
| Axl ECD | TAAGDGPWS | |
| Juxta membrane domain | LPVPLEAWRPGQAQPVHQLVKEPSTPAFSWPWW | 460 |
| (JM) of human Axl ECD | | |

In these experiments, a series of chimeric proteins were recombinantly expressed in which various domains of human MerTK were swapped with corresponding human Axl domains. The resulting chimeic proteins were recombinantly expressed in Expi293 cells and binding of various anti-MerTK antibodies of the present disclosure were then analyzed for their ability to bind the Axl/MerTK domain-swapped chimeras. DNA encoding the domain-swapped chimeras and deletion mutants, with a signal sequence (MGWSCIILFLVATATGVHS (SEQ ID NO:461) in MerTK constructs, and MGWSCIILFLVATATG (SEQ ID NO:462) in Axl constructs) and a linker+His+Avi tag (GGSGHTKHGGGLNDIFEAQKIEWHE; SEQ ID NO:463) were prepared by gene synthesis and cloned into the expression vector pcDNAtopo3.4 (GeneArt, ThermoFisher). Expi293 cells were transfected with 2 μg of plasmids and ExpiFectamine following recommended procedures (ThermoFisher). Transfected cells were grown in 2 mL cultures with shaking at 37C and 5% CO₂ for four days. Cells were pelleted by centrifugation and the supernatants were filtered through 0.2 μM filters by vacuum.

Table 21 shows the configurations of the various Axl/MerTK domain-swapped chimeras that were generated for use in these studies. In Table 21, an M (MerTK) or an A (Axl) indicates from which protein that particular domain was included (i.e., swapped) in the corresponding chimeric protein construct; N (N-terminal domain), Ig1 (immunoglobulin-like domain 1), Ig2 (immunoglobulin-like domain 2), FN1 (fibronectin type III domain 1), FN2 (fibronectin type III domain 2), and JM (juxta-membrane domain).

TABLE 21

| Axl/MerTK Chimeric | N | Ig1 | Ig2 | FN1 | FN2 | JM |
|---|---|---|---|---|---|---|
| Full MerTK | M | M | M | M | M | M |
| MerTK with swapped N | A | M | M | M | M | M |
| MerTK with swapped Ig1 | M | A | M | M | M | M |
| MerTK with swapped Ig2 | M | M | A | M | M | M |
| MerTK with swapped FN1 | M | M | M | A | M | M |
| MerTK with swapped FN2 | M | M | M | M | A | M |
| MerTK with swapped JM | M | M | M | M | M | A |
| Full Axl | A | A | A | A | A | A |
| Axl with swapped N | M | A | A | A | A | A |
| Axl with swapped Ig1 | A | M | A | A | A | A |
| Axl with swapped Ig2 | A | A | M | A | A | A |
| Axl with swapped FN1 | A | A | A | M | A | A |
| Axl with swapped FN2 | A | A | A | A | M | A |
| Axl with swapped JM | A | A | A | A | A | M |
| MerTK with deleted N | | M | M | M | M | M |
| MerTK with deleted N/Ig1 | | | M | M | M | M |
| MerTK with deleted N/Ig1/Ig2 | | | | M | M | M |
| MerTK with deleted N/Ig1/Ig2/FN1 | | | | | M | M |
| MerTK with deleted N/Ig1/Ig2/FN1/FN2 | | | | | | M |

Binding of anti-MerTK antibodies of the present disclosure to these domain-swapped chimera or deletion mutants (prepared as described above) was tested by Surface Plasmon Resonance (SPR) using Carterra LSA. Purified anti-MerTK antibodies were immobilized in duplicate on a HC30M chip (Carterra) by amine coupling, following the manufacturer's instructions (described previously). Supernatants containing the constructs were diluted 1:1 with running buffer containing 0.5 mg/mL BSA (HBS-TE, Carterra) and injected over the immobilized antibodies. The surface was regenerated with 10 mM Glycine pH2.0 after each construct injection. Sensorgrams were analyzed using Carterra Epitope software to identify patterns of construct binding. Loss of binding to MerTK constructs with domains deleted or swapped in from Axl and/or gain of binding to Axl constructs with MerTK domains swapped in were evaluated. Data from these studies was used to construct a domain binding map for anti-MerTK antibodies and their binding to various domains of human MerTK.

In addition to anti-MerTK antibodies of the present disclosure, the following anti-MerTK antibodies were also used in these studies: rat anti-mouse MerTK antibody DS5MMER (eBioscience, Clone ID DS5MMER, rat IgG2a), mouse anti-human MerTK antibody H1 (BioLegend, Clone ID: 590H11G1E3, mouse IgG1), mouse anti-human MerTK antibody H2 (R&D systems, Clone ID: 125518, mouse IgG2b), mouse anti-human MerTK antibody H3 (R&D systems, Clone ID 125508, mouse IgG2b), mouse anti-human MerTK antibody H6 (eBioscience, Clone ID: A3KCAT, mouse IgG1), mouse anti-human MerTK antibody H7 (Sino Biological, Clone ID: 09, Mouse IgG2b), human anti-human MerTK antibody M6 (disclosed in WO2016/106221, huIgG1 LALAPS), and human anti-human MerTK antibody CDX AB3000 (disclosed in WO2020/106461).

TABLE 22

| Antibody | Binding domain |
|---|---|
| H6 | N-terminal |
| MTK-10 | Ig1 |
| MTK-16 | |
| MTK-21 | |
| MTK-25 | |
| MTK-33 | |
| M6 | |
| MTK-22 | |
| MTK-06 | Ig2/FN1 |
| MTK-15 | |
| MTK-29 | |
| MTK-30 | |
| CDX AB3000 | |
| H1 | |
| H2 | |
| H3 | Juxta membrane |
| H7 | domain |

The results of these binding studies are shown above in Table 22. As shown in Table 22, anti-MerTK antibody H6 bound to chimeric protein constructs containing the N-terminal region of human MerTK. Anti-MerTK antibodies MTK-10, MTK-16, MTK-21, MTK-25, MTK-33, and M6 bound to constructs that contained the MerTK Ig 1 domain. Anti-MerTK antibody MTK-22 bound to chimeric protein constructs that contained the MerTK Ig1; however, anti-MerTK antibody MTK-22 binding was affected by the presence of the MerTK FN1 domain. Anti-MerTK antibodies MTK-06, MTK-15, MTK-29, MTK-30, CDX AB3000, H1, and H2 bound only to chimeric protein constructs that contained both the MerTK Ig2 and FN1 domains. Anti-MerTK antibodies H3 and H7 bound to chimeric protein constructs containing the MerTK juxta membrane region. None of the anti-MerTK antibodies tested above bound to the ECD domain of human Axl (data not shown).

These results showed that anti-MetTK antibodies of the present disclosure that blocked binding of both Gas6 and ProS to MerTK generally bound to the Ig1 domain of human MerTK. These results also showed that anti-MerTK antibodies of the present disclosure that blocked binding of only ProS to MerTK generally bound to a combination of both Ig2/FN1 domains.

### Example 24: Effect of Anti-MerTK Antibody Treatment in Transgenic Mouse Colon Cancer Model

The effect of anti-MerTK antibodies on tumor growth in vivo was performed as follows using an in vivo mouse colon cancer model in which the mice were transgenic to include human MerTK. MC38 mouse cancer cells were implanted subcutaneously on the shaved right flank of huMerTK Knock-In (KI) mice (Ozgene, Perth, Australia), where human MerTK was introduced into the mouse genome. When tumor size reached approximately 80-130 mm³ in volume, mice were treated with 10 mg/kg of MTK-33 plus 3 mg/kg of anti-PDL1 (clone BM1), 10 mg/kg of MTK-16 μlus 3 mg/kg of anti-PDL1, 3 mg/kg of anti-PDL1 alone, or 10 mg/kg of control antibody twice a week. Tumor volume was measured three times a week.

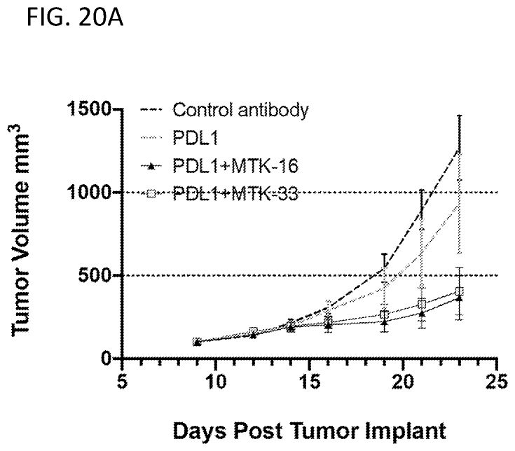

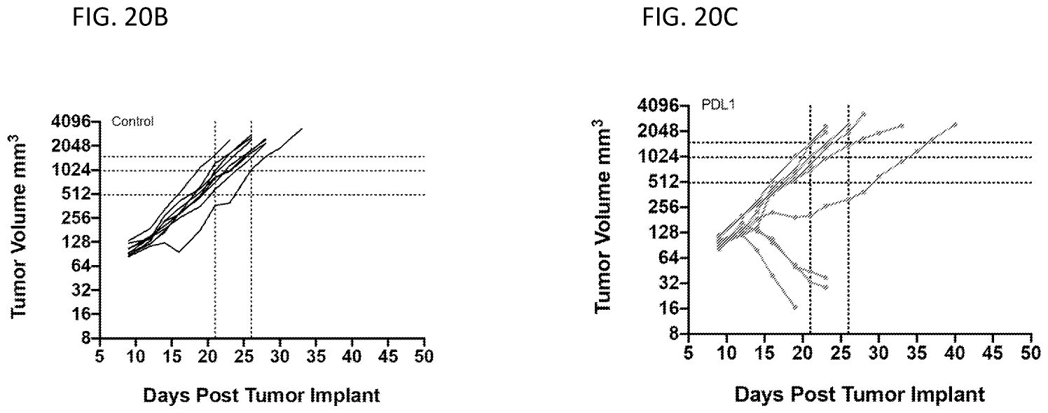

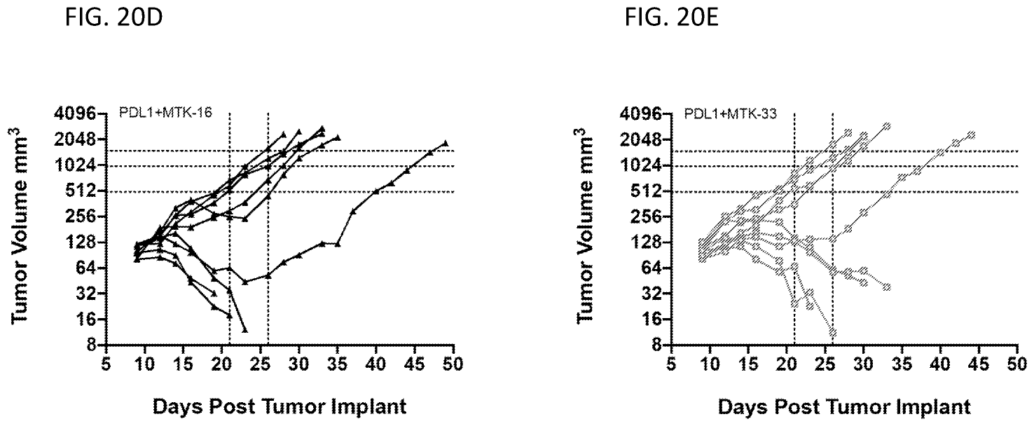

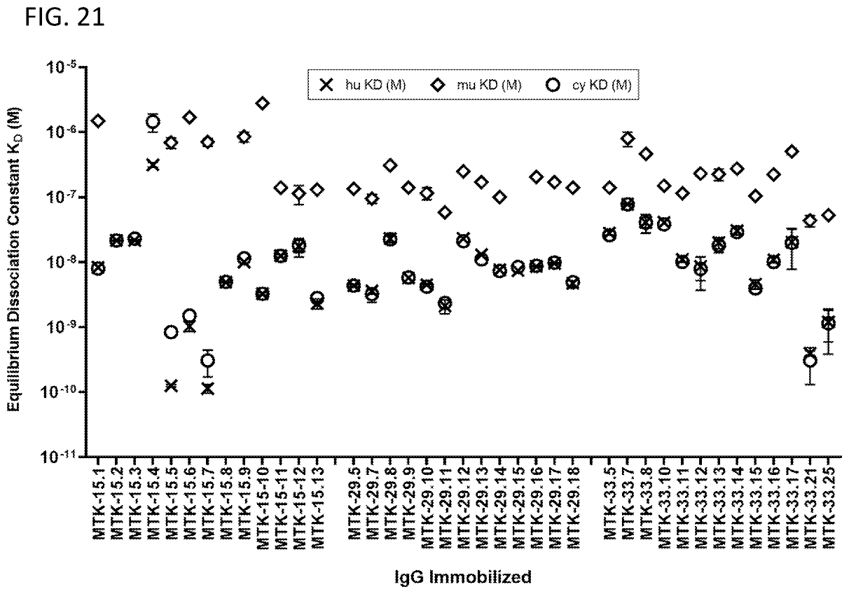

FIG. 20A shows that a combination of anti-PDL1 antibody with either anti-MerTK antibody MTK-16 or anti-MerTK antibody MTK-33 moderately reduced the tumor growth rate compared to that observed in mice treated with either anti-PDL1 antibody alone or with control antibody. FIG. 20B shows the tumor growth of individual mouse in each group and the combination treatment of anti-PDL1 with either MTK-16 or MTK-33 led to the complete regression of tumors from 3 or 4 out of 9 mice. Thus, this data show that human MerTK binding anti-MerTK antibody MTK-16 or MTK-33 in combination with anti-PDL1 antibody improved efficacy in inhibiting tumor growth Example 25: Humanization of murine anti-MERTK mouse antibodies

Humanized variants of certain parental mouse anti-MerTK antibodies of the present disclosure were generated as follows.

The parental mouse anti-MerTK antibody MTK-16 contains a heavy chain variable region comprising the amino acid sequence of:

(SEQ ID NO: 55)
DIVMTQSPKFMSTSVGDRVSITCKASQNVRTAVAWYKKKPGQSPKALINL
ASNRHTGVPDRFTGSGSGTDFTLTISNVQSEDLADYFCLQHWNYPLTFGG
GTKLEIK.

and a light chain variable region comprising the amino acid sequence of:

(SEQ ID NO: 20)
LIQLVQSGPELKKPGETVKISCKASGYTFTNHGMNWVKQDPGKGLKWMGW
INTYTGEPTYADDFKGRFVFSMETSASAAFLQINNLKNEDTATYFCARKG
VTAARYFDYWGQGTTLTVSS,

The parental mouse anti-MerTK antibody MTK-33 contains a heavy chain variable region comprising the amino acid sequence of:

(SEQ ID NO: 37)
QVQLQQPGPELVRPGTSVKLSCKASGYTFTSYWMHWIKQRPGQGLEWIGV
IDPSDNYINYNQKFKGKATLTVDTSSSTAYLQLSSLTSEDSAVYYCAREA
GTRGYFDYWGQGTTLTVSS,

and a light chain variable region comprising the amino acid sequence of:

(SEQ ID NO: 72)
SIVMTQTPKFLLVSAGDRVIITCKASQSVSNTVAWYQQKPGQSPKLLIYY
ASNRYTGVPDRFTGSGYGTDFTFTISTVQAEDLAVYFCQQDYRSPFTFGS
GTQLEMK.

The parental mouse anti-MerTK antibody MTK-15 contains a heavy chain variable region comprising the amino acid sequence of:

(SEQ ID NO: 19)
QIQLVQSGPELKKPGETVKISCKASGYTFTNYGVHWVKQAPGKGLKWMGW
INTYTGEPTYADDFKGRFAFSLETSASTAYLQINNLKNEDMATYFCARGN
RYAYMDYWGQGTSVTVSS,

and a light chain variable region comprising the amino acid sequence of:

(SEQ ID NO: 54)
QIVLSQSPAILSASPGEKVTMTCRASSSVSYMHWYQQKSGSSPKPWIYAT
SNLASGVPARFSGSGSGTSYSLTISRVEAEDAATYYCQQWSSNPRTFGGG
TKLEIK.

The parent mouse anti-MerTK antibody MTK-29 contains a heavy chain variable region comprising the amino acid sequence of:

(SEQ ID NO: 33)
QIQLVQSGPELKKPGETVKISCKSSGFTFTTYGMSWVKQAPGKGLKWMGW
INTYSGVPTYTDDFKGRFAFSLETSASTASLQINNLKNEDTATYFCARYT
NYGYFDYWGQGTTLTVSS,

and a light chain variable region comprising the amino acid sequence of:

(SEQ ID NO: 68)
QIVLSQSPAILSASPGEKVTMTCRATSSVGYMHWYQQKPGSSPKPWIYAT
SNLASGVPARFSGSGSGTSYSLTISRVEAEHAATYYCQQWGSNPFTFGSG
TKLEIK.

One method of humanizing non-human antibodies is to transplant the CDRs from a non-human (e.g., murine) antibody onto a human antibody acceptor framework. Such CDR transplantation may result in attenuation or complete loss of affinity of the humanized antibody to its target due to perturbation in its framework. As a result, certain amino acid residues in the human framework may need to be replaced by amino acid residues from the corresponding positions of the murine antibody framework (referred to as back mutations) in order to restore attenuated or lost affinity as a result of humanization. Therefore, the amino acid residues to be replaced in the context of the selected human antibody germline acceptor framework must be determined so that the humanized antibody substantially retains functions and paratopes. In addition, retained or improved thermal stability and solubility are desired for good manufacturability and downstream development.

Briefly, V_{H} and V_{L} amino acid sequences of the mouse anti-MerTK monoclonal antibodies to be humanized were compared to human V_{L}, V_{H}, LJ, and HJ functional germline amino acid sequences taken from IMGT (http://www.imgt.org/). Pseudo-genes and open reading frames were excluded from these analyses. Per one mouse monoclonal antibody (query), one or two of the most similar V_{H} and one of the most similar V_{L} germline amino acid sequences were selected and combined with the most similar VJ and HJ genes, producing one or two humanized amino acid sequences. The CDRs to be transplanted onto the human framework were defined according to the AbM definition.

Structure-based antibody modeling was applied in the process of humanizing mouse anti-MerTK monoclonal antibodies MTK-15 and MTK-29 utilizing the BioMOE module of MOE (Molecular Operating Environment, Chemical Computing Group, Montreal, Canada). Briefly, V_{H} and V_{L} amino acid sequences of the mouse monoclonal antibodies MTK-15 and MTK-29 were compared to human V_{L}, V_{H}, LJ, HJ functional germline amino acid sequences taken from IMGT (http://www.imgt.org/) as mentioned above. Three of the most similar V_{H} and three of the most similar V_{L} germline amino acid sequences were selected and combined with the most similar VJ and HJ genes, producing five to eight humanized amino acid sequences.

The query and the humanized amino acid sequences were used to create Fv homology models using BioMOE module or the Antibody Modeler module of MOE (Molecular Operating Environment, Chemical Computing Group, Montreal, Canada). AMBER10:EHT force field analysis was used for energy minimization through the entire antibody homology modeling process. Based on the Fv homology models obtained, molecular descriptors such as interaction energy between V_{L} and V_{H}, coordinate-based isoelctric point (3D pI), hydrophobic patch, and charged surface area were calculated, analyzed, and sorted by scoring metrics provided by MOE. These molecular descriptors were utilized to prioritize the humanized monoclonal antibodies for downstream experimental procedures, including protein expression, purification, binding affinity studies, and functional assays.

The BioMOE module of MOE provides a tool, Mutation Site Properties, to visualize and classify potential residues for back-mutation. In this context, back-mutation is defined as amino acid substitution which is reverted to the original query amino acid sequence replacing the humanized amino acid sequence. Using this tool, the original query (reference) was compared individually to the selected humanized variants for both the primary amino acid sequence and the 3D structure of the 3D Fv homology model.

Changes between the reference (i.e., parental) antibody and the humanized variant were classified based on amino acid type difference, interaction potential with CDR residues, impact potential for V_{L}/V_{H} pairing, and potential change in hydrophobic and charged surface area in and near the CDRs.

Mutations near the CDRs or the V_{L}/V_{H} interface having a significant charge difference or containing strong H-bond interactions were individually evaluated and the significantly disrupting mutations were reverted to the original query residues.

Affinity maturation of humanized anti-MerTK antibodies MTK-33, MTK-29, and MTK-16 were also performed. Briefly, certain amino acid residues in the heavy chain or light chain were selectively mutagenized and mutants that improved binding were selected through additional rounds of screening. This process simultaneously improved specificity, species cross-reactivity, and developability profiles. Characterization of the affinity-matured anti-MerTK antibodies described herein included SPR affinity measurements on Carterra LSA and efferocytosis blocking assays on human macrophage. After multiple rounds of affinity maturation, anti-MerTK antibodies with desired affinity were obtained. Amino acid sequences of the variable heavy chains (V_{H}) and variable light (V_{L}) chains are provided below in Table 23 (MTK-15 variants), Table 24 (MTK-16 variants), Table 25 (MTK-29 variants), and Table 26 (MTK-33 variants). In Tables 23, 24, 25, and 26, the hypervariable regions (HVR) in each of the chains are underlined.

Table 27 and Table 28 provide amino acid sequences for the heavy chain HVRs and the light chain HVRs, respectively, for anti-MerTK antibody MTK-15 variants. Table 29 and Table 30 provide amino acid sequences for the heavy chain HVRs and the light chain HVRs, respectively, for anti-MerTK antibody MTK-16 variants. Table 31 and Table 32 provide amino acid sequences for the heavy chain HVRs and the light chain HVRs, respectively, for anti-MerTK antibody MTK-29 variants. Table 33 and Table 34 provide amino acid sequences for the heavy chain HVRs and the light chain HVRs, respectively, for anti-MerTK antibody MTK-33 variants.

TABLE 23

| | | SEQ | | SEQ |
|---|---|---|---|---|
| | | ID | | ID |
| Antibody | Heavy Chain Variable | NO: | Light Chain Variable | NO: |
| MTK-15.1 | QVQLVQSGSELKKPGASVKVSC | 234 | EIVLTQSPATLSLSPGERATLSC | 247 |
| | KASGYTFTNYGVHWVRQAPGQG | | RASSSVSYMHWYQQKPGQAPRPL | |
| | LEWMGWINTYTGEPTYAQGFTG | | IYATSNLASGIPARFSGSGSGTS | |
| | RFVFSLDTSVSTAYLQISSLKA | | YTLTISSLEPEDFAVYYCQQWSS | |
| | EDTAVYYCARGNRYAYQDYWGQ | | NPRTFGGGTKVEIK | |
| | GTLVTVSS | | | |
| MTK-15.2 | QVQLVQSGSELKKPGASVKVSC | 235 | EIVLTQSPATLSLSPGERATLSC | 247 |
| | KASGYTFTNYGVHWVRQAPGQG | | RASSSVSYMHWYQQKPGQAPRPL | |
| | LEWMGWGNTYTGEPTYAQGFTG | | IYATSNLASGIPARFSGSGSGTS | |
| | RFVFSLDTSVSTAYLQISSLKA | | YTLTISSLEPEDFAVYYCQQWSS | |
| | EDTAVYYCARGNRYAYMDYWGQ | | NPRTFGGGTKVEIK | |
| | GTLVTVSS | | | |
| MTK-15.3 | QVQLVQSGSELKKPGASVKVSC | 236 | EIVLTQSPATLSLSPGERATLSC | 248 |
| | KASGYTFTNYGVHWVRQAPGQG | | RASSHVSYMHWYQQKPGQAPRPL | |
| | LEWMGWINTYTGEPTYAQGFTG | | IYATSNLASGIPARFSGSGSGTS | |
| | RFVFSLDTSVSTAYLQISSLKA | | YTLTISSLEPEDFAVYYCQQWSS | |
| | EDTAVYYCARGNRYAYMDYWGQ | | NPRTFGGGTKVEIK | |
| | GTLVTVSS | | | |
| MTK-15.4 | QVQLVQSGSELKKPGASVKVSC | 236 | EIVLTQSPATLSLSPGERATLSC | 249 |
| | KASGYTFTNYGVHWVRQAPGQG | | RASSSVGYMHWYQQKPGQAPRPL | |
| | LEWMGWINTYTGEPTYAQGFTG | | IYATSNLASGIPARFSGSGSGTS | |
| | RFVFSLDTSVSTAYLQISSLKA | | YTLTISSLEPEDFAVYYCQQWSS | |
| | EDTAVYYCARGNRYAYMDYWGQ | | NPRTFGGGTKVEIK | |
| | GTLVTVSS | | | |
| MTK-15.5 | QVQLVQSGSELKKPGASVKVSC | 237 | EIVLTQSPATLSLSPGERATLSC | 249 |
| | KASGYTFTNYGVHWVRQAPGQG | | RASSSVGYMHWYQQKPGQAPRPL | |
| | LEWMGWINTYTGEPTYAQGFTG | | IYATSNLASGIPARFSGSGSGTS | |
| | RFVFSLDTSVSTAYLQISSLKA | | YTLTISSLEPEDFAVYYCQQWSS | |
| | EDTAVYYCTRGTRYAYMDYWGQ | | NPRTFGGGTKVEIK | |
| | GTLVTVSS | | | |
| MTK-15.6 | QVQLVQSGSELKKPGASVKVSC | 238 | EIVLTQSPATLSLSPGERATLSC | 249 |
| | KASGYTFTNYGVHWVRQAPGQG | | RASSSVGYMHWYQQKPGQAPRPL | |
| | LEWMGWINTYTGEPYYAQGFTG | | IYATSNLASGIPARFSGSGSGTS | |
| | RFVFSLDTSVSTAYLQISSLKA | | YTLTISSLEPEDFAVYYCQQWSS | |
| | EDTAVYYCARGNRYAYMDYWGQ | | NPRTFGGGTKVEIK | |
| | GTLVTVSS | | | |
| MTK-15.7 | QVQLVQSGSELKKPGASVKVSC | 239 | EIVLTQSPATLSLSPGERATLSC | 250 |
| | KASGYTFTNYGVHWVRQAPGQG | | RASSGVSYMHWYQQKPGQAPRPL | |
| | LEWMGWINTYTGEPTYAQGFTG | | IYATSNLASGIPARFSGSGSGTS | |
| | RFVFSLDTSVSTAYLQISSLKA | | YTLTISSLEPEDFAVYYCQQWSS | |
| | EDTAVYYCVRGNRYAYMDYWGQ | | NPRTFGGGTKVEIK | |
| | GTLVTVSS | | | |
| MTK-15.8 | QVQLVQSGSELKKPGASVKVSC | 240 | EIVLTQSPATLSLSPGERATLSC | 251 |
| | KASGYTFTNYGVHWVRQAPGQG | | RASSSVGYMHWYQQKPGQAPRPL | |
| | LEWMGWINTYTGEPTYAQGFTG | | IYATSNLASGIPARFSGSGSGTS | |
| | RFVFSLDTSVSTAYLQISSLKA | | YTLTISSPEPEDFAVYYCQQWSS | |
| | EDTAVYYCARGVRYAYMDYWGQ | | NPRTFGGGTKVEIK | |
| | GTLVTVSS | | | |
| MTK-15.9 | QVQLVQSGSELKKPGASVKVSC | 241 | EIVLTQSPATLSLSPGERATLSC | 252 |
| | KASGYTFTNYGVHWVRQAPGQG | | RASSSTSYMHWYQQKPGQAPRPL | |
| | LEWMGWINTYTGEPTYAQGFTG | | IYATSNLASGIPARFSGSGSGTS | |
| | RFVFSLDTSVSTAYLQISSLKA | | YTLTISSLEPEDFAVYYCQQWSS | |
| | EDTAVYYCARGYRYAYMDYWGQ | | NPRTFGGGTKVEIK | |
| | GTLVTVSS | | | |
| MTK-15.10 | QVQLVQSGSELKKPGASVKVSC | 242 | EIVLTQSPATLSLSPGERATLSC | 249 |
| | KASGYTFANYGVHWVRQAPGQG | | RASSSVGYMHWYQQKPGQAPRPL | |
| | LEWMGWINTYQGEPTYAQGFTG | | IYATSNLASGIPARFSGSGSGTS | |
| | RFVFSLDTSVSTAYLQISSLKA | | YTLTISSLEPEDFAVYYCQQWSS | |
| | EDTAVYYCTRGARYAYMDYWGQ | | NPRTFGGGTKVEIK | |
| | GTLVTVSS | | | |
| MTK-15.11 | QVQLVQSGSELKKPGASVKVSC | 243 | EIVLTQSPATLSLSPGERATLSC | 247 |
| | KASGYTFANYGVHWVRQAPGQG | | RASSSVSYMHWYQQKPGQAPRPL | |
| | LEWMGWINTYEGEPTYAQGFTG | | IYATSNLASGIPARFSGSGSGTS | |
| | RFVFSLDTSVSTAYLQISSLKA | | YTLTISSLEPEDFAVYYCQQWSS | |
| | EDTAVYYCARGNRYAYQDYWGQ | | NPRTFGGGTKVEIK | |
| | GTLVTVSS | | | |
| MTK-15.12 | QVQLVQSGSELKKPGASVKVSC | 244 | EIVLTQSPATLSLSPGERATLSC | 251 |
| | KASGYTFANYGVHWVRQAPGQG | | RASSSVGYMHWYQQKPGQAPRPL | |
| | LEWMGWINTYQGEPTYAQGFTG | | IYATSNLASGIPARFSGSGSGTS | |
| | RFVFSLDTSVSTAYLQISSLKA | | YTLTISSPEPEDFAVYYCQQWSS | |
| | EDTAVYYCARGVRYAYMDYWGQ | | NPRTFGGGTKVEIK | |
| | GTLVTVSS | | | |
| MTK-15.13 | QVQLVQSGSELKKPGASVKVSC | 245 | EIVLTQSPATLSLSPGERATLSC | 253 |
| | KASGYTFTNYGVHWVRQAPGQG | | RASSSVQYMHWYQQKPGQAPRPL | |
| | LEWMGWINTYTGEPTYAQGFTG | | IYATSNLASGIPARFSGSGSGTS | |
| | RFVFSLDTSVSTAYLQISSLKA | | YTLTISSLEPEDFAVYYCQQWSS | |
| | EDTAVYYCARGNRYAYEDYWGQ | | NPRTFGGGTKVEIK | |
| | GTLVTVSS | | | |
| MTK-15.14 | QVQLVQSGSELKKPGASVKVSC | 246 | EIVLTQSPATLSLSPGERATLSC | 247 |
| | KASGYTFTNYGVHWVRQAPGQG | | RASSSVSYMHWYQQKPGQAPRPL | |
| | LEWMGWINTYTGEPTYAQGFTG | | IYATSNLASGIPARFSGSGSGTS | |
| | RFVFSLDTSVSTAYLQICSLKA | | YTLTISSLEPEDFAVYYCQQWSS | |
| | EDTAVYYCARGNRYAYMDYWGQ | | NPRTFGGGTKVEIK | |
| | GTLVTVSS | | | |
| MTK-15.15 | QVQLVQSGSELKKPGASVKVSC | 246 | QIVLTQSPGTLSLSPGERATLSC | 254 |
| | KASGYTFTNYGVHWVRQAPGQG | | RASSSVSYMHWYQQKPGQAPRPL | |
| | LEWMGWINTYTGEPTYAQGFTG | | IYATSNLASGIPDRFSGSGSGTS | |
| | RFVFSLDTSVSTAYLQICSLKA | | YTLTISRLEPEDFAVYYCQQWSS | |
| | EDTAVYYCARGNRYAYMDYWGQ | | NPRTFGGGTKVEIK | |
| | GTLVTVSS | | | |

TABLE 24

| | | SEQ | | SEQ |
|---|---|---|---|---|
| | | ID | | ID |
| Antibody | Heavy Chain Variable | NO: | Light Chain Variable | NO: |
| MTK-16.1 | QVQLVQSGSELKKPGASVKVSC | 255 | DIQMTQSPSSLSASVGDRVTITC | 257 |
| | KASGYTFTNHGMNWVRQAPGQG | | KASQNVRTAVAWYQQKPGKAPKR | |
| | LEWMGWINTYTGEPTYAQGFTG | | LIYLASNRHTGVPSRFSGSGSGT | |
| | RFVFSLDTSVSTAYLQISSLKA | | EFTLTISNLQPEDFATYYCLQHW | |
| | EDTAVYYCARKGVTAARYFDYW | | NYPLTFGGGTKVEIK | |
| | GQGTLVTVSS | | | |
| MTK-16.2 | QVQLVQSGSELKKPGASVKVSC | 256 | DIQMTQSPSSLSASVGDRVTITC | 258 |
| | KASGYTFTNHGMNWVRQAPGQG | | RASQNVRTAVAWYQQKPGKAPKR | |
| | LEWMGWINTYTGEPTYADDFKG | | LIYLASNRHTGVPSRFSGSGSGT | |
| | RFVFSLDTSVSTAYLQISSLKA | | EFTLTISNLQPEDFATYYCLQHW | |
| | EDTAVYYCARKGVTAARYFDYW | | NYPLTFGGGTKLEIK | |
| | GQGTLVTVSS | | | |

TABLE 25

| | | SEQ | | SEQ |
|---|---|---|---|---|
| | | ID | | ID |
| Antibody | Heavy Chain Variable | NO: | Light Chain Variable | NO: |
| MTK-29.1 | QVQLVQSGSELKKPGASVKVSC | 259 | EIVLTQSPATLSLSPGERATLSC | 274 |
| | KASGYIFTSYGLSWVRQAPGQG | | RASSSVGYMHWYQQKPGQAPRPL | |
| | LEWMGWINTYSGVPTYAQGFTG | | IYATSNLASGIPARFSGSGSGTD | |
| | RFVFSLDTSVSTAYLQISSLKA | | FTLTISRLEPEDFAVYYCQQWGS | |
| | EDTAVYYCARYTNYGIFDYWGQ | | WPFTFGQGTKLEIK | |
| | GTLVTVSS | | | |
| MTK-29.2 | QVQLVQSGSELKKPGASVKVSC | 260 | DIQLTQSPSFLSASVGDRVTITC | 275 |
| | KASGFTFTTYGMSWVRQAPGQG | | RASSSVGYMHWYQQKPGKAPKPL | |
| | LEWMGWINTYSGVPTYTDDFKG | | IYATSNLASGVPSRFSGSGSGTE | |
| | RFVFSLDTSVSTAYLQISSLKA | | FTLTISSLQPEDFATYYCQQWGS | |
| | EDTAVYYCARYTNYGIFDYWGQ | | IPFTFGGGTKVEIK | |
| | GTLVTVSS | | | |
| MTK-29.3 | QVQLVQSGSELKKPGASVKVSC | 261 | DIQLTQSPSFLSASVGDRVTITC | 276 |
| | KASGFTFTTYGMSWVRQAPGQG | | RASSSVGYMHWYQQKPGKAPKLL | |
| | LEWMGWINTYSGVPAYTDDFKG | | IYATSNLASGVPSRFSGSGSGTE | |
| | RFVFSLDTSVSTAYLQISSLKA | | FTLTISSLQPEDFATYYCQQWGS | |
| | EDTAVYYCARYTNYGVFDYWGQ | | LPFTFGQGTKLEIK | |
| | GTLVTVSS | | | |
| MTK-29.4 | QVQLVQSGSELKKPGASVKVSC | 262 | DIQLTQSPSFLSASVGDRVTITC | 277 |
| | KASSYTFTTYGMSWVRQAPGQG | | RASSSVGYMHWYQQKPGKAPKLL | |
| | LEWMGWINTYSGVPTYTDDFKG | | IYATSNLAQGVPSRFSGSGSGTE | |
| | RFVFSLDTSVSTAYLQISSLKA | | FTLTISSLQPEDFATYYCQQWGS | |
| | EDTAVYYCARYTNYGVFDYWGQ | | LPFTFGQGTKLEIK | |
| | GTLVTVSS | | | |
| MTK-29.5 | QVQLVQSGSELKKPGASVKVSC | 263 | DIQLTQSPSFLSASVGDRVTITC | 277 |
| | KASGYIFTSYGLSWVRQAPGQG | | RASSSVGYMHWYQQKPGKAPKLL | |
| | LEWMGWINTYSGVPTYTDDFKG | | IYATSNLAQGVPSRFSGSGSGTE | |
| | RFVFSLDTSVSTAYLQISPLKA | | FTLTISSLQPEDFATYYCQQWGS | |
| | EDTAVYYCARYTNYGVFDYWGQ | | LPFTFGQGTKLEIK | |
| | GTLVTVSS | | | |
| MTK-29.6 | QVQLVQSGSELKKPGASVKVSC | 264 | DIQLTQSPSFLSASVGDRVTITC | 277 |
| | KASSYTFTTYGMSWVRQAPGQG | | RASSSVGYMHWYQQKPGKAPKLL | |
| | LEWMGWINTMSGVPTYTDDFKG | | IYATSNLAQGVPSRFSGSGSGTE | |
| | RFVFSLDTSVSTAYLQISSLKA | | FTLTISSLQPEDFATYYCQQWGS | |
| | EDTAVYYCARYTNYGVFDYWGQ | | LPFTFGQGTKLEIK | |
| | GTLVTVSS | | | |
| MTK-29.7 | QVQLVQSGSELKKPGASVKVSC | 265 | DIQLTQSPSFLSASVGDRVTITC | 277 |
| | KASSYTFTTYGMSWVRQAPGQG | | RASSSVGYMHWYQQKPGKAPKLL | |
| | LEWMGWINTYSGVPSYTDDFKA | | IYATSNLAQGVPSRFSGSGSGTE | |
| | RFVFSLDTSVSTAYLQISSLKA | | FTLTISSLQPEDFATYYCQQWGS | |
| | EDTAVYYCARYTNYGVFDYWGQ | | LPFTFGQGTKLEIK | |
| | GTLVTVSS | | | |
| MTK-29.8 | QVQLVQSGSELKKPGASVKVSC | 266 | DIQLTQSPSFLSASVGDRVTITC | 277 |
| | KASGYIFTTYGLSWVRQAPGQG | | RASSSVGYMHWYQQKPGKAPKLL | |
| | LEWMGWINTYSGVPTYTDDFKG | | IYATSNLAQGVPSRFSGSGSGTE | |
| | RFVFSLDTNVSTAYLQISSLKA | | FTLTISSLQPEDFATYYCQQWGS | |
| | EDTAVYYCARYTNYGVFDYWGQ | | LPFTFGQGTKLEIK | |
| | GTLVTVSS | | | |
| MTK-29.9 | QVQLVQSGSELKKPGASVKVSC | 267 | DIQLTQSPSFLSASVGDRVTITC | 278 |
| | KASGYIFTSYGLSWVRQAPGQG | | RASSSVGYMHWYQQKPGKAPKLL | |
| | LEWMGWINTYSGVPTYTDDFKG | | IYATSNLAQGVPSRFSGSGSGTG | |
| | RFVFSLDTSVSTAYLQISSLKA | | FTLTISSLQPEDFATYYCQQWGS | |
| | EDTAVYYCARYTNYGVFDYWGQ | | LPFTFGQGTKLEIK | |
| | GTLVTVSS | | | |
| MTK-29.10 | QVQLVQSGSELKKPGASVKVSC | 268 | EIVLTQSPATLSLSPGERATLSC | 279 |
| | KTSSYTFTTYGMSWVRQAPGQG | | RASSSVGYMHWYQQKPGQAPRLL | |
| | LEWMGWINTYSGVPTYTDDFKG | | IYATSNLASGIPARFSGSGSGTD | |
| | RFVFSLDTSVSTAYLQISSLKA | | FTLTISRLEPEDFAVYYCQQWGS | |
| | EDTAVYYCARYTNYGVFDYWGQ | | LPFTFGGGTKLEIK | |
| | GTLVTVSS | | | |
| MTK-29.11 | QVQLVQSGSELKKPGASVKVSC | 269 | EIVLTQSPATLSLSPGERATLSC | 279 |
| | KASSYTFTTYGMSWVRQAPGQG | | RASSSVGYMHWYQQKPGQAPRLL | |
| | LEWMGWINTDSGVPTYTDDFKG | | IYATSNLASGIPARFSGSGSGTD | |
| | RFVFSLDTSVSTAYLQISSLKA | | FTLTISRLEPEDFAVYYCQQWGS | |
| | EDTAVYYCARYTNYGVFDYWGQ | | LPFTFGGGTKLEIK | |
| | GTLVTVSS | | | |
| MTK-29.12 | QVQLVQSGSELKKPGASVKVSC | 264 | DIQLTQSPGFLSASVGDRVTITC | 280 |
| | KASSYTFTTYGMSWVRQAPGQG | | RASSSVGYMHWYQQKPGKAPKLL | |
| | LEWMGWINTMSGVPTYTDDFKG | | IYATSNLAQGVPSRFSGSGSGTE | |
| | RFVFSLDTSVSTAYLQISSLKA | | FTLTISSLQPEDFATYYCQQWGS | |
| | EDTAVYYCARYTNYGVFDYWGQ | | LPFTFGQGTKLEIK | |
| | GTLVTVSS | | | |
| MTK-29.13 | QVQLVQSGSELKKPGASVKVSC | 270 | DIQLTQSPSFLSASVGDRVTITC | 281 |
| | KASSYTFTTYGMSWVRQAPGQG | | RASSSVGYMHWYQQEPGKAPKLL | |
| | LEWMGWINTASGVPTYTDDFKG | | IYATSNLAQGVPSRFSGSGSGTE | |
| | RFVFSLDTSVSTAYLQISSLKA | | FTLTISSLQPEDFATYYCQQWGS | |
| | EDTAVYYCARYTNYGVFDYWGQ | | LPFTFGQGTKLEIK | |
| | GTLVTVSS | | | |
| MTK-29.14 | QVQLVQSGSELKKPGASVKVSC | 265 | DIQLTQSPSFLSASVGDRVTITC | 282 |
| | KASSYTFTTYGMSWVRQAPGQG | | RASSSVGYMHWYQQKPGKAPKLL | |
| | LEWMGWINTYSGVPSYTDDFKA | | IYATSNLAQGVPSRFSGSGSGTE | |
| | RFVFSLDTSVSTAYLQISSLKA | | FTLTIFSLQPEDFATYYCQQWGS | |
| | EDTAVYYCARYTNYGVFDYWGQ | | LPFTFGQGTKLEIK | |
| | GTLVTVSS | | | |
| MTK-29.15 | QVQLVQSGSELKKPGASVKVSC | 264 | DIQLTQSPSFPSASVGDRVTITC | 283 |
| | KASSYTFTTYGMSWVRQAPGQG | | RASSSVGYMHWYQQKPGKAPKLL | |
| | LEWMGWINTMSGVPTYTDDFKG | | IYATSNLAQGVPSRFSGSGSGTE | |
| | RFVFSLDTSVSTAYLQISSLKA | | FTLTISSLQPEDFATYYCQQWGS | |
| | EDTAVYYCARYTNYGVFDYWGQ | | LPFTFGQGTKLEIK | |
| | GTLVTVSS | | | |
| MTK-29.16 | QVQLVQSGSELKKPGASVKVSC | 271 | DIQLTQSPSFLSASVGDRVTITC | 284 |
| | KASGYIFTSYGLSWVRQAPGQG | | RASSSVGYMHWYQQKPGKAPKLL | |
| | LEWMGWVNTYSGVPTYTDDFKG | | IYATSNLAQGVPSRFSGSGSGTE | |
| | RFVFSLDTSVSTAYLQISSLKA | | FTLTISSLQPEDFATYYCQQWGR | |
| | EDTAVYYCARYTNYGVFDYWGQ | | LPFTFGQGTKLEIK | |
| | GTLVTVSS | | | |
| MTK-29.17 | QVQLVQSGSELKKPGASVKVSC | 272 | DIQLTQSPSFLSASVGDRVTITC | 285 |
| | KASGYIFTSYGLSWVRQAPGQG | | RSSSSVGYMHWYQQKPGKAPKLL | |
| | LEWMGWINTYSGVPTYAQGFTG | | IYATSNLAQGVPSRFSGSGSGTE | |
| | RFVFSLDTSVSTAYLQISSLKA | | FTLTISSLQPEDFATYYCQQWGS | |
| | EDTAVYYCARYTNYGVFDYWGQ | | LPFTFGQGTKLEIK | |
| | GTLVTVSS | | | |
| MTK-29.18 | QVQLVQSGSELKKPGASVKVSC | 271 | EIVLTQSPATLSLSPGERATLSC | 286 |
| | KASGYIFTSYGLSWVRQAPGQG | | RASSTVGYMHWYQQKPGQAPRLL | |
| | LEWMGWVNTYSGVPTYTDDFKG | | IYATSNLASGIPARFSGSGSGTD | |
| | RFVFSLDTSVSTAYLQISSLKA | | FTLTISRLEPEDFAVYYCQQWGS | |
| | EDTAVYYCARYTNYGVFDYWGQ | | LPFTFGGGTKLEIK | |
| | GTLVTVSS | | | |
| MTK-29.19 | QVQLVQSGSELKKPGASVKVSC | 273 | QIVLTQSPGTLSLSPGERATLSC | 287 |
| | KASGFTFTTYGMSWVRQAPGQG | | RATSSVGYMHWYQQKPGQAPRPL | |
| | LEWMGWINTYSGVPTYAQGFTG | | IYATSNLASGIPDRFSGSGSGTS | |
| | RFVFSLDTSVSTAYLQICSLKA | | YTLTISRLEPEDFAVYYCQQWGS | |
| | EDTAVYYCARYTNYGYFDYWGQ | | NPFTFGQGTKLEIK | |
| | GTLVTVSS | | | |
| MTK-29.20 | QVQLVQSGSELKKPGASVKVSC | 273 | QIVLTQSPATLSLSPGERATLSC | 288 |
| | KASGFTFTTYGMSWVRQAPGQG | | RATSSVGYMHWYQQKPGLAPRPL | |
| | LEWMGWINTYSGVPTYAQGFTG | | IYATSNLASGIPDRFSGSGSGTS | |
| | RFVFSLDTSVSTAYLQICSLKA | | YTLTISRLEPEDFAVYYCQQWGS | |
| | EDTAVYYCARYTNYGYFDYWGQ | | NPFTFGQGTKLEIK | |
| | GTLVTVSS | | | |
| MTK-29.21 | QVQLVQSGSELKKPGASVKVSC | 273 | QIVLTQSPATLSLSPGERATLSC | 289 |
| | KASGFTFTTYGMSWVRQAPGQG | | RATSSVGYMHWYQQKPGQAPRPL | |
| | LEWMGWINTYSGVPTYAQGFTG | | IYATSNLASGIPARFSGSGSGTS | |
| | RFVFSLDTSVSTAYLQICSLKA | | YTLTISSLEPEDFAVYYCQQWGS | |
| | EDTAVYYCARYTNYGYFDYWGQ | | NPFTFGQGTKLEIK | |
| | GTLVTVSS | | | |

TABLE 26

| | | SEQ | | SEQ |
|---|---|---|---|---|
| | | ID | | ID |
| Antibody | Heavy Chain Variable | NO: | Light Chain Variable | NO: |
| MTK-33.1 | QVQLVQSGAEVKKPGASVKVSC | 290 | DIQMTQSPSSLSASVGDRVTITC | 309 |
| | KASGYTFTSYWMHWVRQAPGQG | | QASQSVSNTVAWYQQKPGKAPKL | |
| | LEWIGVIDPSDNYINYNQKFQG | | LIYYASLRYTGVPSRFSGSGSGT | |
| | RVTMTRDTSTSTVYMELSSLRS | | DFTFTISSLQPEDIATYYCQQDY | |
| | EDTAVYYCAREAWTRGYFNYWG | | RSPFTFGQGTKLEIK | |
| | QGTLVTVSS | | | |
| MTK-33.2 | QVQLVQSGAEVKKPGASVKVSC | 291 | DIQMTQSPSSLSASVGDRVTITC | 310 |
| | KASGYTSTSYWMHWVRQAPGQG | | QASGSVSNTVAWYQQKPGKAPKL | |
| | LEWIGVIDPSDNYINYNQKFQG | | LIYYASNRYTGVPSRFSGSGSGT | |
| | RVTMTRDTSTSTVYMELSSLRS | | DFTFTISSLQPEDIATYYCQQDY | |
| | EDTAVYYCAREAGTRGYFDYWG | | RSPFTFGQGTKLEIK | |
| | QGTLVTVSS | | | |
| MTK-33.3 | QVQLVQSGAEVKKPGASVKVSC | 292 | DIQMTQSPSSLSASVGDRVTITC | 311 |
| | KASGYTFTSYWMHWVRQAPGQG | | QASRSVSNTVAWYQQKPGKAPKL | |
| | LEWIGVIDPSDNYINYNQKFQG | | LIYYASNRYTGVPSRFSGSGSGT | |
| | RVTMTRDTSTSTVYMELSSLRS | | DFTFTISSLQPEDIATYYCQQDY | |
| | EDTAVYYCAREAWTRGYFDYWG | | RSPFTFGQGTKLEIK | |
| | QGTLVTVSS | | | |
| MTK-33.4 | QVQLVQSGAEVKKPGASVKVSC | 293 | DIQMTQSPSSLSASVGDRVTITC | 312 |
| | KASGYTFTSYWMHWVRQAPGQG | | QASQSVSNTVAWYQQKPGKAPKL | |
| | LEWIGVIDPSDNYINMNQKFQG | | LIYYASNRYTGVPSRFSGSGSGT | |
| | RVTMTRDTSTSTVYMELSSLRS | | DFTFTISSLQPEDIATYYCQQDR | |
| | EDTAVYYCAREAGTRGYFDYWG | | RSPFTFGQGTKLEIK | |
| | QGTLVTVSS | | | |
| MTK-33.5 | QVQLVQSGAEVKKPGASVKVSC | 294 | DIQMTQSPSSLSASVGDRVTITC | 313 |
| | MASGYTFGSYWMHWVRQAPGQG | | QASQSVSNTVAWYQQKPGKAPKL | |
| | LEWIGVIDPSDNYINYNQKFQG | | LIYYASNRETGVPSRFSGSGSGT | |
| | RVTMTRDTSTSTVYMELSSLRS | | DFTFTISSLQPEDIGTYYCQQDY | |
| | EDTAVYYCAREAGTRGYFDYWG | | RSPFTFGQGTKLEIK | |
| | QGTLVTVSS | | | |
| MTK-33.6 | QVQLVQSGAEVKKPGASVKVSC | 295 | DIQMTQSPSSLSASVGDRVTITC | 314 |
| | MASGYTFRSYWMHWVRQAPGQG | | QASRSVSNTMAWYQQKPGKAPKL | |
| | LEWIGVIDPSDNYINYNQKFQG | | LIYYASNRYTGVPSRFSGSGSGT | |
| | RVTMTRDTSTSTVYMELSSLRS | | DFTFTISSLQPEDIATYYCQQDY | |
| | EDTAVYYCAREAGTRGYFDYWG | | RSPFTFGQGTKLEIK | |
| | QGTLVTVSS | | | |
| MTK-33.7 | QVQLVQSGAEVKKPGASVKVSC | 296 | DIQMTQSPSSLSASVGDRVTITC | 315 |
| | KASGYTFTSYWMHWVRQAPGQG | | GASQSVSNTVAWYQQKPGKAPKL | |
| | LEWIGVIDPSDNYINYNAKFQG | | LIYYASNRYTGVPSRFSGSGSGT | |
| | RVTMTRDTSTSTVYMELSSLRS | | DFTFTISSLQPEDIATYYCQQDY | |
| | EDTAVYYCAREAGTRGYFDYWG | | RSPFTFGQGTKLEIK | |
| | QGTLVTVSS | | | |
| MTK-33.8 | QVQLVQSGAEVKKPGASVKVSC | 290 | DIQMTQSPSSLSASVGDRVTITC | 316 |
| | KASGYTFTSYWMHWVRQAPGQG | | QASQSVSRTVAWYQQKPGKAPKL | |
| | LEWIGVIDPSDNYINYNQKFQG | | LIYYASNRYTGVPSRFSGSGSGT | |
| | RVTMTRDTSTSTVYMELSSLRS | | DFTFTISSLQPEDIATYYCQQDY | |
| | EDTAVYYCAREAWTRGYFNYWG | | RSPFTFGQGTKLEIK | |
| | QGTLVTVSS | | | |
| MTK-33.9 | QVQLVQSGAEVKKPGASVKVSC | 297 | DIQMTQSPSSLSASVGDRVTITC | 317 |
| | KASGYTFTSYWMHWVRQAPGQG | | QASASVSNTVAWYQQKPGKAPKL | |
| | LEWIGVIDPSDRYINYNQKFQG | | LIYYASNRYTGVPSRFSGSGSGT | |
| | RVTMTRDTSTSTVYMELSSLRS | | DFTFTISSLQPEDIATYYCQQDY | |
| | EDTAVYYCAREAGTRGYFDYWG | | RSPFTFGQGTKLEIK | |
| | QGTLVTVSS | | | |
| MTK-33.10 | QVQLVQSGAEVKKPGASVKVSC | 298 | DIQMTQSPSSLSASVGDRVTITC | 318 |
| | KASSYSFTSYWMHWVRQAPGQG | | QAGQSVSNTVAWYQQKPGKAPKL | |
| | LEWIGVIDPSDNYINYNQKFRG | | LIYYASNRYTGVPSRFSGSGSGT | |
| | RVTMTRDTSTSTVYMELSSLRS | | DFTFTISSLQPEDIATYYCQQDY | |
| | EDTAVYYCAREAGTRGYFDYWG | | RSPFTFGQGTKLEIK | |
| | QGTLVTVSS | | | |
| MTK-33.11 | QVQLVQSGAEVKKPGASVKVSC | 292 | DIQMTQSPSSLSASVGDRVTITC | 319 |
| | KASGYTFTSYWMHWVRQAPGQG | | QASRSVRNTVAWYQQKPGKAPKL | |
| | LEWIGVIDPSDNYINYNQKFQG | | LIYYASNRYTGVPSRFSGSGSGT | |
| | RVTMTRDTSTSTVYMELSSLRS | | DFTFTISSLQPEDIATYYCQQDY | |
| | EDTAVYYCAREAWTRGYFDYWG | | RSPFTFGQGTKLEIK | |
| | QGTLVTVSS | | | |
| MTK-33.12 | QVQLVQSGAEVKKPGASVKVSC | 299 | DIQMTQSPSSLSASVGDRVTITC | 320 |
| | KASGYTFVSYWMHWVRQAPGQG | | QASQSVSNTVAWYQQKPGKAPKL | |
| | LEWIGVIDPSDNYINYNQKFRG | | LIYYASNRYTGVPSRFSGSGSGT | |
| | RVTMTRDTSTSTVYMELSSLRS | | DFTFTISSLQPEDIATYYCRQDT | |
| | EDTAVYYCAREAGTRGYFDYWG | | RSPFTFGQGTKLELK | |
| | QGTLVTVSS | | | |
| MTK-33.13 | QVQLVQSGAEVKKPGASVKVSC | 300 | DIQMTQSPSSLSASVGDRVTITC | 311 |
| | KASGYTFTSYWMHWVRQAPGQG | | QASRSVSNTVAWYQQKPGKAPKL | |
| | LEWIGVIDPSDNYINYNQKFQG | | LIYYASNRYTGVPSRFSGSGSGT | |
| | RVTMTRDTSTSTVYMELSSLRS | | DFTFTISSLQPEDIATYYCQQDY | |
| | EDTAVYYCAREAGTRGYFDYWG | | RSPFTFGQGTKLEIK | |
| | QGTLVTVSS | | | |
| MTK-33.14 | QVQLVQSGAEVKKPGASVKVSC | 301 | DIQMTQSPSSLSASVGDRVTITC | 321 |
| | KASGYTFTSYWMHWVRQAPGQG | | QASQSVSNTVAWYQQKPGKAPKL | |
| | LEWIGVIDPSDNYINYNQKFRG | | LIYYASNRYTGVPSRFSGSGSGT | |
| | RVTMTRDTSTSTVYMELSSLRS | | DFTFTISSLQPEDIATYYCQQDY | |
| | EDTAVYYCAREAGTRGYFDYWG | | RSPFTFGQGTKLEIK | |
| | QGTLVTVSS | | | |
| MTK-33.15 | QVQLVQSGAEVKKPGASVKVSC | 302 | DIQMTQSPSSLSASVGDRVTITC | 322 |
| | KASGYTFTSYWMHWVRQAPGQG | | QASRSVSNTVAWYQQKPGKAPKL | |
| | LEWIGVIDPSDNYINYNQKFQV | | LIYYASNRYTGVPSRFSGSGSGT | |
| | RVTMTRDTSTSTVYMELSSLRS | | DFTFTISSLQPEDIATYYCQQDM | |
| | EDTAVYYCAREAGTRGYFDYWG | | RSPFTFGQGTKLEIK | |
| | QGTLVTVSS | | | |
| MTK-33.16 | QVQLVQSGAEVKKPGASVKVSC | 303 | DIQMTQSPSSLSASVGDRVTITC | 311 |
| | KASWYTFTSYWMHWVRQAPGQG | | QASRSVSNTVAWYQQKPGKAPKL | |
| | LEWIGVIDPSDNYINYNQKFQG | | LIYYASNRYTGVPSRFSGSGSGT | |
| | RVTMTRDTSTSTVYMELSSLRS | | DFTFTISSLQPEDIATYYCQQDY | |
| | EDTAVYYCAREAGTRGYFDYWG | | RSPFTFGQGTKLEIK | |
| | QGTLVTVSS | | | |
| MTK-33.17 | QVQLVQSGAEVKKPGASVKVSC | 304 | DIQMTQSPSSLSASVGDRVTITC | 322 |
| | KASGYTFTSYWMHWVRQAPGQG | | QASRSVSNTVAWYQQKPGKAPKL | |
| | LEWIGVIAPSDNYINYNQKFRG | | LIYYASNRYTGVPSRFSGSGSGT | |
| | RVTMTRDTSTSTVYMELSSLRS | | DFTFTISSLQPEDIATYYCQQDM | |
| | EDTAVYYCAREAGTRGYFDYWG | | RSPFTFGQGTKLEIK | |
| | QGTLVTVSS | | | |
| MTK-33.18 | QVQLVQSGAEVKKPGASVKVSC | 305 | DIQMTQSPSSLSASVGDRVTITC | 322 |
| | KASGYTFTSYWMHWVRQAPGQG | | QASRSVSNTVAWYQQKPGKAPKL | |
| | LEWIGVISPSDNYINYNQKFRG | | LIYYASNRYTGVPSRFSGSGSGT | |
| | RVTMTRDTSTSTVYMELSSLRS | | DFTFTISSLQPEDIATYYCQQDM | |
| | EDTAVYYCAREAGTRGYFDYWG | | RSPFTFGQGTKLEIK | |
| | QGTLVTVSS | | | |
| MTK-33.19 | QVQLVQSGAEVKKPGASVKVSC | 301 | DIQMTQSPSSLSASVGDRVTITC | 323 |
| | KASGYTFTSYWMHWVRQAPGQG | | QASRSVSATVAWYQQKPGKAPKL | |
| | LEWIGVIDPSDNYINYNQKFRG | | LIYYASNRYTGVPSRFSGSGSGT | |
| | RVTMTRDTSTSTVYMELSSLRS | | DFTFTISSLQPEDIATYYCQQDM | |
| | EDTAVYYCAREAGTRGYFDYWG | | RSPFTFGQGTKLEIK | |
| | QGTLVTVSS | | | |
| MTK-33.20 | QVQLVQSGAEVKKPGASVKVSC | 301 | DIQMTQSPSSLSASVGDRVTITC | 324 |
| | KASGYTFTSYWMHWVRQAPGQG | | QASRSVSNTVAWYQQKPGKAPKL | |
| | LEWIGVIDPSDNYINYNQKFRG | | LIYYASNRYTGVPSRFSGSGSGT | |
| | RVTMTRDTSTSTVYMELSSLRS | | DFTFTISSLQPEDIATYYCAQDM | |
| | EDTAVYYCAREAGTRGYFDYWG | | RSPFTFGQGTKLEIK | |
| | QGTLVTVSS | | | |
| MTK-33.21 | QVQLVQSGAEVKKPGASVKVSC | 301 | DIQMTQSPSSLSASVGGRVTITC | 325 |
| | KASGYTFTSYWMHWVRQAPGQG | | QASRSVSNTVAWYQQKPGKAPKL | |
| | LEWIGVIDPSDNYINYNQKFRG | | LIYYVSNRYTGVPSRFSGSGSGT | |
| | RVTMTRDTSTSTVYMELSSLRS | | DFTFTISSLQPEDIATYYCQQDM | |
| | EDTAVYYCAREAGTRGYFDYWG | | RSPFTFGQGTKLEIK | |
| | QGTLVTVSS | | | |
| MTK-33.22 | QVQLVQSGAEVKKPGASVKVSC | 306 | DIQMTQSPSSLSASVGDRVTITC | 326 |
| | KASGYTFTSYWMHWVRQAPGQG | | QASRSVSNTVAWYQQKPGKAPKL | |
| | LEWIGVIDPSDNYINYNQKLRG | | LIYYASNRRTGVPSRFSGSGSGT | |
| | RVTMTRDTSTSTVYMELSSLRS | | DFTFTISSLQPEDIATYYCQQDM | |
| | EDTAVYYCAREAGTRGYFDYWG | | RSPFTFGQGTKLEIK | |
| | QGTLVTVSS | | | |
| MTK-33.23 | QVQLVQSGAEVKKPGASVKVSC | 307 | DIQMTQSPSSLSASVGDRVTITC | 327 |
| | KASGYTFTSYWMHWVRQAPGQG | | IASRSVSNTVAWYQQKPGKAPKL | |
| | LEWIGVIDPSDNYINYNQKFRG | | LIYYASNRYTGVPSRFSGSGSGT | |
| | RVTMTRDTSTSTVYMELSSLRS | | DFTFTISSLQPEDIATYYCQQDM | |
| | EDTAVYYCAREAYTRGYFDYWG | | RSPFTFGQGTKLEIK | |
| | QGTLVTVSS | | | |
| MTK-33.24 | QVQLVQSGAEVKKPGASVKVSC | 308 | DIQMTQSPSSLSASVGDRVTITC | 328 |
| | KASSYTFTSYWMHWVRQAPGQG | | QASRSVSRTVAWYQQKPGKAPKL | |
| | LEWIGVIDPSDNYINYNQKFRG | | LIYYASNRYTGVPSRFSGSGSGT | |
| | RVTMTRDTSTSTVYMELSSLRS | | DFTFTISSLQPEDIATYYCQQDM | |
| | EDTAVYYCAREAGTRGYFDYWG | | RSPFTFGQGTKLEIK | |
| | QGTLVTVSS | | | |

TABLE 27

| | | SEQ | | SEQ | | SEQ |
|---|---|---|---|---|---|---|
| | | ID | | ID | | ID |
| Antibody | HVR-H1 | NO: | HVR-H2 | NO: | HVR-H3 | NO: |
| MTK-15.1 | NYGVH | 83 | WINTYTGEPT | 329 | GNRYAYQDY | 334 |
| | | | YAQGFTG | | | |
| MTK-15.2 | NYGVH | 83 | WGNTYTGEPT | 330 | GNRYAYMDY | 138 |
| | | | YAQGFTG | | | |
| MTK-15.3 | NYGVH | 83 | WINTYTGEPT | 329 | GNRYAYMDY | 138 |
| | | | YAQGFTG | | | |
| MTK-15.4 | NYGVH | 83 | WINTYTGEPT | 329 | GNRYAYMDY | 138 |
| | | | YAQGFTG | | | |
| MTK-15.5 | NYGVH | 83 | WINTYTGEPT | 329 | GTRYAYMDY | 335 |
| | | | YAQGFTG | | | |
| MTK-15.6 | NYGVH | 83 | WINTYTGEPY | 331 | GNRYAYMDY | 138 |
| | | | YAQGFTG | | | |
| MTK-15.7 | NYGVH | 83 | WINTYTGEPT | 329 | GNRYAYMDY | 138 |
| | | | YAQGFTG | | | |
| MTK-15.8 | NYGVH | 83 | WINTYTGEPT | 329 | GVRYAYMDY | 336 |
| | | | YAQGFTG | | | |
| MTK-15.9 | NYGVH | 83 | WINTYTGEPT | 329 | GYRYAYMDY | 337 |
| | | | YAQGFTG | | | |
| MTK-15.10 | NYGVH | 83 | WINTYQGEPT | 332 | GARYAYMDY | 338 |
| | | | YAQGFTG | | | |
| MTK-15.11 | NYGVH | 83 | WINTYEGEPT | 333 | GNRYAYQDY | 334 |
| | | | YAQGFTG | | | |
| MTK-15.12 | NYGVH | 83 | WINTYQGEPT | 332 | GVRYAYMDY | 336 |
| | | | YAQGFTG | | | |
| MTK-15.13 | NYGVH | 83 | WINTYTGEPT | 329 | GNRYAYEDY | 339 |
| | | | YAQGFTG | | | |
| MTK-15.14 | NYGVH | 83 | WINTYTGEPT | 329 | GNRYAYMDY | 138 |
| | | | YAQGFTG | | | |
| MTK-15.15 | NYGVH | 83 | WINTYTGEPT | 329 | GNRYAYMDY | 138 |
| | | | YAQGFTG | | | |

TABLE 28

| | | SEQ | | SEQ | | SEQ |
|---|---|---|---|---|---|---|
| | | ID | | ID | | ID |
| Antibody | HVR-L1 | NO: | HVR-L2 | NO: | HVR-L3 | NO: |
| MTK-15.1 | RASSSVSYMH | 158 | ATSNLAS | 187 | QQWSSNPRT | 210 |
| MTK-15.2 | RASSSVSYMH | 158 | ATSNLAS | 187 | QQWSSNPRT | 210 |
| MTK-15.3 | RASSHVSYMH | 340 | ATSNLAS | 187 | QQWSSNPRT | 210 |
| MTK-15.4 | RASSSVGYMH | 341 | ATSNLAS | 187 | QQWSSNPRT | 210 |
| MTK-15.5 | RASSSVGYMH | 341 | ATSNLAS | 187 | QQWSSNPRT | 210 |
| MTK-15.6 | RASSSVGYMH | 341 | ATSNLAS | 187 | QQWSSNPRT | 210 |
| MTK-15.7 | RASSGVSYMH | 342 | ATSNLAS | 187 | QQWSSNPRT | 210 |
| MTK-15.8 | RASSSVGYMH | 341 | ATSNLAS | 187 | QQWSSNPRT | 210 |
| MTK-15.9 | RASSSTSYMH | 343 | ATSNLAS | 187 | QQWSSNPRT | 210 |
| MTK-15.10 | RASSSVGYMH | 341 | ATSNLAS | 187 | QQWSSNPRT | 210 |
| MTK-15.11 | RASSSVSYMH | 158 | ATSNLAS | 187 | QQWSSNPRT | 210 |
| MTK-15.12 | RASSSVGYMH | 341 | ATSNLAS | 187 | QQWSSNPRT | 210 |
| MTK-15.13 | RASSSVQYMH | 344 | ATSNLAS | 187 | QQWSSNPRT | 210 |
| MTK-15.14 | RASSSVSYMH | 158 | ATSNLAS | 187 | QQWSSNPRT | 210 |
| MTK15.15 | RASSSVSYMH | 158 | ATSNLAS | 187 | QQWSSNPRT | 210 |

TABLE 29

| | | SEQ | | SEQ | | SEQ |
|---|---|---|---|---|---|---|
| | | ID | | ID | | ID |
| Antibody | HVR-H1 | NO: | HVR-H2 | NO: | HVR-H3 | NO: |
| MTK-16.1 | NHGMN | 84 | WINTYTGEPT | 329 | KGVTAARYF | 139 |
| | | | YAQGFTG | | DY | |
| MTK-16.2 | NHGMN | 84 | WINTYTGEPT | 99 | KGVTAARYF | 139 |
| | | | YADDFKG | | DY | |

TABLE 30

| | | SEQ ID | | SEQ ID | | SEQ ID |
|---|---|---|---|---|---|---|
| Antibody | HVR-L1 | NO: | HVR-L2 | NO: | HVR-L3 | NO: |
| MTK-16.1 | KASQNVRTAVA | 169 | LASNRHT | 195 | LQHWNYPLT | 219 |
| MTK-16.2 | RASQNVRTAVA | 345 | LASNRHT | 195 | LQHWNYPLT | 219 |

TABLE 31

| | | | | SEQ | | SEQ |
|---|---|---|---|---|---|---|
| | | SEQ ID | | ID | | ID |
| Antibody | HVR-H1 | NO: | HVR-H2 | NO: | HVR-H3 | NO: |
| MTK-29.1 | SYGLS | 346 | WINTYSGVPT | 348 | YTNYGIFDY | 355 |
| | | | YAQGFTG | | | |
| MTK-29.2 | TYGMS | 95 | WINTYSGVPT | 119 | YTNYGIFDY | 355 |
| | | | YTDDFKG | | | |
| MTK-29.3 | TYGMS | 95 | WINTYSGVPA | 349 | YTNYGVFDY | 356 |
| | | | YTDDFKG | | | |
| MTK-29.4 | TYGMS | 95 | WINTYSGVPT | 119 | YTNYGVFDY | 356 |
| | | | YTDDFKG | | | |
| MTK-29.5 | SYGLS | 346 | WINTYSGVPT | 119 | YTNYGVFDY | 356 |
| | | | YTDDFKG | | | |
| MTK-29.6 | TYGMS | 95 | WINTMSGVPT | 350 | YTNYGVFDY | 356 |
| | | | YTDDFKG | | | |
| MTK-29.7 | TYGMS | 95 | WINTYSGVPS | 351 | YTNYGVFDY | 356 |
| | | | YTDDFKA | | | |
| MTK-29.8 | TYGLS | 347 | WINTYSGVPT | 119 | YTNYGVFDY | 356 |
| | | | YTDDFKG | | | |
| MTK-29.9 | SYGLS | 346 | WINTYSGVPT | 119 | YTNYGVFDY | 356 |
| | | | YTDDFKG | | | |
| MTK-29.10 | TYGMS | 95 | WINTYSGVPT | 119 | YTNYGVFDY | 356 |
| | | | YTDDFKG | | | |
| MTK-29.11 | TYGMS | 95 | WINTDSGVPT | 352 | YTNYGVFDY | 356 |
| | | | YTDDFKG | | | |
| MTK-29.12 | TYGMS | 95 | WINTMSGVPT | 350 | YTNYGVFDY | 356 |
| | | | YTDDFKG | | | |
| MTK-29.13 | TYGMS | 95 | WINTASGVPT | 353 | YTNYGVFDY | 356 |
| | | | YTDDFKG | | | |
| MTK-29.14 | TYGMS | 95 | WINTYSGVPS | 351 | YTNYGVFDY | 356 |
| | | | YTDDFKA | | | |
| MTK-29.15 | TYGMS | 95 | WINTMSGVPT | 350 | YTNYGVFDY | 356 |
| | | | YTDDFKG | | | |
| MTK-29.16 | SYGLS | 346 | WVNTYSGVPT | 354 | YTNYGVFDY | 356 |
| | | | YTDDFKG | | | |
| MTK-29.17 | SYGLS | 346 | WINTYSGVPT | 348 | YTNYGVFDY | 356 |
| | | | YAQGFTG | | | |
| MTK-29.18 | SYGLS | 346 | WVNTYSGVPT | 354 | YTNYGVFDY | 356 |
| | | | YTDDFKG | | | |
| MTK-29.19 | TYGMS | 95 | WINTYSGVPT | 348 | YTNYGYFDY | 151 |
| | | | YAQGFTG | | | |
| MTK-29.20 | TYGMS | 95 | WINTYSGVPT | 348 | YTNYGYFDY | 151 |
| | | | YAQGFTG | | | |
| MTK-29.21 | TYGMS | 95 | WINTYSGVPT | 348 | YTNYGYFDY | 151 |
| | | | YAQGFTG | | | |

TABLE 32

| Antibody | HVR-L1 | SEQ ID NO: | HVR-L2 | SEQ ID NO: | HVR-L3 | SEQ ID NO: |
|---|---|---|---|---|---|---|
| MTK-29.1 | RASSSVGYMH | 341 | ATSNLAS | 187 | QQWGSWPFT | 360 |
| MTK-29.2 | RASSSVGYMH | 341 | ATSNLAS | 187 | QQWGSIPFT | 361 |
| MTK-29.3 | RASSSVGYMH | 341 | ATSNLAS | 187 | QQWGSLPFT | 362 |
| MTK-29.4 | RASSSVGYMH | 341 | ATSNLAQ | 359 | QQWGSLPFT | 362 |
| MTK-29.5 | RASSSVGYMH | 341 | ATSNLAQ | 359 | QQWGSLPFT | 362 |
| MTK-29.6 | RASSSVGYMH | 341 | ATSNLAQ | 359 | QQWGSLPFT | 362 |
| MTK-29.7 | RASSSVGYMH | 341 | ATSNLAQ | 359 | QQWGSLPFT | 362 |
| MTK-29.8 | RASSSVGYMH | 341 | ATSNLAQ | 359 | QQWGSLPFT | 362 |
| MTK-29.9 | RASSSVGYMH | 341 | ATSNLAQ | 359 | QQWGSLPFT | 362 |
| MTK-29.10 | RASSSVGYMH | 341 | ATSNLAS | 187 | QQWGSLPFT | 362 |
| MTK-29.11 | RASSSVGYMH | 341 | ATSNLAS | 187 | QQWGSLPFT | 362 |
| MTK-29.12 | RASSSVGYMH | 341 | ATSNLAQ | 359 | QQWGSLPFT | 362 |
| MTK-29.13 | RASSSVGYMH | 341 | ATSNLAQ | 359 | QQWGSLPFT | 362 |
| MTK-29.14 | RASSSVGYMH | 341 | ATSNLAQ | 359 | QQWGSLPFT | 362 |
| MTK-29.15 | RASSSVGYMH | 341 | ATSNLAQ | 359 | QQWGSLPFT | 362 |
| MTK-29.16 | RASSSVGYMH | 341 | ATSNLAQ | 359 | QQWGRLPFT | 363 |
| MTK-29.17 | RSSSSVGYMH | 357 | ATSNLAQ | 359 | QQWGSLPFT | 362 |
| MTK-29.18 | RASSTVGYMH | 358 | ATSNLAS | 187 | QQWGSLPFT | 362 |
| MTK-29.19 | RATSSVGYMH | 181 | ATSNLAS | 187 | QQWGSNPFT | 208 |
| MTK-29.20 | RATSSVGYMH | 181 | ATSNLAS | 187 | QQWGSNPFT | 208 |
| MTK-29.21 | RATSSVGYMH | 181 | ATSNLAS | 187 | QQWGSNPFT | 208 |

TABLE 33

| | | SEQ | | SEQ | | SEQ |
|---|---|---|---|---|---|---|
| Anti- | HVR- | ID | | ID | | ID |
| body | H1 | NO: | HVR-H2 | NO: | HVR-H3 | NO: |
| MTK- | SYWMH | 90 | VIDPSDNYIN | 364 | EAWTRGYFN | 373 |
| 33.1 | | | YNQKFQG | | Y | |
| MTK- | SYWMH | 90 | VIDPSDNYIN | 364 | EAGTRGYFD | 155 |
| 33.2 | | | YNQKFQG | | Y | |
| MTK- | SYWMH | 90 | VIDPSDNYIN | 364 | EAWTRGYFD | 374 |
| 33.3 | | | YNQKFQG | | Y | |
| MTK- | SYWMH | 90 | VIDPSDNYIN | 365 | EAGTRGYFD | 155 |
| 33.4 | | | MNQKFQG | | Y | |
| MTK- | SYWMH | 90 | VIDPSDNYIN | 364 | EAGTRGYFD | 155 |
| 33.5 | | | YNQKFQG | | Y | |
| MTK- | SYWMH | 90 | VIDPSDNYIN | 364 | EAGTRGYFD | 155 |
| 33.6 | | | YNQKFQG | | Y | |
| MTK- | SYWMH | 90 | VIDPSDNYIN | 366 | EAGTRGYFD | 155 |
| 33.7 | | | YNAKFQG | | Y | |
| MTK- | SYWMH | 90 | VIDPSDNYIN | 364 | EAWTRGYFN | 373 |
| 33.8 | | | YNQKFQG | | Y | |
| MTK- | SYWMH | 90 | VIDPSDRYIN | 367 | EAGTRGYFD | 155 |
| 33.9 | | | YNQKFQG | | Y | |
| MTK- | SYWMH | 90 | VIDPSDNYIN | 368 | EAGTRGYFD | 155 |
| 33.10 | | | YNQKFRG | | Y | |
| MTK- | SYWMH | 90 | VIDPSDNYIN | 364 | EAWTRGYFD | 374 |
| 33.11 | | | YNQKFQG | | Y | |
| MTK- | SYWMH | 90 | VIDPSDNYIN | 368 | EAGTRGYFD | 155 |
| 33.12 | | | YNQKFRG | | Y | |
| MTK- | SYWMH | 90 | VIDPSDNYIN | 364 | EAGTRGYFD | 155 |
| 33.13 | | | YNQKFQG | | Y | |
| MTK- | SYWMH | 90 | VIDPSDNYIN | 368 | EAGTRGYFD | 155 |
| 33.14 | | | YNQKFRG | | Y | |
| MTK- | SYWMH | 90 | VIDPSDNYIN | 369 | EAGTRGYFD | 155 |
| 33.15 | | | YNQKFQV | | Y | |
| MTK- | SYWMH | 90 | VIDPSDNYIN | 364 | EAGTRGYFD | 155 |
| 33.16 | | | YNQKFQG | | Y | |
| MTK- | SYWMH | 90 | VIAPSDNYIN | 370 | EAGTRGYFD | 155 |
| 33.17 | | | YNQKFRG | | Y | |
| MTK- | SYWMH | 90 | VISPSDNYIN | 371 | EAGTRGYFD | 155 |
| 33.18 | | | YNQKFRG | | Y | |
| MTK- | SYWMH | 90 | VIDPSDNYIN | 368 | EAGTRGYFD | 155 |
| 33.19 | | | YNQKFRG | | Y | |
| MTK- | SYWMH | 90 | VIDPSDNYIN | 368 | EAGTRGYFD | 155 |
| 33.20 | | | YNQKFRG | | Y | |
| MTK- | SYWMH | 90 | VIDPSDNYIN | 368 | EAGTRGYFD | 155 |
| 33.21 | | | YNQKFRG | | Y | |
| MTK- | SYWMH | 90 | VIDPSDNYIN | 372 | EAGTRGYFD | 155 |
| 33.22 | | | YNQKLRG | | Y | |
| MTK- | SYWMH | 90 | VIDPSDNYIN | 368 | EAYTRGYFD | 375 |
| 33.23 | | | YNQKFRG | | Y | |
| MTK- | SYWMH | 90 | VIDPSDNYIN | 368 | EAGTRGYFD | 155 |
| 33.24 | | | YNQKFRG | | Y | |

TABLE 34

| | | SEQ ID | | SEQ ID | | SEQ ID |
|---|---|---|---|---|---|---|
| Antibody | HVR-L1 | NO: | HVR-L2 | NO: | HVR-L3 | NO: |
| MTK-33.1 | QASQSVSNTVA | 376 | YASLRYT | 388 | QQDYRSPFT | 231 |
| MTK-33.2 | QASGSVSNTVA | 377 | YASNRYT | 203 | QQDYRSPFT | 231 |
| MTK-33.3 | QASRSVSNTVA | 378 | YASNRYT | 203 | QQDYRSPFT | 231 |
| MTK-33.4 | QASQSVSNTVA | 376 | YASNRYT | 203 | QQDRRSPFT | 392 |
| MTK-33.5 | QASQSVSNTVA | 376 | YASNRET | 389 | QQDYRSPFT | 231 |
| MTK-33.6 | QASRSVSNTMA | 379 | YASNRYT | 203 | QQDYRSPFT | 231 |
| MTK-33.7 | GASQSVSNTVA | 380 | YASNRYT | 203 | QQDYRSPFT | 231 |
| MTK-33.8 | QASQSVSRTVA | 381 | YASNRYT | 203 | QQDYRSPFT | 231 |
| MTK-33.9 | QASASVSNTVA | 382 | YASNRYT | 203 | QQDYRSPFT | 231 |
| MTK-33.10 | QAGQSVSNTVA | 383 | YASNRYT | 203 | QQDYRSPFT | 231 |
| MTK-33.11 | QASRSVRNTVA | 384 | YASNRYT | 203 | QQDYRSPFT | 231 |
| MTK-33.12 | QASQSVSNTVA | 376 | YASNRYT | 203 | RQDTRSPFT | 393 |
| MTK-33.13 | QASRSVSNTVA | 378 | YASNRYT | 203 | QQDYRSPFT | 231 |
| MTK-33.14 | QASQSVSNTVA | 376 | YASNRYT | 203 | QQDYRSPFT | 231 |
| MTK-33.15 | QASRSVSNTVA | 378 | YASNRYT | 203 | QQDMRSPFT | 394 |
| MTK-33.16 | QASRSVSNTVA | 378 | YASNRYT | 203 | QQDYRSPFT | 231 |
| MTK-33.17 | QASRSVSNTVA | 378 | YASNRYT | 203 | QQDMRSPFT | 394 |
| MTK-33.18 | QASRSVSNTVA | 378 | YASNRYT | 203 | QQDMRSPFT | 394 |
| MTK-33.19 | QASRSVSATVA | 385 | YASNRYT | 203 | QQDMRSPFT | 394 |
| MTK-33.20 | QASRSVSNTVA | 378 | YASNRYT | 203 | AQDMRSPFT | 395 |
| MTK-33.21 | QASRSVSNTVA | 378 | YVSNRYT | 390 | QQDMRSPFT | 394 |
| MTK-33.22 | QASRSVSNTVA | 378 | YASNRRT | 391 | QQDMRSPFT | 394 |
| MTK-33.23 | IASRSVSNTVA | 386 | YASNRYT | 203 | QQDMRSPFT | 394 |
| MTK-33.24 | QASRSVSRTVA | 387 | YASNRYT | 203 | QQDMRSPFT | 394 |

### Example 26: Blocking Efferocytosis with Humanized and Affinity-Matured Anti-MerTK Antibodies

The ability of humanized and affinity-matured anti-MerTK antibodies of the present disclosure to block efferocytosis was evaluated using methods as described above in Example 11 with the following modifications. Differentiated human macrophages were treated with 100 nM Dexamethasone (Sigma-Aldrich) and 10 μg/ml huM-CSF (R&D systems) for 2 days. 50,000 cells of polarized macrophages were seeded in 96-well plate. Humanized or affinity-matured anti-MerTK antibodies were titrated to a final concentration range of between 66.6 nM to 4 pM and then each serially diluted antibody was added to each well for 30 min at 37° C.

Staurosporin-induced apoptotic Jurkat cells were labeled with pHrodo (ThermoFisher) and then added into each well at 1:4 ratio (1 macrophage: 4 Jurkat cells) for 1 hour. The plates were washed with PBS and then the cells were stained with APC-conjugated anti-human CD14 for 30 minutes on ice in the dark. Cells were then washed twice in FACS buffer (PBS+2% FBS), and flow cytometry was performed on a BD FACS Canto IL. Data were analyzed using FlowJo software. In these experiments, baseline efferocytosis levels were established using macrophages cultured with media alone and this was set to 10000 efferocytosis activity. Relative efferocytosis activity was calculated as a percent of efferocytosis observed in cells treated with media alone compared to that observed in cells treated with anti-MerTK antibodies. The results of these studies are shown below in Table 35.

TABLE 35

| | Efferocytosis blocking |
|---|---|
| | activity average IC50 |
| | (nM) from at least |
| Antibody | three different donors |
| MTK-15.1 | 0.594 |
| MTK-15.2 | 0.867 |
| MTK-15.3 | 1.006 |
| MTK-15.4 | 0.764 |
| MTK-15.5 | 0.556 |
| MTK-15.6 | 0.506 |
| MTK-15.7 | 0.789 |
| MTK-15.8 | 0.648 |
| MTK-15.9 | 2.432 |
| MTK-15.10 | 0.579 |
| MTK-15.11 | 1.014 |
| MTK-15.12 | 1.016 |
| MTK-15.13 | 0.722 |
| MTK-15.14 | 0.902 |
| MTK-15.15 | 0.228 |
| MTK-16.1 | 0.363 |
| MTK-16.2 | 1.010 |
| MTK-29.5 | 0.572 |
| MTK-29.7 | 0.768 |
| MTK-29.8 | 0.844 |
| MTK-29.9 | 0.632 |
| MTK-29.10 | 0.823 |
| MTK-29.11 | 1.006 |
| MTK-29.12 | 1.119 |
| MTK-29.13 | 0.974 |
| MTK-29.14 | 0.719 |
| MTK-29.15 | 0.963 |
| MTK-29.16 | 0.433 |
| MTK-29.17 | 0.963 |
| MTK-29.18 | 0.697 |
| MTK-29.19 | 0.705 |
| MTK-29.20 | 0.500 |
| MTK-29.21 | 0.819 |
| MTK-33.1 | 5.5 |
| MTK-33.5 | 2.248 |
| MTK-33.7 | 7.542 |
| MTK-33.8 | 23.69 |
| MTK-33.10 | 8.850 |
| MTK-33.11 | 0.453 |
| MTK-33.12 | 0.257 |
| MTK-33.13 | 1.641 |
| MTK-33.14 | 3.568 |
| MTK-33.15 | 0.480 |
| MTK-33.16 | 0.807 |
| MTK-33.17 | 7.494 |
| MTK-33.19 | 0.348 |
| MTK-33.20 | 0.457 |
| MTK-33.21 | 0.534 |
| MTK-33.22 | 0.629 |
| MTK-33.23 | 0.405 |
| MTK-33.24 | 0.480 |
| MTK-33.25 | 0.457 |

As shown in Table 35, humanized and affinity-matured anti-MerTK antibodies of the present disclosure were effective at blocking efferocytosis by human macrophages, displaying various IC50 values. Certain affinity-matured variants of anti-MerTK antibodies MTK-15, MTK-16, and MTK-33 displayed similar IC50 values to the corresponding parental anti-MerTK antibodies (see Example 11 above). Certain other affinity matured variants of anti-MerTK antibodies displayed lower IC50 values for blocking efferocytosis (e.g., MTK-15.1, MTK-15.4, MTK-15.5, MTK-15.6, MTK-15.7, MTK-15.8, MTK-15.10, MTK-15.13, MTK-15.15, MTK-16.1, and MTK-33.15) Affinity-matured variants of MTK-15 showed a range of IC50 values of 0.228 nM to 2.4 nM; affinity-matured variants of MTK-16 showed a range of IC50 values of 0.36 nM to 1.01 nM affinity-matured variants of MTK-29 showed a range of IC50 values of 0.5 nM to 1.12 nM; affinity-matured variants of MTK-33 showed a range of IC50 values of 0.41 nM to 23.9 nM.

### Example 27: Binding Kinetics of Humanized and Affinity-Matured Anti-MerTK Antibodies

Binding kinetics of humanized and affinity-matured anti-MerTK antibodies of the present disclosure to human, murine, and cynomolgus MerTK proteins were evaluated as follows. Briefly, anti-MerTK antibodies were prepared by diluting to 10 μg/ml in 10 mM Acetate, pH 4.25 (Carterra, Salt Lake City, Utah), at 300 μl/well. A HC200M sensor chip (Carterra) was activated using the single channel flow cell with a 7-minute injection of a 1:1:1 mixture of 100 mM MES pH 5.5, 100 mM sulfo-NHS, 400 mM EDC (all reconstituted in MES pH 5.5; 100 μl of each mixed in vial immediately before running assay). After switching to the multi-channel array flow cell, the antibodies were injected over the activated chip in a 96-spot array for 15 minutes. The remaining unconjugated active groups on the chip were then blocked by injecting 1M Ethanolamine pH 8.5 (Carterra) for 7 minutes using the single channel flow cell.

After priming with running buffer (HBS-TE, Carterra) with 0.5 mg/ml BSA (Sigma), the immobilized anti-MerTK antibodies were tested for their ability to bind to several forms of recombinant MerTK extracellular domain, including human, cynomolgus, and mouse orthologs as described above.

Estimates of affinity were generated by injecting each analyte over the entire antibody array using the single channel flow cell. MerTK analytes were diluted to 33.3, 100, and 300 nM in running buffer, and injected in serial from lowest to highest concentration without regeneration. Two buffer blanks were run between each series (one species per series). Data were processed and analyzed using NextGenKIT high-throughput kinetics analysis software (Carterra). In these experiments, the anti-MerTK antibodies were human IgG1 isotype containing the Fc variant LALAPS.

The equilibrium dissociation constants (K_{D}) were then calculated from the filled association and dissociation rate constants (k-on and k-off) for anti-MerTK antibodies of the present disclosure. The K_{D} values obtained from these experiments are summarized in Table 36 below.

TABLE 36

| | K_{D} (nM) | | |
|---|---|---|---|
| Antibody | huMerTK | moMerTK | cynoMerTK |
| MTK-15.1 | 8.3 | 1500 | 8.0 |
| MTK-15.2 | 21.5 | NB | 21.5 |
| MTK-15.3 | 21.5 | NB | 23 |
| MTK-15.4 | NF | NB | 1450 |
| MTK-15.5 | 0.1 | 690 | 0.8 |
| MTK-15.6 | 1.0 | 1700 | 1.5 |
| MTK-15.7 | 0.1 | 705 | 0.3 |
| MTK-15.8 | 4.9 | NB | 5.0 |
| MTK-15.9 | 10 | 850 | 11.5 |
| MTK-15.10 | 3.3 | 2800 | 3.3 |
| MTK-15.11 | 12.5 | 140 | 12.5 |
| MTK-15.12 | 17.5 | 114 | 18 |
| MTK-15.13 | 2.3 | 130 | 2.8 |
| MTK-16.1 | 86 | NB | 170 |
| MTK-16.2 | 80 | NB | 160 |
| MTK-29.5 | 4.4 | 135 | 4.4 |
| MTK-29.7 | 3.6 | 95 | 3.2 |
| MTK-29.8 | 23 | 310 | 22.5 |
| MTK-29.9 | 5.7 | 140 | 5.8 |
| MTK-29.10 | 4.5 | 116 | 4.2 |
| MTK-29.11 | 2.1 | 59 | 2.4 |
| MTK-29.12 | 23 | 250 | 21 |
| MTK-29.13 | 13 | 170 | 11 |
| MTK-29.14 | 7.6 | 100 | 7.3 |
| MTK-29.15 | 7.4 | NB | 8.4 |
| MTK-29.16 | 8.6 | 205 | 8.8 |
| MTK-29.17 | 9.6 | 170 | 9.9 |
| MTK-29.18 | 4.7 | 140 | 4.9 |
| MTK-33.5 | 28 | 140 | 26 |
| MTK-33.7 | 78 | 800 | 78 |
| MTK-33.8 | 41 | 465 | 41 |
| MTK-33.10 | 41 | 150 | 38 |
| MTK-33.11 | 11 | 115 | 10.1 |
| MTK-33.12 | 8.6 | 230 | 7.9 |
| MTK-33.13 | 20 | 225 | 18 |
| MTK-33.14 | 30.5 | 275 | 29 |
| MTK-33.15 | 4.6 | 105 | 4.0 |
| MTK-33.16 | 10.9 | 225 | 10.1 |
| MTK-33.17 | 20.4 | 505 | 19.9 |
| MTK-33.21 | 0.4 | 44 | 0.3 |
| MTK-33.25 | 1.2 | 53 | 1.1 |

NB = No binding
NF = No fit, meaning the data obtained did not fit a 1:1 binding equilibrium model

These results showed that humanized and affinity-matured anti-MerTK antibodies of the present disclosure displayed a range of affinities to human, cynomolgus, and murine MerTK protein.

These results also showed that humanized and affinity-matured anti-MerTK antibodies of the present disclosure variously displayed species binding specificity and cross-reactivity to human, murine, and cynomolgus MerTK. In particular, affinity of anti-MerTK antibodies of the present disclosure for binding to human MerTK ranged from 0.1 nM to 86 nM; affinity of anti-MerTK antibodies of the present disclosure for binding to cyno MerTK ranged from 0.3 nM to 1450 nM; and affinity of anti-MerTK antibodies of the present disclosure for binding to murine MerTK ranged from 44 nM to 2800 nM. Compared to the parental anti-MerTK antibody MTK-15, certain affinity-matured anti-MerTK antibody MTK-15 variants gained binding activity to mouse MerTK, which ranged from 114 nM to 2800 nM.

Table 37 below sets forth amino acid sequences of various human heavy chain immunoglobulin Fc variants.

TABLE 37

| Human Ig | | |
|---|---|---|
| Heavy Chain | | SEQ ID |
| Fc variants | Sequence | NO |
| huIgG1 | ASTKGPSVFPLAPSSKSTSGGTAALGCLVKDYFPEPVTVSWNSGALTSGV | 396 |
| WT Fc with | HTFPAVLQSSGLYSLSSVVTVPSSSLGTQTYICNVNHKPSNTKVDKKVEP | |
| C-terminal | KSCDKTHTCPPCPAPELLGGPSVFLFPPKPKDTLMISRTPEVTCVVVDVS | |
| lysine | HEDPEVKFNWYVDGVEVHNAKTKPREEQYNSTYRVVSVLTVLHQDWLNGK | |
| | EYKCKVSNKALPAPIEKTISKAKGQPREPQVYTLPPSRDELTKNQVSLTC | |
| | LVKGFYPSDIAVEWESNGQPENNYKTTPPVLDSDGSFFLYSKLTVDKSRW | |
| | QQGNVFSCSVMHEALHNHYTQKSLSLSPGK | |
| huIgG1 | ASTKGPSVFPLAPSSKSTSGGTAALGCLVKDYFPEPVTVSWNSGALTSGV | 397 |
| WT Fc | HTFPAVLQSSGLYSLSSVVTVPSSSLGTQTYICNVNHKPSNTKVDKKVEP | |
| without C- | KSCDKTHTCPPCPAPELLGGPSVFLFPPKPKDTLMISRTPEVTCVVVDVS | |
| terminal | HEDPEVKFNWYVDGVEVHNAKTKPREEQYNSTYRVVSVLTVLHQDWLNGK | |
| lysine | EYKCKVSNKALPAPIEKTISKAKGQPREPQVYTLPPSRDELTKNQVSLTC | |
| | LVKGFYPSDIAVEWESNGQPENNYKTTPPVLDSDGSFFLYSKLTVDKSRW | |
| | QQGNVFSCSVMHEALHNHYTQKSLSLSPG | |
| huIgG1 | ASTKGPSVFPLAPSSKSTSGGTAALGCLVKDYFPEPVTVSWNSGALTSGV | 398 |
| LALAPS Fc | HTFPAVLQSSGLYSLSSVVTVPSSSLGTQTYICNVNHKPSNTKVDKKVEP | |
| with C- | KSCDKTHTCPPCPAPEAAGGPSVFLFPPKPKDTLMISRTPEVTCVVVDVS | |
| terminal | HEDPEVKFNWYVDGVEVHNAKTKPREEQYNSTYRVVSVLTVLHQDWLNGK | |
| lysine | EYKCKVSNKALPASIEKTISKAKGQPREPQVYTLPPSRDELTKNQVSLTC | |
| | LVKGFYPSDIAVEWESNGQPENNYKTTPPVLDSDGSFFLYSKLTVDKSRW | |
| | QQGNVFSCSVMHEALHNHYTQKSLSLSPGK | |
| huIgG1 | ASTKGPSVFPLAPSSKSTSGGTAALGCLVKDYFPEPVTVSWNSGALTSGV | 399 |
| LALAPS Fc | HTFPAVLQSSGLYSLSSVVTVPSSSLGTQTYICNVNHKPSNTKVDKKVEP | |
| without C- | KSCDKTHTCPPCPAPEAAGGPSVFLFPPKPKDTLMISRTPEVTCVVVDVS | |
| terminal | HEDPEVKFNWYVDGVEVHNAKTKPREEQYNSTYRVVSVLTVLHQDWLNGK | |
| lysine | EYKCKVSNKALPASIEKTISKAKGQPREPQVYTLPPSRDELTKNQVSLTC | |
| | LVKGFYPSDIAVEWESNGQPENNYKTTPPVLDSDGSFFLYSKLTVDKSRW | |
| | QQGNVFSCSVMHEALHNHYTQKSLSLSPG | |
| huIgG1 | ASTKGPSVFPLAPSSKSTSGGTAALGCLVKDYFPEPVTVSWNSGALTSGV | 400 |
| NSLF Fc with | HTFPAVLQSSGLYSLSSVVTVPSSSLGTQTYICNVNHKPSNTKVDKKVEP | |
| C-terminal | KSCDKTHTCPPCPAPELLGGPSVFLFPPKPKDTLMISRTPEVTCVVVDVS | |
| lysine | HEDPEVKFNWYVDGVEVHNAKTKPREEQYNSTYRVVSVLTVLHQDWLNGK | |
| | EYKCKVSSKAFPAPIEKTISKAKGQPREPQVYTLPPSRDELTKNQVSLTC | |
| | LVKGFYPSDIAVEWESNGQPENNYKTTPPVLDSDGSFFLYSKLTVDKSRW | |
| | QQGNVFSCSVMHEALHNHYTQKSLSLSPGK | |
| huIgG1 | ASTKGPSVFPLAPSSKSTSGGTAALGCLVKDYFPEPVTVSWNSGALTSGV | 401 |
| NSLF Fc | HTFPAVLQSSGLYSLSSVVTVPSSSLGTQTYICNVNHKPSNTKVDKKVEP | |
| without C- | KSCDKTHTCPPCPAPELLGGPSVFLFPPKPKDTLMISRTPEVTCVVVDVS | |
| terminal | HEDPEVKFNWYVDGVEVHNAKTKPREEQYNSTYRVVSVLTVLHQDWLNGK | |
| lysine | EYKCKVSSKAFPAPIEKTISKAKGQPREPQVYTLPPSRDELTKNQVSLTC | |
| | LVKGFYPSDIAVEWESNGQPENNYKTTPPVLDSDGSFFLYSKLTVDKSRW | |
| | QQGNVFSCSVMHEALHNHYTQKSLSLSPG | |
| huIgG1 | ASTKGPSVFPLAPSSKSTSGGTAALGCLVKDYFPEPVTVSWNSGALTSGV | 402 |
| YTE Fc with | HTFPAVLQSSGLYSLSSVVTVPSSSLGTQTYICNVNHKPSNTKVDKKVEP | |
| C-terminal | KSCDKTHTCPPCPAPEAAGGPSVFLFPPKPKDTLYITREPEVTCVVVDVS | |
| lysine | HEDPEVKFNWYVDGVEVHNAKTKPREEQYNSTYRVVSVLTVLHQDWLNGK | |
| | EYKCKVSNKALPASIEKTISKAKGQPREPQVYTLPPSRDELTKNQVSLTC | |
| | LVKGFYPSDIAVEWESNGQPENNYKTTPPVLDSDGSFFLYSKLTVDKSRW | |
| | QQGNVFSCSVMHEALHNHYTQKSLSLSPGK | |
| huIgG1 | ASTKGPSVFPLAPSSKSTSGGTAALGCLVKDYFPEPVTVSWNSGALTSGV | 403 |
| YTE Fc | HTFPAVLQSSGLYSLSSVVTVPSSSLGTQTYICNVNHKPSNTKVDKKVEP | |
| without C- | KSCDKTHTCPPCPAPEAAGGPSVFLFPPKPKDTLYITREPEVTCVVVDVS | |
| terminal | HEDPEVKFNWYVDGVEVHNAKTKPREEQYNSTYRVVSVLTVLHQDWLNGK | |
| lysine | EYKCKVSNKALPASIEKTISKAKGQPREPQVYTLPPSRDELTKNQVSLTC | |
| | LVKGFYPSDIAVEWESNGQPENNYKTTPPVLDSDGSFFLYSKLTVDKSRW | |
| | QQGNVFSCSVMHEALHNHYTQKSLSLSPG | |
| huIgG1 | ASTKGPSVFPLAPSSKSTSGGTAALGCLVKDYFPEPVTVSWNSGALTSGV | 404 |
| LS Fc with C- | HTFPAVLQSSGLYSLSSVVTVPSSSLGTQTYICNVNHKPSNTKVDKKVEP | |
| terminal | KSCDKTHTCPPCPAPEAAGGPSVFLFPPKPKDTLMISRTPEVTCVVVDVS | |
| lysine | HEDPEVKFNWYVDGVEVHNAKTKPREEQYNSTYRVVSVLTVLHQDWLNGK | |
| | EYKCKVSNKALPASIEKTISKAKGQPREPQVYTLPPSRDELTKNQVSLTC | |
| | LVKGFYPSDIAVEWESNGQPENNYKTTPPVLDSDGSFFLYSKLTVDKSRW | |
| | QQGNVFSCSVLHEALHSHYTQKSLSLSPGK | |
| huIgG1 | ASTKGPSVFPLAPSSKSTSGGTAALGCLVKDYFPEPVTVSWNSGALTSGV | 405 |
| LS Fc | HTFPAVLQSSGLYSLSSVVTVPSSSLGTQTYICNVNHKPSNTKVDKKVEP | |
| without C- | KSCDKTHTCPPCPAPEAAGGPSVFLFPPKPKDTLMISRTPEVTCVVVDVS | |
| terminal | HEDPEVKFNWYVDGVEVHNAKTKPREEQYNSTYRVVSVLTVLHQDWLNGK | |
| lysine | EYKCKVSNKALPASIEKTISKAKGQPREPQVYTLPPSRDELTKNQVSLTC | |
| | LVKGFYPSDIAVEWESNGQPENNYKTTPPVLDSDGSFFLYSKLTVDKSRW | |
| | QQGNVFSCSVLHEALHSHYTQKSLSLSPG | |
| huIgG1 | ASTKGPSVFPLAPSSKSTSGGTAALGCLVKDYFPEPVTVSWNSGALTSGV | 406 |
| LV5-112 Fc | HTFPAVLQSSGLYSLSSVVTVPSSSLGTQTYICNVNHKPSNTKVDKKVEP | |
| with C- | KSCDKTHTCPPCPAPEAAGGPSVFLFPPKPKDTLMISRTPEVTCVVVDVS | |
| terminal | HEDPEVKFNWYVDGVEVHNAKTKPREEQYNSTYRVVSVLTVLHQDWLNGK | |
| lysine | EYKCKVSNKALPASIEKTISKAKGQPREPQVYTLPPSRDELTKNQVSLTC | |
| | LVKGFYPSDIAVEWESNGGCALYPTNCGGGQPENNYKTTPPVLDSDGSFF | |
| | LYSKLTVDKSRWQQGNVFSCSVMHEALHNHYTQKSLSLSPGK | |
| huIgG1 | ASTKGPSVFPLAPSSKSTSGGTAALGCLVKDYFPEPVTVSWNSGALTSGV | 407 |
| LV5-112 Fc | HTFPAVLQSSGLYSLSSVVTVPSSSLGTQTYICNVNHKPSNTKVDKKVEP | |
| without C- | KSCDKTHTCPPCPAPEAAGGPSVFLFPPKPKDTLMISRTPEVTCVVVDVS | |
| terminal | HEDPEVKFNWYVDGVEVHNAKTKPREEQYNSTYRVVSVLTVLHQDWLNGK | |
| lysine | EYKCKVSNKALPASIEKTISKAKGQPREPQVYTLPPSRDELTKNQVSLTC | |
| | LVKGFYPSDIAVEWESNGGCALYPTNCGGGQPENNYKTTPPVLDSDGSFF | |
| | LYSKLTVDKSRWQQGNVFSCSVMHEALHNHYTQKSLSLSPG | |
| huIgG2 | ASTKGPSVFPLAPCSRSTSESTAALGCLVKDYFPEPVTVSWNSGALTSGV | 408 |
| WT Fc with | HTFPAVLQSSGLYSLSSVVTVPSSNFGTQTYTCNVDHKPSNTKVDKTVER | |
| C-terminal | KCCVECPPCPAPPVAGPSVFLFPPKPKDTLMISRTPEVTCVVVDVSHEDP | |
| lysine | EVQFNWYVDGVEVHNAKTKPREEQFNSTFRVVSVLTVVHQDWLNGKEYKC | |
| | KVSNKGLPAPIEKTISKTKGQPREPQVYTLPPSREEMTKNQVSLTCLVKG | |
| | FYPSDIAVEWESNGQPENNYKTTPPMLDSDGSFFLYSKLTVDKSRWQQGN | |
| | VFSCSVMHEALHNHYTQKSLSLSPGK | |
| huIgG2 | ASTKGPSVFPLAPCSRSTSESTAALGCLVKDYFPEPVTVSWNSGALTSGV | 409 |
| WT Fc | HTFPAVLQSSGLYSLSSVVTVPSSNFGTQTYTCNVDHKPSNTKVDKTVER | |
| without C- | KCCVECPPCPAPPVAGPSVFLFPPKPKDTLMISRTPEVTCVVVDVSHEDP | |
| terminal | EVQFNWYVDGVEVHNAKTKPREEQFNSTFRVVSVLTVVHQDWLNGKEYKC | |
| lysine | KVSNKGLPAPIEKTISKTKGQPREPQVYTLPPSREEMTKNQVSLTCLVKG | |
| | FYPSDIAVEWESNGQPENNYKTTPPMLDSDGSFFLYSKLTVDKSRWQQGN | |
| | VFSCSVMHEALHNHYTQKSLSLSPG | |
| huIgG4 | ASTKGPSVFPLAPCSRSTSESTAALGCLVKDYFPEPVTVSWNSGALTSGV | 410 |
| WT Fc with | HTFPAVLQSSGLYSLSSVVTVPSSSLGTKTYTCNVDHKPSNTKVDKRVES | |
| C-terminal | KYGPPCPSCPAPEFLGGPSVFLFPPKPKDTLMISRTPEVTCVVVDVSQED | |
| lysine | PEVQFNWYVDGVEVHNAKTKPREEQFNSTYRVVSVLTVLHQDWLNGKEYK | |
| | CKVSNKGLPSSIEKTISKAKGQPREPQVYTLPPSQEEMTKNQVSLTCLVK | |
| | GFYPSDIAVEWESNGQPENNYKTTPPVLDSDGSFFLYSRLTVDKSRWQEG | |
| | NVFSCSVMHEALHNHYTQKSLSLSLGK | |
| huIgG4 | ASTKGPSVFPLAPCSRSTSESTAALGCLVKDYFPEPVTVSWNSGALTSGV | 411 |
| WT Fc | HTFPAVLQSSGLYSLSSVVTVPSSSLGTKTYTCNVDHKPSNTKVDKRVES | |
| without C- | KYGPPCPSCPAPEFLGGPSVFLFPPKPKDTLMISRTPEVTCVVVDVSQED | |
| terminal | PEVQFNWYVDGVEVHNAKTKPREEQFNSTYRVVSVLTVLHQDWLNGKEYK | |
| lysine | CKVSNKGLPSSIEKTISKAKGQPREPQVYTLPPSQEEMTKNQVSLTCLVK | |
| | GFYPSDIAVEWESNGQPENNYKTTPPVLDSDGSFFLYSRLTVDKSRWQEG | |
| | NVFSCSVMHEALHNHYTQKSLSLSLG | |

Human IgG1 light chain constant region amino acid sequence is set forth below:

(SEQ ID NO: 412)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

Full-length heavy chain amino acid sequences of certain anti-MerTK antibodies of the present disclosure are shown below in Table 38.

TABLE 38

| | | SEQ ID |
|---|---|---|
| Antibody | Sequence | NO: |
| MTK-16.1 | QVQLVQSGSELKKPGASVKVSCKASGYTFTNHGMNWVRQAPGQGLEWMGWI | 413 |
| with | NTYTGEPTYAQGFTGRFVFSLDTSVSTAYLQISSLKAEDTAVYYCARKGVT | |
| huIgG1 | AARYFDYWGQGTLVTVSSASTKGPSVFPLAPSSKSTSGGTAALGCLVKDYF | |
| WT Fc with | PEPVTVSWNSGALTSGVHTFPAVLQSSGLYSLSSVVTVPSSSLGTQTYICN | |
| C-terminal | VNHKPSNTKVDKKVEPKSCDKTHTCPPCPAPELLGGPSVFLFPPKPKDTLM | |
| lysine | ISRTPEVTCVVVDVSHEDPEVKFNWYVDGVEVHNAKTKPREEQYNSTYRVV | |
| | SVLTVLHQDWLNGKEYKCKVSNKALPAPIEKTISKAKGQPREPQVYTLPPS | |
| | RDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| MTK-16.1 | QVQLVQSGSELKKPGASVKVSCKASGYTFTNHGMNWVRQAPGQGLEWMGWI | 414 |
| with | NTYTGEPTYAQGFTGRFVFSLDTSVSTAYLQISSLKAEDTAVYYCARKGVT | |
| huIgG1 | AARYFDYWGQGTLVTVSSASTKGPSVFPLAPSSKSTSGGTAALGCLVKDYF | |
| WT Fc | PEPVTVSWNSGALTSGVHTFPAVLQSSGLYSLSSVVTVPSSSLGTQTYICN | |
| without C- | VNHKPSNTKVDKKVEPKSCDKTHTCPPCPAPELLGGPSVFLFPPKPKDTLM | |
| terminal | ISRTPEVTCVVVDVSHEDPEVKFNWYVDGVEVHNAKTKPREEQYNSTYRVV | |
| lysine | SVLTVLHQDWLNGKEYKCKVSNKALPAPIEKTISKAKGQPREPQVYTLPPS | |
| | RDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| MTK-16.1 | QVQLVQSGSELKKPGASVKVSCKASGYTFTNHGMNWVRQAPGQGLEWMGWI | 415 |
| with | NTYTGEPTYAQGFTGRFVFSLDTSVSTAYLQISSLKAEDTAVYYCARKGVT | |
| huIgG1 | AARYFDYWGQGTLVTVSSASTKGPSVFPLAPSSKSTSGGTAALGCLVKDYF | |
| LALAPS | PEPVTVSWNSGALTSGVHTFPAVLQSSGLYSLSSVVTVPSSSLGTQTYICN | |
| Fc with C- | VNHKPSNTKVDKKVEPKSCDKTHTCPPCPAPEAAGGPSVFLFPPKPKDTLM | |
| terminal | ISRTPEVTCVVVDVSHEDPEVKFNWYVDGVEVHNAKTKPREEQYNSTYRVV | |
| lysine | SVLTVLHQDWLNGKEYKCKVSNKALPASIEKTISKAKGQPREPQVYTLPPS | |
| | RDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| MTK-16.1 | QVQLVQSGSELKKPGASVKVSCKASGYTFTNHGMNWVRQAPGQGLEWMGWI | 416 |
| with | NTYTGEPTYAQGFTGRFVFSLDTSVSTAYLQISSLKAEDTAVYYCARKGVT | |
| hulgG1 | AARYFDYWGQGTLVTVSSASTKGPSVFPLAPSSKSTSGGTAALGCLVKDYF | |
| LALAPS | PEPVTVSWNSGALTSGVHTFPAVLQSSGLYSLSSVVTVPSSSLGTQTYICN | |
| Fc without | VNHKPSNTKVDKKVEPKSCDKTHTCPPCPAPEAAGGPSVFLFPPKPKDTLM | |
| C-terminal | ISRTPEVTCVVVDVSHEDPEVKFNWYVDGVEVHNAKTKPREEQYNSTYRVV | |
| lysine | SVLTVLHQDWLNGKEYKCKVSNKALPASIEKTISKAKGQPREPQVYTLPPS | |
| | RDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| MTK-16.1 | QVQLVQSGSELKKPGASVKVSCKASGYTFTNHGMNWVRQAPGQGLEWMGWI | 417 |
| with | NTYTGEPTYAQGFTGRFVFSLDTSVSTAYLQISSLKAEDTAVYYCARKGVT | |
| huIgG1 | AARYFDYWGQGTLVTVSSASTKGPSVFPLAPSSKSTSGGTAALGCLVKDYF | |
| NSLF Fc | PEPVTVSWNSGALTSGVHTFPAVLQSSGLYSLSSVVTVPSSSLGTQTYICN | |
| with C- | VNHKPSNTKVDKKVEPKSCDKTHTCPPCPAPELLGGPSVFLFPPKPKDTLM | |
| terminal | ISRTPEVTCVVVDVSHEDPEVKFNWYVDGVEVHNAKTKPREEQYNSTYRVV | |
| lysine | SVLTVLHQDWLNGKEYKCKVSSKAFPAPIEKTISKAKGQPREPQVYTLPPS | |
| | RDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| MTK-16.1 | QVQLVQSGSELKKPGASVKVSCKASGYTFTNHGMNWVRQAPGQGLEWMGWI | 418 |
| with | NTYTGEPTYAQGFTGRFVFSLDTSVSTAYLQISSLKAEDTAVYYCARKGVT | |
| hulgG1 | AARYFDYWGQGTLVTVSSASTKGPSVFPLAPSSKSTSGGTAALGCLVKDYF | |
| NSLF Fc | PEPVTVSWNSGALTSGVHTFPAVLQSSGLYSLSSVVTVPSSSLGTQTYICN | |
| without C- | VNHKPSNTKVDKKVEPKSCDKTHTCPPCPAPELLGGPSVFLFPPKPKDTLM | |
| terminal | ISRTPEVTCVVVDVSHEDPEVKFNWYVDGVEVHNAKTKPREEQYNSTYRVV | |
| lysine | SVLTVLHQDWLNGKEYKCKVSSKAFPAPIEKTISKAKGQPREPQVYTLPPS | |
| | RDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| MTK-16.1 | QVQLVQSGSELKKPGASVKVSCKASGYTFTNHGMNWVRQAPGQGLEWMGWI | 419 |
| with | NTYTGEPTYAQGFTGRFVFSLDTSVSTAYLQISSLKAEDTAVYYCARKGVT | |
| hulgG4 Fc | AARYFDYWGQGTLVTVSSASTKGPSVFPLAPCSRSTSESTAALGCLVKDYF | |
| with C- | PEPVTVSWNSGALTSGVHTFPAVLQSSGLYSLSSVVTVPSSSLGTKTYTCN | |
| terminal | VDHKPSNTKVDKRVESKYGPPCPSCPAPEFLGGPSVFLFPPKPKDTLMISR | |
| lysine | TPEVTCVVVDVSQEDPEVQFNWYVDGVEVHNAKTKPREEQFNSTYRVVSVL | |
| | TVLHQDWLNGKEYKCKVSNKGLPSSIEKTISKAKGQPREPQVYTLPPSQEE | |
| | MTKNQVSLTCLVKGFYPSDIAVEWESNGQPENNYKTTPPVLDSDGSFFLYS | |
| | RLTVDKSRWQEGNVFSCSVMHEALHNHYTQKSLSLSLGK | |
| MTK-16.1 | NTYTGEPTYAQGFTGRFVFSLDTSVSTAYLQISSLKAEDTAVYYCARKGVT | 420 |
| with | AARYFDYWGQGTLVTVSSASTKGPSVFPLAPCSRSTSESTAALGCLVKDYF | |
| hulgG4 Fc | PEPVTVSWNSGALTSGVHTFPAVLQSSGLYSLSSVVTVPSSSLGTKTYTCN | |
| without C- | VDHKPSNTKVDKRVESKYGPPCPSCPAPEFLGGPSVFLFPPKPKDTLMISR | |
| terminal | TPEVTCVVVDVSQEDPEVQFNWYVDGVEVHNAKTKPREEQFNSTYRVVSVL | |
| lysine | TVLHQDWLNGKEYKCKVSNKGLPSSIEKTISKAKGQPREPQVYTLPPSQEE | |
| | MTKNQVSLTCLVKGFYPSDIAVEWESNGQPENNYKTTPPVLDSDGSFFLYS | |
| | RLTVDKSRWQEGNVFSCSVMHEALHNHYTQKSLSLSLG | |

MTK-16.1 light chain amino acid sequence is set forth below:

(SEQ ID NO: 421)
DIQMTQSPSSLSASVGDRVTITCKASQNVRTAVAWYQQKPGKAPKRLIYL
ASNRHTGVPSRFSGSGSGTEFTLTISNLQPEDFATYYCLQHWNYPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Full-length heavy chain amino acid sequences of certain anti-MerTK antibodies of the present disclosure are shown below in Table 39.

TABLE 39

| | | SEQ ID |
|---|---|---|
| Antibody | Sequence | NO: |
| MTK-16.2 | QVQLVQSGSELKKPGASVKVSCKASGYTFTNHGMNWVRQAPGQGLEWMGWI | 422 |
| with | NTYTGEPTYADDFKGRFVFSLDTSVSTAYLQISSLKAEDTAVYYCARKGVT | |
| huIgG1 | AARYFDYWGQGTLVTVSSASTKGPSVFPLAPSSKSTSGGTAALGCLVKDYF | |
| WT Fc with | PEPVTVSWNSGALTSGVHTFPAVLQSSGLYSLSSVVTVPSSSLGTQTYICN | |
| C-terminal | VNHKPSNTKVDKKVEPKSCDKTHTCPPCPAPELLGGPSVFLFPPKPKDTLM | |
| lysine | ISRTPEVTCVVVDVSHEDPEVKFNWYVDGVEVHNAKTKPREEQYNSTYRVV | |
| | SVLTVLHQDWLNGKEYKCKVSNKALPAPIEKTISKAKGQPREPQVYTLPPS | |
| | RDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| MTK-16.2 | QVQLVQSGSELKKPGASVKVSCKASGYTFTNHGMNWVRQAPGQGLEWMGWI | 423 |
| with | NTYTGEPTYADDFKGRFVFSLDTSVSTAYLQISSLKAEDTAVYYCARKGVT | |
| huIgG1 | AARYFDYWGQGTLVTVSSASTKGPSVFPLAPSSKSTSGGTAALGCLVKDYF | |
| WT Fc | PEPVTVSWNSGALTSGVHTFPAVLQSSGLYSLSSVVTVPSSSLGTQTYICN | |
| without C- | VNHKPSNTKVDKKVEPKSCDKTHTCPPCPAPELLGGPSVFLFPPKPKDTLM | |
| terminal | ISRTPEVTCVVVDVSHEDPEVKFNWYVDGVEVHNAKTKPREEQYNSTYRVV | |
| lysine | SVLTVLHQDWLNGKEYKCKVSNKALPAPIEKTISKAKGQPREPQVYTLPPS | |
| | RDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| MTK-16.2 | QVQLVQSGSELKKPGASVKVSCKASGYTFTNHGMNWVRQAPGQGLEWMGWI | 424 |
| with | NTYTGEPTYADDFKGRFVFSLDTSVSTAYLQISSLKAEDTAVYYCARKGVT | |
| huIgG1 | AARYFDYWGQGTLVTVSSASTKGPSVFPLAPSSKSTSGGTAALGCLVKDYF | |
| LALAPS | PEPVTVSWNSGALTSGVHTFPAVLQSSGLYSLSSVVTVPSSSLGTQTYICN | |
| Fc with C- | VNHKPSNTKVDKKVEPKSCDKTHTCPPCPAPEAAGGPSVFLFPPKPKDTLM | |
| terminal | ISRTPEVTCVVVDVSHEDPEVKFNWYVDGVEVHNAKTKPREEQYNSTYRVV | |
| lysine | SVLTVLHQDWLNGKEYKCKVSNKALPASIEKTISKAKGQPREPQVYTLPPS | |
| | RDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| MTK-16.2 | QVQLVQSGSELKKPGASVKVSCKASGYTFTNHGMNWVRQAPGQGLEWMGWI | 425 |
| with | NTYTGEPTYADDFKGRFVFSLDTSVSTAYLQISSLKAEDTAVYYCARKGVT | |
| huIgG1 | AARYFDYWGQGTLVTVSSASTKGPSVFPLAPSSKSTSGGTAALGCLVKDYF | |
| LALAPS | PEPVTVSWNSGALTSGVHTFPAVLQSSGLYSLSSVVTVPSSSLGTQTYICN | |
| Fc without | VNHKPSNTKVDKKVEPKSCDKTHTCPPCPAPEAAGGPSVFLFPPKPKDTLM | |
| C-terminal | ISRTPEVTCVVVDVSHEDPEVKFNWYVDGVEVHNAKTKPREEQYNSTYRVV | |
| lysine | SVLTVLHQDWLNGKEYKCKVSNKALPASIEKTISKAKGQPREPQVYTLPPS | |
| | RDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| MTK-16.2 | QVQLVQSGSELKKPGASVKVSCKASGYTFTNHGMNWVRQAPGQGLEWMGWI | 426 |
| with | NTYTGEPTYADDFKGRFVFSLDTSVSTAYLQISSLKAEDTAVYYCARKGVT | |
| huIgG1 | AARYFDYWGQGTLVTVSSASTKGPSVFPLAPSSKSTSGGTAALGCLVKDYF | |
| NSLF Fc | PEPVTVSWNSGALTSGVHTFPAVLQSSGLYSLSSVVTVPSSSLGTQTYICN | |
| with C- | VNHKPSNTKVDKKVEPKSCDKTHTCPPCPAPELLGGPSVFLFPPKPKDTLM | |
| terminal | ISRTPEVTCVVVDVSHEDPEVKFNWYVDGVEVHNAKTKPREEQYNSTYRVV | |
| lysine | SVLTVLHQDWLNGKEYKCKVSSKAFPAPIEKTISKAKGQPREPQVYTLPPS | |
| | RDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| MTK-16.2 | QVQLVQSGSELKKPGASVKVSCKASGYTFTNHGMNWVRQAPGQGLEWMGWI | 427 |
| with | NTYTGEPTYADDFKGRFVFSLDTSVSTAYLQISSLKAEDTAVYYCARKGVT | |
| huIgG1 | AARYFDYWGQGTLVTVSSASTKGPSVFPLAPSSKSTSGGTAALGCLVKDYF | |
| NSLF Fc | PEPVTVSWNSGALTSGVHTFPAVLQSSGLYSLSSVVTVPSSSLGTQTYICN | |
| without C- | VNHKPSNTKVDKKVEPKSCDKTHTCPPCPAPELLGGPSVFLFPPKPKDTLM | |
| terminal | ISRTPEVTCVVVDVSHEDPEVKFNWYVDGVEVHNAKTKPREEQYNSTYRVV | |
| lysine | SVLTVLHQDWLNGKEYKCKVSSKAFPAPIEKTISKAKGQPREPQVYTLPPS | |
| | RDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| MTK-16.2 | QVQLVQSGSELKKPGASVKVSCKASGYTFTNHGMNWVRQAPGQGLEWMGWI | 428 |
| with | NTYTGEPTYADDFKGRFVFSLDTSVSTAYLQISSLKAEDTAVYYCARKGVT | |
| huIgG4 Fc | AARYFDYWGQGTLVTVSSASTKGPSVFPLAPCSRSTSESTAALGCLVKDYF | |
| with C- | PEPVTVSWNSGALTSGVHTFPAVLQSSGLYSLSSVVTVPSSSLGTKTYTCN | |
| terminal | VDHKPSNTKVDKRVESKYGPPCPSCPAPEFLGGPSVFLFPPKPKDTLMISR | |
| lysine | TPEVTCVVVDVSQEDPEVQFNWYVDGVEVHNAKTKPREEQFNSTYRVVSVL | |
| | TVLHQDWLNGKEYKCKVSNKGLPSSIEKTISKAKGQPREPQVYTLPPSQEE | |
| | MTKNQVSLTCLVKGFYPSDIAVEWESNGQPENNYKTTPPVLDSDGSFFLYS | |
| | RLTVDKSRWQEGNVFSCSVMHEALHNHYTQKSLSLSLGK | |
| MTK-16.2 | QVQLVQSGSELKKPGASVKVSCKASGYTFTNHGMNWVRQAPGQGLEWMGWI | 429 |
| with | NTYTGEPTYADDFKGRFVFSLDTSVSTAYLQISSLKAEDTAVYYCARKGVT | |
| huIgG4 Fc | AARYFDYWGQGTLVTVSS | |
| without C- | ASTKGPSVFPLAPCSRSTSESTAALGCLVKDYFPEPVTVSWNSGALTSGVH | |
| terminal | TFPAVLQSSGLYSLSSVVTVPSSSLGTKTYTCNVDHKPSNTKVDKRVESKY | |
| lysine | GPPCPSCPAPEFLGGPSVFLFPPKPKDTLMISRTPEVTCVVVDVSQEDPEV | |
| | QFNWYVDGVEVHNAKTKPREEQFNSTYRVVSVLTVLHQDWLNGKEYKCKVS | |
| | NKGLPSSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGNVFSCS | |
| | VMHEALHNHYTQKSLSLSLGK | |

MTK-16.2 light chain amino acid sequence is set forth below:

(SEQ ID NO: 430)
DIQMTQSPSSLSASVGDRVTITCRASQNVRTAVAWYQQKPGKAPKRLIYL
ASNRHTGVPSRFSGSGSGTEFTLTISNLQPEDFATYYCLQHWNYPLTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Full-length heavy chain amino acid sequences of certain anti-MerTK antibodies of the present disclosure are shown below in Table 40.

TABLE 40

| | | SEQ ID |
|---|---|---|
| Antibody | Sequence | NO: |
| MTK-33.1 | QVQLVQSGAEVKKPGASVKVSCKASGYTFTSYWMHWVRQAPGQGLEWIGVI | 431 |
| with | DPSDNYINYNQKFQGRVTMTRDTSTSTVYMELSSLRSEDTAVYYCAREAWT | |
| huIgG1 | RGYFNYWGQGTLVTVSSASTKGPSVFPLAPSSKSTSGGTAALGCLVKDYFP | |
| WT Fc with | EPVTVSWNSGALTSGVHTFPAVLQSSGLYSLSSVVTVPSSSLGTQTYICNV | |
| C-terminal | NHKPSNTKVDKKVEPKSCDKTHTCPPCPAPELLGGPSVFLFPPKPKDTLMI | |
| lysine | SRTPEVTCVVVDVSHEDPEVKFNWYVDGVEVHNAKTKPREEQYNSTYRVVS | |
| | VLTVLHQDWLNGKEYKCKVSNKALPAPIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPGK | |
| MTK-33.1 | QVQLVQSGAEVKKPGASVKVSCKASGYTFTSYWMHWVRQAPGQGLEWIGVI | 432 |
| with | DPSDNYINYNQKFQGRVTMTRDTSTSTVYMELSSLRSEDTAVYYCAREAWT | |
| huIgG1 | RGYFNYWGQGTLVTVSSASTKGPSVFPLAPSSKSTSGGTAALGCLVKDYFP | |
| WT Fc | EPVTVSWNSGALTSGVHTFPAVLQSSGLYSLSSVVTVPSSSLGTQTYICNV | |
| without C- | NHKPSNTKVDKKVEPKSCDKTHTCPPCPAPELLGGPSVFLFPPKPKDTLMI | |
| terminal | SRTPEVTCVVVDVSHEDPEVKFNWYVDGVEVHNAKTKPREEQYNSTYRVVS | |
| lysine | VLTVLHQDWLNGKEYKCKVSNKALPAPIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPG | |
| MTK-33.1 | QVQLVQSGAEVKKPGASVKVSCKASGYTFTSYWMHWVRQAPGQGLEWIGVI | 433 |
| with | DPSDNYINYNQKFQGRVTMTRDTSTSTVYMELSSLRSEDTAVYYCAREAWT | |
| huIgG1 | RGYFNYWGQGTLVTVSSASTKGPSVFPLAPSSKSTSGGTAALGCLVKDYFP | |
| LALAPS | EPVTVSWNSGALTSGVHTFPAVLQSSGLYSLSSVVTVPSSSLGTQTYICNV | |
| Fc with C- | NHKPSNTKVDKKVEPKSCDKTHTCPPCPAPEAAGGPSVFLFPPKPKDTLMI | |
| terminal | SRTPEVTCVVVDVSHEDPEVKFNWYVDGVEVHNAKTKPREEQYNSTYRVVS | |
| lysine | VLTVLHQDWLNGKEYKCKVSNKALPASIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPGK | |
| MTK-33.1 | QVQLVQSGAEVKKPGASVKVSCKASGYTFTSYWMHWVRQAPGQGLEWIGVI | 434 |
| with | DPSDNYINYNQKFQGRVTMTRDTSTSTVYMELSSLRSEDTAVYYCAREAWT | |
| huIgG1 | RGYFNYWGQGTLVTVSSASTKGPSVFPLAPSSKSTSGGTAALGCLVKDYFP | |
| LALAPS | EPVTVSWNSGALTSGVHTFPAVLQSSGLYSLSSVVTVPSSSLGTQTYICNV | |
| Fc without | NHKPSNTKVDKKVEPKSCDKTHTCPPCPAPEAAGGPSVFLFPPKPKDTLMI | |
| C-terminal | SRTPEVTCVVVDVSHEDPEVKFNWYVDGVEVHNAKTKPREEQYNSTYRVVS | |
| lysine | VLTVLHQDWLNGKEYKCKVSNKALPASIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPG | |
| MTK-33.1 | QVQLVQSGAEVKKPGASVKVSCKASGYTFTSYWMHWVRQAPGQGLEWIGVI | 435 |
| with | DPSDNYINYNQKFQGRVTMTRDTSTSTVYMELSSLRSEDTAVYYCAREAWT | |
| huIgG1 | RGYFNYWGQGTLVTVSSASTKGPSVFPLAPSSKSTSGGTAALGCLVKDYFP | |
| NSLF Fc | EPVTVSWNSGALTSGVHTFPAVLQSSGLYSLSSVVTVPSSSLGTQTYICNV | |
| with C- | NHKPSNTKVDKKVEPKSCDKTHTCPPCPAPELLGGPSVFLFPPKPKDTLMI | |
| terminal | SRTPEVTCVVVDVSHEDPEVKFNWYVDGVEVHNAKTKPREEQYNSTYRVVS | |
| lysine | VLTVLHQDWLNGKEYKCKVSSKAFPAPIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPGK | |
| MTK-33.1 | QVQLVQSGAEVKKPGASVKVSCKASGYTFTSYWMHWVRQAPGQGLEWIGVI | 436 |
| with | DPSDNYINYNQKFQGRVTMTRDTSTSTVYMELSSLRSEDTAVYYCAREAWT | |
| huIgG1 | RGYFNYWGQGTLVTVSSASTKGPSVFPLAPSSKSTSGGTAALGCLVKDYFP | |
| NSLF Fc | EPVTVSWNSGALTSGVHTFPAVLQSSGLYSLSSVVTVPSSSLGTQTYICNV | |
| without C- | NHKPSNTKVDKKVEPKSCDKTHTCPPCPAPELLGGPSVFLFPPKPKDTLMI | |
| terminal | SRTPEVTCVVVDVSHEDPEVKFNWYVDGVEVHNAKTKPREEQYNSTYRVVS | |
| lysine | VLTVLHQDWLNGKEYKCKVSSKAFPAPIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPG | |
| MTK-33.1 | QVQLVQSGAEVKKPGASVKVSCKASGYTFTSYWMHWVRQAPGQGLEWIGVI | 437 |
| with | DPSDNYINYNQKFQGRVTMTRDTSTSTVYMELSSLRSEDTAVYYCAREAWT | |
| huIgG4 Fc | RGYFNYWGQGTLVTVSSASTKGPSVFPLAPCSRSTSESTAALGCLVKDYFP | |
| with C- | EPVTVSWNSGALTSGVHTFPAVLQSSGLYSLSSVVTVPSSSLGTKTYTCNV | |
| terminal | DHKPSNTKVDKRVESKYGPPCPSCPAPEFLGGPSVFLFPPKPKDTLMISRT | |
| lysine | PEVTCVVVDVSQEDPEVQFNWYVDGVEVHNAKTKPREEQFNSTYRVVSVLT | |
| | VLHQDWLNGKEYKCKVSNKGLPSSIEKTISKAKGQPREPQVYTLPPSQEEM | |
| | TKNQVSLTCLVKGFYPSDIAVEWESNGQPENNYKTTPPVLDSDGSFFLYSR | |
| | LTVDKSRWQEGNVFSCSVMHEALHNHYTQKSLSLSLGK | |
| MTK-33.1 | QVQLVQSGAEVKKPGASVKVSCKASGYTFTSYWMHWVRQAPGQGLEWIGVI | 438 |
| with | DPSDNYINYNQKFQGRVTMTRDTSTSTVYMELSSLRSEDTAVYYCAREAWT | |
| huIgG4 Fc | RGYFNYWGQGTLVTVSSASTKGPSVFPLAPCSRSTSESTAALGCLVKDYFP | |
| without C- | EPVTVSWNSGALTSGVHTFPAVLQSSGLYSLSSVVTVPSSSLGTKTYTCNV | |
| terminal | DHKPSNTKVDKRVESKYGPPCPSCPAPEFLGGPSVFLFPPKPKDTLMISRT | |
| lysine | PEVTCVVVDVSQEDPEVQFNWYVDGVEVHNAKTKPREEQFNSTYRVVSVLT | |
| | VLHQDWLNGKEYKCKVSNKGLPSSIEKTISKAKGQPREPQVYTLPPSQEEM | |
| | TKNQVSLTCLVKGFYPSDIAVEWESNGQPENNYKTTPPVLDSDGSFFLYSR | |
| | LTVDKSRWQEGNVFSCSVMHEALHNHYTQKSLSLSLG | |

MTK-33.1 light chain amino acid sequence is set forth below:

(SEQ ID NO: 439)
DIQMTQSPSSLSASVGDRVTITCQASQSVSNTVAWYQQKPGKAPKLLIYY
ASLRYTGVPSRFSGSGSGTDFTFTISSLQPEDIATYYCQQDYRSPFTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Full-length heavy chain amino acid sequences of certain anti-MerTK antibodies of the present disclosure are shown below in Table 41.

TABLE 41

| | | SEQ ID |
|---|---|---|
| Antibody | Sequence | NO: |
| MTK-33.12 | QVQLVQSGAEVKKPGASVKVSCKASGYTFVSYWMHWVRQAPGQGLEWIGVI | 440 |
| with | DPSDNYINYNQKFRGRVTMTRDTSTSTVYMELSSLRSEDTAVYYCAREAGT | |
| huIgG1 | RGYFDYWGQGTLVTVSSASTKGPSVFPLAPSSKSTSGGTAALGCLVKDYFP | |
| WT Fc with | EPVTVSWNSGALTSGVHTFPAVLQSSGLYSLSSVVTVPSSSLGTQTYICNV | |
| C-terminal | NHKPSNTKVDKKVEPKSCDKTHTCPPCPAPELLGGPSVFLFPPKPKDTLMI | |
| lysine | SRTPEVTCVVVDVSHEDPEVKFNWYVDGVEVHNAKTKPREEQYNSTYRVVS | |
| | VLTVLHQDWLNGKEYKCKVSNKALPAPIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPGK | |
| MTK-33.12 | QVQLVQSGAEVKKPGASVKVSCKASGYTFVSYWMHWVRQAPGQGLEWIGVI | 441 |
| with | DPSDNYINYNQKFRGRVTMTRDTSTSTVYMELSSLRSEDTAVYYCAREAGT | |
| huIgG1 | RGYFDYWGQGTLVTVSSASTKGPSVFPLAPSSKSTSGGTAALGCLVKDYFP | |
| WT Fc | EPVTVSWNSGALTSGVHTFPAVLQSSGLYSLSSVVTVPSSSLGTQTYICNV | |
| without C- | NHKPSNTKVDKKVEPKSCDKTHTCPPCPAPELLGGPSVFLFPPKPKDTLMI | |
| terminal | SRTPEVTCVVVDVSHEDPEVKFNWYVDGVEVHNAKTKPREEQYNSTYRVVS | |
| lysine | VLTVLHQDWLNGKEYKCKVSNKALPAPIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPG | |
| MTK-33.12 | QVQLVQSGAEVKKPGASVKVSCKASGYTFVSYWMHWVRQAPGQGLEWIGVI | 442 |
| with | DPSDNYINYNQKFRGRVTMTRDTSTSTVYMELSSLRSEDTAVYYCAREAGT | |
| huIgG1 | RGYFDYWGQGTLVTVSSASTKGPSVFPLAPSSKSTSGGTAALGCLVKDYFP | |
| LALAPS | EPVTVSWNSGALTSGVHTFPAVLQSSGLYSLSSVVTVPSSSLGTQTYICNV | |
| Fc with C- | NHKPSNTKVDKKVEPKSCDKTHTCPPCPAPEAAGGPSVFLFPPKPKDTLMI | |
| terminal | SRTPEVTCVVVDVSHEDPEVKFNWYVDGVEVHNAKTKPREEQYNSTYRVVS | |
| lysine | VLTVLHQDWLNGKEYKCKVSNKALPASIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPGK | |
| MTK-33.12 | QVQLVQSGAEVKKPGASVKVSCKASGYTFVSYWMHWVRQAPGQGLEWIGVI | 443 |
| with | DPSDNYINYNQKFRGRVTMTRDTSTSTVYMELSSLRSEDTAVYYCAREAGT | |
| huIgG1 | RGYFDYWGQGTLVTVSSASTKGPSVFPLAPSSKSTSGGTAALGCLVKDYFP | |
| LALAPS | EPVTVSWNSGALTSGVHTFPAVLQSSGLYSLSSVVTVPSSSLGTQTYICNV | |
| Fc without | NHKPSNTKVDKKVEPKSCDKTHTCPPCPAPEAAGGPSVFLFPPKPKDTLMI | |
| C-terminal | SRTPEVTCVVVDVSHEDPEVKFNWYVDGVEVHNAKTKPREEQYNSTYRVVS | |
| lysine | VLTVLHQDWLNGKEYKCKVSNKALPASIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPG | |
| MTK-33.12 | QVQLVQSGAEVKKPGASVKVSCKASGYTFVSYWMHWVRQAPGQGLEWIGVI | 444 |
| with | DPSDNYINYNQKFRGRVTMTRDTSTSTVYMELSSLRSEDTAVYYCAREAGT | |
| huIgG1 | RGYFDYWGQGTLVTVSSASTKGPSVFPLAPSSKSTSGGTAALGCLVKDYFP | |
| NSLF Fc | EPVTVSWNSGALTSGVHTFPAVLQSSGLYSLSSVVTVPSSSLGTQTYICNV | |
| with C- | NHKPSNTKVDKKVEPKSCDKTHTCPPCPAPELLGGPSVFLFPPKPKDTLMI | |
| terminal | SRTPEVTCVVVDVSHEDPEVKFNWYVDGVEVHNAKTKPREEQYNSTYRVVS | |
| lysine | VLTVLHQDWLNGKEYKCKVSSKAFPAPIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPGK | |
| MTK-33.12 | DPSDNYINYNQKFRGRVTMTRDTSTSTVYMELSSLRSEDTAVYYCAREAGT | 445 |
| with | RGYFDYWGQGTLVTVSSASTKGPSVFPLAPSSKSTSGGTAALGCLVKDYFP | |
| huIgG1 | EPVTVSWNSGALTSGVHTFPAVLQSSGLYSLSSVVTVPSSSLGTQTYICNV | |
| NSLF Fc | NHKPSNTKVDKKVEPKSCDKTHTCPPCPAPELLGGPSVFLFPPKPKDTLMI | |
| without C- | SRTPEVTCVVVDVSHEDPEVKFNWYVDGVEVHNAKTKPREEQYNSTYRVVS | |
| terminal | VLTVLHQDWLNGKEYKCKVSSKAFPAPIEKTISKAKGQPREPQVYTLPPSR | |
| lysine | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPG | |
| MTK-33.12 | QVQLVQSGAEVKKPGASVKVSCKASGYTFVSYWMHWVRQAPGQGLEWIGVI | 446 |
| with | DPSDNYINYNQKFRGRVTMTRDTSTSTVYMELSSLRSEDTAVYYCAREAGT | |
| huIgG4 Fc | RGYFDYWGQGTLVTVSSASTKGPSVFPLAPCSRSTSESTAALGCLVKDYFP | |
| with C- | EPVTVSWNSGALTSGVHTFPAVLQSSGLYSLSSVVTVPSSSLGTKTYTCNV | |
| terminal | DHKPSNTKVDKRVESKYGPPCPSCPAPEFLGGPSVFLFPPKPKDTLMISRT | |
| lysine | PEVTCVVVDVSQEDPEVQFNWYVDGVEVHNAKTKPREEQFNSTYRVVSVLT | |
| | VLHQDWLNGKEYKCKVSNKGLPSSIEKTISKAKGQPREPQVYTLPPSQEEM | |
| | TKNQVSLTCLVKGFYPSDIAVEWESNGQPENNYKTTPPVLDSDGSFFLYSR | |
| | LTVDKSRWQEGNVFSCSVMHEALHNHYTQKSLSLSLGK | |
| MTK-33.12 | QVQLVQSGAEVKKPGASVKVSCKASGYTFVSYWMHWVRQAPGQGLEWIGVI | 447 |
| with | DPSDNYINYNQKFRGRVTMTRDTSTSTVYMELSSLRSEDTAVYYCAREAGT | |
| huIgG4 Fc | RGYFDYWGQGTLVTVSSASTKGPSVFPLAPCSRSTSESTAALGCLVKDYFP | |
| without C- | EPVTVSWNSGALTSGVHTFPAVLQSSGLYSLSSVVTVPSSSLGTKTYTCNV | |
| terminal | DHKPSNTKVDKRVESKYGPPCPSCPAPEFLGGPSVFLFPPKPKDTLMISRT | |
| lysine | PEVTCVVVDVSQEDPEVQFNWYVDGVEVHNAKTKPREEQFNSTYRVVSVLT | |
| | VLHQDWLNGKEYKCKVSNKGLPSSIEKTISKAKGQPREPQVYTLPPSQEEM | |
| | TKNQVSLTCLVKGFYPSDIAVEWESNGQPENNYKTTPPVLDSDGSFFLYSR | |
| | LTVDKSRWQEGNVFSCSVMHEALHNHYTQKSLSLSLG | |

MTK-33.12 light chain amino acid sequence is set forth below:

(SEQ ID NO: 448)
DIQMTQSPSSLSASVGDRVTITCQASQSVSNTVAWYQQKPGKAPKLLIYY
ASNRYTGVPSRFSGSGSGTDFTFTISSLQPEDIATYYCRQDTRSPFTFGQ
GTKLELKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Full-length heavy chain amino acid sequences of certain anti-MerTK antibodies of the present disclosure are shown below in Table 42.

TABLE 42

| | | SEQ ID |
|---|---|---|
| Antibody | Sequence | NO: |
| MTK-33.10 | QVQLVQSGAEVKKPGASVKVSCKASSYSFTSYWMHWVRQAPGQGLEWIGVI | 464 |
| with | DPSDNYINYNQKFRGRVTMTRDTSTSTVYMELSSLRSEDTAVYYCAREAGT | |
| huIgG1 | RGYFDYWGQGTLVTVSSASTKGPSVFPLAPSSKSTSGGTAALGCLVKDYFP | |
| WT Fc with | EPVTVSWNSGALTSGVHTFPAVLQSSGLYSLSSVVTVPSSSLGTQTYICNV | |
| C-terminal | NHKPSNTKVDKKVEPKSCDKTHTCPPCPAPELLGGPSVFLFPPKPKDTLMI | |
| lysine | SRTPEVTCVVVDVSHEDPEVKFNWYVDGVEVHNAKTKPREEQYNSTYRVVS | |
| | VLTVLHQDWLNGKEYKCKVSNKALPAPIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPGK | |
| MTK-33.10 | QVQLVQSGAEVKKPGASVKVSCKASSYSFTSYWMHWVRQAPGQGLEWIGVI | 465 |
| with | DPSDNYINYNQKFRGRVTMTRDTSTSTVYMELSSLRSEDTAVYYCAREAGT | |
| huIgG1 | RGYFDYWGQGTLVTVSSASTKGPSVFPLAPSSKSTSGGTAALGCLVKDYFP | |
| WT Fc | EPVTVSWNSGALTSGVHTFPAVLQSSGLYSLSSVVTVPSSSLGTQTYICNV | |
| without C- | NHKPSNTKVDKKVEPKSCDKTHTCPPCPAPELLGGPSVFLFPPKPKDTLMI | |
| terminal | SRTPEVTCVVVDVSHEDPEVKFNWYVDGVEVHNAKTKPREEQYNSTYRVVS | |
| lysine | VLTVLHQDWLNGKEYKCKVSNKALPAPIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPG | |
| MTK-33.10 | DPSDNYINYNQKFRGRVTMTRDTSTSTVYMELSSLRSEDTAVYYCAREAGT | 466 |
| with | RGYFDYWGQGTLVTVSSASTKGPSVFPLAPSSKSTSGGTAALGCLVKDYFP | |
| huIgG1 | EPVTVSWNSGALTSGVHTFPAVLQSSGLYSLSSVVTVPSSSLGTQTYICNV | |
| LALAPS | NHKPSNTKVDKKVEPKSCDKTHTCPPCPAPEAAGGPSVFLFPPKPKDTLMI | |
| Fc with C- | SRTPEVTCVVVDVSHEDPEVKFNWYVDGVEVHNAKTKPREEQYNSTYRVVS | |
| terminal | VLTVLHQDWLNGKEYKCKVSNKALPASIEKTISKAKGQPREPQVYTLPPSR | |
| lysine | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPGK | |
| MTK-33.10 | QVQLVQSGAEVKKPGASVKVSCKASSYSFTSYWMHWVRQAPGQGLEWIGVI | 467 |
| with | DPSDNYINYNQKFRGRVTMTRDTSTSTVYMELSSLRSEDTAVYYCAREAGT | |
| huIgG1 | RGYFDYWGQGTLVTVSSASTKGPSVFPLAPSSKSTSGGTAALGCLVKDYFP | |
| LALAPS | EPVTVSWNSGALTSGVHTFPAVLQSSGLYSLSSVVTVPSSSLGTQTYICNV | |
| Fc without | NHKPSNTKVDKKVEPKSCDKTHTCPPCPAPEAAGGPSVFLFPPKPKDTLMI | |
| C-terminal | SRTPEVTCVVVDVSHEDPEVKFNWYVDGVEVHNAKTKPREEQYNSTYRVVS | |
| lysine | VLTVLHQDWLNGKEYKCKVSNKALPASIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPG | |
| MTK-33.10 | QVQLVQSGAEVKKPGASVKVSCKASSYSFTSYWMHWVRQAPGQGLEWIGVI | 468 |
| with | DPSDNYINYNQKFRGRVTMTRDTSTSTVYMELSSLRSEDTAVYYCAREAGT | |
| huIgG1 | RGYFDYWGQGTLVTVSSASTKGPSVFPLAPSSKSTSGGTAALGCLVKDYFP | |
| NSLF Fc | EPVTVSWNSGALTSGVHTFPAVLQSSGLYSLSSVVTVPSSSLGTQTYICNV | |
| with C- | NHKPSNTKVDKKVEPKSCDKTHTCPPCPAPELLGGPSVFLFPPKPKDTLMI | |
| terminal | SRTPEVTCVVVDVSHEDPEVKFNWYVDGVEVHNAKTKPREEQYNSTYRVVS | |
| lysine | VLTVLHQDWLNGKEYKCKVSSKAFPAPIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPGK | |
| MTK-33.10 | QVQLVQSGAEVKKPGASVKVSCKASSYSFTSYWMHWVRQAPGQGLEWIGVI | 469 |
| with | DPSDNYINYNQKFRGRVTMTRDTSTSTVYMELSSLRSEDTAVYYCAREAGT | |
| huIgG1 | RGYFDYWGQGTLVTVSSASTKGPSVFPLAPSSKSTSGGTAALGCLVKDYFP | |
| NSLF Fc | EPVTVSWNSGALTSGVHTFPAVLQSSGLYSLSSVVTVPSSSLGTQTYICNV | |
| without C- | NHKPSNTKVDKKVEPKSCDKTHTCPPCPAPELLGGPSVFLFPPKPKDTLMI | |
| terminal | SRTPEVTCVVVDVSHEDPEVKFNWYVDGVEVHNAKTKPREEQYNSTYRVVS | |
| lysine | VLTVLHQDWLNGKEYKCKVSSKAFPAPIEKTISKAKGQPREPQVYTLPPSR | |
| | DELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPG | |
| MTK-33.10 | QVQLVQSGAEVKKPGASVKVSCKASSYSFTSYWMHWVRQAPGQGLEWIGVI | 470 |
| with | DPSDNYINYNQKFRGRVTMTRDTSTSTVYMELSSLRSEDTAVYYCAREAGT | |
| huIgG4 Fc | RGYFDYWGQGTLVTVSSASTKGPSVFPLAPCSRSTSESTAALGCLVKDYFP | |
| with C- | EPVTVSWNSGALTSGVHTFPAVLQSSGLYSLSSVVTVPSSSLGTKTYTCNV | |
| terminal | DHKPSNTKVDKRVESKYGPPCPSCPAPEFLGGPSVFLFPPKPKDTLMISRT | |
| lysine | PEVTCVVVDVSQEDPEVQFNWYVDGVEVHNAKTKPREEQFNSTYRVVSVLT | |
| | VLHQDWLNGKEYKCKVSNKGLPSSIEKTISKAKGQPREPQVYTLPPSQEEM | |
| | TKNQVSLTCLVKGFYPSDIAVEWESNGQPENNYKTTPPVLDSDGSFFLYSR | |
| | LTVDKSRWQEGNVFSCSVMHEALHNHYTQKSLSLSLGK | |
| MTK-33.10 | QVQLVQSGAEVKKPGASVKVSCKASSYSFTSYWMHWVRQAPGQGLEWIGVI | 471 |
| with | DPSDNYINYNQKFRGRVTMTRDTSTSTVYMELSSLRSEDTAVYYCAREAGT | |
| huIgG4 Fc | RGYFDYWGQGTLVTVSSASTKGPSVFPLAPCSRSTSESTAALGCLVKDYFP | |
| without C- | EPVTVSWNSGALTSGVHTFPAVLQSSGLYSLSSVVTVPSSSLGTKTYTCNV | |
| terminal | DHKPSNTKVDKRVESKYGPPCPSCPAPEFLGGPSVFLFPPKPKDTLMISRT | |
| lysine | PEVTCVVVDVSQEDPEVQFNWYVDGVEVHNAKTKPREEQFNSTYRVVSVLT | |
| | VLHQDWLNGKEYKCKVSNKGLPSSIEKTISKAKGQPREPQVYTLPPSQEEM | |
| | TKNQVSLTCLVKGFYPSDIAVEWESNGQPENNYKTTPPVLDSDGSFFLYSR | |
| | LTVDKSRWQEGNVFSCSVMHEALHNHYTQKSLSLSLG | |

MTK-33.10 light chain amino acid sequence is set forth below:

(SEQ ID NO: 472)
DIQMTQSPSSLSASVGDRVTITCQAGQSVSNTVAWYQQKPGKAPKLLIY
YASNRYTGVPSRFSGSGSGTDFTFTISSLQPEDIATYYCQQDYRSPFTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

1-112. (canceled)

113. A method of treating an individual having cancer, the method comprising administering to an individual in need thereof a therapeutically effective amount an antibody that binds to human MerTK, wherein the antibody comprises an HVR-H1, HVR-H2, HVR-H3, HVR-L1, HVR-L2, and HVR-L3 comprising the amino acid sequences of:
(i) SEQ ID NOs: 75, 99, 125, 158, 187, and 207, respectively;
(ii) SEQ ID NOs: 76, 100, 126, 159, 187, and 208, respectively;
(iii) SEQ ID NOs: 77, 101, 127, 160, 188, and 209, respectively;
(iv) SEQ ID NOs: 75, 99, 128, 158, 187, and 210, respectively;
(v) SEQ ID NOs: 78, 102, 129, 161, 189, and 211, respectively;
(vi) SEQ ID NOs: 75, 103, 130, 158, 187, and 212, respectively;
(vii) SEQ ID NOs: 77, 101, 127, 162, 188, and 209, respectively;
(viii) SEQ ID NOs: 75, 99, 131, 163, 187, and 213, respectively;
(ix) SEQ ID NOs: 79, 104, 132, 164, 190, and 214, respectively;
(x) SEQ ID NOs: 80, 105, 133, 165, 191, and 215, respectively;
(xi) SEQ ID NOs: 81, 106, 134, 166, 192, and 216, respectively;
(xii) SEQ ID NOs: 82, 107, 135, 167, 193, and 217, respectively;
(xiii) SEQ ID NOs: 77, 108, 136, 167, 194, and 209, respectively;
(xiv) SEQ ID NOs: 75, 109, 137, 168, 187, and 218, respectively;
(xv) SEQ ID NOs: 83, 99, 138, 158, 187, and 210, respectively;
(xvi) SEQ ID NOs: 84, 99, 139, 169, 195, and 219, respectively;
(xvii) SEQ ID NOs: 85, 99, 140, 170, 196, and 220, respectively;
(xviii) SEQ ID NOs: 86, 99, 141, 171, 191, and 221, respectively;
(xix) SEQ ID NOs:87, 110, 142, 172, 197, and 222, respectively;
(xx) SEQ ID NOs: 88, 99, 143, 173, 191, and 223, respectively;
(xxi) SEQ ID NOs: 89, 111, 143, 173, 198, and 221, respectively;
(xxii) SEQ ID NOs: 90, 112, 144, 174, 199, and 224, respectively;
(xxiii) SEQ ID NOs: 91, 113, 145, 175, 200, and 225, respectively;
(xxiv) SEQ ID NOs: 90, 114, 146, 176, 201, and 226, respectively;
(xxv) SEQ ID NOs: 92, 115, 147, 177, 195, and 227, respectively;
(xxvi) SEQ ID NOs: 93, 116, 148, 178, 188, and 209, respectively;
(xxvii) SEQ ID NOs: 94, 117, 149, 179, 195, and 228, respectively;
(xxviii) SEQ ID NOs: 95, 118, 150, 180, 187, and 229, respectively;
(xxix) SEQ ID NOs: 95, 119, 151, 181, 187, and 208, respectively;
(xxx) SEQ ID NOs: 90, 120, 152, 182, 202, and 230, respectively;
(xxxi) SEQ ID NOs: 96, 118, 153, 183, 202, and 230, respectively;
(xxxii) SEQ ID NOs: 96, 121, 154, 181, 187, and 210, respectively;
(xxxiii) SEQ ID NOs: 90, 122, 155, 184, 203, and 231, respectively;
(xxxiv) SEQ ID NOs: 90, 122, 155, 184, 203, and 231, respectively;
(xxxv) SEQ ID NOs: 97, 123, 156, 185, 204, and 232, respectively;
(xxxvi) SEQ ID NOs: 98, 124, 157, 186, 205, and 233, respectively;
(xxxvii) SEQ ID NOs: 83, 329, 334, 158, 187, and 210, respectively;
(xxxviii) SEQ ID NOs: 83, 330, 138, 158, 187, and 210, respectively;
(xxxix) SEQ ID NOs: 83, 329, 138, 340, 187, and 210, respectively;
(xl) SEQ ID NOs: 83, 329, 138, 341, 187, and 210, respectively;
(xli) SEQ ID NOs: 83, 329, 335, 341, 187, and 210, respectively;
(xlii) SEQ ID NOs: 83, 331, 138, 341, 187, and 210, respectively;
(xliii) SEQ ID NOs: 83, 329, 138, 342, 187, and 210, respectively;
(xliv) SEQ ID NOs: 83, 329, 336, 341, 187, and 210, respectively;
(xlv) SEQ ID NOs: 83, 329, 337, 343, 187, and 210, respectively;
(xlvi) SEQ ID NOs: 83, 332, 338, 341, 187, and 210, respectively;
(xlvii) SEQ ID NOs: 83, 333, 334, 158, 187, and 210, respectively;
(xlviii) SEQ ID NOs: 83, 332, 336, 341, 187, and 210, respectively;
(xlix) SEQ ID NOs: 83, 329, 339, 344, 187, and 210, respectively;
(1) SEQ ID NOs: 83, 329, 138, 158, 187, and 210, respectively;
(li) SEQ ID NOs: 84, 329, 139, 169, 195, and 219, respectively;
(lii) SEQ ID NOs: 84, 99, 139, 345, 195, and 219, respectively;
(liii) SEQ ID NOs: 346, 348, 355, 341, 187, and 360, respectively;
(liv) SEQ ID NOs: 95, 119, 355, 341, 187, and 361, respectively;
(lv) SEQ ID NOs: 95, 349, 356, 341, 187, and 362, respectively;
(lvi) SEQ ID NOs: 95, 119, 356, 341, 359, and 362, respectively;
(lvii) SEQ ID NOs: 346, 119, 356, 341, 359, and 362, respectively;
(lviii) SEQ ID NOs: 95, 350, 356, 341, 359, and 362, respectively;
(lix) SEQ ID NOs: 95, 351, 356, 341, 359, and 362, respectively;
(lx) SEQ ID NOs: 347, 119, 356, 341, 359, and 362, respectively;
(lxi) SEQ ID NOs: 346, 119, 356, 341, 359, and 362, respectively;
(lxii) SEQ ID NOs: 95, 119, 356, 341, 187, and 362, respectively;
(lxiii) SEQ ID NOs: 95, 352, 356, 341, 187, and 362, respectively;
(lxiv) SEQ ID NOs: 95, 350, 356, 341, 359, and 362, respectively;
(lxv) SEQ ID NOs: 95, 353, 356, 341, 359, and 362, respectively;
(lxvi) SEQ ID NOs: 95, 351, 356, 341, 359, and 362, respectively;
(lxvii) SEQ ID NOs: 95, 350, 356, 341, 359, and 362, respectively;
(lxviii) SEQ ID NOs: 346, 354, 356, 341, 359, and 363, respectively;
(lxix) SEQ ID NOs: 346, 348, 356, 357, 359, and 362, respectively;
(lxx) SEQ ID NOs: 346, 354, 356, 358, 187, and 362, respectively;
(lxxi) SEQ ID NOs: 95, 348, 151, 181, 187, and 208, respectively;
(lxxii) SEQ ID NOs: 90, 364, 373, 376, 388, and 231, respectively;
(lxxiii) SEQ ID NOs: 90, 364, 155, 377, 203, and 231, respectively;
(lxxiv) SEQ ID NOs: 90, 364, 374, 378, 203, and 231, respectively;
(lxxv) SEQ ID NOs: 90, 365, 155, 376, 203, and 392, respectively;
(lxxvi) SEQ ID NOs: 90, 364, 155, 376, 389, and 231, respectively;
(lxxvii) SEQ ID NOs: 90, 364, 155, 379, 203, and 231, respectively;
(lxxviii) SEQ ID NOs: 90, 366, 155, 380, 203, and 231, respectively;
(lxxix) SEQ ID NOs: 90, 364, 373, 381, 203, and 231, respectively;
(lxxx) SEQ ID NOs: 90, 367, 155, 382, 203, and 231, respectively;
(lxxxi) SEQ ID NOs: 90, 368, 155, 383, 203, and 231, respectively;
(lxxxii) SEQ ID NOs: 90, 364, 374, 384, 203, and 231, respectively;
(lxxxiii) SEQ ID NOs: 90, 368, 155, 376, 203, and 293, respectively;
(lxxxiv) SEQ ID NOs: 90, 364, 155, 378, 203, and 231, respectively;
(lxxxv) SEQ ID NOs: 90, 368, 155, 376, 203, and 231, respectively;
(lxxxvi) SEQ ID NOs: 90, 369, 155, 378, 203, and 394, respectively;
(lxxxvii) SEQ ID NOs: 90, 364, 155, 378, 203, and 231, respectively;
(lxxxviii) SEQ ID NOs: 90, 370, 155, 378, 203, and 394, respectively;
(lxxxix) SEQ ID NOs: 90, 371, 155, 378, 203, and 394, respectively;
(xc) SEQ ID NOs: 90, 368, 155, 385, 203, and 394, respectively;
(xci) SEQ ID NOs: 90, 368, 155, 378, 203, and 395, respectively;
(xcii) SEQ ID NOs: 90, 368, 155, 378, 390, and 394, respectively;
(xciii) SEQ ID NOs: 90, 372, 155, 378, 391, and 394, respectively;
(xciv) SEQ ID NOs: 90, 368, 375, 386, 203, and 394, respectively; or
(xcv) SEQ ID NOs: 90, 368, 155, 387, 203, and 394, respectively.

114. The method of claim 113, wherein the cancer is colon cancer, ovarian cancer, liver cancer, or endometrial cancer.

115. The method of claim 113, wherein the administration does not lead to a retinal pathology in the individual.

116. The method of claim 113, further comprising administering an anti-PD-L1 antibody, an anti-PD-L2 antibody, or anti-PD antibody to the individual.

117. A method for detecting MerTK in a sample comprising contacting said sample with the antibody of claim 113.

118. The method of claim 113, wherein the antibody comprises a heavy chain variable region and a light chain variable region comprising the amino acid sequences of SEQ ID NOs:5 and 40, respectively; SEQ ID NOs:6 and 41, respectively; SEQ ID NOs:7 and 42, respectively; SEQ ID NOs:8 and 43, respectively; SEQ ID NOs:9 and 44, respectively; SEQ ID NOs:10 and 45, respectively; SEQ ID NOs:11 and 46, respectively; SEQ ID NOs:12 and 47, respectively; SEQ ID NOs:13 and 48, respectively; SEQ ID NOs:14 and 49, respectively; SEQ ID NOs:15 and 50, respectively; SEQ ID NOs:16 and 51, respectively; SEQ ID NOs:17 and 52, respectively; SEQ ID NOs:18 and 53, respectively; SEQ ID NOs:19 and 54, respectively; SEQ ID NOs:20 and 55, respectively; SEQ ID NOs:21 and 56, respectively; SEQ ID NOs:22 and 57, respectively; SEQ ID NOs:23 and 58, respectively; SEQ ID NOs:24 and 59, respectively; SEQ ID NOs:25 and 60, respectively; SEQ ID NOs:26 and 61, respectively; SEQ ID NOs:27 and 62, respectively; SEQ ID NOs:28 and 63, respectively; SEQ ID NOs:29 and 64, respectively; SEQ ID NOs:30 and 65, respectively; SEQ ID NOs:31 and 66, respectively; SEQ ID NOs:32 and 67, respectively; SEQ ID NOs:33 and 68, respectively; SEQ ID NOs:34 and 69, respectively; SEQ ID NOs:35 and 70, respectively; SEQ ID NOs:36 and 71, respectively; SEQ ID NOs:37 and 72, respectively; SEQ ID NOs:38 and 73, respectively; and SEQ ID NOs:39 and 74, respectively; SEQ ID NOs:234 and 247, respectively; SEQ ID NOs:235 and 247, respectively; SEQ ID NOs:236 and 248, respectively; SEQ ID NOs:236 and 249, respectively; SEQ ID NOs:237 and 249, respectively; SEQ ID NOs:238 and 249, respectively; SEQ ID NOs:239 and 250, respectively; SEQ ID NOs:240 and 251, respectively; SEQ ID NOs:241 and 252, respectively; SEQ ID NOs:242 and 249, respectively; SEQ ID NOs:243 and 247, respectively; SEQ ID NOs:244 and 251, respectively; SEQ ID NOs:245 and 253, respectively; SEQ ID NOs:246 and 247, respectively; SEQ ID NOs:246 and 254, respectively; SEQ ID NOs:255 and 257, respectively; SEQ ID NOs:256 and 258, respectively; SEQ ID NOs:259 and 274, respectively; SEQ ID NOs:260 and 275, respectively; SEQ ID NOs:261 and 276, respectively; SEQ ID NOs:262 and 277, respectively; SEQ ID NOs:263 and 277, respectively; SEQ ID NOs:264 and 277, respectively; SEQ ID NOs:265 and 277, respectively; SEQ ID NOs:266 and 277, respectively; SEQ ID NOs:267 and 278, respectively; SEQ ID NOs:268 and 279, respectively; SEQ ID NOs:269 and 279, respectively; SEQ ID NOs:264 and 280, respectively; SEQ ID NOs:270 and 281, respectively; SEQ ID NOs:265 and 282, respectively; SEQ ID NOs:264 and 283, respectively; SEQ ID NOs:271 and 284, respectively; SEQ ID NOs:272 and 285, respectively; SEQ ID NOs:271 and 286, respectively; SEQ ID NOs:273 and 287, respectively; SEQ ID NOs:273 and 288, respectively; SEQ ID NOs:273 and 289, respectively; SEQ ID NOs:290 and 309, respectively; SEQ ID NOs:291 and 310, respectively; SEQ ID NOs:292 and 311, respectively SEQ ID NOs:293 and 312, respectively; SEQ ID NOs:294 and 313, respectively; SEQ ID NOs:295 and 314, respectively; SEQ ID NOs:296 and 315, respectively; SEQ ID NOs:290 and 316, respectively; SEQ ID NOs:297 and 317, respectively; SEQ ID NOs:298 and 318, respectively; SEQ ID NOs:292 and 319, respectively; SEQ ID NOs:299 and 320, respectively; SEQ ID NOs:300 and 311, respectively; SEQ ID NOs:301 and 321, respectively; SEQ ID NOs:302 and 322, respectively; SEQ ID NOs:303 and 311, respectively; SEQ ID NOs:304 and 322, respectively; SEQ ID NOs:305 and 322, respectively; SEQ ID NOs:301 and 323, respectively; SEQ ID NOs:301 and 324, respectively; SEQ ID NOs:301 and 325, respectively; SEQ ID NOs:306 and 326, respectively; SEQ ID NOs:307 and 327, respectively; or SEQ ID NOs:308 and 328, respectively.

119. The method of claim 118, wherein the antibody comprises (a) a heavy chain comprising the amino acid sequence of SEQ ID NO:466 or 464 and a light chain comprising the amino acid sequence of SEQ ID NO:472; (b) a heavy chain comprising the amino acid sequence of SEQ ID NO:467, 465, 468, or 469 and a light chain comprising the amino acid sequence of SEQ ID NO:472; (c) a heavy chain comprising the amino acid sequence of SEQ ID NO:422, 423, 424, 425, or 426 and a light chain comprising the amino acid sequence of SEQ ID NO:430; (d) a heavy chain comprising the amino acid sequence of SEQ ID NO:440, 441, 442, 443, 444, or 445 and a light chain comprising the amino acid sequence of SEQ ID NO:448; or (e) a heavy chain comprising the amino acid sequence of SEQ ID NO:413, 414, 415, 416, 417, or 418 and a light chain comprising the amino acid sequence of SEQ ID NO:421.

120. The method of claim 113, wherein the antibody is a murine antibody, a humanized antibody, a bispecific antibody, a monoclonal antibody, a multivalent antibody, a conjugated antibody, or a chimeric antibody.

121. The method of claim 113, wherein the antibody is of the IgG class, the IgM class, or the IgA class.

122. The method of claim 121, wherein the antibody is of the IgG class and has an IgG1, and IgG2, or an IgG4 isotype.

123. The method of claim 113, wherein the antibody is a full-length antibody.

124. The method of claim 113, wherein the antibody is an antibody fragment.

125. The method of claim 124, wherein the antibody fragment is a Fab, Fab′, Fab′-SH, F(ab′)2, Fv or scFv.

126. The method of claim 113, wherein the antibody is an scFv.

127. The method of claim 118, wherein the antibody is a bispecific antibody, a monoclonal antibody, a multivalent antibody, or a conjugated antibody.

128. The method of claim 118, wherein the antibody is of the IgG class.

129. The method of claim 128, wherein the antibody is of the IgG class and has an IgG1, and IgG2, or an IgG4 isotype.

130. The method of claim 118, wherein the antibody is a full-length antibody.

131. The method of claim 118, wherein the antibody is an antibody fragment.

132. The method of claim 131, wherein the antibody fragment is a Fab, Fab′, Fab′-SH, F(ab′)2, Fv or scFv.


SEQUENCE LISTING (472 sequences)

SEQ ID NO: 1 - protein, 999 aa, Homo sapiens
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 2 - protein, 505 aa, Homo sapiens
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLII

SEQ ID NO: 3 - protein, 507 aa, synthetic construct
  REGION 1..507: note = Cyno MerTK ECD
MGLAPLPLPLLLGLFLPALWSRAITEAREEAKPYPLFPGPLPGSLQTDHTSLLSLPHTSGYQPALMFSPTQPGRPYTGNVAIPRVTSAGSKLLPPLAFKHTVGHIILSEHKDVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIHNSTAHSILISWVPGFDGYSPFRNCSVQVKEVDPLSNGSVMIFNTSASPHMYQIKQLQALANYSIGVSCMNEIGWSAVSPWILASTTEGAPSVAPLNVTVFLNESRDNVDIRWMKPLTKRQAGELVGYRISHVWQSAGISKELLEEVGQNNSRAQISVQVHNATCTVRIAAVTKGGVGPFSDPVKIFIPAHGWVDHAPSSTPAPGNADPVLII

SEQ ID NO: 4 - protein, 497 aa, synthetic construct
  REGION 1..497: note = Murine MerTK ECD
MVLAPLLLGLLLLPALWSGGTAEKWEETELDQLFSGPLPGRLPVNHRPFSAPHSSRDQLPPPQTGRSHPAHTAAPQVTSTASKLLPPVAFNHTIGHIVLSEHKNVKFNCSINIPNTYQETAGISWWKDGKELLGAHHSITQFYPDEEGVSIIALFSIASVQRSDNGSYFCKMKVNNREIVSDPIYVEVQGLPYFIKQPESVNVTRNTAFNLTCQAVGPPEPVNIFWVQNSSRVNEKPERSPSVLTVPGLTETAVFSCEAHNDKGLTVSKGVHINIKVIPSPPTEVHILNSTAHSILVSWVPGFDGYSPLQNCSIQVKEADRLSNGSVMVFNTSASPHLYEIQQLQALANYSIAVSCRNEIGWSAVSPWILASTTEGAPSVAPLNITVFLNESNNILDIRWTKPPIKRQDGELVGYRISHVWESAGTYKELSEEVSQNGSWAQIPVQIHNATCTVRIAAITKGGIGPFSEPVNIIIPEHSKVDYAPSSTPAPGNTDSM

SEQ ID NO: 5 - protein, 118 aa, synthetic construct
  REGION 1..118: note = MTK-01 Heavy Chain Variable
QIQLVQSGPELKKPGETVKISCKASGYTFTNYGMNWVKQAPGKGLKCMGWINTYTGEPTYADDFKGRFAFSLETSATTAYLQINNLKNEDTATYFCARRDRYTWFAYWGQGTLVTVSA

SEQ ID NO: 6 - protein, 118 aa, synthetic construct
  REGION 1..118: note = MTK-02 Heavy Chain Variable
QIQLVQSGPELKQPGETVKISCKASGYTFTDYGVNWVKQAPGKGLKWMGWINTYSGEPTYAGDFKGRFAFSLESSASTAFLQINNLKNEDMATYFCARRVRYWYFDVWGAGTSVTVSS

SEQ ID NO: 7 - protein, 120 aa, synthetic construct
  REGION 1..120: note = MTK-03 Heavy Chain Variable
DVQLQESGPGLVKPSQSLSLTCSVTGYSITSGYYWNWIRQFPGNTLEWMGYMSFDGDNKFNPSLKNRISITRDTSKNQFFLRLNSVTTEDTATYYCARGGYNYGSTEANWGQGTLVTVSA

SEQ ID NO: 8 - protein, 118 aa, synthetic construct
  REGION 1..118: note = MTK-04 Heavy Chain Variable
QIQLVQSGPELKKPGETVKISCKASGYTFTNYGMNWVKQAPGKGLKCMGWINTYTGEPTYADDFKGRFAFSLETSASTAYLQINNLKNEDMATYFCAKYDNYAWFAYWGQGTLVTVSA

SEQ ID NO: 9 - protein, 116 aa, synthetic construct
  REGION 1..116: note = MTK-05 Heavy Chain Variable
QVQLQQSGAELVQPGTSVRLSCKTSGYTFTSYWIQWVKQRPGQGLGWIGEIFPGTGTTYYNEKFKGKATLTIDTSSSTAYMQLSSLTSEDSAVYFCARDGAYFDVWGAGTTVTVSS

SEQ ID NO: 10 - protein, 118 aa, synthetic construct
  REGION 1..118: note = MTK-06 Heavy Chain Variable
QIQLVQSGPELKKPGETVKISCKASGYTFTNYGMNWVKQAPGKGLKWMGWINTNTGEPTYAEEFKGRFAFSLETSASTAYLQINNLKNEDTATYFCARYYNYWYFDVWGAGTTVTVSS

SEQ ID NO: 11 - protein, 120 aa, synthetic construct
  REGION 1..120: note = MTK-07 Heavy Chain Variable
DVQLQESGPGLVKPSQSLSLTCSVTGYSITSGYYWNWIRQFPGNTLEWMGYMSFDGDNKFNPSLKNRISITRDTSKNQFFLRLNSVTTEDTATYYCARGGYNYGSTEANWGQGTLVTVST

SEQ ID NO: 12 - protein, 118 aa, synthetic construct
  REGION 1..118: note = MTK-08 Heavy Chain Variable
QIQLVQSGPELKKPGETVKISCKASGYTFTNYGMNWVKQAPGKGLKWMGWINTYTGEPTYADDFKGRFAFSLETSASTAYLQINNLKNEDTATYFCAREVRYWYFDVWGAGTTVTVSS

SEQ ID NO: 13 - protein, 123 aa, synthetic construct
  REGION 1..123: note = MTK-09 Heavy Chain Variable
QVQLQQSGAELVRPGASVKISCKAFGYTFTNHHIKWVKQRPGQGLDWIGYIDPYNDYTTYNQNFKGKATLTVDKSSSTAYMELSSLTSEDSAVYYCARRAYDGYYVDWYFDVWGAGTTVTVSS

SEQ ID NO: 14 - protein, 119 aa, synthetic construct
  REGION 1..119: note = MTK-10 Heavy Chain Variable
QVTLKESGPGILQPSQTLSLTCSFSGFSLTTSGMGVGWIRQPSGKGLEWLAHIWSDDDKRSNPALKSRLTISQDSSTNQVFLKIASVDTADSATYYCSHLTPVREFAYWGPGTLVTVSE

SEQ ID NO: 15 - protein, 118 aa, synthetic construct
  REGION 1..118: note = MTK-11 Heavy Chain Variable
QVQLKQSGPGLVQPSQSLSITCTVSGFSLTDYGVHWVRQSPGKGLEWLGVIWSSGTTDYNEAFISRLSISKDNSKSHVFFKMNSLQAIDTAIYYCARKGHDPYAMDYWGQGTSVTVSS

SEQ ID NO: 16 - protein, 122 aa, synthetic construct
  REGION 1..122: note = MTK-12 Heavy Chain Variable
QVQLQQSGPQLVRPGASVKISCKASGYSFTSHWMHWVKQRPGQGLEWIGMIDPSDGESRLNQKFKDKATLTVDKSSSTAYMQLSSPTFEDSAVYYCARGIYYYGITYAMDYWGQGTSVTVSS

SEQ ID NO: 17 - protein, 119 aa, synthetic construct
  REGION 1..119: note = MTK-13 Heavy Chain Variable
DVQLQESGPGLVKPSQSLSLTCSVTGYSITSGYYWNWIRQFPGNKLEWMGNIDYDGSNKYNPSLKNRISITRDTSKNHFFLKLNSVTPEDTATYFCARDGGNYRSFAYWGQGTLVTVSA

SEQ ID NO: 18 - protein, 118 aa, synthetic construct
  REGION 1..118: note = MTK-14 Heavy Chain Variable
QIQLVQSGPELKKPGETVKISCKASGYTFTNYGMNWVKQAPGKDLQWMGWINTYTGEPTYADDFTGRFAFSLETSASTAYLQINNLKNEDTATYFCAKGGHYAWFAYWGQGTLVTVSA

SEQ ID NO: 19 - protein, 118 aa, synthetic construct
  REGION 1..118: note = MTK-15 Heavy Chain Variable
QIQLVQSGPELKKPGETVKISCKASGYTFTNYGVHWVKQAPGKGLKWMGWINTYTGEPTYADDFKGRFAFSLETSASTAYLQINNLKNEDMATYFCARGNRYAYMDYWGQGTSVTVSS

SEQ ID NO: 20 - protein, 120 aa, synthetic construct
  REGION 1..120: note = MTK-16 Heavy Chain Variable
LIQLVQSGPELKKPGETVKISCKASGYTFTNHGMNWVKQDPGKGLKWMGWINTYTGEPTYADDFKGRFVFSMETSASAAFLQINNLKNEDTATYFCARKGVTAARYFDYWGQGTTLTVSS

SEQ ID NO: 21 - protein, 120 aa, synthetic construct
  REGION 1..120: note = MTK-17 Heavy Chain Variable
QIQLVQSGPELKKPGETVKISCKASGYTFTNFGMNWVKQAPGKGLKWMGWINTYTGEPTYADDFKGRFAFSLETSASTAYLQINNLKNEDTATYFCSRGAVLRAGAMDCWGQGTSVTVSS

SEQ ID NO: 22 - protein, 121 aa, synthetic construct
  REGION 1..121: note = MTK-18 Heavy Chain Variable
QIQLVQSGPELKKPGETVKISCKASGYTFTHYGMTWVKQAPGKGLKWMGWINTYTGEPTYADDFKGRFAFSLETSASTAYLQINNLKNEDTATYFCARGGGQLGLRPLDYWGQGTTLTVSS

SEQ ID NO: 23 - protein, 125 aa, synthetic construct
  REGION 1..125: note = MTK-19 Heavy Chain Variable
EVQLQQSGPELVKPGASVKISCKTSGYTFTEYTMHWVKQSHGKSLEWLGGFNPNNVITSYNQRFQGRATLTVDKSSSTAYMELRSLTSDDSAVYYCTRGDLLWSLLLPGNYFDYWGQGTTLTVSS

SEQ ID NO: 24 - protein, 121 aa, synthetic construct
  REGION 1..121: note = MTK-20 Heavy Chain Variable
QIQLVQSGPELKKPGETVKISCKASGYTFTNYGMTWVKQAPGKGLKWMGWINTYTGEPTYADDFKGRFALSLETSASTAYLQINNLKNEDTATYFCARGGGRLGLRPLDYWGQGTTLTVSS

SEQ ID NO: 25 - protein, 121 aa, synthetic construct
  REGION 1..121: note = MTK-21 Heavy Chain Variable
QIQLVQSGPELKKPGETVKISCKASGYTFTNFGMTWVRQAPGMDLKWMGWINTYTGEPKYADDFKGRFALSLETSASTAYLQITNFKNEDTATYFCARGGGRLGLRPLDYWGQGTTLTVSS

SEQ ID NO: 26 - protein, 120 aa, synthetic construct
  REGION 1..120: note = MTK-22 Heavy Chain Variable
QVQLQQPGADLVEPGASVKLSCKASGYTFTSYWMHWVKQRPGQGLEWIGEIDPSDSSSNYNQKFKGKATLTVDKSSSTAYMHLNSLTSEDSAVYYCARRYYYGSLYFDNWGQGTTLTVSS

SEQ ID NO: 27 - protein, 123 aa, synthetic construct
  REGION 1..123: note = MTK-23 Heavy Chain Variable
QVQLQQSGADLVRPGASVKISCKAFGYTFTNHHINWVKQRPGQGLDWIGNVDPYNDYSTYNQKFKGKATLTVDKSSSTAYMELSSLTSEDSAVYYCARRVYDGFYVDWYFDVWGAGTTVTVSS

SEQ ID NO: 28 - protein, 121 aa, synthetic construct
  REGION 1..121: note = MTK-24 Heavy Chain Variable
QVQLQQPGAELVKPGASVKLSCKASGYTFTSYWMHWVKQRPGQGLEWIGMIHPNINTNYNEKFKSKATLTVDISSSTAYMQLSSLTSEDSAVYYCAKRSPYSNYDWYFDVWGTGTTVTVSS

SEQ ID NO: 29 - protein, 117 aa, synthetic construct
  REGION 1..117: note = MTK-25 Heavy Chain Variable
EVKLVESEGGLVQPGSSMKLSCTASGFTFSDYYMAWVRQVPEKGLEWVANINYEGSSTYYLGSLKSRFIISRDNAENILYLQMSSLKSEDTATYYCARYYYGSVDYWGQGTTLTVSS

SEQ ID NO: 30 - protein, 119 aa, synthetic construct
  REGION 1..119: note = MTK-26 Heavy Chain Variable
AVQFQESGPGLVKLSQSLSLTCSVTGYSITSGYYWDWIRQFPGNKLEWMGYISYDGSNNYNPSLKNRISITRDTSKNQFFLKLNSVTTEDTATYYCAREGGYYRSMDYWGQGTSVTVSS

SEQ ID NO: 31 - protein, 117 aa, synthetic construct
  REGION 1..117: note = MTK-27 Heavy Chain Variable
EVQLQQSGTELVRPGASVKLSCTASGFNIKDDYMHWVQQRPEQGLEWIGWIDPENGNTEYASKFQGKATITADASSNTAYLQLSSLTSEDTAVYYCSTLFSNYFDYWGQGTTLTVSS

SEQ ID NO: 32 - protein, 118 aa, synthetic construct
  REGION 1..118: note = MTK-28 Heavy Chain Variable
QIQLVQSGPELKKPGETVKISCKASGYTFTTYGMSWVKQAPGKGLKWMGWINTYSGVPTYADDFKGRFAFSLETSASTAYLQINTLKNEDTTTYFCARFLRYYYFDYWGQGTTLTVSS

SEQ ID NO: 33 - protein, 118 aa, synthetic construct
  REGION 1..118: note = MTK-29 Heavy Chain Variable
QIQLVQSGPELKKPGETVKISCKSSGFTFTTYGMSWVKQAPGKGLKWMGWINTYSGVPTYTDDFKGRFAFSLETSASTASLQINNLKNEDTATYFCARYTNYGYFDYWGQGTTLTVSS

SEQ ID NO: 34 - protein, 117 aa, synthetic construct
  REGION 1..117: note = MTK-30 Heavy Chain Variable
QVQLQQPGTELVRPGASVKLSCKASGYTFTSYWMHWVKQRPGQGLEWIGNLNPNNGGTYYNEKFKSKATLTVDKSSSTAYMQLSSLTSEDSAVYYCARQILLYFDYWGQGTTLTVSS

SEQ ID NO: 35 - protein, 122 aa, synthetic construct
  REGION 1..122: note = MTK-31 Heavy Chain Variable
QIQLVQSGPELKKSGETVKISCKASGYTFTSYGMSWVKQAPGKGLKWMGWINTYSGVPTYADDFKGRFAFSLETSASTAYLQINNLKNEDTATYFCASLSYDGSLYHAMDYWGQGTSVTVSS

SEQ ID NO: 36 - protein, 118 aa, synthetic construct
  REGION 1..118: note = MTK-32 Heavy Chain Variable
QIQLVQSGPELKKPGETVKISCKASGYTFTSYGMSWVHQAPGKGLKWLGWINTYSGVPTYADDFRGRFAFSLDTSVSTASLEINNLQNEDTATYFCAREDNWAWFAYWGQGTLVTVST

SEQ ID NO: 37 - protein, 119 aa, synthetic construct
  REGION 1..119: note = MTK-33, 34 Heavy Chain Variable
QVQLQQPGPELVRPGTSVKLSCKASGYTFTSYWMHWIKQRPGQGLEWIGVIDPSDNYINYNQKFKGKATLTVDTSSSTAYLQLSSLTSEDSAVYYCAREAGTRGYFDYWGQGTTLTVSS

SEQ ID NO: 38 - protein, 120 aa, synthetic construct
  REGION 1..120: note = MTK-35 Heavy Chain Variable
QVLLQQSGPELVKPGASVQLSCKASDYTFTSYDIHWVKQRPGQGLEWIGWIYPRDGYTKYNEIFKGKATLTVDTSSTTAYMELHSLTSEDSAVYFCARAYYTNWYYFDYWGQGTTLTVSS

SEQ ID NO: 39 - protein, 122 aa, synthetic construct
  REGION 1..122: note = MTK-36 Heavy Chain Variable
QVTLKESGPGILQPSQTLSLTCSFSGFSLSTFGMGVGWIRQPSGKGLEWLAHIWWYDDKYYEPALKSRLTISKDSSKNQVFLKIANVDTADTATYYCARIYYGTSYRYFDVWGTGTTVTVSS

SEQ ID NO: 40 - protein, 106 aa, synthetic construct
  REGION 1..106: note = MTK-01 Light Chain Variable
QIVLSQSPAILSASPGEKVTMTCRASSSVSYMHWYQQKPGSSPKPWIYATSNLASGVPARFSGSGSGTSYSLTISRVEAEDAATYYCQQWSSNPPTFGGGTKLEIK

SEQ ID NO: 41 - protein, 106 aa, synthetic construct
  REGION 1..106: note = MTK-02 Light Chain Variable
QIVLSQSPAILSASPGERVTMTCRANSSVSYMHWYQQKPGSSPKPWIYATSNLASGVPARFSASGSGTSYSLTVSRVEAEDAATYYCQQWGSNPFTFGSGTKLEIK

SEQ ID NO: 42 - protein, 106 aa, synthetic construct
  REGION 1..106: note = MTK-03 Light Chain Variable
QIVLTQSPAIMSAFPGEKVTITCSASSSVNYMHWFQQKPGTSPKLWIYSTSNLASGVPARFSGSGSGTSYSLTISRMEAEDAATYYCQQRSSYPFTFGSGTKLEIK

SEQ ID NO: 43 - protein, 106 aa, synthetic construct
  REGION 1..106: note = MTK-04 Light Chain Variable
QIVLSQSPAFLSASPGEKVTMTCRASSSVSYMHWYLQKPGSSPKPWIYATSNLASGVPVRFSGSGSGTSYSLTISRVEAEDAATYYCQQWSSNPRTFGGGTKLEIR

SEQ ID NO: 44 - protein, 107 aa, synthetic construct
  REGION 1..107: note = MTK-05 Light Chain Variable
DIQMTQSPASLSASVGETVTFTCRASENIFSYLAWYQQKQGKSPQLLVYNAKTLAEGVPSRFSGSGSGTHFSLKINSLQPEDFGSYYCLHHYGTPLTFGAGTKLELK

SEQ ID NO: 45 - protein, 106 aa, synthetic construct
  REGION 1..106: note = MTK-06 Light Chain Variable
QIVLSQSPAILSASPGEKVTMTCRASSSVSYMHWYQQKPGSSPKPWIYATSNLASGVPARFSGSGSGTSYSLTISRVEAEDAATYYCQQWSSKPPTFGGGTKLEIK

SEQ ID NO: 46 - protein, 106 aa, synthetic construct
  REGION 1..106: note = MTK-07 Light Chain Variable
QIVLTQSPAIMSAFPGEKVTITCSASSSVTYMHWFQQKPGTSPKLWIFSTSNLASGVPARFSGSGSGTSYSLTISRMEAEDAATYYCQQRSSYPFTFGSGTKLEIK

SEQ ID NO: 47 - protein, 106 aa, synthetic construct
  REGION 1..106: note = MTK-08 Light Chain Variable
QIVLSQSPAILSASPGEKVTMTCRATSSVSYMHWFQQKPGSSPKPWIYATSNLASGVPARFSGSGSGTSYSLTISRVEAEDAAAYYCQHWSGNPRTFGGGTKLEIK

SEQ ID NO: 48 - protein, 107 aa, synthetic construct
  REGION 1..107: note = MTK-09 Light Chain Variable
DIVMTQSHKFMSTSVGDRVSITCKASQDVTTSVAWYHQKPGQSPELLIYSASYRYTGVPDRFTGSGSGTDFTFTISSVQAEDLAIYYCQQHYSTPLTFGSGTKLQLK

SEQ ID NO: 49 - protein, 107 aa, synthetic construct
  REGION 1..107: note = MTK-10 Light Chain Variable
DIQMTQTTSSLSASLGDRVTISCSASQGISNYLNWFQQKPDGTVKLLIYYTSRLHSGVPSRFSGSGSGTDFSLTISNLEPEDIATYYCQQYTKLPYTFGGGTKLEIK

SEQ ID NO: 50 - protein, 108 aa, synthetic construct
  REGION 1..108: note = MTK-11 Light Chain Variable
QIVLTQSPAIMSASLGERVTMTCTASSSISSSYFHWYQQKPGSSPKLWIYSTSNLPSGVPARFSGSGSGTSYSLTISSMEAEDAATYYCHQYYRSPLTFGAGTKLELK

SEQ ID NO: 51 - protein, 106 aa, synthetic construct
  REGION 1..106: note = MTK-12 Light Chain Variable
QIVLTQSPAIMSASPGEKVTMTCSASSSVSYMYWYQQKPRSSPRLLIYDTSNLASGVPVRFSGSGSGTSYSLTISRMEAEDAATYYCQQWSSYPLTFGAGTKLELK

SEQ ID NO: 52 - protein, 106 aa, synthetic construct
  REGION 1..106: note = MTK-13 Light Chain Variable
QIVLTQSPPIMSASPGEKVTITCSASSSVSYMYWFQQKPGTSPKLWIYSTFNLASGVPARFSGSGSGTSYSLTISRMEAEDVATYYCQQRSSYPFTFGSGTKLEIK

SEQ ID NO: 53 - protein, 106 aa, synthetic construct
  REGION 1..106: note = MTK-14 Light Chain Variable
QIVLSQSPAILSASPGEKVTMTCRSSSSVSYMHWYQQKPGSSPKPWIYATSNLASGVPGRFSGSGSGTSYSLTISRVEAEDAATYYCQQWGSNPRTFGGGTKLEIK

SEQ ID NO: 54 - protein, 106 aa, synthetic construct
  REGION 1..106: note = MTK-15 Light Chain Variable
QIVLSQSPAILSASPGEKVTMTCRASSSVSYMHWYQQKSGSSPKPWIYATSNLASGVPARFSGSGSGTSYSLTISRVEAEDAATYYCQQWSSNPRTFGGGTKLEIK

SEQ ID NO: 55 - protein, 107 aa, synthetic construct
  REGION 1..107: note = MTK-16 Light Chain Variable
DIVMTQSPKFMSTSVGDRVSITCKASQNVRTAVAWYKKKPGQSPKALINLASNRHTGVPDRFTGSGSGTDFTLTISNVQSEDLADYFCLQHWNYPLTFGGGTKLEIK

SEQ ID NO: 56 - protein, 107 aa, synthetic construct
  REGION 1..107: note = MTK-17 Light Chain Variable
DIQMTQSSSYLSVSLGGRVTITCKASDHINNWLAWYQQKPGNAPRLLISGATSLETGVPSRFSGSGSGKDYTLSITSLQTEDVATYYCQQYWSTPRTFGGGTKLEIK

SEQ ID NO: 57 - protein, 107 aa, synthetic construct
  REGION 1..107: note = MTK-18 Light Chain Variable
DIQMTQTTSSLSASLGDRVTFSCRASQAISNYLNWYQQKPDGTVKLLIYYTSRLHSGVPSRFSGSGSGTDYSLTISNLEQEDIATYFCQQGNTLPWTFGGGTKLEIK

SEQ ID NO: 58 - protein, 112 aa, synthetic construct
  REGION 1..112: note = MTK-19 Light Chain Variable
DVVMTQSSLTLSVTIGQPASISCKSSQSLLDSDGKTYLNWLLQRPGQSPKRLIYLVSKLDSGVPDRFTGSGSGTDFTLKISRVEAEDLGVYYCWQGTHFPWTFGGGTKLEIK

SEQ ID NO: 59 - protein, 107 aa, synthetic construct
  REGION 1..107: note = MTK-20 Light Chain Variable
DIQMTQTTSSLSASLGDRVTISCRASQDINNYLNWYQQKPDGTVKLLIYYTSRLHSGVPSRFSGSGSGTDYSLTISNLEQEDIATYFCQQGNTLPWTFGGGTKLEIK

SEQ ID NO: 60 - protein, 107 aa, synthetic construct
  REGION 1..107: note = MTK-21 Light Chain Variable
DIQMTQTTSSLSASLGDRVTISCRASQDINNYLNWYQQKPDGTVKLLIYYTSSLHSGVPSRFSGSGSGTDYSLTITNLEQEDIATYFCQQGNTLPWTFGGGTRLEIK

SEQ ID NO: 61 - protein, 107 aa, synthetic construct
  REGION 1..107: note = MTK-22 Light Chain Variable
DVVMTQSHKFMSTSVGDRVSITCKASQDVGTAIAWYQQKPGQSPKLLIYWASTRHTGVPDRFTGSRSGTVFTLTISNVQSEDLADYFCQQYTSYPLTFGSGTKLEIK

SEQ ID NO: 62 - protein, 107 aa, synthetic construct
  REGION 1..107: note = MTK-23 Light Chain Variable
DIVMTQSHKFMSTPVGDRVSITCKASRDVSTAVAWFHQKPGQSPKLLIYSASYRSTGVPDRFTGSGSGTDFTFTISSVQAEDLAVYYCQQHYSAPLTFGAGTKLELK

SEQ ID NO: 63 - protein, 107 aa, synthetic construct
  REGION 1..107: note = MTK-24 Light Chain Variable
DIQMTQSPASLSVSVGETVTITCRASENIYSNLAWYQQKQGKSPQLLVYAATNLADGVPSRFSGSGSGTQYSLKINSLQSEDFGSYYCQHFWGTPLTFGAGTKLELK

SEQ ID NO: 64 - protein, 107 aa, synthetic construct
  REGION 1..107: note = MTK-25 Light Chain Variable
DIVMTQSQKFMSTSVGDRVSITCKASQNVRTTVAWYQQKPGQSPKTLIYLASNRHTGVPDRFTGSGSGTDFTLTISNVQSEDLADYFCLQLWNYPWTFGGGTKLEIK

SEQ ID NO: 65 - protein, 106 aa, synthetic construct
  REGION 1..106: note = MTK-26 Light Chain Variable
QIVLTQSPAIMSASPGEKVTIACSASSSVSFMHWFQQKPGTSPRLWIYSTSNLASGVPARFSGSGSGTSYSLTISRMEAEDAATYYCQQRSSYPFTFGSGTKLEIK

SEQ ID NO: 66 - protein, 107 aa, synthetic construct
  REGION 1..107: note = MTK-27 Light Chain Variable
DIVMTQSQKFMSTSVGDRVSITCKASQNVRTSVAWYQQKPGQSPKALIYLASNRHTGVPDRFTGSGSGTDFTLTISNVRSEDLADYFCLQHWNYPYTFGGGTKLEIK

SEQ ID NO: 67 - protein, 105 aa, synthetic construct
  REGION 1..105: note = MTK-28 Light Chain Variable
QIVLSQSPVILSASPGEKVTMTCRASSSVTYMHWYQQQPGSSPKPWIYATSNLASGVPARFSGSGSGTSYSLTISRVETEDAATYYCHQWSGNPTFGGGTKLEIN

SEQ ID NO: 68 - protein, 106 aa, synthetic construct
  REGION 1..106: note = MTK-29 Light Chain Variable
QIVLSQSPAILSASPGEKVTMTCRATSSVGYMHWYQQKPGSSPKPWIYATSNLASGVPARFSGSGSGTSYSLTISRVEAEHAATYYCQQWGSNPFTFGSGTKLEIK

SEQ ID NO: 69 - protein, 112 aa, synthetic construct
  REGION 1..112: note = MTK-30 Light Chain Variable
DVLMTQTPLSLPVSLGDQASISCRSSQSIVHSNGDTYLEWYLQKPGQSPKLLIYKVSNRFSGVPDRFSGSGSGTDFTLKISRVEAEDLGVYYCFQGSHVPWTFGGGTKLEIK

SEQ ID NO: 70 - protein, 112 aa, synthetic construct
  REGION 1..112: note = MTK-31 Light Chain Variable
DVSMTQTPLSLPVSLGDQASISSRSSQTIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 71 - protein, 106 aa, synthetic construct
  REGION 1..106: note = MTK-32 Light Chain Variable
QIVLSQSPAILSASPGEKVTMTCRATSSVGYMHWYQRKPGSSPKPWIYATSNLASGVPARFSGSGSGTSYSLTISRVEAEDAATYYCQQWSSNPRTFGGGTKLEIK

SEQ ID NO: 72 - protein, 107 aa, synthetic construct
  REGION 1..107: note = MTK-33, 34 Light Chain Variable
SIVMTQTPKFLLVSAGDRVIITCKASQSVSNTVAWYQQKPGQSPKLLIYYASNRYTGVPDRFTGSGYGTDFTFTISTVQAEDLAVYFCQQDYRSPFTFGSGTQLEMK

SEQ ID NO: 73 - protein, 108 aa, synthetic construct
  REGION 1..108: note = MTK-35 Light Chain Variable
EIVLTQSPTTMAASPGEKITITCRASSSISSHYLHWYQQKPGFSPKLLIYRTSNLASGVPARFSGSGSGTSYSLTIGTMEAEDVATYYCQQGSTIPLTFGAGTKLVLK

SEQ ID NO: 74 - protein, 106 aa, synthetic construct
  REGION 1..106: note = MTK-36 Light Chain Variable
QIVLSQSPAILSAFPGEKVTMTCRATSSVRYMHWYQQKPGSSPKPWIYATYNLTSGVPARFSGSGSGTSYSLTISRVEAEDAATYYCHQWSSNPYTFGGGTKLEIK

SEQ ID NO: 75 - protein, 5 aa, synthetic construct
  REGION 1..5: note = MTK-01, 04, 06, 08, 14 HVR-H1
NYGMN

SEQ ID NO: 76 - protein, 5 aa, synthetic construct
  REGION 1..5: note = MTK-02 HVR-H1
DYGVN

SEQ ID NO: 77 - protein, 6 aa, synthetic construct
  REGION 1..6: note = MTK-03, 07, 13 HVR-H1
SGYYWN

SEQ ID NO: 78 - protein, 5 aa, synthetic construct
  REGION 1..5: note = MTK-05 HVR-H1
SYWIQ

SEQ ID NO: 79 - protein, 5 aa, synthetic construct
  REGION 1..5: note = MTK-09 HVR-H1
NHHIK

SEQ ID NO: 80 - protein, 7 aa, synthetic construct
  REGION 1..7: note = MTK-10 HVR-H1
TSGMGVG

SEQ ID NO: 81 - protein, 5 aa, synthetic construct
  REGION 1..5: note = MTK-11 HVR-H1
DYGVH

SEQ ID NO: 82 - protein, 5 aa, synthetic construct
  REGION 1..5: note = MTK-12 HVR-H1
SHWMH

SEQ ID NO: 83 - protein, 5 aa, synthetic construct
  REGION 1..5: note = MTK-15, 15.1, 15.2, 15.3, 15.4, 15.5, 15.6, 15.7, 15.8, 15.9, 15.10, 15.11, 15.12, 15.13, 15.14, 15.15 HVR-H1
NYGVH

SEQ ID NO: 84 - protein, 5 aa, synthetic construct
  REGION 1..5: note = MTK-16, 16.1, 16.2 HVR-H1
NHGMN

SEQ ID NO: 85 - protein, 5 aa, synthetic construct
  REGION 1..5: note = MTK-17 HVR-H1
NFGMN

SEQ ID NO: 86 - protein, 5 aa, synthetic construct
  REGION 1..5: note = MTK-18 HVR-H1
HYGMT

SEQ ID NO: 87 - protein, 5 aa, synthetic construct
  REGION 1..5: note = MTK-19 HVR-H1
EYTMH

SEQ ID NO: 88 - protein, 5 aa, synthetic construct
  REGION 1..5: note = MTK-20 HVR-H1
NYGMT

SEQ ID NO: 89 - protein, 5 aa, synthetic construct
  REGION 1..5: note = MTK-21 HVR-H1
NFGMT

SEQ ID NO: 90 - protein, 5 aa, synthetic construct
  REGION 1..5: note = MTK-22, 24, 30, 33, 33.1, 33.2, 33.3, 33.4, 33.5, 33.6, 33.7, 33.8, 33.9, 33.10, 33.11, 33.12, 33.13, 33.14, 33.15, 33.16, 33.17, 33.18, 33.19, 33.20, 33.21, 33.22, 33.23, 33.24, 34 HVR-H1
SYWMH

SEQ ID NO: 91 - protein, 5 aa, synthetic construct
  REGION 1..5: note = MTK-23 HVR-H1
NHHIN

SEQ ID NO: 92 - protein, 5 aa, synthetic construct
  REGION 1..5: note = MTK-25 HVR-H1
DYYMA

SEQ ID NO: 93 - protein, 6 aa, synthetic construct
  REGION 1..6: note = MTK-26 HVR-H1
SGYYWD

SEQ ID NO: 94 - protein, 5 aa, synthetic construct
  REGION 1..5: note = MTK-27 HVR-H1
DDYMH

SEQ ID NO: 95 - protein, 5 aa, synthetic construct
  REGION 1..5: note = MTK-28, 29, 29.2, 29.3, 29.4, 29.6, 29.7, 29.10, 29.11, 29.12, 29.13, 29.14, 29.15, 29.19, 29.20, 29.21 HVR-H1
TYGMS

SEQ ID NO: 96 - protein, 5 aa, synthetic construct
  REGION 1..5: note = MTK-31, 32 HVR-H1
SYGMS

SEQ ID NO: 97 - protein, 5 aa, synthetic construct
  REGION 1..5: note = MTK-35 HVR-H1
SYDIH

SEQ ID NO: 98 - protein, 7 aa, synthetic construct
  REGION 1..7: note = MTK-36 HVR-H1
TFGMGVG

SEQ ID NO: 99 - protein, 17 aa, synthetic construct
  REGION 1..17: note = MTK-01, 04, 08, 15, 16, 16.2, 17, 18, 20 HVR-H2
WINTYTGEPTYADDFKG

SEQ ID NO: 100 - protein, 17 aa, synthetic construct
  REGION 1..17: note = MTK-02 HVR-H2
WINTYSGEPTYAGDFKG

SEQ ID NO: 101 - protein, 16 aa, synthetic construct
  REGION 1..16: note = MTK-03, 07 HVR-H2
YMSFDGDNKFNPSLKN

SEQ ID NO: 102 - protein, 17 aa, synthetic construct
  REGION 1..17: note = MTK-05 HVR-H2
EIFPGTGTTYYNEKFKG

SEQ ID NO: 103 - protein, 17 aa, synthetic construct
  REGION 1..17: note = MTK-06 HVR-H2
WINTNTGEPTYAEEFKG

SEQ ID NO: 104 - protein, 17 aa, synthetic construct
  REGION 1..17: note = MTK-09 HVR-H2
YIDPYNDYTTYNQNFKG

SEQ ID NO: 105 - protein, 16 aa, synthetic construct
  REGION 1..16: note = MTK-10 HVR-H2
HIWSDDDKRSNPALKS

SEQ ID NO: 106 - protein, 16 aa, synthetic construct
  REGION 1..16: note = MTK-11 HVR-H2
VIWSSGTTDYNEAFIS

SEQ ID NO: 107 - protein, 17 aa, synthetic construct
  REGION 1..17: note = MTK-12 HVR-H2
MIDPSDGESRLNQKFKD

SEQ ID NO: 108 - protein, 16 aa, synthetic construct
  REGION 1..16: note = MTK-13 HVR-H2
NIDYDGSNKYNPSLKN

SEQ ID NO: 109 - protein, 17 aa, synthetic construct
  REGION 1..17: note = MTK-14 HVR-H2
WINTYTGEPTYADDFTG

SEQ ID NO: 110 - protein, 17 aa, synthetic construct
  REGION 1..17: note = MTK-19 HVR-H2
GFNPNNVITSYNQRFQG

SEQ ID NO: 111 - protein, 17 aa, synthetic construct
  REGION 1..17: note = MTK-21 HVR-H2
WINTYTGEPKYADDFKG

SEQ ID NO: 112 - protein, 17 aa, synthetic construct
  REGION 1..17: note = MTK-22 HVR-H2
EIDPSDSSSNYNQKFKG

SEQ ID NO: 113 - protein, 17 aa, synthetic construct
  REGION 1..17: note = MTK-23 HVR-H2
NVDPYNDYSTYNQKFKG

SEQ ID NO: 114 - protein, 16 aa, synthetic construct
  REGION 1..16: note = MTK-24 HVR-H2
MIHPNINTNYNEKFKS

SEQ ID NO: 115 - protein, 17 aa, synthetic construct
  REGION 1..17: note = MTK-25 HVR-H2
NINYEGSSTYYLGSLKS

SEQ ID NO: 116 - protein, 16 aa, synthetic construct
  REGION 1..16: note = MTK-26 HVR-H2
YISYDGSNNYNPSLKN

SEQ ID NO: 117 - protein, 17 aa, synthetic construct
  REGION 1..17: note = MTK-27 HVR-H2
WIDPENGNTEYASKFQG

SEQ ID NO: 118 - protein, 17 aa, synthetic construct
  REGION 1..17: note = MTK-28, 31 HVR-H2
WINTYSGVPTYADDFKG

SEQ ID NO: 119 - protein, 17 aa, synthetic construct
  REGION 1..17: note = MTK-29, 29.2, 29.4, 29.5, 29.8, 29.9, 29.10 HVR-H2
WINTYSGVPTYTDDFKG

SEQ ID NO: 120 - protein, 17 aa, synthetic construct
  REGION 1..17: note = MTK-30 HVR-H2
NLNPNNGGTYYNEKFKS

SEQ ID NO: 121 - protein, 17 aa, synthetic construct
  REGION 1..17: note = MTK-32 HVR-H2
WINTYSGVPTYADDFRG

SEQ ID NO: 122 - protein, 17 aa, synthetic construct
  REGION 1..17: note = MTK-33, 34 HVR-H2
VIDPSDNYINYNQKFKG

SEQ ID NO: 123 - protein, 17 aa, synthetic construct
  REGION 1..17: note = MTK-35 HVR-H2
WIYPRDGYTKYNEIFKG

SEQ ID NO: 124 - protein, 16 aa, synthetic construct
  REGION 1..16: note = MTK-36 HVR-H2
HIWWYDDKYYEPALKS

SEQ ID NO: 125 - protein, 9 aa, synthetic construct
  REGION 1..9: note = MTK-01 HVR-H3
RDRYTWFAY

SEQ ID NO: 126 - protein, 9 aa, synthetic construct
  REGION 1..9: note = MTK-02 HVR-H3
RVRYWYFDV

SEQ ID NO: 127 - protein, 11 aa, synthetic construct
  REGION 1..11: note = MTK-03, 07 HVR-H3
GGYNYGSTEAN

SEQ ID NO: 128 - protein, 9 aa, synthetic construct
  REGION 1..9: note = MTK-04 HVR-H3
YDNYAWFAY

SEQ ID NO: 129 - protein, 7 aa, synthetic construct
  REGION 1..7: note = MTK-05 HVR-H3
DGAYFDV

SEQ ID NO: 130 - protein, 9 aa, synthetic construct
  REGION 1..9: note = MTK-06 HVR-H3
YYNYWYFDV

SEQ ID NO: 131 - protein, 9 aa, synthetic construct
  REGION 1..9: note = MTK-08 HVR-H3
EVRYWYFDV

SEQ ID NO: 132 - protein, 14 aa, synthetic construct
  REGION 1..14: note = MTK-09 HVR-H3
RAYDGYYVDWYFDV

SEQ ID NO: 133 - protein, 9 aa, synthetic construct
  REGION 1..9: note = MTK-10 HVR-H3
LTPVREFAY

SEQ ID NO: 134 - protein, 10 aa, synthetic construct
  REGION 1..10: note = MTK-11 HVR-H3
KGHDPYAMDY

SEQ ID NO: 135 - protein, 13 aa, synthetic construct
  REGION 1..13: note = MTK-12 HVR-H3
GIYYYGITYAMDY

SEQ ID NO: 136 - protein, 10 aa, synthetic construct
  REGION 1..10: note = MTK-13 HVR-H3
DGGNYRSFAY

SEQ ID NO: 137 - protein, 9 aa, synthetic construct
  REGION 1..9: note = MTK-14 HVR-H3
GGHYAWFAY

SEQ ID NO: 138 - protein, 9 aa, synthetic construct
  REGION 1..9: note = MTK-15, 15.2, 15.3, 15.4, 15.6, 15.7, 15.14, 15.15 HVR-H3
GNRYAYMDY

SEQ ID NO: 139 - protein, 11 aa, synthetic construct
  REGION 1..11: note = MTK-16, 16.1, 16.2 HVR-H3
KGVTAARYFDY

SEQ ID NO: 140 - protein, 11 aa, synthetic construct
  REGION 1..11: note = MTK-17 HVR-H3
GAVLRAGAMDC

SEQ ID NO: 141 - protein, 12 aa, synthetic construct
  REGION 1..12: note = MTK-18 HVR-H3
GGGQLGLRPLDY

SEQ ID NO: 142 - protein, 16 aa, synthetic construct
  REGION 1..16: note = MTK-19 HVR-H3
GDLLWSLLLPGNYFDY

SEQ ID NO: 143 - protein, 12 aa, synthetic construct
  REGION 1..12: note = MTK-20, 21 HVR-H3
GGGRLGLRPLDY

SEQ ID NO: 144 - protein, 11 aa, synthetic construct
  REGION 1..11: note = MTK-22 HVR-H3
RYYYGSLYFDN

SEQ ID NO: 145 - protein, 14 aa, synthetic construct
  REGION 1..14: note = MTK-23 HVR-H3
RVYDGFYVDWYFDV

SEQ ID NO: 146 - protein, 13 aa, synthetic construct
  REGION 1..13: note = MTK-24 HVR-H3
RSPYSNYDWYFDV

SEQ ID NO: 147 - protein, 8 aa, synthetic construct
  REGION 1..8: note = MTK-25 HVR-H3
YYYGSVDY

SEQ ID NO: 148 - protein, 10 aa, synthetic construct
  REGION 1..10: note = MTK-26 HVR-H3
EGGYYRSMDY

SEQ ID NO: 149 - protein, 8 aa, synthetic construct
  REGION 1..8: note = MTK-27 HVR-H3
LFSNYFDY

SEQ ID NO: 150 - protein, 9 aa, synthetic construct
  REGION 1..9: note = MTK-28 HVR-H3
FLRYYYFDY

SEQ ID NO: 151 - protein, 9 aa, synthetic construct
  REGION 1..9: note = MTK-29, 29.19, 29.20, 29.21 HVR-H3
YTNYGYFDY

SEQ ID NO: 152 - protein, 8 aa, synthetic construct
  REGION 1..8: note = MTK-30 HVR-H3
QILLYFDY

SEQ ID NO: 153 - protein, 13 aa, synthetic construct
  REGION 1..13: note = MTK-31 HVR-H3
LSYDGSLYHAMDY

SEQ ID NO: 154 - protein, 9 aa, synthetic construct
  REGION 1..9: note = MTK-32 HVR-H3
EDNWAWFAY

SEQ ID NO: 155 - protein, 10 aa, synthetic construct
  REGION 1..10: note = MTK-33, 33.2, 33.4, 33.5, 33.6, 33.7, 33.9, 33.10, 33.12, 33.13, 33.14, 33.15, 33.16, 33.17, 33.18, 33.19, 33.20, 33.21, 33.22, 33.24, 34 HVR-H3
EAGTRGYFDY

SEQ ID NO: 156 - protein, 11 aa, synthetic construct
  REGION 1..11: note = MTK-35 HVR-H3
AYYTNWYYFDY

SEQ ID NO: 157 - protein, 12 aa, synthetic construct
  REGION 1..12: note = MTK-36 HVR-H3
IYYGTSYRYFDV

SEQ ID NO: 158 - protein, 10 aa, synthetic construct
  REGION 1..10: note = MTK-01, 04, 06, 15, 15.1, 15.2, 15.11, 15.14, 15.15 HVR-L1
RASSSVSYMH

SEQ ID NO: 159 - protein, 10 aa, synthetic construct
  REGION 1..10: note = MTK-02 HVR-L1
RANSSVSYMH

SEQ ID NO: 160 - protein, 10 aa, synthetic construct
  REGION 1..10: note = MTK-03 HVR-L1
SASSSVNYMH

SEQ ID NO: 161 - protein, 11 aa, synthetic construct
  REGION 1..11: note = MTK-05 HVR-L1
RASENIFSYLA

SEQ ID NO: 162 - protein, 10 aa, synthetic construct
  REGION 1..10: note = MTK-07 HVR-L1
SASSSVTYMH

SEQ ID NO: 163 - protein, 10 aa, synthetic construct
  REGION 1..10: note = MTK-08 HVR-L1
RATSSVSYMH

SEQ ID NO: 164 - protein, 11 aa, synthetic construct
  REGION 1..11: note = MTK-09 HVR-L1
KASQDVTTSVA

SEQ ID NO: 165 - protein, 11 aa, synthetic construct
  REGION 1..11: note = MTK-10 HVR-L1
SASQGISNYLN

SEQ ID NO: 166 - protein, 12 aa, synthetic construct
  REGION 1..12: note = MTK-11 HVR-L1
TASSSISSSYFH

SEQ ID NO: 167 - protein, 10 aa, synthetic construct
  REGION 1..10: note = MTK-12, 13 HVR-L1
SASSSVSYMY

SEQ ID NO: 168 - protein, 10 aa, synthetic construct
  REGION 1..10: note = MTK-14 HVR-L1
RSSSSVSYMH

SEQ ID NO: 169 - protein, 11 aa, synthetic construct
  REGION 1..11: note = MTK-16, 16.1 HVR-L1
KASQNVRTAVA

SEQ ID NO: 170 - protein, 11 aa, synthetic construct
  REGION 1..11: note = MTK-17 HVR-L1
KASDHINNWLA

SEQ ID NO: 171 - protein, 11 aa, synthetic construct
  REGION 1..11: note = MTK-18 HVR-L1
RASQAISNYLN

SEQ ID NO: 172 - protein, 16 aa, synthetic construct
  REGION 1..16: note = MTK-19 HVR-L1
KSSQSLLDSDGKTYLN

SEQ ID NO: 173 - protein, 11 aa, synthetic construct
  REGION 1..11: note = MTK-20, 21 HVR-L1
RASQDINNYLN

SEQ ID NO: 174 - protein, 11 aa, synthetic construct
  REGION 1..11: note = MTK-22 HVR-L1
KASQDVGTAIA

SEQ ID NO: 175 - protein, 11 aa, synthetic construct
  REGION 1..11: note = MTK-23 HVR-L1
KASRDVSTAVA

SEQ ID NO: 176 - protein, 11 aa, synthetic construct
  REGION 1..11: note = MTK-24 HVR-L1
RASENIYSNLA

SEQ ID NO: 177 - protein, 11 aa, synthetic construct
  REGION 1..11: note = MTK-25 HVR-L1
KASQNVRTTVA

SEQ ID NO: 178 - protein, 10 aa, synthetic construct
  REGION 1..10: note = MTK-26 HVR-L1
SASSSVSFMH

SEQ ID NO: 179 - protein, 11 aa, synthetic construct
  REGION 1..11: note = MTK-27 HVR-L1
KASQNVRTSVA

SEQ ID NO: 180 - protein, 10 aa, synthetic construct
  REGION 1..10: note = MTK-28 HVR-L1
RASSSVTYMH

SEQ ID NO: 181 - protein, 10 aa, synthetic construct
  REGION 1..10: note = MTK-29, 29.19, 29.20, 29.21, 32 HVR-L1
RATSSVGYMH

SEQ ID NO: 182 - protein, 16 aa, synthetic construct
  REGION 1..16: note = MTK-30 HVR-L1
RSSQSIVHSNGDTYLE

SEQ ID NO: 183 - protein, 16 aa, synthetic construct
  REGION 1..16: note = MTK-31 HVR-L1
RSSQTIVHSNGNTYLE

SEQ ID NO: 184 - protein, 11 aa, synthetic construct
  REGION 1..11: note = MTK-33, 34 HVR-L1
KASQSVSNTVA

SEQ ID NO: 185 - protein, 12 aa, synthetic construct
  REGION 1..12: note = MTK-35 HVR-L1
RASSSISSHYLH

SEQ ID NO: 186 - protein, 10 aa, synthetic construct
  REGION 1..10: note = MTK-36 HVR-L1
RATSSVRYMH

SEQ ID NO: 187 - protein, 7 aa, synthetic construct
  REGION 1..7: note = MTK-01, 02, 04, 06, 08, 14, 15, 15.1, 15.2, 15.3, 15.4, 15.5, 15.6, 15.7, 15.8, 15.9, 15.10, 15.11, 15.12, 15.13, 15.14, 15.15, 28, 29, 29.1, 29.2, 29.3, 29.10, 29.11, 29.18, 29.19, 29.20, 29.21, 32 HVR-L2
ATSNLAS

SEQ ID NO: 188 - protein, 7 aa, synthetic construct
  REGION 1..7: note = MTK-03, 07, 26 HVR-L2
STSNLAS

SEQ ID NO: 189 - protein, 7 aa, synthetic construct
  REGION 1..7: note = MTK-05 HVR-L2
NAKTLAE

SEQ ID NO: 190 - protein, 7 aa, synthetic construct
  REGION 1..7: note = MTK-09 HVR-L2
SASYRYT

SEQ ID NO: 191 - protein, 7 aa, synthetic construct
  REGION 1..7: note = MTK-10, 18, 20 HVR-L2
YTSRLHS

SEQ ID NO: 192 - protein, 7 aa, synthetic construct
  REGION 1..7: note = MTK-11 HVR-L2
STSNLPS

SEQ ID NO: 193 - protein, 7 aa, synthetic construct
  REGION 1..7: note = MTK-12 HVR-L2
DTSNLAS

SEQ ID NO: 194 - protein, 7 aa, synthetic construct
  REGION 1..7: note = MTK-13 HVR-L2
STFNLAS

SEQ ID NO: 195 - protein, 7 aa, synthetic construct
  REGION 1..7: note = MTK-16, 16.1, 16.2, 25, 27 HVR-L2
LASNRHT

SEQ ID NO: 196 - protein, 7 aa, synthetic construct
  REGION 1..7: note = MTK-17 HVR-L2
GATSLET

SEQ ID NO: 197 - protein, 7 aa, synthetic construct
  REGION 1..7: note = MTK-19 HVR-L2
LVSKLDS

SEQ ID NO: 198 - protein, 7 aa, synthetic construct
  REGION 1..7: note = MTK-21 HVR-L2
YTSSLHS

SEQ ID NO: 199 - protein, 7 aa, synthetic construct
  REGION 1..7: note = MTK-22 HVR-L2
WASTRHT

SEQ ID NO: 200 - protein, 7 aa, synthetic construct
  REGION 1..7: note = MTK-23 HVR-L2
SASYRST

SEQ ID NO: 201 - protein, 7 aa, synthetic construct
  REGION 1..7: note = MTK-24 HVR-L2
AATNLAD

SEQ ID NO: 202 - protein, 7 aa, synthetic construct
  REGION 1..7: note = MTK-30, 31 HVR-L2
KVSNRFS

SEQ ID NO: 203 - protein, 7 aa, synthetic construct
  REGION 1..7: note = MTK-33, 33.2, 33.3, 33.4, 33.6, 33.7, 33.8, 33.9, 33.10, 33.11, 33.12, 33.13, 33.14, 33.15, 33.16, 33.17, 33.18, 33.19, 33.20, 33.23, 33.24, 34 HVR-L2
YASNRYT

SEQ ID NO: 204 - protein, 7 aa, synthetic construct
  REGION 1..7: note = MTK-35 HVR-L2
RTSNLAS

SEQ ID NO: 205 - protein, 7 aa, synthetic construct
  REGION 1..7: note = MTK-36 HVR-L2
ATYNLTS

SEQ ID NO: 206 - skipped

SEQ ID NO: 207 - protein, 9 aa, synthetic construct
  REGION 1..9: note = MTK-01 HVR-L3
QQWSSNPPT

SEQ ID NO: 208 - protein, 9 aa, synthetic construct
  REGION 1..9: note = MTK-02, 29, 29.19, 29.20, 29.21 HVR-L3
QQWGSNPFT

SEQ ID NO: 209 - protein, 9 aa, synthetic construct
  REGION 1..9: note = MTK-03, 07, 13, 26 HVR-L3
QQRSSYPFT

SEQ ID NO: 210 - protein, 9 aa, synthetic construct
  REGION 1..9: note = MTK-04, 15, 15.1, 15.2, 15.3, 15.4, 15.5, 15.6, 15.7, 15.8, 15.9, 15.10, 15.11, 15.12, 15.13, 15.14, 15.15, 32 HVR-L3
QQWSSNPRT

SEQ ID NO: 211 - protein, 9 aa, synthetic construct
  REGION 1..9: note = MTK-05 HVR-L3
LHHYGTPLT

SEQ ID NO: 212 - protein, 9 aa, synthetic construct
  REGION 1..9: note = MTK-06 HVR-L3
QQWSSKPPT

SEQ ID NO: 213 - protein, 9 aa, synthetic construct
  REGION 1..9: note = MTK-08 HVR-L3
QHWSGNPRT

SEQ ID NO: 214 - protein, 9 aa, synthetic construct
  REGION 1..9: note = MTK-09 HVR-L3
QQHYSTPLT

SEQ ID NO: 215 - protein, 9 aa, synthetic construct
  REGION 1..9: note = MTK-10 HVR-L3
QQYTKLPYT

SEQ ID NO: 216 - protein, 9 aa, synthetic construct
  REGION 1..9: note = MTK-11 HVR-L3
HQYYRSPLT

SEQ ID NO: 217 - protein, 9 aa, synthetic construct
  REGION 1..9: note = MTK-12 HVR-L3
QQWSSYPLT

SEQ ID NO: 218 - protein, 9 aa, synthetic construct
  REGION 1..9: note = MTK-14 HVR-L3
QQWGSNPRT

SEQ ID NO: 219 - protein, 9 aa, synthetic construct
  REGION 1..9: note = MTK-16, 16.1, 16.2 HVR-L3
LQHWNYPLT

SEQ ID NO: 220 - protein, 9 aa, synthetic construct
  REGION 1..9: note = MTK-17 HVR-L3
QQYWSTPRT

SEQ ID NO: 221 - protein, 9 aa, synthetic construct
  REGION 1..9: note = MTK-18, 21 HVR-L3
QQGNTLPWT

SEQ ID NO: 222 - protein, 9 aa, synthetic construct
  REGION 1..9: note = MTK-19 HVR-L3
WQGTHFPWT

SEQ ID NO: 223 - protein, 8 aa, synthetic construct
  REGION 1..8: note = MTK-20 HVR-L3
QQGNTLPW

SEQ ID NO: 224 - protein, 9 aa, synthetic construct
  REGION 1..9: note = MTK-22 HVR-L3
QQYTSYPLT

SEQ ID NO: 225 - protein, 9 aa, synthetic construct
  REGION 1..9: note = MTK-23 HVR-L3
QQHYSAPLT

SEQ ID NO: 226 - protein, 9 aa, synthetic construct
  REGION 1..9: note = MTK-24 HVR-L3
QHFWGTPLT

SEQ ID NO: 227 - protein, 9 aa, synthetic construct
  REGION 1..9: note = MTK-25 HVR-L3
LQLWNYPWT

SEQ ID NO: 228 - protein, 9 aa, synthetic construct
  REGION 1..9: note = MTK-27 HVR-L3
LQHWNYPYT

SEQ ID NO: 229 - protein, 8 aa, synthetic construct
  REGION 1..8: note = MTK-28 HVR-L3
HQWSGNPT

SEQ ID NO: 230 - protein, 9 aa, synthetic construct
  REGION 1..9: note = MTK-30, 31 HVR-L3
FQGSHVPWT

SEQ ID NO: 231 - protein, 9 aa, synthetic construct
  REGION 1..9: note = MTK-33, 33.1, 33.2, 33.3, 33.5, 33.6, 33.7, 33.8, 33.9, 33.10, 33.11, 33.13, 33.14, 33.16, 34 HVR-L3
QQDYRSPFT

SEQ ID NO: 232 - protein, 9 aa, synthetic construct
  REGION 1..9: note = MTK-35 HVR-L3
QQGSTIPLT

SEQ ID NO: 233 - protein, 9 aa, synthetic construct
  REGION 1..9: note = MTK-36 HVR-L3
HQWSSNPYT

SEQ ID NO: 234 - protein, 118 aa, synthetic construct
  REGION 1..118: note = MTK-15.1 Heavy Chain Variable
QVQLVQSGSELKKPGASVKVSCKASGYTFTNYGVHWVRQAPGQGLEWMGWINTYTGEPTYAQGFTGRFVFSLDTSVSTAYLQISSLKAEDTAVYYCARGNRYAYQDYWGQGTLVTVSS

SEQ ID NO: 235 - protein, 118 aa, synthetic construct
  REGION 1..118: note = MTK-15.2 Heavy Chain Variable
QVQLVQSGSELKKPGASVKVSCKASGYTFTNYGVHWVRQAPGQGLEWMGWGNTYTGEPTYAQGFTGRFVFSLDTSVSTAYLQISSLKAEDTAVYYCARGNRYAYMDYWGQGTLVTVSS

SEQ ID NO: 236 - protein, 118 aa, synthetic construct
  REGION 1..118: note = MTK-15.3, 15.4 Heavy Chain Variable
QVQLVQSGSELKKPGASVKVSCKASGYTFTNYGVHWVRQAPGQGLEWMGWINTYTGEPTYAQGFTGRFVFSLDTSVSTAYLQISSLKAEDTAVYYCARGNRYAYMDYWGQGTLVTVSS

SEQ ID NO: 237 - protein, 118 aa, synthetic construct
  REGION 1..118: note = MTK-15.5 Heavy Chain Variable
QVQLVQSGSELKKPGASVKVSCKASGYTFTNYGVHWVRQAPGQGLEWMGWINTYTGEPTYAQGFTGRFVFSLDTSVSTAYLQISSLKAEDTAVYYCTRGTRYAYMDYWGQGTLVTVSS

SEQ ID NO: 238 - protein, 118 aa, synthetic construct
  REGION 1..118: note = MTK-15.6 Heavy Chain Variable
QVQLVQSGSELKKPGASVKVSCKASGYTFTNYGVHWVRQAPGQGLEWMGWINTYTGEPYYAQGFTGRFVFSLDTSVSTAYLQISSLKAEDTAVYYCARGNRYAYMDYWGQGTLVTVSS

SEQ ID NO: 239 - protein, 118 aa, synthetic construct
  REGION 1..118: note = MTK-15.7 Heavy Chain Variable
QVQLVQSGSELKKPGASVKVSCKASGYTFTNYGVHWVRQAPGQGLEWMGWINTYTGEPTYAQGFTGRFVFSLDTSVSTAYLQISSLKAEDTAVYYCVRGNRYAYMDYWGQGTLVTVSS

SEQ ID NO: 240 - protein, 118 aa, synthetic construct
  REGION 1..118: note = MTK-15.8 Heavy Chain Variable
QVQLVQSGSELKKPGASVKVSCKASGYTFTNYGVHWVRQAPGQGLEWMGWINTYTGEPTYAQGFTGRFVFSLDTSVSTAYLQISSLKAEDTAVYYCARGVRYAYMDYWGQGTLVTVSS

SEQ ID NO: 241 - protein, 118 aa, synthetic construct
  REGION 1..118: note = MTK-15.9 Heavy Chain Variable
QVQLVQSGSELKKPGASVKVSCKASGYTFTNYGVHWVRQAPGQGLEWMGWINTYTGEPTYAQGFTGRFVFSLDTSVSTAYLQISSLKAEDTAVYYCARGYRYAYMDYWGQGTLVTVSS

SEQ ID NO: 242 - protein, 118 aa, synthetic construct
  REGION 1..118: note = MTK-15.10 Heavy Chain Variable
QVQLVQSGSELKKPGASVKVSCKASGYTFANYGVHWVRQAPGQGLEWMGWINTYQGEPTYAQGFTGRFVFSLDTSVSTAYLQISSLKAEDTAVYYCTRGARYAYMDYWGQGTLVTVSS

SEQ ID NO: 243 - protein, 118 aa, synthetic construct
  REGION 1..118: note = MTK-15.11 Heavy Chain Variable
QVQLVQSGSELKKPGASVKVSCKASGYTFANYGVHWVRQAPGQGLEWMGWINTYEGEPTYAQGFTGRFVFSLDTSVSTAYLQISSLKAEDTAVYYCARGNRYAYQDYWGQGTLVTVSS

SEQ ID NO: 244 - protein, 118 aa, synthetic construct
  REGION 1..118: note = MTK-15.12 Heavy Chain Variable
QVQLVQSGSELKKPGASVKVSCKASGYTFANYGVHWVRQAPGQGLEWMGWINTYQGEPTYAQGFTGRFVFSLDTSVSTAYLQISSLKAEDTAVYYCARGVRYAYMDYWGQGTLVTVSS

SEQ ID NO: 245 - protein, 118 aa, synthetic construct
  REGION 1..118: note = MTK-15.13 Heavy Chain Variable
QVQLVQSGSELKKPGASVKVSCKASGYTFTNYGVHWVRQAPGQGLEWMGWINTYTGEPTYAQGFTGRFVFSLDTSVSTAYLQISSLKAEDTAVYYCARGNRYAYEDYWGQGTLVTVSS

SEQ ID NO: 246 - protein, 118 aa, synthetic construct
  REGION 1..118: note = MTK-15.14, 15.15 Heavy Chain Variable
QVQLVQSGSELKKPGASVKVSCKASGYTFTNYGVHWVRQAPGQGLEWMGWINTYTGEPTYAQGFTGRFVFSLDTSVSTAYLQICSLKAEDTAVYYCARGNRYAYMDYWGQGTLVTVSS

SEQ ID NO: 247 - protein, 106 aa, synthetic construct
  REGION 1..106: note = MTK-15.1, 15.2, 15.11, 15.14 Light Chain Variable
EIVLTQSPATLSLSPGERATLSCRASSSVSYMHWYQQKPGQAPRPLIYATSNLASGIPARFSGSGSGTSYTLTISSLEPEDFAVYYCQQWSSNPRTFGGGTKVEIK

SEQ ID NO: 248 - protein, 106 aa, synthetic construct
  REGION 1..106: note = MTK-15.3 Light Chain Variable
EIVLTQSPATLSLSPGERATLSCRASSHVSYMHWYQQKPGQAPRPLIYATSNLASGIPARFSGSGSGTSYTLTISSLEPEDFAVYYCQQWSSNPRTFGGGTKVEIK

SEQ ID NO: 249 - protein, 106 aa, synthetic construct
  REGION 1..106: note = MTK-15.4, 15.5, 15.6, 15.10 Light Chain Variable
EIVLTQSPATLSLSPGERATLSCRASSSVGYMHWYQQKPGQAPRPLIYATSNLASGIPARFSGSGSGTSYTLTISSLEPEDFAVYYCQQWSSNPRTFGGGTKVEIK

SEQ ID NO: 250 - protein, 106 aa, synthetic construct
  REGION 1..106: note = MTK-15.7 Light Chain Variable
EIVLTQSPATLSLSPGERATLSCRASSGVSYMHWYQQKPGQAPRPLIYATSNLASGIPARFSGSGSGTSYTLTISSLEPEDFAVYYCQQWSSNPRTFGGGTKVEIK

SEQ ID NO: 251 - protein, 106 aa, synthetic construct
  REGION 1..106: note = MTK-15.8, 15.12 Light Chain Variable
EIVLTQSPATLSLSPGERATLSCRASSSVGYMHWYQQKPGQAPRPLIYATSNLASGIPARFSGSGSGTSYTLTISSPEPEDFAVYYCQQWSSNPRTFGGGTKVEIK

SEQ ID NO: 252 - protein, 106 aa, synthetic construct
  REGION 1..106: note = MTK-15.9 Light Chain Variable
EIVLTQSPATLSLSPGERATLSCRASSSTSYMHWYQQKPGQAPRPLIYATSNLASGIPARFSGSGSGTSYTLTISSLEPEDFAVYYCQQWSSNPRTFGGGTKVEIK

SEQ ID NO: 253 - protein, 106 aa, synthetic construct
  REGION 1..106: note = MTK-15.13 Light Chain Variable
EIVLTQSPATLSLSPGERATLSCRASSSVQYMHWYQQKPGQAPRPLIYATSNLASGIPARFSGSGSGTSYTLTISSLEPEDFAVYYCQQWSSNPRTFGGGTKVEIK

SEQ ID NO: 254 - protein, 106 aa, synthetic construct
  REGION 1..106: note = MTK-15.15 Light Chain Variable
QIVLTQSPGTLSLSPGERATLSCRASSSVSYMHWYQQKPGQAPRPLIYATSNLASGIPDRFSGSGSGTSYTLTISRLEPEDFAVYYCQQWSSNPRTFGGGTKVEIK

SEQ ID NO: 255 - protein, 120 aa, synthetic construct
  REGION 1..120: note = MTK-16.1 Heavy Chain Variable
QVQLVQSGSELKKPGASVKVSCKASGYTFTNHGMNWVRQAPGQGLEWMGWINTYTGEPTYAQGFTGRFVFSLDTSVSTAYLQISSLKAEDTAVYYCARKGVTAARYFDYWGQGTLVTVSS

SEQ ID NO: 256 - protein, 120 aa, synthetic construct
  REGION 1..120: note = MTK-16.2 Heavy Chain Variable
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSS

SEQ ID NO: 257 - protein, 107 aa, synthetic construct
  REGION 1..107: note = MTK-16.1 Light Chain Variable
DIQMTQSPSSLSASVGDRVTITCKASQNVRTAVAWYQQKPGKAPKRLIYLASNRHTGVPSRFSGSGSGTEFTLTISNLQPEDFATYYCLQHWNYPLTFGGGTKVEIK

SEQ ID NO: 258 - protein, 107 aa, synthetic construct
  REGION 1..107: note = MTK-16.2 Light Chain Variable
DIQMTQSPSSLSASVGDRVTITCRASQNVRTAVAWYQQKPGKAPKRLIYLASNRHTGVPSRFSGSGSGTEFTLTISNLQPEDFATYYCLQHWNYPLTFGGGTKLEIK

SEQ ID NO: 259 - protein, 118 aa, synthetic construct
  REGION 1..118: note = MTK-29.1 Heavy Chain Variable
QVQLVQSGSELKKPGASVKVSCKASGYIFTSYGLSWVRQAPGQGLEWMGWINTYSGVPTYAQGFTGRFVFSLDTSVSTAYLQISSLKAEDTAVYYCARYTNYGIFDYWGQGTLVTVSS

SEQ ID NO: 260 - protein, 118 aa, synthetic construct
  REGION 1..118: note = MTK-29.2 Heavy Chain Variable
QVQLVQSGSELKKPGASVKVSCKASGFTFTTYGMSWVRQAPGQGLEWMGWINTYSGVPTYTDDFKGRFVFSLDTSVSTAYLQISSLKAEDTAVYYCARYTNYGIFDYWGQGTLVTVSS

SEQ ID NO: 261 - protein, 118 aa, synthetic construct
  REGION 1..118: note = MTK-29.3 Heavy Chain Variable
QVQLVQSGSELKKPGASVKVSCKASGFTFTTYGMSWVRQAPGQGLEWMGWINTYSGVPAYTDDFKGRFVFSLDTSVSTAYLQISSLKAEDTAVYYCARYTNYGVFDYWGQGTLVTVSS

SEQ ID NO: 262 - protein, 118 aa, synthetic construct
  REGION 1..118: note = MTK-29.4 Heavy Chain Variable
QVQLVQSGSELKKPGASVKVSCKASSYTFTTYGMSWVRQAPGQGLEWMGWINTYSGVPTYTDDFKGRFVFSLDTSVSTAYLQISSLKAEDTAVYYCARYTNYGVFDYWGQGTLVTVSS

SEQ ID NO: 263 - protein, 118 aa, synthetic construct
  REGION 1..118: note = MTK-29.5 Heavy Chain Variable
QVQLVQSGSELKKPGASVKVSCKASGYIFTSYGLSWVRQAPGQGLEWMGWINTYSGVPTYTDDFKGRFVFSLDTSVSTAYLQISPLKAEDTAVYYCARYTNYGVFDYWGQGTLVTVSS

SEQ ID NO: 264 - protein, 118 aa, synthetic construct
  REGION 1..118: note = MTK-29.6, 29.12, 29.15 Heavy Chain Variable
QVQLVQSGSELKKPGASVKVSCKASSYTFTTYGMSWVRQAPGQGLEWMGWINTMSGVPTYTDDFKGRFVFSLDTSVSTAYLQISSLKAEDTAVYYCARYTNYGVFDYWGQGTLVTVSS

SEQ ID NO: 265 - protein, 118 aa, synthetic construct
  REGION 1..118: note = MTK-29.7, 29.14 Heavy Chain Variable
QVQLVQSGSELKKPGASVKVSCKASSYTFTTYGMSWVRQAPGQGLEWMGWINTYSGVPSYTDDFKARFVFSLDTSVSTAYLQISSLKAEDTAVYYCARYTNYGVFDYWGQGTLVTVSS

SEQ ID NO: 266 - protein, 118 aa, synthetic construct
  REGION 1..118: note = MTK-29.8 Heavy Chain Variable
QVQLVQSGSELKKPGASVKVSCKASGYIFTTYGLSWVRQAPGQGLEWMGWINTYSGVPTYTDDFKGRFVFSLDTNVSTAYLQISSLKAEDTAVYYCARYTNYGVFDYWGQGTLVTVSS

SEQ ID NO: 267 - protein, 118 aa, synthetic construct
  REGION 1..118: note = MTK-29.9 Heavy Chain Variable
QVQLVQSGSELKKPGASVKVSCKASGYIFTSYGLSWVRQAPGQGLEWMGWINTYSGVPTYTDDFKGRFVFSLDTSVSTAYLQISSLKAEDTAVYYCARYTNYGVFDYWGQGTLVTVSS

SEQ ID NO: 268 - protein, 118 aa, synthetic construct
  REGION 1..118: note = MTK-29.10 Heavy Chain Variable
QVQLVQSGSELKKPGASVKVSCKTSSYTFTTYGMSWVRQAPGQGLEWMGWINTYSGVPTYTDDFKGRFVFSLDTSVSTAYLQISSLKAEDTAVYYCARYTNYGVFDYWGQGTLVTVSS

SEQ ID NO: 269 - protein, 118 aa, synthetic construct
  REGION 1..118: note = MTK-29.11 Heavy Chain Variable
QVQLVQSGSELKKPGASVKVSCKASSYTFTTYGMSWVRQAPGQGLEWMGWINTDSGVPTYTDDFKGRFVFSLDTSVSTAYLQISSLKAEDTAVYYCARYTNYGVFDYWGQGTLVTVSS

SEQ ID NO: 270 - protein, 118 aa, synthetic construct
  REGION 1..118: note = MTK-29.13 Heavy Chain Variable
QVQLVQSGSELKKPGASVKVSCKASSYTFTTYGMSWVRQAPGQGLEWMGWINTASGVPTYTDDFKGRFVFSLDTSVSTAYLQISSLKAEDTAVYYCARYTNYGVFDYWGQGTLVTVSS

SEQ ID NO: 271 - protein, 118 aa, synthetic construct
  REGION 1..118: note = MTK-29.16, 29.18 Heavy Chain Variable
QVQLVQSGSELKKPGASVKVSCKASGYIFTSYGLSWVRQAPGQGLEWMGWVNTYSGVPTYTDDFKGRFVFSLDTSVSTAYLQISSLKAEDTAVYYCARYTNYGVFDYWGQGTLVTVSS

SEQ ID NO: 272 - protein, 118 aa, synthetic construct
  REGION 1..118: note = MTK-29.17 Heavy Chain Variable
QVQLVQSGSELKKPGASVKVSCKASGYIFTSYGLSWVRQAPGQGLEWMGWINTYSGVPTYAQGFTGRFVFSLDTSVSTAYLQISSLKAEDTAVYYCARYTNYGVFDYWGQGTLVTVSS

SEQ ID NO: 273 - protein, 118 aa, synthetic construct
  REGION 1..118: note = MTK-29.19, 29.20, 29.21 Heavy Chain Variable
QVQLVQSGSELKKPGASVKVSCKASGFTFTTYGMSWVRQAPGQGLEWMGWINTYSGVPTYAQGFTGRFVFSLDTSVSTAYLQICSLKAEDTAVYYCARYTNYGYFDYWGQGTLVTVSS

SEQ ID NO: 274 - protein, 106 aa, synthetic construct
  REGION 1..106: note = MTK-29.1 Light Chain Variable
EIVLTQSPATLSLSPGERATLSCRASSSVGYMHWYQQKPGQAPRPLIYATSNLASGIPARFSGSGSGTDFTLTISRLEPEDFAVYYCQQWGSWPFTFGQGTKLEIK

SEQ ID NO: 275 - protein, 106 aa, synthetic construct
  REGION 1..106: note = MTK-29.2 Light Chain Variable
DIQLTQSPSFLSASVGDRVTITCRASSSVGYMHWYQQKPGKAPKPLIYATSNLASGVPSRFSGSGSGTEFTLTISSLQPEDFATYYCQQWGSIPFTFGGGTKVEIK

SEQ ID NO: 276 - protein, 106 aa, synthetic construct
  REGION 1..106: note = MTK-29.3 Light Chain Variable
DIQLTQSPSFLSASVGDRVTITCRASSSVGYMHWYQQKPGKAPKLLIYATSNLASGVPSRFSGSGSGTEFTLTISSLQPEDFATYYCQQWGSLPFTFGQGTKLEIK

SEQ ID NO: 277 - protein, 106 aa, synthetic construct
  REGION 1..106: note = MTK-29.4, 29.5, 29.6, 29.7, 29.8 Light Chain Variable
DIQLTQSPSFLSASVGDRVTITCRASSSVGYMHWYQQKPGKAPKLLIYATSNLAQGVPSRFSGSGSGTEFTLTISSLQPEDFATYYCQQWGSLPFTFGQGTKLEIK

SEQ ID NO: 278 - protein, 106 aa, synthetic construct
  REGION 1..106: note = MTK-29.9 Light Chain Variable
DIQLTQSPSFLSASVGDRVTITCRASSSVGYMHWYQQKPGKAPKLLIYATSNLAQGVPSRFSGSGSGTGFTLTISSLQPEDFATYYCQQWGSLPFTFGQGTKLEIK

SEQ ID NO: 279 - protein, 106 aa, synthetic construct
  REGION 1..106: note = MTK-29.10, 29.11 Light Chain Variable
EIVLTQSPATLSLSPGERATLSCRASSSVGYMHWYQQKPGQAPRLLIYATSNLASGIPARFSGSGSGTDFTLTISRLEPEDFAVYYCQQWGSLPFTFGGGTKLEIK

SEQ ID NO: 280 - protein, 106 aa, synthetic construct
  REGION 1..106: note = MTK-29.12 Light Chain Variable
DIQLTQSPGFLSASVGDRVTITCRASSSVGYMHWYQQKPGKAPKLLIYATSNLAQGVPSRFSGSGSGTEFTLTISSLQPEDFATYYCQQWGSLPFTFGQGTKLEIK

SEQ ID NO: 281 - protein, 106 aa, synthetic construct
  REGION 1..106: note = MTK-29.13 Light Chain Variable
DIQLTQSPSFLSASVGDRVTITCRASSSVGYMHWYQQEPGKAPKLLIYATSNLAQGVPSRFSGSGSGTEFTLTISSLQPEDFATYYCQQWGSLPFTFGQGTKLEIK

SEQ ID NO: 282 - protein, 106 aa, synthetic construct
  REGION 1..106: note = MTK-29.14 Light Chain Variable
DIQLTQSPSFLSASVGDRVTITCRASSSVGYMHWYQQKPGKAPKLLIYATSNLAQGVPSRFSGSGSGTEFTLTIFSLQPEDFATYYCQQWGSLPFTFGQGTKLEIK

SEQ ID NO: 283 - protein, 106 aa, synthetic construct
  REGION 1..106: note = MTK-29.15 Light Chain Variable
DIQLTQSPSFPSASVGDRVTITCRASSSVGYMHWYQQKPGKAPKLLIYATSNLAQGVPSRFSGSGSGTEFTLTISSLQPEDFATYYCQQWGSLPFTFGQGTKLEIK

SEQ ID NO: 284 - protein, 106 aa, synthetic construct
  REGION 1..106: note = MTK-29.16 Light Chain Variable
DIQLTQSPSFLSASVGDRVTITCRASSSVGYMHWYQQKPGKAPKLLIYATSNLAQGVPSRFSGSGSGTEFTLTISSLQPEDFATYYCQQWGRLPFTFGQGTKLEIK

SEQ ID NO: 285 - protein, 106 aa, synthetic construct
  REGION 1..106: note = MTK-29.17 Light Chain Variable
DIQLTQSPSFLSASVGDRVTITCRSSSSVGYMHWYQQKPGKAPKLLIYATSNLAQGVPSRFSGSGSGTEFTLTISSLQPEDFATYYCQQWGSLPFTFGQGTKLEIK

SEQ ID NO: 286 - protein, 106 aa, synthetic construct
  REGION 1..106: note = MTK-29.18 Light Chain Variable
EIVLTQSPATLSLSPGERATLSCRASSTVGYMHWYQQKPGQAPRLLIYATSNLASGIPARFSGSGSGTDFTLTISRLEPEDFAVYYCQQWGSLPFTFGGGTKLEIK

SEQ ID NO: 287 - protein, 106 aa, synthetic construct
  REGION 1..106: note = MTK-29.19 Light Chain Variable
QIVLTQSPGTLSLSPGERATLSCRATSSVGYMHWYQQKPGQAPRPLIYATSNLASGIPDRFSGSGSGTSYTLTISRLEPEDFAVYYCQQWGSNPFTFGQGTKLEIK

SEQ ID NO: 288 - protein, 106 aa, synthetic construct
  REGION 1..106: note = MTK-29.20 Light Chain Variable
QIVLTQSPATLSLSPGERATLSCRATSSVGYMHWYQQKPGLAPRPLIYATSNLASGIPDRFSGSGSGTSYTLTISRLEPEDFAVYYCQQWGSNPFTFGQGTKLEIK

SEQ ID NO: 289 - protein, 106 aa, synthetic construct
  REGION 1..106: note = MTK-29.21 Light Chain Variable
QIVLTQSPATLSLSPGERATLSCRATSSVGYMHWYQQKPGQAPRPLIYATSNLASGIPARFSGSGSGTSYTLTISSLEPEDFAVYYCQQWGSNPFTFGQGTKLEIK

SEQ ID NO: 290 - protein, 119 aa, synthetic construct
  REGION 1..119: note = MTK-33.1, 33.8 Heavy Chain Variable
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFNYWGQGTLVTVSS

SEQ ID NO: 291 - protein, 119 aa, synthetic construct
  REGION 1..119: note = MTK-33.2 Heavy Chain Variable
QVQLVQSGAEVKKPGASVKVSCKASGYTSTSYWMHWVRQAPGQGLEWIGVIDPSDNYINYNQKFQGRVTMTRDTSTSTVYMELSSLRSEDTAVYYCAREAGTRGYFDYWGQGTLVTVSS

SEQ ID NO: 292 - protein, 119 aa, synthetic construct
  REGION 1..119: note = MTK-33.3, 33.11 Heavy Chain Variable
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFDYWGQGTLVTVSS

SEQ ID NO: 293 - protein, 119 aa, synthetic construct
  REGION 1..119: note = MTK-33.4 Heavy Chain Variable
QVQLVQSGAEVKKPGASVKVSCKASGYTFTSYWMHWVRQAPGQGLEWIGVIDPSDNYINMNQKFQGRVTMTRDTSTSTVYMELSSLRSEDTAVYYCAREAGTRGYFDYWGQGTLVTVSS

SEQ ID NO: 294 - protein, 119 aa, synthetic construct
  REGION 1..119: note = MTK-33.5 Heavy Chain Variable
QVQLVQSGAEVKKPGASVKVSCMASGYTFGSYWMHWVRQAPGQGLEWIGVIDPSDNYINYNQKFQGRVTMTRDTSTSTVYMELSSLRSEDTAVYYCAREAGTRGYFDYWGQGTLVTVSS

SEQ ID NO: 295 - protein, 119 aa, synthetic construct
  REGION 1..119: note = MTK-33.6 Heavy Chain Variable
QVQLVQSGAEVKKPGASVKVSCMASGYTFRSYWMHWVRQAPGQGLEWIGVIDPSDNYINYNQKFQGRVTMTRDTSTSTVYMELSSLRSEDTAVYYCAREAGTRGYFDYWGQGTLVTVSS

SEQ ID NO: 296 - protein, 119 aa, synthetic construct
  REGION 1..119: note = MTK-33.7 Heavy Chain Variable
QVQLVQSGAEVKKPGASVKVSCKASGYTFTSYWMHWVRQAPGQGLEWIGVIDPSDNYINYNAKFQGRVTMTRDTSTSTVYMELSSLRSEDTAVYYCAREAGTRGYFDYWGQGTLVTVSS

SEQ ID NO: 297 - protein, 119 aa, synthetic construct
  REGION 1..119: note = MTK-33.9 Heavy Chain Variable
QVQLVQSGAEVKKPGASVKVSCKASGYTFTSYWMHWVRQAPGQGLEWIGVIDPSDRYINYNQKFQGRVTMTRDTSTSTVYMELSSLRSEDTAVYYCAREAGTRGYFDYWGQGTLVTVSS

SEQ ID NO: 298 - protein, 119 aa, synthetic construct
  REGION 1..119: note = MTK-33.10 Heavy Chain Variable
QVQLVQSGAEVKKPGASVKVSCKASSYSFTSYWMHWVRQAPGQGLEWIGVIDPSDNYINYNQKFRGRVTMTRDTSTSTVYMELSSLRSEDTAVYYCAREAGTRGYFDYWGQGTLVTVSS

SEQ ID NO: 299 - protein, 119 aa, synthetic construct
  REGION 1..119: note = MTK-33.12 Heavy Chain Variable
QVQLVQSGAEVKKPGASVKVSCKASGYTFVSYWMHWVRQAPGQGLEWIGVIDPSDNYINYNQKFRGRVTMTRDTSTSTVYMELSSLRSEDTAVYYCAREAGTRGYFDYWGQGTLVTVSS

SEQ ID NO: 300 - protein, 119 aa, synthetic construct
  REGION 1..119: note = MTK-33.13 Heavy Chain Variable
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAGTRGYFDYWGQGTLVTVSS

SEQ ID NO: 301 - protein, 119 aa, synthetic construct
  REGION 1..119: note = MTK-33.14, 33.19, 33.20, 33.21 Heavy Chain Variable
QVQLVQSGAEVKKPGASVKVSCKASGYTFTSYWMHWVRQAPGQGLEWIGVIDPSDNYINYNQKFRGRVTMTRDTSTSTVYMELSSLRSEDTAVYYCAREAGTRGYFDYWGQGTLVTVSS

SEQ ID NO: 302 - protein, 119 aa, synthetic construct
  REGION 1..119: note = MTK-33.15 Heavy Chain Variable
QVQLVQSGAEVKKPGASVKVSCKASGYTFTSYWMHWVRQAPGQGLEWIGVIDPSDNYINYNQKFQVRVTMTRDTSTSTVYMELSSLRSEDTAVYYCAREAGTRGYFDYWGQGTLVTVSS

SEQ ID NO: 303 - protein, 119 aa, synthetic construct
  REGION 1..119: note = MTK-33.16 Heavy Chain Variable
QVQLVQSGAEVKKPGASVKVSCKASWYTFTSYWMHWVRQAPGQGLEWIGVIDPSDNYINYNQKFQGRVTMTRDTSTSTVYMELSSLRSEDTAVYYCAREAGTRGYFDYWGQGTLVTVSS

SEQ ID NO: 304 - protein, 119 aa, synthetic construct
  REGION 1..119: note = MTK-33.17 Heavy Chain Variable
QVQLVQSGAEVKKPGASVKVSCKASGYTFTSYWMHWVRQAPGQGLEWIGVIAPSDNYINYNQKFRGRVTMTRDTSTSTVYMELSSLRSEDTAVYYCAREAGTRGYFDYWGQGTLVTVSS

SEQ ID NO: 305 - protein, 119 aa, synthetic construct
  REGION 1..119: note = MTK-33.18 Heavy Chain Variable
QVQLVQSGAEVKKPGASVKVSCKASGYTFTSYWMHWVRQAPGQGLEWIGVISPSDNYINYNQKFRGRVTMTRDTSTSTVYMELSSLRSEDTAVYYCAREAGTRGYFDYWGQGTLVTVSS

SEQ ID NO: 306 - protein, 119 aa, synthetic construct
  REGION 1..119: note = MTK-33.22 Heavy Chain Variable
QVQLVQSGAEVKKPGASVKVSCKASGYTFTSYWMHWVRQAPGQGLEWIGVIDPSDNYINYNQKLRGRVTMTRDTSTSTVYMELSSLRSEDTAVYYCAREAGTRGYFDYWGQGTLVTVSS

SEQ ID NO: 307 - protein, 119 aa, synthetic construct
  REGION 1..119: note = MTK-33.23 Heavy Chain Variable
QVQLVQSGAEVKKPGASVKVSCKASGYTFTSYWMHWVRQAPGQGLEWIGVIDPSDNYINYNQKFRGRVTMTRDTSTSTVYMELSSLRSEDTAVYYCAREAYTRGYFDYWGQGTLVTVSS

SEQ ID NO: 308 - protein, 119 aa, synthetic construct
  REGION 1..119: note = MTK-33.24 Heavy Chain Variable
QVQLVQSGAEVKKPGASVKVSCKASSYTFTSYWMHWVRQAPGQGLEWIGVIDPSDNYINYNQKFRGRVTMTRDTSTSTVYMELSSLRSEDTAVYYCAREAGTRGYFDYWGQGTLVTVSS

SEQ ID NO: 309 - protein, 107 aa, synthetic construct
  REGION 1..107: note = MTK-33.1 Light Chain Variable
DIQMTQSPSSLSASVGDRVTITCQASQSVSNTVAWYQQKPGKAPKLLIYYASLRYTGVPSRFSGSGSGTDFTFTISSLQPEDIATYYCQQDYRSPFTFGQGTKLEIK

SEQ ID NO: 310 - protein, 107 aa, synthetic construct
  REGION 1..107: note = MTK-33.2 Light Chain Variable
DIQMTQSPSSLSASVGDRVTITCQASGSVSNTVAWYQQKPGKAPKLLIYYASNRYTGVPSRFSGSGSGTDFTFTISSLQPEDIATYYCQQDYRSPFTFGQGTKLEIK

SEQ ID NO: 311 - protein, 107 aa, synthetic construct
  REGION 1..107: note = MTK-33.3, 33.13, 33.16 Light Chain Variable
DIQMTQSPSSLSASVGDRVTITCQASRSVSNTVAWYQQKPGKAPKLLIYYASNRYTGVPSRFSGSGSGTDFTFTISSLQPEDIATYYCQQDYRSPFTFGQGTKLEIK

SEQ ID NO: 312 - protein, 107 aa, synthetic construct
  REGION 1..107: note = MTK-33.4 Light Chain Variable
DIQMTQSPSSLSASVGDRVTITCQASQSVSNTVAWYQQKPGKAPKLLIYYASNRYTGVPSRFSGSGSGTDFTFTISSLQPEDIATYYCQQDRRSPFTFGQGTKLEIK

SEQ ID NO: 313 - protein, 107 aa, synthetic construct
  REGION 1..107: note = MTK-33.5 Light Chain Variable
DIQMTQSPSSLSASVGDRVTITCQASQSVSNTVAWYQQKPGKAPKLLIYYASNRETGVPSRFSGSGSGTDFTFTISSLQPEDIGTYYCQQDYRSPFTFGQGTKLEIK

SEQ ID NO: 314 - protein, 107 aa, synthetic construct
  REGION 1..107: note = MTK-33.6 Light Chain Variable
DIQMTQSPSSLSASVGDRVTITCQASRSVSNTMAWYQQKPGKAPKLLIYYASNRYTGVPSRFSGSGSGTDFTFTISSLQPEDIATYYCQQDYRSPFTFGQGTKLEIK

SEQ ID NO: 315 - protein, 107 aa, synthetic construct
  REGION 1..107: note = MTK-33.7 Light Chain Variable
DIQMTQSPSSLSASVGDRVTITCGASQSVSNTVAWYQQKPGKAPKLLIYYASNRYTGVPSRFSGSGSGTDFTFTISSLQPEDIATYYCQQDYRSPFTFGQGTKLEIK

SEQ ID NO: 316 - protein, 107 aa, synthetic construct
  REGION 1..107: note = MTK-33.8 Light Chain Variable
DIQMTQSPSSLSASVGDRVTITCQASQSVSRTVAWYQQKPGKAPKLLIYYASNRYTGVPSRFSGSGSGTDFTFTISSLQPEDIATYYCQQDYRSPFTFGQGTKLEIK

SEQ ID NO: 317 - protein, 107 aa, synthetic construct
  REGION 1..107: note = MTK-33.9 Light Chain Variable
DIQMTQSPSSLSASVGDRVTITCQASASVSNTVAWYQQKPGKAPKLLIYYASNRYTGVPSRFSGSGSGTDFTFTISSLQPEDIATYYCQQDYRSPFTFGQGTKLEIK

SEQ ID NO: 318 - protein, 107 aa, synthetic construct
  REGION 1..107: note = MTK-33.10 Light Chain Variable
DIQMTQSPSSLSASVGDRVTITCQAGQSVSNTVAWYQQKPGKAPKLLIYYASNRYTGVPSRFSGSGSGTDFTFTISSLQPEDIATYYCQQDYRSPFTFGQGTKLEIK

SEQ ID NO: 319 - protein, 107 aa, synthetic construct
  REGION 1..107: note = MTK-33.11 Light Chain Variable
DIQMTQSPSSLSASVGDRVTITCQASRSVRNTVAWYQQKPGKAPKLLIYYASNRYTGVPSRFSGSGSGTDFTFTISSLQPEDIATYYCQQDYRSPFTFGQGTKLEIK

SEQ ID NO: 320 - protein, 107 aa, synthetic construct
  REGION 1..107: note = MTK-33.12 Light Chain Variable
DIQMTQSPSSLSASVGDRVTITCQASQSVSNTVAWYQQKPGKAPKLLIYYASNRYTGVPSRFSGSGSGTDFTFTISSLQPEDIATYYCRQDTRSPFTFGQGTKLELK

SEQ ID NO: 321 - protein, 107 aa, synthetic construct
  REGION 1..107: note = MTK-33.14 Light Chain Variable
DIQMTQSPSSLSASVGDRVTITCQASQSVSNTVAWYQQKPGKAPKLLIYYASNRYTGVPSRFSGSGSGTDFTFTISSLQPEDIATYYCQQDYRSPFTFGQGTKLEIK

SEQ ID NO: 322 - protein, 107 aa, synthetic construct
  REGION 1..107: note = MTK-33.15, 33.17, 33.18 Light Chain Variable
DIQMTQSPSSLSASVGDRVTITCQASRSVSNTVAWYQQKPGKAPKLLIYYASNRYTGVPSRFSGSGSGTDFTFTISSLQPEDIATYYCQQDMRSPFTFGQGTKLEIK

SEQ ID NO: 323 - protein, 107 aa, synthetic construct
  REGION 1..107: note = MTK-33.19 Light Chain Variable
DIQMTQSPSSLSASVGDRVTITCQASRSVSATVAWYQQKPGKAPKLLIYYASNRYTGVPSRFSGSGSGTDFTFTISSLQPEDIATYYCQQDMRSPFTFGQGTKLEIK

SEQ ID NO: 324 - protein, 107 aa, synthetic construct
  REGION 1..107: note = MTK-33.20 Light Chain Variable
DIQMTQSPSSLSASVGDRVTITCQASRSVSNTVAWYQQKPGKAPKLLIYYASNRYTGVPSRFSGSGSGTDFTFTISSLQPEDIATYYCAQDMRSPFTFGQGTKLEIK

SEQ ID NO: 325 - protein, 107 aa, synthetic construct
  REGION 1..107: note = MTK-33.21 Light Chain Variable
DIQMTQSPSSLSASVGGRVTITCQASRSVSNTVAWYQQKPGKAPKLLIYYVSNRYTGVPSRFSGSGSGTDFTFTISSLQPEDIATYYCQQDMRSPFTFGQGTKLEIK

SEQ ID NO: 326 - protein, 107 aa, synthetic construct
  REGION 1..107: note = MTK-33.22 Light Chain Variable
DIQMTQSPSSLSASVGDRVTITCQASRSVSNTVAWYQQKPGKAPKLLIYYASNRRTGVPSRFSGSGSGTDFTFTISSLQPEDIATYYCQQDMRSPFTFGQGTKLEIK

SEQ ID NO: 327 - protein, 107 aa, synthetic construct
  REGION 1..107: note = MTK-33.23 Light Chain Variable
DIQMTQSPSSLSASVGDRVTITCIASRSVSNTVAWYQQKPGKAPKLLIYYASNRYTGVPSRFSGSGSGTDFTFTISSLQPEDIATYYCQQDMRSPFTFGQGTKLEIK

SEQ ID NO: 328 - protein, 107 aa, synthetic construct
  REGION 1..107: note = MTK-33.24 Light Chain Variable
DIQMTQSPSSLSASVGDRVTITCQASRSVSRTVAWYQQKPGKAPKLLIYYASNRYTGVPSRFSGSGSGTDFTFTISSLQPEDIATYYCQQDMRSPFTFGQGTKLEIK

SEQ ID NO: 329 - protein, 17 aa, synthetic construct
  REGION 1..17: note = MTK-15.1, 15.3, 15.4, 15.5, 15.7, 15.8, 15.9, 15.13, 15.14, 15.15, 16.1 HVR-H2
WINTYTGEPTYAQGFTG

SEQ ID NO: 330 - protein, 17 aa, synthetic construct
  REGION 1..17: note = MTK-15.2 HVR-H2
WGNTYTGEPTYAQGFTG

SEQ ID NO: 331 - protein, 17 aa, synthetic construct
  REGION 1..17: note = MTK-15.6 HVR-H2
WINTYTGEPYYAQGFTG

SEQ ID NO: 332 - protein, 17 aa, synthetic construct
  REGION 1..17: note = MTK-15.10, 15.12 HVR-H2
WINTYQGEPTYAQGFTG

SEQ ID NO: 333 - protein, 17 aa, synthetic construct
  REGION 1..17: note = MTK-15.11 HVR-H2
WINTYEGEPTYAQGFTG

SEQ ID NO: 334 - protein, 9 aa, synthetic construct
  REGION 1..9: note = MTK-15.1, 15.11 HVR-H3
GNRYAYQDY

SEQ ID NO: 335 - protein, 9 aa, synthetic construct
  REGION 1..9: note = MTK-15.5 HVR-H3
GTRYAYMDY

SEQ ID NO: 336 - protein, 9 aa, synthetic construct
  REGION 1..9: note = MTK-15.8, 15.12 HVR-H3
GVRYAYMDY

SEQ ID NO: 337 - protein, 9 aa, synthetic construct
  REGION 1..9: note = MTK-15.9 HVR-H3
GYRYAYMDY

SEQ ID NO: 338 - protein, 9 aa, synthetic construct
  REGION 1..9: note = MTK-15.10 HVR-H3
GARYAYMDY

SEQ ID NO: 339 - protein, 9 aa, synthetic construct
  REGION 1..9: note = MTK-15.13 HVR-H3
GNRYAYEDY

SEQ ID NO: 340 - protein, 10 aa, synthetic construct
  REGION 1..10: note = MTK-15.3 HVR-L1
RASSHVSYMH

SEQ ID NO: 341 - protein, 10 aa, synthetic construct
  REGION 1..10: note = MTK-15.4, 15.5, 15.6, 15.8, 15.10, 15.12, 29.1, 29.2, 29.3, 29.4, 29.5, 29.6, 29.7, 29.8, 29.9, 29.10, 29.11, 29.12, 29.13, 29.14, 29.15, 29.16 HVR-L1
RASSSVGYMH

SEQ ID NO: 342 - protein, 10 aa, synthetic construct
  REGION 1..10: note = MTK-15.7 HVR-L1
RASSGVSYMH

SEQ ID NO: 343 - protein, 10 aa, synthetic construct
  REGION 1..10: note = MTK-15.9 HVR-L1
RASSSTSYMH

SEQ ID NO: 344 - protein, 10 aa, synthetic construct
  REGION 1..10: note = MTK-15.13 HVR-L1
RASSSVQYMH

SEQ ID NO: 345 - protein, 11 aa, synthetic construct
  REGION 1..11: note = MTK-16.2 HVR-L1
RASQNVRTAVA

SEQ ID NO: 346 - protein, 5 aa, synthetic construct
  REGION 1..5: note = MTK-29.1, 29.5, 29.9, 29.16, 29.17, 29.18 HVR-H1
SYGLS

SEQ ID NO: 347 - protein, 5 aa, synthetic construct
  REGION 1..5: note = MTK-29.8 HVR-H1
TYGLS

SEQ ID NO: 348 - protein, 17 aa, synthetic construct
  REGION 1..17: note = MTK-29.1, 29.17, 29.19, 29.20, 29.21 HVR-H2
WINTYSGVPTYAQGFTG

SEQ ID NO: 349 - protein, 17 aa, synthetic construct
  REGION 1..17: note = MTK-29.3 HVR-H2
WINTYSGVPAYTDDFKG

SEQ ID NO: 350 - protein, 17 aa, synthetic construct
  REGION 1..17: note = MTK-29.6, 29.12, 29.15 HVR-H2
WINTMSGVPTYTDDFKG

SEQ ID NO: 351 - protein, 17 aa, synthetic construct
  REGION 1..17: note = MTK-29.7, 29.14 HVR-H2
WINTYSGVPSYTDDFKA

SEQ ID NO: 352 - protein, 17 aa, synthetic construct
  REGION 1..17: note = MTK-29.11 HVR-H2
WINTDSGVPTYTDDFKG

SEQ ID NO: 353 - protein, 17 aa, synthetic construct
  REGION 1..17: note = MTK-29.13 HVR-H2
WINTASGVPTYTDDFKG

SEQ ID NO: 354 - protein, 17 aa, synthetic construct
  REGION 1..17: note = MTK-29.16, 29.18 HVR-H2
WVNTYSGVPTYTDDFKG

SEQ ID NO: 355 - protein, 17 aa, synthetic construct
  REGION 1..17: note = MTK-29.1, 29.2 HVR-H3
WINTYSGVPTYAQGFTG

SEQ ID NO: 356 - protein, 9 aa, synthetic construct
  REGION 1..9: note = MTK-29.3, 29.4, 29.5, 29.6, 29.7, 29.8, 29.9, 29.10, 29.11, 29.12, 29.13, 29.14, 29.15, 29.16, 29.17, 29.18 HVR-H3
YTNYGVFDY

SEQ ID NO: 357 - protein, 10 aa, synthetic construct
  REGION 1..10: note = MTK-29.17 HVR-L1
RSSSSVGYMH

SEQ ID NO: 358 - protein, 10 aa, synthetic construct
  REGION 1..10: note = MTK-29.18 HVR-L1
RASSTVGYMH

SEQ ID NO: 359 - protein, 7 aa, synthetic construct
  REGION 1..7: note = MTK-29.4, 29.5, 29.6, 29.7, 29.8, 29.9, 29.12, 29.13, 29.14, 29.15, 29.16, 29.17 HVR-L2
ATSNLAQ

SEQ ID NO: 360 - protein, 9 aa, synthetic construct
  REGION 1..9: note = MTK-29.1 HVR-L3
QQWGSWPFT

SEQ ID NO: 361 - protein, 9 aa, synthetic construct
  REGION 1..9: note = MTK-29.2 HVR-L3
QQWGSIPFT

SEQ ID NO: 362 - protein, 9 aa, synthetic construct
  REGION 1..9: note = MTK-29.3, 29.4, 29.5, 29.6, 29.7, 29.8, 29.9, 29.10, 29.11, 29.12, 29.13, 29.14, 29.15, 29.17, 29.18 HVR-L3
QQWGSLPFT

SEQ ID NO: 363 - protein, 9 aa, synthetic construct
  REGION 1..9: note = MTK-29.16 HVR-L3
QQWGRLPFT

SEQ ID NO: 364 - protein, 17 aa, synthetic construct
  REGION 1..17: note = MTK-33.1, 33.2, 33.3, 33.5, 33.6, 33.8, 33.11, 33.13, 33.16 HVR-H2
VIDPSDNYINYNQKFQG

SEQ ID NO: 365 - protein, 17 aa, synthetic construct
  REGION 1..17: note = MTK-33.4 HVR-H2
VIDPSDNYINMNQKFQG

SEQ ID NO: 366 - protein, 17 aa, synthetic construct
  REGION 1..17: note = MTK-33.7 HVR-H2
VIDPSDNYINYNAKFQG

SEQ ID NO: 367 - protein, 17 aa, synthetic construct
  REGION 1..17: note = MTK-33.9 HVR-H2
VIDPSDRYINYNQKFQG

SEQ ID NO: 368 - protein, 17 aa, synthetic construct
  REGION 1..17: note = MTK-33.10, 33.12, 33.14, 33.19, 33.20, 33.21, 33.23, 33.24 HVR-H2
VIDPSDNYINYNQKFRG

SEQ ID NO: 369 - protein, 17 aa, synthetic construct
  REGION 1..17: note = MTK-33.15 HVR-H2
VIDPSDNYINYNQKFQV

SEQ ID NO: 370 - protein, 17 aa, synthetic construct
  REGION 1..17: note = MTK-33.17 HVR-H2
VIAPSDNYINYNQKFRG

SEQ ID NO: 371 - protein, 17 aa, synthetic construct
  REGION 1..17: note = MTK-33.18 HVR-H2
VISPSDNYINYNQKFRG

SEQ ID NO: 372 - protein, 17 aa, synthetic construct
  REGION 1..17: note = MTK-33.22 HVR-H2
VIDPSDNYINYNQKLRG

SEQ ID NO: 373 - protein, 10 aa, synthetic construct
  REGION 1..10: note = MTK-33.1, 33.8 HVR-H3
EAWTRGYFNY

SEQ ID NO: 374 - protein, 10 aa, synthetic construct
  REGION 1..10: note = MTK-33.3, 33.11 HVR-H3
EAWTRGYFDY

SEQ ID NO: 375 - protein, 10 aa, synthetic construct
  REGION 1..10: note = MTK-33.23 HVR-H3
EAYTRGYFDY

SEQ ID NO: 376 - protein, 11 aa, synthetic construct
  REGION 1..11: note = MTK-33.1, 33.4, 33.5, 33.12, 33.14 HVR-L1
QASQSVSNTVA

SEQ ID NO: 377 - protein, 11 aa, synthetic construct
  REGION 1..11: note = MTK-33.2 HVR-L1
QASGSVSNTVA

SEQ ID NO: 378 - protein, 11 aa, synthetic construct
  REGION 1..11: note = MTK-33.3, 33.13, 33.15, 33.16, 33.17, 33.18, 33.20, 33.21, 33.22 HVR-L1
QASRSVSNTVA

SEQ ID NO: 379 - protein, 11 aa, synthetic construct
  REGION 1..11: note = MTK-33.6 HVR-L1
QASRSVSNTMA

SEQ ID NO: 380 - protein, 11 aa, synthetic construct
  REGION 1..11: note = MTK-33.7 HVR-L1
GASQSVSNTVA

SEQ ID NO: 381 - protein, 11 aa, synthetic construct
  REGION 1..11: note = MTK-33.8 HVR-L1
QASQSVSRTVA

SEQ ID NO: 382 - protein, 11 aa, synthetic construct
  REGION 1..11: note = MTK-33.9 HVR-L1
QASASVSNTVA

SEQ ID NO: 383 - protein, 11 aa, synthetic construct
  REGION 1..11: note = MTK-33.10 HVR-L1
QAGQSVSNTVA

SEQ ID NO: 384 - protein, 11 aa, synthetic construct
  REGION 1..11: note = MTK-33.11 HVR-L1
QASRSVRNTVA

SEQ ID NO: 385 - protein, 11 aa, synthetic construct
  REGION 1..11: note = MTK-33.19 HVR-L1
QASRSVSATVA

SEQ ID NO: 386 - protein, 11 aa, synthetic construct
  REGION 1..11: note = MTK-33.23 HVR-L1
IASRSVSNTVA

SEQ ID NO: 387 - protein, 11 aa, synthetic construct
  REGION 1..11: note = MTK-33.24 HVR-L1
QASRSVSRTVA

SEQ ID NO: 388 - protein, 7 aa, synthetic construct
  REGION 1..7: note = MTK-33.1 HVR-L2
YASLRYT

SEQ ID NO: 389 - protein, 7 aa, synthetic construct
  REGION 1..7: note = MTK-33.5 HVR-L2
YASNRET

SEQ ID NO: 390 - protein, 7 aa, synthetic construct
  REGION 1..7: note = MTK-33.21 HVR-L2
YVSNRYT

SEQ ID NO: 391 - protein, 7 aa, synthetic construct
  REGION 1..7: note = MTK-33.22 HVR-L2
YASNRRT

SEQ ID NO: 392 - protein, 9 aa, synthetic construct
  REGION 1..9: note = MTK-33.4 HVR-L3
QQDRRSPFT

SEQ ID NO: 393 - protein, 9 aa, synthetic construct
  REGION 1..9: note = MTK-33.12 HVR-L3
RQDTRSPFT

SEQ ID NO: 394 - protein, 9 aa, synthetic construct
  REGION 1..9: note = MTK-33.15, 33.17, 33.18, 33.19, 33.21, 33.22, 33.23, 33.24 HVR-L3
QQDMRSPFT

SEQ ID NO: 395 - protein, 9 aa, synthetic construct
  REGION 1..9: note = MTK-33.20 HVR-L3
AQDMRSPFT

SEQ ID NO: 396 - protein, 330 aa, synthetic construct [sequence omitted]

SEQ ID NO: 397 - protein, 329 aa, synthetic construct
  REGION 1..329: note = huIgG1 WT Fc without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 398 - protein, 330 aa, synthetic construct
  REGION 1..330: note = huIgG1 LALAPS Fc with C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 399 - protein, 329 aa, synthetic construct
  REGION 1..329: note = huIgG1 LALAPS Fc without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 400 - protein, 330 aa, synthetic construct
  REGION 1..330: note = huIgG1 NSLF Fc with C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 401 - protein, 329 aa, synthetic construct
  REGION 1..329: note = huIgG1 NSLF Fc without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 402 - protein, 330 aa, synthetic construct
  REGION 1..330: note = huIgG1 YTE Fc with C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 403 - protein, 329 aa, synthetic construct
  REGION 1..329: note = huIgG1 YTE Fc without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 404 - protein, 330 aa, synthetic construct
  REGION 1..330: note = huIgG1 LS Fc with C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 405 - protein, 329 aa, synthetic construct
  REGION 1..329: note = huIgG1 LS Fc without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 406 - protein, 342 aa, synthetic construct
  REGION 1..342: note = huIgG1 LV5-112 Fc with C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGGCALYPTNCGGGQPENNYKTTPPVLDSDGSFFLYSKLTVDKSRWQQGNVFSCSVMHEALHNHYTQKSLSLSPGK

SEQ ID NO: 407 - protein, 341 aa, synthetic construct
  REGION 1..341: note = huIgG1 LV5-112 Fc without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGGCALYPTNCGGGQPENNYKTTPPVLDSDGSFFLYSKLTVDKSRWQQGNVFSCSVMHEALHNHYTQKSLSLSPG

SEQ ID NO: 408 - protein, 326 aa, synthetic construct
  REGION 1..326: note = huIgG2 WT Fc with C-terminal lysine
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 409 - protein, 325 aa, synthetic construct
  REGION 1..325: note = huIgG2 WT Fc without C-terminal lysine
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 410 - protein, 327 aa, synthetic construct
  REGION 1..327: note = huIgG4 WT Fc with C-terminal lysine
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 411 - protein, 326 aa, synthetic construct
  REGION 1..326: note = huIgG4 WT Fc without C-terminal lysine
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 412 - protein, 107 aa, synthetic construct [sequence omitted]

SEQ ID NO: 413 - protein, 450 aa, synthetic construct
  REGION 1..450: note = MTK-16.1 with huIgG1 WT Fc with C-terminal lysine
QVQLVQSGSELKKPGASVKVSCKASGYTFTNHGMNWVRQAPGQGLEWMGWINTYTGEPTYAQGFTGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 414 - protein, 449 aa, synthetic construct
  REGION 1..449: note = MTK-16.1 with huIgG1 WT Fc without C-terminal lysine
QVQLVQSGSELKKPGASVKVSCKASGYTFTNHGMNWVRQAPGQGLEWMGWINTYTGEPTYAQGFTGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 415 - protein, 450 aa, synthetic construct
  REGION 1..450: note = MTK-16.1 with huIgG1 LALAPS Fc with C-terminal lysine
QVQLVQSGSELKKPGASVKVSCKASGYTFTNHGMNWVRQAPGQGLEWMGWINTYTGEPTYAQGFTGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 416 - protein, 449 aa, synthetic construct
  REGION 1..449: note = MTK-16.1 with huIgG1 LALAPS Fc without C-terminal lysine
QVQLVQSGSELKKPGASVKVSCKASGYTFTNHGMNWVRQAPGQGLEWMGWINTYTGEPTYAQGFTGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 417 - protein, 450 aa, synthetic construct
  REGION 1..450: note = MTK-16.1 with huIgG1 NSLF Fc with C-terminal lysine
QVQLVQSGSELKKPGASVKVSCKASGYTFTNHGMNWVRQAPGQGLEWMGWINTYTGEPTYAQGFTGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 418 - protein, 449 aa, synthetic construct
  REGION 1..449: note = MTK-16.1 with huIgG1 NSLF Fc without C-terminal lysine
QVQLVQSGSELKKPGASVKVSCKASGYTFTNHGMNWVRQAPGQGLEWMGWINTYTGEPTYAQGFTGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 419 - protein, 447 aa, synthetic construct
  REGION 1..447: note = MTK-16.1 with huIgG4 Fc with C-terminal lysine
QVQLVQSGSELKKPGASVKVSCKASGYTFTNHGMNWVRQAPGQGLEWMGWINTYTGEPTYAQGFTGRFVFSLDTSVSTAYLQISSLKAEDTAVYYCARKGVTAARYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 420 - protein, 446 aa, synthetic construct
  REGION 1..446: note = MTK-16.1 with huIgG4 Fc without C-terminal lysine
QVQLVQSGSELKKPGASVKVSCKASGYTFTNHGMNWVRQAPGQGLEWMGWINTYTGEPTYAQGFTGRFVFSLDTSVSTAYLQISSLKAEDTAVYYCARKGVTAARYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 421 - protein, 214 aa, synthetic construct
  REGION 1..214: note = MTK-16.1 light chain
DIQMTQSPSSLSASVGDRVTITCKASQNVRTAVAWYQQKPGKAPKRLIYLASNRHTGVPSRFSGSGSGTEFTLTISNLQPEDFATYYCLQHWNYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 422 - protein, 450 aa, synthetic construct
  REGION 1..450: note = MTK-16.2 with huIgG1 WT Fc with C-terminal lysine
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 423 - protein, 449 aa, synthetic construct
  REGION 1..449: note = MTK-16.2 with huIgG1 WT Fc without C-terminal lysine
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 424 - protein, 450 aa, synthetic construct
  REGION 1..450: note = MTK-16.2 with huIgG1 LALAPS Fc with C-terminal lysine
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 425 - protein, 449 aa, synthetic construct
  REGION 1..449: note = MTK-16.2 with huIgG1 LALAPS Fc without C-terminal lysine
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 426 - protein, 450 aa, synthetic construct
  REGION 1..450: note = MTK-16.2 with huIgG1 NSLF Fc with C-terminal lysine
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 427 - protein, 449 aa, synthetic construct
  REGION 1..449: note = MTK-16.2 with huIgG1 NSLF Fc without C-terminal lysine
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 428 - protein, 447 aa, synthetic construct
  REGION 1..447: note = MTK-16.2 with huIgG4 Fc with C-terminal lysine
QVQLVQSGSELKKPGASVKVSCKASGYTFTNHGMNWVRQAPGQGLEWMGWINTYTGEPTYADDFKGRFVFSLDTSVSTAYLQISSLKAEDTAVYYCARKGVTAARYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 429 - protein, 447 aa, synthetic construct; identical to SEQ ID NO: 428
  REGION 1..447: note = MTK-16.2 with huIgG4 Fc without C-terminal lysine

SEQ ID NO: 430 - protein, 214 aa, synthetic construct
  REGION 1..214: note = MTK-16.2 light chain
DIQMTQSPSSLSASVGDRVTITCRASQNVRTAVAWYQQKPGKAPKRLIYLASNRHTGVPSRFSGSGSGTEFTLTISNLQPEDFATYYCLQHWNYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 431 - protein, 449 aa, synthetic construct
  REGION 1..449: note = MTK-33.1 with huIgG1 WT Fc with C-terminal lysine
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 432 - protein, 448 aa, synthetic construct
  REGION 1..448: note = MTK-33.1 with huIgG1 WT Fc without C-terminal lysine
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 433 - protein, 449 aa, synthetic construct
  REGION 1..449: note = MTK-33.1 with huIgG1 LALAPS Fc with C-terminal lysine
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 434 - protein, 448 aa, synthetic construct
  REGION 1..448: note = MTK-33.1 with huIgG1 LALAPS Fc without C-terminal lysine
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 435 - protein, 449 aa, synthetic construct
  REGION 1..449: note = MTK-33.1 with huIgG1 NSLF Fc with C-terminal lysine
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 436 - protein, 448 aa, synthetic construct
  REGION 1..448: note = MTK-33.1 with huIgG1 NSLF Fc without C-terminal lysine
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 437 - protein, 446 aa, synthetic construct
  REGION 1..446: note = MTK-33.1 with huIgG4 Fc with C-terminal lysine
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 438 - protein, 445 aa, synthetic construct
  REGION 1..445: note = MTK-33.1 with huIgG4 Fc without C-terminal lysine
QVQLVQSGAEVKKPGASVKVSCKASGYTFTSYWMHWVRQAPGQGLEWIGVIDPSDNYINYNQKFQGRVTMTRDTSTSTVYMELSSLRSEDTAVYYCAREAWTRGYFNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 439 - protein, 214 aa, synthetic construct
  REGION 1..214: note = MTK-33.1 light chain
DIQMTQSPSSLSASVGDRVTITCQASQSVSNTVAWYQQKPGKAPKLLIYYASLRYTGVPSRFSGSGSGTDFTFTISSLQPEDIATYYCQQDYRSPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 440 - protein, 449 aa, synthetic construct
  REGION 1..449: note = MTK-33.12 with huIgG1 WT Fc with C-terminal lysine
QVQLVQSGAEVKKPGASVKVSCKASGYTFVSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 441 - protein, 448 aa, synthetic construct
  REGION 1..448: note = MTK-33.12 with huIgG1 WT Fc without C-terminal lysine
QVQLVQSGAEVKKPGASVKVSCKASGYTFVSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 442 - protein, 449 aa, synthetic construct
  REGION 1..449: note = MTK-33.12 with huIgG1 LALAPS Fc with C-terminal lysine
QVQLVQSGAEVKKPGASVKVSCKASGYTFVSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 443 - protein, 448 aa, synthetic construct
  REGION 1..448: note = MTK-33.12 with huIgG1 LALAPS Fc without C-terminal lysine
QVQLVQSGAEVKKPGASVKVSCKASGYTFVSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 444 - protein, 449 aa, synthetic construct
  REGION 1..449: note = MTK-33.12 with huIgG1 NSLF Fc with C-terminal lysine
QVQLVQSGAEVKKPGASVKVSCKASGYTFVSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 445 - protein, 448 aa, synthetic construct
  REGION 1..448: note = MTK-33.12 with huIgG1 NSLF Fc without C-terminal lysine
QVQLVQSGAEVKKPGASVKVSCKASGYTFVSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 446 - protein, 446 aa, synthetic construct
  REGION 1..446: note = MTK-33.12 with huIgG4 Fc with C-terminal lysine
QVQLVQSGAEVKKPGASVKVSCKASGYTFVSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 447 - protein, 445 aa, synthetic construct
  REGION 1..445: note = MTK-33.12 with huIgG4 Fc without C-terminal lysine
QVQLVQSGAEVKKPGASVKVSCKASGYTFVSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 448 - protein, 214 aa, synthetic construct
  REGION 1..214: note = MTK-33.12 light chain
DIQMTQSPSSLSASVGDRVTITCQASQSVSNTVAWYQQKPGKAPKLLIYYASNRYTGVPSRFSGSGSGTDFTFTISSLQPEDIATYYCRQDTRSPFTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 449 - protein, 67 aa, synthetic construct
  REGION 1..67: note = N-terminal domain (N) of human MerTK ECD
AITEAREEAKPYPLFPGPFPGSLQTDHTPLLSLPHASGYQPALMFSPTQPGRPHTGNVAIPQVTSVE

SEQ ID NO: 450 - protein, 107 aa, synthetic construct
  REGION 1..107: note = Immunoglobulin-like domain (Ig1) of human MerTK ECD
SKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQ

SEQ ID NO: 451 - protein, 85 aa, synthetic construct
  REGION 1..85: note = Immunoglobulin-like domain (Ig2) of human MerTK ECD
GLPHFTKQPESMNVTRNTAFNLTCQAVGPPEPVNIFWVQNSSRVNEQPEKSPSVLTVPGLTEMAVFSCEAHNDKGLTVSKGVQIN

SEQ ID NO: 452 - protein, 99 aa, synthetic construct
  REGION 1..99: note = Fibronectin type III domain (FN1) of human MerTK ECD
IKAIPSPPTEVSIRNSTAHSILISWVPGFDGYSPFRNCSIQVKEADPLSNGSVMIFNTSALPHLYQIKQLQALANYSIGVSCMNEIGWSAVSPWILAST

SEQ ID NO: 453 - protein, 98 aa, synthetic construct
  REGION 1..98: note = Fibronectin type III domain (FN2) of human MerTK ECD
TEGAPSVAPLNVTVFLNESSDNVDIRWMKPPTKQQDGELVGYRISHVWQSAGISKELLEEVGQNGSRARISVQVHNATCTVRIAAVTRGGVGPFSDPV

SEQ ID NO: 454 - protein, 29 aa, synthetic construct
  REGION 1..29: note = Juxta membrane domain region (JM) of human MerTK ECD
KIFIPAHGWVDYAPSSTPAPGNADPVLII

SEQ ID NO: 455 - protein, 27 aa, synthetic construct
  REGION 1..27: note = N-terminal domain (N) of human Axl ECD
APRGTQAEESPFVGNPGNITGARGLTG

SEQ ID NO: 456 - protein, 84 aa, synthetic construct
  REGION 1..84: note = Immunoglobulin-like domain (Ig1) of human Axl ECD
TLRCQLQVQGEPPEVHWLRDGQILELADSTQTQVPLGEDEQDDWIVVSQLRITSLQLSDTGQYQCLVFLGHQTFVSQPGYVGLE

SEQ ID NO: 457 - protein, 89 aa, synthetic construct
  REGION 1..89: note = Immunoglobulin-like domain (Ig2) of human Axl ECD
GLPYFLEEPEDRTVAANTPFNLSCQAQGPPEPVDLLWLQDAVPLATAPGHGPQRSLHVPGLNKTSSFSCEAHNAKGVTTSRTATITVLP

SEQ ID NO: 458 - protein, 106 aa, synthetic construct
  REGION 1..106: note = Fibronectin type III domain (FN1) of human Axl ECD
QQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEG

SEQ ID NO: 459 - protein, 87 aa, synthetic construct
  REGION 1..87: note = Fibronectin type III domain (FN2) of human Axl ECD
VPLGPPENISATRNGSQAFVHWQEPRAPLQGTLLGYRLAYQGQDTPEVLMDIGLRQEVTLELQGDGSVSNLTVCVAAYTAAGDGPWS

SEQ ID NO: 460 - protein, 33 aa, synthetic construct
  REGION 1..33: note = Juxta membrane domain (JM) of human Axl ECD
LPVPLEAWRPGQAQPVHQLVKEPSTPAFSWPWW

SEQ ID NO: 461 - protein, 19 aa, synthetic construct
  REGION 1..19: note = MerTK construct signal sequence
MGWSCIILFLVATATGVHS

SEQ ID NO: 462 - protein, 16 aa, synthetic construct
  REGION 1..16: note = Axl construct signal sequence
MGWSCIILFLVATATG

SEQ ID NO: 463 - protein, 27 aa, synthetic construct
  REGION 1..27: note = Linker+His+Avi tag
GGSGHHHHHHGGGLNDIFEAQKIEWHE

SEQ ID NO: 464 - protein, 449 aa, synthetic construct
  REGION 1..449: note = MTK-33.10 with huIgG1 WT Fc with C-terminal lysine
QVQLVQSGAEVKKPGASVKVSCKASSYSFTSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 465 - protein, 448 aa, synthetic construct
  REGION 1..448: note = MTK-33.10 with huIgG1 WT Fc without C-terminal lysine
QVQLVQSGAEVKKPGASVKVSCKASSYSFTSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 466 - protein, 449 aa, synthetic construct
  REGION 1..449: note = MTK-33.10 with huIgG1 LALAPS Fc with C-terminal lysine
QVQLVQSGAEVKKPGASVKVSCKASSYSFTSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 467 - protein, 448 aa, synthetic construct
  REGION 1..448: note = MTK-33.10 with huIgG1 LALAPS Fc without C-terminal lysine
QVQLVQSGAEVKKPGASVKVSCKASSYSFTSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 468 - protein, 449 aa, synthetic construct
  REGION 1..449: note = MTK-33.10 with huIgG1 NSLF Fc with C-terminal lysine
QVQLVQSGAEVKKPGASVKVSCKASSYSFTSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 469 - protein, 448 aa, synthetic construct
  REGION 1..448: note = MTK-33.10 with huIgG1 NSLF Fc without C-terminal lysine
QVQLVQSGAEVKKPGASVKVSCKASSYSFTSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 470 - protein, 446 aa, synthetic construct
  REGION 1..446: note = MTK-33.10 with huIgG4 Fc with C-terminal lysine
QVQLVQSGAEVKKPGASVKVSCKASSYSFTSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 471 - protein, 445 aa, synthetic construct
  REGION 1..445: note = MTK-33.10 with huIgG4 Fc without C-terminal lysine
QVQLVQSGAEVKKPGASVKVSCKASSYSFTSYWMHWVRQAPGQGLEWIGVIDPSDNYINYNQKFRGRVTMTRDTSTSTVYMELSSLRSEDTAVYYCAREAGTRGYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 472 - protein, 214 aa, synthetic construct
  REGION 1..214: note = MTK-33.10 light chain
DIQMTQSPSSLSASVGDRVTITCQAGQSVSNTVAWYQQKPGKAPKLLIYYASNRYTGVPSRFSGSGSGTDFTFTISSLQPEDIATYYCQQDYRSPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC